SEQUENCE LISTING (12 sequences)

SEQ ID NO: 1 - DNA, 45 bp, Homo sapiens
aagcgcgcggaattcataaatatggagaacttccaaaaggtggaa

SEQ ID NO: 2 - DNA, 34 bp, Homo sapiens
ctcgacaagcttattagagtcgaagatggggtac

SEQ ID NO: 3 - DNA, 62 bp, Homo sapiens
cccgggatctcgagataaatatgcatcatcatcatcattcaagacgaagtagccgtttacaa

SEQ ID NO: 4 - DNA, 39 bp, Homo sapiens
cccggtaccgcatgcttagtgttttcctggtggtttttc

SEQ ID NO: 5 - DNA, 23 bp, Homo sapiens
gcgatgcagaaatacgagaaact

SEQ ID NO: 6 - DNA, 22 bp, Homo sapiens
ccccactgtctcaccctctcaa

SEQ ID NO: 7 - DNA, 22 bp, Homo sapiens
cggtgagcggttttatccctcc

SEQ ID NO: 8 - DNA, 24 bp, Homo sapiens
gcattgaatccttgagccatgacg

SEQ ID NO: 9 - DNA, 31 bp, Homo sapiens
cgggatccatggcccagcccccaccggccca

SEQ ID NO: 10 - DNA, 26 bp, Homo sapiens
ccaagctttcaccgatccaggcctag

SEQ ID NO: 11 - DNA, 36 bp, Homo sapiens
cgggatccgccaccatgcagaaatacgagaaactgg

SEQ ID NO: 12 - DNA, 27 bp, Homo sapiens
ggactagtctagggcggacagaagtcg

(19) United States
(12) Patent Application Publication
(10) Pub. No.: US 2004/0147561 A1
(43) Pub. Date: Jul. 29, 2004

(54) Pyrid-2-one derivatives and methods of use

(76) Inventors: Wenge Zhong, Thousand Oaks, CA (US); Mark Henry Norman, Thousand Oaks, CA (US); Matthew Kaller, Ventura, CA (US); Thomas Nguyen, Thousand Oaks, CA (US); Robert Michael Rzasa, Ventura, CA (US); Christopher Tegley, Thousand Oaks, CA (US); Hui-Ling Wang, Thousand Oaks, CA (US)

Correspondence Address: AMGEN INC., U.S. Patent Operations/JWB, Dept. 4300, M/S 27-4-A, One Amgen Center Drive, Thousand Oaks, CA, 91320-1799, US

(21) Appl. No.: 10/736,289

(22) Filed: Dec. 12, 2003

Related U.S. Application Data
(60) Provisional application No. 60/436,787, filed on Dec. 27, 2002.

(51) Int. Cl. C07D 211/84; A61K 31/4439

(52) U.S. Cl. 514/340; 514/345; 546/268.1; 546/300

(57) ABSTRACT

Selected compounds are effective for treatment of diseases, such as cell proliferation or apoptosis mediated diseases. The invention encompasses novel compounds, analogs, prodrugs and pharmaceutically acceptable derivatives thereof, pharmaceutical compositions and methods for prophylaxis and treatment of diseases and other maladies or conditions involving stroke, cancer and the like. The subject invention also relates to processes for making such compounds as well as to intermediates useful in such processes.

This application claims the benefit of U.S. Provisional Application No. 60/436,787 filed Dec. 27, 2002, which is hereby incorporated by reference.

### FIELD OF THE INVENTION

This invention is in the field of pharmaceutical agents and specifically relates to compounds, compositions, uses and methods for treating cell proliferation-related disorders, cell death and apoptosis-related disorders.

### BACKGROUND OF THE INVENTION

Identification of therapeutic agents effective in the treatment of neoplastic diseases or for the treatment of neurological disorders is the subject of significant research efforts.

Protein kinases represent a large family of proteins that play a central role in the regulation of a wide variety of cellular processes and maintaining control over cellular function. A partial list of such kinases includes ab1, Akt, bcr-ab1, Blk, Brk, Btk, c-kit, c-met, c-src, CDK1, CDK2, CDK3, CDK4, CDK5, CDK6, CDK7, CDK8, CDK9, CDK10, cRaf1, CSF1R, CSK, EGFR, ErbB2, ErbB3, ErbB4, Erk, Fak, fes, FGFR1, FGFR2, FGFR3, FGFR4, FGFR5, Fgr, FLK-4, flt-1, Fps, Frk, Fyn, GSK, Hck, IGF-1R, INS-R, Jak, KDR, Lck, Lyn, MEK, p38, PDGFR, PIK, PKC, PYK2, ros, tie, tie2, TRK, Yes, and Zap70. As such, inhibition of kinases has become an important therapeutic target.

Cell proliferation is the rapid reproduction of cells, such as by cell division. The cell cycle, which controls cell proliferation, is itself controlled by a family of serine-threonine kinases called cyclin dependent kinases (CDKs). The regulation of CDK activation is complex, and requires the association of the CDK with a member of the cyclin family of regulatory subunits. A further level of regulation occurs through both activating and inactivating phosphorylations of the CDK subunit. The coordinate activation and inactivation of different cyclin/CDK complexes is necessary for normal progression through the cell cycle. Both the critical G1-S and G2-M transitions are controlled by the activation of different cyclin/CDK activities. Loss of control of CDK regulation is a frequent event in hyperproliferative diseases and cancer (T. Noguchi et al., Am. J. Pathol., 156:2135-2147 (2000)). As such, inhibition of CDKs has become an important target in the study of chemotherapeutics (A. Senderowicz and E. Sausville, J. Nat. Canc. Inst., 92:376-387 (2000)).

Kinases have also been implicated in diseases and disorders of the central nervous system. For example, patients suffering from stroke, Alzheimer's disease or Parkinson's disease would benefit from the inhibition of kinases. CDK5 has been shown to be involved in Alzheimer's pathology (R. Maccioni, et al., Eur. J. Biochem., 268:1518-1527 (2001)) and with neuronal development (G. Paglini and A. Caceres, Eur. J. Biochem., 268:1528-1533 (2001)).

Protein kinases also control programmed cell death, also known as apoptosis. Apoptosis is a ubiquitous physiological process used to eliminate damaged or unwanted cells in multicellular organisms. Disregulation of apoptosis is believed to be involved in the pathogenesis of many human diseases. The failure of apoptotic cell death has been implicated in various cancers, as well as autoimmune disorders. Conversely, increased apoptosis is associated with a variety of diseases involving cell loss such as neurodegenerative disorders and AIDS. As such, inhibition of apoptosis has become an important therapeutic target. CDK5 has been shown to be involved in apoptosis pathology (A. Catania et al., Neuro-Oncology, 3(2):89-98 (April 2001)).

Pyrid-2-one derivatives are known in the art. J. Michael et al., Egypt J. Chem., 31:117-124 (1988) describe substituted 5,6-dihydro-2-oxo-4-phenyl-benzo[h]quinolines. Von H. Schafer and K. Gewadld, J. F. Prakt. Chem., 316:684-692 (1974) describe 4,6-dimethyl-2-hydroxy-3-(4-phenylthiazol-2-yl)pyridine. EP154190, published 11 Sep. 1985, describes substituted pyridone compounds. U.S. Pat. No. 3,074,954, issued 22 Jan. 1963, describes 2-(2-hydroxy-6-methylpyridyl)-4-(5-nitrofuryl)thiazole as an antibiotic. A. Erian, et al., (Phosphorus, Sulfur and Silicon and the Related Elements, 133:127-139 (1998)) describe thiadiazolylpyridones. S. Zayed et al., (Phosphorus, Sulfur and Silicon and the Related Elements, 102(1-4):51-57 (1995)) describe N-[5-(2-thioxo-3-pyridinyl)-1,3,4-thiadiazol-2-yl]-benzamides. V. Chuiguk and K. Fedotov, Ukrainskii Khimicheskii Zhurnal (Russian Edition) 46:1306-1310 (1980). [CA# 94:208680] describe 4,6-dimethyl-3-(4-phenyl-2-thiazolyl)-2(1H)-pyridinone. U.S. Pat. No. 5,643,932, issued 1 Jul. 1997, describes substituted thiazoles as superoxide radical inhibitors.

However, compounds of the current invention have not been described as inhibitors of cell proliferation or apoptosis such as for the treatment of cancer or stroke.

### DESCRIPTION OF THE INVENTION

A class of compounds useful in treating cell proliferative disorders, neurological disorders and apoptosis is defined by Formula I

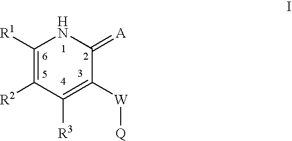

wherein A is O or S, and

preferably O;

wherein Q is selected from —N(R⁵)₂, —NR⁵C(O)R⁵, —(C₁-C₈)alkyl-OR⁵, —(C₁-C₈)alkyl-S(O)ₙR⁶,

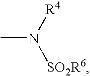

substituted aryl, an unsubstituted or substituted monocyclic or bicyclic, non-aromatic carbocyclic ring, an unsubstituted or substituted monocyclic or bicyclic, heteroaryl ring, and an unsubstituted or substituted monocyclic or bicyclic, non-aromatic heterocyclic ring,

preferably R⁶SO₂—(C₁-C₆)alkyl-,

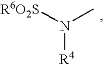

substituted phenyl, and substituted or unsubstituted 5-6 membered heteroaryl;

more preferably phenylsulfonylamino, N-methyl-N-(2-pyridylsulfonyl)amino, N-methyl-N-(3-pyridylsulfonyl)amino, N-methyl-N-(4-pyridylsulfonyl)amino, N-methyl-N-(2-thienylsulfonyl)amino, N-methyl-N-(phenylsulfonyl)amino, 2-pyridylsulfonylmethyl, 3-pyridylsulfonylmethyl, 4-pyridylsulfonylmethyl, 2-thienylsulfonylmethyl, phenylsulfonylmethyl, (1-methyl)-1-(phenylsulfonyl)ethyl, 4-chlorophenylsulfonylmethyl, 2-furylmethylsulfonylmethyl, 3-trifluoromethylbenzyl-sulfonylmethyl, methylsulfonylmethyl, tert-butyl-sulfonylmethyl, 4-fluorobenzylsulfonylmethyl, 4-chlorophenyl-methylsulfonylmethyl, 2-thienyl, 3-(4-chlorophenylsulfonylmethyl)-2-thienyl, phenyl substituted with one or more substituents selected

from hydroxyl, chloro, fluoro, methoxy, —O—CH₂—O—, amino, aminomethyl, methylsulfonyl, methyl, cyano, trifluoromethyl, and pyrrolyl,

unsubstituted pyridyl, and

4-pyridyl substituted with one or more substituents selected from chloro, fluoro, methyl, ethyl, —NH₂, methoxy, ethoxy, —OH, —CO₂H, phenoxyethylamino, methylamino, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl; and

particularly N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl, phenyl substituted with one or more substituents selected

from chloro, fluoro, and —O—CH₂—O—,

unsubstituted pyridyl, and

4-pyridyl substituted with one or more substituents selected from chloro, fluoro, —NH₂, methoxy, ethoxy, phenoxyethylamino, methylamino, methyl, ethyl, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl;

wherein each aryl, monocyclic or bicyclic non-aromatic carbocyclic, a monocyclic or bicyclic heteroaryl, or a monocyclic or bicyclic non-aromatic heterocyclic ring is unsubstituted or substituted with one or more groups selected from halo, (C₁-C₈)alkyl, (C₂-C₈)alkynyl, (C₂-C₈)alkenyl, —OR⁵, —O—(CH₂)₁₋₂—O—, —N(R⁵)₂, —(C₁-C₈)alkyl-N(R⁵)₂, (C₁-C₈)haloalkyl, lower cyanoalkyl, —(C₁-C₈)alkyl-OR⁵, lower alkylaminoalkoxy, lower aminoalkoxyalkyl, —(C₁-C₈)alkyl-S(O)ₙR⁵, —N(R⁵)—(C₁-C₈)alkyl-N(R⁵)₂, —N(R⁵)—(C₁-C₈)alkyl-N(R⁵)—C(O)R⁵, —N(R⁵)—(C₁-C₈)alkyl-OR⁵, —N(R⁵)—(C₁-C₈)alkyl-NHC(O)R⁵, —N(R⁵)—(C₁-C₈)alkyl-C(O)N(R⁵)₂, lower alkoxyalkyl, —S(O)ₙR⁵, —SO₂NR⁵R⁵, —NR⁵S(O)ₙR⁵, cyano, nitro, optionally substituted (C₃-C₁₀)cycloalkyl, optionally substituted aryl, optionally substituted 4-7 membered heterocyclyl, optionally substituted phenoxyalkyl, optionally substituted heterocyclyloxyalkyl, —C(O)N(R⁵)₂, —CO₂R⁵, —CO₂N(R⁵)₂, —SO₂NHC(O)R⁵, optionally substituted phenylalkyl, optionally substituted heterocyclylalkyl, —NR⁵C(O)N(R⁵) ₂, —NR⁵C(O)R⁵, —NR⁵CO₂R⁵ and —C(O)R⁵;

preferably H, halo, phenyl, (C₁-C₆)-alkyl, —OR⁵, —N(R⁵)₂, —(C₁-C₆)alkyl-N(R⁵)₂, lower alkoxyalkyl, R⁵—SO₂—, R⁵-sulfonyl-(C₁-C₆)-alkyl, cyano, lower cyanoalkyl, lower alkylaminoalkoxy, lower aminoalkoxyalkyl (C₃-C₆)cycloalkyl, nitro, optionally substituted 4-7 membered heterocyclyl, optionally substituted phenoxyalkyl, optionally substituted heterocyclyloxyalkyl, —SO₂NR⁵R⁵, —NR⁵SO₂R⁵, —C(O)N(R⁵)₂, —CO₂R⁵, —CO₂NR⁵R⁵, —SO₂NHC(O)R⁵, optionally substituted phenylalkyl, optionally substituted heterocyclylalkyl, —NR⁵C(O)N(R⁵)₂, —NR⁵C(O)R⁵, —NR⁵CO₂R⁵ and —C(O)R⁵;

wherein W is selected from

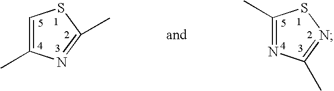

preferably thiazol-4-yl;

wherein n is 0, 1 or 2;

preferably 2;

wherein R¹ is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵)₂, —CO₂R⁵, —(CR⁵₂)₁₋₈aryl, —(CR⁵₂)₁₋₈heterocyclyl, —NR⁵C(O)N(R)₂, —NR⁵C(O)R⁵, —NR⁵CO₂R⁵, and —C(O)R⁵;

preferably (C₁-C₆)alkyl, —(C₁-C₄)alkyl-N(R⁵)₂, —(C₁-C₄)alkyl-OR⁵, —(C₃-C₅)cycloalkyl, and —CF₃;

more preferably methyl, ethyl, propyl, isopropyl, hydroxyethyl, dimethylaminomethyl, benzyloxymethyl, 4-methoxy-benzyloxymethyl, methoxymethyl, cyclopropyl, and —CF₃;

particularly methyl, ethyl, propyl, isopropyl, dimethylaminomethyl, hydroxyethyl, benzyloxymethyl, 4-methoxy-benzyloxymethyl, methoxymethyl, cyclopropyl, and —CF₃;

wherein R² is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵)₂, —CO₂R⁵, —(CR⁵₂)₁₋₈aryl, —(CR⁵₂)₁₋₈heterocyclyl, —NR⁵C(O)N(R⁵)₂, —NR⁵C(O)R⁵, —NR⁵CO₂R⁵, and —C(O)R⁵;

preferably H, halo, (C₁-C₃)alkyl, —NR⁵₂, —OR⁶, —(C₁-C₃)alkyl-OR⁵, —C(O)N(R⁵)₂, —CO₂R⁵, (CH₂)₁₋₃-(5-6 membered saturated or partially unsaturated heterocyclyl, —NHC(O)R⁵, and —C(O)R⁵;

more preferably H, bromo, methyl, amino, isobutylamino, hydroxymethyl, aminocarbonyl, 4-methoxybenzylaminocarbonyl, 2-pyridylmethylaminocarbonyl, ethylaminoethylaminocarbonyl, isopropylaminoethylaminocarbonyl, cyclopropylmethylaminocarbonyl, isobutylaminocarbonyl, ethoxycarbonyl, tert-butoxycarbonyl, 4-morpholinylethoxycarbonyl, 1-pyrrolidinylethoxycarbonyl, 1-piperidylethoxycarbonyl, diethylaminopropoxycarbonyl, carboxyl, 1,2,5,6-tetrahydro-1-pyridylmethyl, 1-piperidylmethyl, 1-methyl-4-piperazinylmethyl, methylcarbonylamino, isobutylcarbonylamino, and 1-methyl-4-piperazinylcarbonyl;

wherein R¹ and R² may be joined to form a 5-10 membered saturated or partially unsaturated carbocyclic or heterocyclic ring;

preferably wherein R¹ and R² may be joined together with the pyridone ring to form optionally substituted 2-oxo-1,5,7,8-tetrahydro-2H-[1,6]naphthyridine, optionally substituted 5,6,7,8-tetrahydro-1H-[1,6]naphthyridin-2-one, optionally substituted 5,6,7,8-tetrahydro-1H-[1,7]naphthyridin-2-one, optionally substituted 5,6,7,8-tetrahydro-1H-quinolin-2-one, optionally substituted 7,8-dihydro-1H-quinolin-2-one, 7,8-dihydro-(1H,6H)-quinoline-2,5-dione or 1,5,7,8-tetrahydro-pyrano[4,3-b]pyridin-2-one;

more preferably 6-benzyloxycarbonyl-2-oxo-1,5,7,8-tetrahydro-2H-[1,6]naphthyridine, 5,6,7,8-tetrahydro-1H-[1,6]naphthyridin-2-one, 7-Boc-5,6,7,8-tetrahydro-1H-[1,7]naphthyridin-2-one, 7-ethyl-5,6,7,8-tetrahydro-1H-[1,7]naphthyridin-2-one, 5-methyl-7,8-dihydro-1H-quinolin-2-one, 5-propylamino-5,6,7,8-tetrahydro-1H-quinolin-2-one, 5-propylimino-5,6,7,8-tetrahydro-1H-quinolin-2-one, 7,8-dihydro-(1H,6H)-quinoline-2,5-dione or 1,5,7,8-tetrahydro-pyrano[4,3-b]pyridin-2-one;

wherein R³ is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵)₂—CO₂R⁵, —(CR⁵₂)₁₋₈aryl, —(CR⁵₂)₁₋₈heterocyclyl, —NR⁵C(O)N(R⁵)₂, —NR⁵C(O)R⁵, —NR⁵CO₂R⁵, and —C(O)R⁵;

preferably H;

wherein R² and R³ may be joined to form a 5-10 membered saturated or partially unsaturated carbocyclic or heterocyclic ring;

wherein R⁴ is independently selected from H, and (C₁-C₆)alkyl;

preferably H, and (C₁-C₂)alkyl;

wherein R⁵ is independently selected from H, lower alkyl, optionally substituted aryl, optionally substituted aralkyl, optionally substituted heterocyclyl, optionally substituted heterocyclylalkyl, optionally substituted C₃-C₆ cycloalkyl, optionally substituted C₃-C₆ cycloalkyl-alkyl, lower alkylamino-lower alkyl, aryloxyalkyl, alkylcarbonylalkyl, and lower perfluoroalkyl;

preferably H, C₁-C₄-alkyl, optionally substituted phenyl, optionally substituted benzyl, optionally substituted heterocyclyl selected from piperazinyl, morpholinyl, pyrrolidinyl, and piperidyl, optionally substituted pyridyl-(C₁-C₃)-alkyl, optionally substituted piperazinyl-(C₁-C₃)-alkyl, 4-morpholinyl-(C₁-C₃)-alkyl, pyrrolidinyl-(C₁-C₃)-alkyl, 1-piperidyl-(C₁-C₃)-alkyl, optionally substituted C₃-C₆ cycloalkyl-(C₁-C₃)-alkyl, —(C₁-C₃)-alkyl-N-((C₁-C₃)-alkyl)₂ and —(C₁-C₃)-alkyl-NH—(C₁-C₃)-alkyl; and

wherein R⁶ is independently selected from lower alkyl, optionally substituted aryl, optionally substituted aralkyl, optionally substituted heterocyclyl, optionally substituted heterocyclylalkyl, optionally substituted C₃-C₆ cycloalkyl, optionally substituted C₃-C₆ cycloalkyl-alkyl, lower alkylamino-lower alkyl, aryloxyalkyl, alkylcarbonylalkyl, and lower perfluoroalkyl;

preferably (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₃-C₃)alkyl-, (C₁-C₂)alkylcarbonyl-(C₁-C₂)alkyl- and optionally substituted heterocyclyl selected from pyridyl and thienyl;

wherein each alkyl, aryl, heteroaryl, heterocyclyl, cycloalkyl, alkynyl, alkynyl, and alkoxy moiety of any R¹, R², R³, R⁴, R⁵ or R⁶ can optionally join with another adjacent or vicinal R¹, R², R³, R⁴, R⁵ or R⁶, to form a 3-7 membered ring; and

wherein each aryl, heteroaryl, cycloalkyl, and heterocyclyl, moiety of any R¹, R², R³, R⁴, R⁵, R⁶, Q and W is optionally substituted with one or more groups selected from halo, —NH₂, —OH, —CO₂H, (C₁-C₄)alkylamino, (C₁-C₆)alkoxy, (C₁-C₆)alkoxyalkyl, (C₁-C₄)alkyl, di(C₁-C₄)alkylamino, phenyl and heterocyclyl;

preferably halo, —NH₂, —OH, —CO₂H, (C₁-C₄)alkylamino, (C₁-C₄)alkyl, di(C₁-C₄)alkylamino, (C₁-C₂)alkoxy, (C₁-C₂)alkoxyalkyl, pyrrolidinyl, piperazinyl, piperidinyl, morpholinyl, and azetidinyl;

more preferably chloro, fluoro, —NH₂, —OH, —CO₂H, (C₁-C₂)alkylamino, (C₁-C₂)alkyl, di(C₁-C₂)alkylamino, methoxymethyl, pyrrolidinyl, piperazinyl, piperidinyl, morpholinyl, and azetidinyl;

and pharmaceutically acceptable derivatives thereof;

provided R¹ is not CF₃ when R² is ethoxycarbonyl, when R³ is H, when W is thiazol-4-yl and when Q is 4-pyridyl or 2-chloro-4-pyridyl; further provided Q is not 4-pyridyl, when W is thiazol-2-yl, when R¹, R³, and R² are H; further provided Q is not 2-nitro-5-furyl when W is thiazol-2-yl, when R¹ is methyl, when R³ is H, and when R² is H; further provided Q is not phenyl when W is thiazol-2-yl, when R¹ is methyl, when R³ is methyl, and when R² is H; further provided Q is not phenyl, 3,4-diacetylphenyl or 3,4-dihydroxyphenyl, when W is thiazol-2-yl, when R¹ is H, when R³ is H, and when R² is H; and further provided Q is not 3-cyano-6-methyl-2-oxo-1,2-dihydro-5-pyridyl, when W is thiazol-2-yl, when R¹ is methyl, when R³ is H, and when R² is acetyl.

The invention also relates to compounds of Formula II

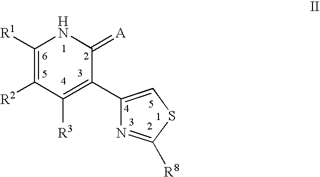

wherein R⁷ is selected from —(C₁-C₃)alkyl, —(C₁-C₃)alkyl-N(R¹⁰)₂, —(C₁-C₃)alkyl-OR¹⁰, —(C₃-C₅)cycloalkyl, and —CF₃;

preferably methyl, ethyl, propyl, isopropyl, dimethylaminomethyl, benzyloxymethyl, hydroxyethyl, 4-methoxy-benzyloxymethyl, methoxymethyl, cyclopropyl, and —CF₃;

wherein R⁸ is selected from R¹⁰SO₂—(C₁-C₆)alkyl-, R¹¹SO₂NH—

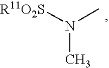

substituted phenyl, and substituted or unsubstituted 5-6 membered heteroaryl;

preferably N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl, phenyl substituted with one or more substituents selected from

chloro, fluoro, and —O—CH₂—O—,

unsubstituted pyridyl, and

4-pyridyl substituted with one or more substituents selected from chloro, fluoro, —NH₂, methoxy, ethoxy, phenoxyethylamino, methylamino, methyl, ethyl, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl;

wherein R⁹ is selected from H, halo, (C₁-C₃)alkyl, —NR¹⁰₂, —(C₁-C₃)alkyl-OR¹⁰, —C(O)N(R¹⁰)₂, —CO₂R¹⁰, (CH₂)₁₋₃-(5-6 membered saturated or partially unsaturated heterocyclyl, —NHC(O)R¹⁰, and —C(O)R¹⁰;

preferably H, bromo, methyl, amino, isobutylamino, hydroxymethyl, aminocarbonyl, 4-methoxybenzylaminocarbonyl, 2-pyridylmethylaminocarbonyl, ethylaminoethylaminocarbonyl, isopropylaminoethylaminocarbonyl, cyclopropylmethylaminocarbonyl, isobutylaminocarbonyl, ethoxycarbonyl, tert-butoxycarbonyl, 4-morpholinylethoxycarbonyl, 1-pyrrolidinylethoxycarbonyl, 1-piperidylethoxycarbonyl, diethylaminopropoxycarbonyl, carboxyl, 1,2,5,6-tetrahydro-1-pyridylmethyl, 1-piperidylmethyl, 1-methyl-4-piperazinylmethyl, methylcarbonylamino, isobutylcarbonylamino, and 1-methyl-4-piperazinylcarbonyl;

wherein R¹⁰ is independently selected from H, (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl- and optionally substituted heterocyclyl selected from pyridyl and thienyl;

preferably H, methyl, propyl, isobutyl, tert-butyl, phenyl, 4-chlorophenyl, 4-methoxybenzyl, furylmethyl, cyclopropylmethyl, cyclopentylmethyl, methylaminoethyl, phenyloxymethyl, ethylcarbonylmethyl and optionally substituted pyridyl and optionally substituted thienyl; and

wherein R¹¹ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl, and optionally substituted heterocyclyl selected from pyridyl and thienyl;

preferably H, methyl, propyl, isobutyl, tert-butyl, phenyl, 4-chlorophenyl, 4-methoxybenzyl, furylmethyl, cyclopropylmethyl, cyclopentylmethyl, methylaminoethyl, phenyloxymethyl, ethylcarbonylmethyl and optionally substituted pyridyl and optionally substituted thienyl;

and pharmaceutically acceptable derivatives thereof;

provided R⁷ is not CF₃, when R⁹ is ethoxycarbonyl and when R⁸ is 4-pyridyl or 2-chloro-4-pyridyl.

The invention also relates to compounds of Formula III

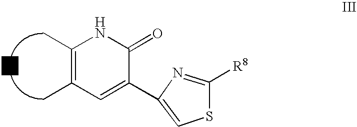

wherein R⁸ is selected from R¹¹SO₂—(C₁-C₆)alkyl-, R¹¹SO₂NH—

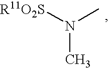

substituted phenyl, and substituted or unsubstituted 5-6 membered heteroaryl;

preferably N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl, phenyl substituted with one or more substituents selected from

chloro, fluoro, and —O—CH₂—O—,

unsubstituted pyridyl, and

4-pyridyl substituted with one or more substituents selected from chloro, fluoro, —NH₂, methoxy, ethoxy, phenoxyethylamino, methylamino, methyl, ethyl, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl;

wherein ring A together with the pyridone ring forms optionally substituted 2-oxo-1,5,7,8-tetrahydro-2H-[1,6]naphthyridine, optionally substituted 5,6,7,8-tetrahydro-1H-[1,6]naphthyridin-2-one, optionally substituted 5,6,7,8-tetrahydro-1H-quinolin-2-one, optionally substituted 5,6,7,8-tetrahydro-1H-[1,7]naphthyridin-2-one, or 1,5,7,8-tetrahydro-pyrano[4,3-b]pyridin-2-one; and

wherein R¹¹ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl, (C₁-C₂)alkylcarbonyl-(C₁-C₂)alkyl, and optionally substituted heterocyclyl selected from pyridyl and thienyl;

and pharmaceutically acceptable derivatives thereof.

A family of specific compounds of particular interest within Formula I consists of compounds and pharmaceutically-acceptable salts thereof as follows:

ethyl 2-ethyl-6-oxo-5-(2-(4-pyridinyl)-1,3-thiazol-4-yl)-1,6-dihydro-pyridine-3-carboxylate;

ethyl-2-ethyl-6-oxo-5-{2-[(thienylsulfonyl)methyl](1,3-thiazol-4-yl)}-1,6-dihydro-pyridine-3-carboxylate;

ethyl-2-ethyl-6-oxo-5-{2-[(phenylsulfonyl)methyl](1,3-thiazol-4-yl)}-1,6-dihydro-pyridine-3-carboxylate;

ethyl-6-oxo-5-{2-[(phenylsulfonyl)methyl](1,3-thiazol-4-yl)}-2-(trifluoromethyl)-1,6-dihydro-pyridine-3-carboxylate;

ethyl-6-oxo-5-{2-[(2-pyridylsulfonyl)methyl](1,3-thiazol-4-yl)}-2-(trifluoromethyl)-1,6-dihydro-pyridine-3-carboxylate;

ethyl-6-oxo-5-{2-[(2-thienylsulfonyl)methyl](1,3-thiazol-4-yl)}-2-(trifluoromethyl)-1,6-dihydro-pyridine-3-carboxylate;

ethyl 2-isopropyl-6-oxo-5-(2-(4-pyridyl)(1,3-thiazol-4-yl)-1,6-dihydro-pyridine-3-carboxylate;

ethyl 2-isopropyl-6-oxo-5-{2-[(thienylsulfonyl)methyl](1,3-thiazol-4-yl)}-1,6-dihydro-pyridine-3-carboxylate;

ethyl 2-isopropyl-6-oxo-5-{2-[(phenylsulfonyl)methyl](1,3-thiazol-4-yl)}-1,6-dihydro-pyridine-3-carboxylate;

ethyl 2-propyl-6-oxo-5-(2-(4-pyridyl)(1,3-thiazol-4-yl)-1,6-dihydro-pyridine-3-carboxylate;

ethyl 2-propyl-6-oxo-5-{(2-[(phenylsulfonyl)methyl](1,3-thiazol-4-yl)}-1,6-dihydro-pyridine-3-carboxylate;

ethyl 2-propyl-6-oxo-5-{2-[(thienylsulfonyl)methyl](1,3-thiazol-4-yl)}-1,6-dihydro-pyridine-3-carboxylate;

ethyl 6-oxo-2-[(phenylmethoxy)methyl]-5-(2-(4-pyridyl)(1,3-thiazol-4-yl))-1,6-dihydro-pyridine-3-carboxylate;

ethyl 6-oxo-2-[(phenylmethoxy)methyl]-5-{2-[(phenylsulfonyl)methyl](1,3-thiazol-4-yl)}-1,6-dihydro-pyridine-3-carboxylate;

phenylmethyl 2-oxo-3-(2-(4-pyridyl)(1,3-thiazol-4-yl))-1,5,6,7,8-pentahydropyridino[3,2-c]pyridine-6-carboxylate;

3-(2-(4-pyridyl)-1,3-thiazol-4-yl)-1,7,8-trihydro-5H-pyrano[4,3-b]pyridin-2-one;

ethyl 2-methyl-6-oxo-5-{2-[(2-thienylsulfonyl)methyl](1,3-thiazol-4-yl)}-1,6-dihydro-pyridine-3-carboxylate;

ethyl 5-[2-({[(4-fluorophenyl)methyl]sulfonyl}methyl)(1,3-thiazol-4-yl)]-2-methyl-6-oxo-1,6-dihydro-pyridine-3-carboxylate;

ethyl 5-[2-({[(4-fluorophenyl)methyl]sulfonyl}methyl)(1,3-thiazol-4-yl)]-2-methyl-6-oxo-1,6-dihydro-pyridine-3-carboxylate;

(ethyl 2-methyl-6-oxo-5-{2-[(2-thienylsulfonyl)methyl]methyl](1,3-thiazol-4-yl)}-1,6-dihydro-pyridine-3-carboxylate;

ethyl 2-methyl-6-oxo-5-{2-(phenylthiomethyl)(1,3-thiazol-4-yl)}-1,6-dihydro-pyridine-3-carboxylate;

ethyl 5-[2-(2-chloro-4-pyridyl)(1,3-thiazol-4-yl)-2-methyl-6-oxo-1,6-dihydro-pyridine-3-carboxylate;

ethyl 5-(2-{[(2-furylmethyl)sulfonyl]methyl}(1,3-thiazol-4-yl))-2-methyl-6-oxo-1,6-dihydro-pyridine-3-carboxylate;

ethyl 5-[2-(2-ethyl(4-pyridyl))(1,3-thiazol-4-yl)-2-methyl-6-oxo-1,6-dihydro-pyridine-3-carboxylate;

ethyl 5-[2-(3,5-dichloro-pyridin-4-yl)-thiazol-4-yl]-2-methyl-6-oxo-1,6-dihydro-pyridine-3-carboxylate;

ethyl 2-methyl-5-(2-(2-((2-methylpropyl)amino)-4-pyridinyl)-1,3-thiazol-4-yl)-6-oxo-1,6-dihydro-pyridine-3-carboxylate;

ethyl 2-methyl-6-oxo-5-(2-(2-((3-pyridinylmethyl)amino)-4-pyridinyl)-1,3-thiazol-4-yl)-1,6-dihydro-pyridine-3-carboxylate;

ethyl 2-methyl-6-oxo-5-(2-(2-((phenylmethyl)amino)-4-pyridinyl)-1,3-thiazol-4-yl)-1,6-dihydro-pyridine-3-carboxylate;

ethyl 2-methyl-5-(2-(2-((2-((1-methylethyl)amino)ethyl)amino)-4-pyridinyl)-1,3-thiazol-4-yl)-6-oxo-1,6-dihydro-pyridine-3-carboxylate;

ethyl 5-(2-(2-((2-(diethylamino)ethyl)amino)-4-pyridinyl)-1,3-thiazol-4-yl)-2-methyl-6-oxo-1,6-dihydro-pyridine-3-carboxylate;

ethyl 5-(2-{2-[(fur-2-ylmethyl)-amino]-pyridin-4-yl}-thiazol-4-yl)-2-methyl-6-oxo-1,6-dihydro-pyridine-3-carboxylate;

ethyl 5-{2-[2-(2-thien-2-yl-ethylamino)-pyridin-4-yl]-thiazol-4-yl}-2-methyl-6-oxo-1,6-dihydro-pyridine-3-carboxylate;

ethyl 5-[2-(2-butylamino-pyridin-4-yl)-thiazol-4-yl]-2-methyl-6-oxo-1,6-dihydro-pyridine-3-carboxylate;

ethyl 5-{2-[2-(carbamoylmethyl-amino)-pyridin-4-yl]-thiazol-4-yl}-2-methyl-6-oxo-1,6-dihydro-pyridine-3-carboxylate;

ethyl 5-{2-[2-acetylamino-ethylamino)-pyridin-4-yl]-thiazol-4-yl}-2-methyl-6-oxo-1,6-dihydro-pyridine-3-carboxylate;

5-{2-[2-(cyclopropylmethylamino)-pyridin-4-yl]-thiazol-4-yl}-2-methyl-6-oxo-1,6-dihydro-pyridine-3-carboxylic acid cyclopropylmethylamide;

ethyl 5-{2-[2-(cyclopropylmethyl-amino)-pyridin-4-yl]-thiazol-4-yl}-2-methyl-6-oxo-1,6-dihydro-pyridine-3-carboxylate;

ethyl 5-{2-[2-(cyclopropylmethyl-amino)-pyridin-4-yl]-thiazol-4-yl}-2-methyl-6-oxo-1,6-dihydro-pyridine-3-carboxylate hydrochloride;

ethyl 5-{2-[2-(cyclopentyl)methylamino-pyridin-4-yl]-thiazol-4-yl}-2-methyl-6-oxo-1,6-dihydro-pyridine-3-carboxylate;

5-{2-[2-(4-methoxy-benzyamino)-pyridin-4-yl]-thiazol-4-yl}-2-methyl-6-oxo-1,6-dihydro-pyridine-3-carboxylic acid 4-methoxy-benzylamide;

ethyl 2-methyl-6-oxo-5-(2-(2-(amino)-4-pyridinyl)-1,3-thiazol-4-yl)-1,6-dihydro-pyridine-3-carboxylate;

ethyl 2-methyl-5-[2-(methylamino)(1,3-thiazol-4-yl)]-6-oxo-1,6-dihydro-pyridine-3-carboxylate;

6-methyl-3-(2-(4-pyridyl)(1,3-thiazol-4-yl))-1,6-dihydro-pyridin-2-one;

ethyl 2-methyl-5-(2-(2-(methoxy)-4-pyridinyl)-1,3-thiazol-4-yl)-6-oxo-1,6-dihydro-pyridine-3-carboxylate;

ethyl 2-methyl-6-oxo-5-{2-[(phenylsulfonyl)methyl](1,3-thiazol-4-yl)}-1,6-dihydro-pyridine-3-carboxylate;

ethyl 2-methyl-6-oxo-5-(2-(4-pyridyl)(1,3-thiazol-4-yl))-1,6-dihydro-pyridine-3-carboxylate;

ethyl 2-methyl-6-oxo-5-{2-[(2-pyridylsulfonyl)methyl](1,3-thiazol-4-yl)}-1,6-dihydro-pyridine-3-carboxylate;

ethyl 2-methyl-5-(2-(1-methyl-1-(phenylsulfonyl)ethyl)-1,3-thiazol-4-yl)-6-oxo-1,6-dihydro-pyridine-3-carboxylate;

ethyl 2-cyclopropyl-6-oxo-5-(2-(4-pyridinyl)-1,3-thiazol-4-yl)-1,6-dihydro-pyridine-3-carboxylate;

ethyl 2-cyclopropyl-6-oxo-5-(2-((phenylsulfonyl)methyl)-1,3-thiazol-4-yl)-1,6-dihydro-pyridine-3-carboxylate;

5-bromo-6-methyl-3-(2-(4-pyridinyl)-1,3-thiazol-4-yl)-2(1H)-pyridinone;

ethyl 2-methyl-5-(2-(2-(methylamino)-4-pyridinyl)-1,3-thiazol-4-yl)-6-oxo-1,6-dihydro-pyridine-3-carboxylate;

5-amino-6-ethyl-3-(2-(4-pyridinyl)-1,3-thiazol-4-yl)-2(1H)-pyridinone;

2-methyl-6-oxo-N-(2-pyridinylmethyl)-5-(2-(2-((2-pyridinylmethyl)amino)-4-pyridinyl)-1,3-thiazol-4-yl)-1,6-dihydro-pyridine-3-carboxamide;

6-methyl-3-(2-(2-((2-pyridinylmethyl)amino)-4-pyridinyl)-1,3-thiazol-4-yl)-2(1H)-pyridinone;

ethyl 2-methyl-6-oxo-5-(2-(2-((2-pyridinylmethyl)amino)-4-pyridinyl)-1,3-thiazol-4-yl)-1,6-dihydro-pyridine-3-carboxylate;

ethyl 5-[2-(methylamino-pyridin-4-yl)-thiazol-4-yl]-2-isopropyl-6-oxo-1,6-dihydro-pyridine-3-carboxylate;

1,1-dimethylethyl 2-methyl-6-oxo-5-(2-(4-pyridinyl)-1,3-thiazol-4-yl)-1,6-dihydro-pyridine-3-carboxylate;

2-(1-pyrrolidinyl)ethyl 2-ethyl-6-oxo-5-(2-(4-pyridinyl)-1,3-thiazol-4-yl)-1,6-dihydro-pyridine-3-carboxylate;

6-ethyl-3-(2-pyridin-4-yl-thiazol-4-yl)-1H-pyridin-2-one;

6-isopropyl-3-(2-pyridin-4-yl-thiazol-4-yl)-1H-pyridin-2-one;

3-(diethylamino)propyl 2-ethyl-6-oxo-5-(2-(4-pyridinyl)-1,3-thiazol-4-yl)-1,6-dihydro-pyridine-3-carboxylate;

3-(diethylamino)propyl 2-(1-methylethyl)-6-oxo-5-(2-(4-pyridinyl)-1,3-thiazol-4-yl)-1,6-dihydro-pyridine-3-carboxylate; and

5-hydroxymethyl-6-methyl-3-(2-pyridin-4-yl-thiazol-4-yl)-1H-pyridin-2-one.

The invention also relates to compounds of Formula I′

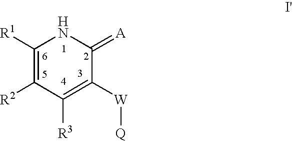

wherein A is O or S;

wherein Q is selected from —N(R⁵)₂, —NR⁵C(O)R⁵, —(C₁-C₈)alkyl-OR⁵, —(C₁-C₈)alkyl-S(O)ₙR⁶,

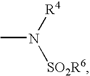

substituted aryl, an unsubstituted or substituted monocyclic or bicyclic, non-aromatic carbocyclic ring, an unsubstituted or substituted monocyclic or bicyclic, heteroaryl ring, and an unsubstituted or substituted monocyclic or bicyclic, non-aromatic heterocyclic ring,

wherein a ring is unsubstituted or substituted with one or more groups selected from halo, (C₁-C₈)alkyl, (C₂-C₈)alkynyl, (C₂-C₈)alkenyl, —OR⁵, —O—(CH₂)₁₋₂—O—, —N(R⁵)₂, —(C₁-C₈)alkyl-N(R⁵)₂, (C₁-C₈)haloalkyl, lower cyanoalkyl, —(C₁-C₈)alkyl-OR⁵, lower alkylaminoalkoxy, lower aminoalkoxyalkyl, —(C₁-C₈)alkyl-S(O)ₙR⁵, —N(R⁵)—(C₁-C₈)alkyl-N(R⁵)₂, —N(R⁵)—(C₁-C₈)alkyl-OR⁵, —N(R⁵)—(C₁-C₈)alkyl-NHC(O)R⁵, —N(R⁵)—(C₁-C₈)alkyl-C(O)N(R⁵)₂, lower alkoxyalkyl, —S(O)ₙR⁵, —SO₂NR⁵R⁵, —NR⁵S(O)ₙR⁵ cyano, nitro, optionally substituted (C₃-C₁₀)cycloalkyl, optionally substituted aryl, optionally substituted 4-7 membered heterocyclyl, optionally substituted phenoxyalkyl, optionally substituted heterocyclyloxyalkyl, —C(O)N(R⁵)₂, —CO₂R⁵, —CO₂N(R⁵)₂, —SO₂NHC(O)R⁵, optionally substituted phenylalkyl, optionally substituted heterocyclylalkyl, —NR⁵C(O)N(R⁵)₂, —NR⁵C(O)R⁵, —NR⁵CO₂R⁵ and —C(O)R⁵;

wherein W is selected from

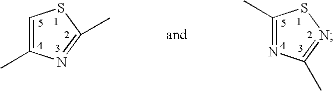

wherein n is 0, 1 or 2;

wherein R¹ is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵) 2, —CO₂R⁵, —(CR⁵₂)₁₋₈aryl, —(CR⁵₂)₁₋₈heterocyclyl, —NR⁵C(O)N(R⁵) 2, —NR⁵C(O)R⁵, —NR⁵CO₂R⁵, and —C(O)R⁵; wherein R¹ and R² may be joined to form a 5-10 membered saturated or partially unsaturated carbocyclic or heterocyclic ring;

wherein R² is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵) ₂, —CO₂R⁵, —(CR⁵₂)₁₋₈aryl, —(CR⁵₂)₁₋₈heterocyclyl, NR⁵C(O)N(R⁵)₂, —NR⁵C(O)R⁵, —NR⁵CO₂R⁵, and —C(O)R;

wherein R³ is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵)₂, —CO₂R⁵, —(CR⁵₂)₁₋₈aryl, —(CR⁵₂)₁₋₈heterocyclyl, NR⁵C(O)N(R⁵)₂, —NR⁵C(O)R⁵, —NR⁵CO₂R⁵, and —C(O)R⁵; wherein R² and R³ may be joined to form a 5-10 membered saturated or partially unsaturated carbocyclic or heterocyclic ring;

wherein R⁴ is independently selected from H, and (C₁-C₆)alkyl;

wherein R⁵ is independently selected from H, lower alkyl, optionally substituted aryl, optionally substituted aralkyl, optionally substituted heterocyclyl, optionally substituted heterocyclylalkyl, optionally substituted C₃-C₆ cycloalkyl, optionally substituted C₃-C₆ cycloalkyl-alkyl, lower aminoalkyl, aryl-(C₁-C₆)alkylamino-(C₁-C₆)alkyl, (C₁-C₆)alkylamino-(C₁-C₆)alkyl, aryloxyalkyl, alkylcarbonylalkyl, and lower perfluoroalkyl; and

wherein R⁶ is independently selected from lower alkyl, optionally substituted aryl, optionally substituted aryl-(C₁-C₆)alkyl, optionally substituted heterocyclyl, optionally substituted heterocyclyl-(C₁-C₆)alkyl, optionally substituted C₃-C₆ cycloalkyl, optionally substituted C₃-C₆ cycloalkyl-(C₁-C₆)alkyl, (C₁-C₆)alkylamino-(C₁-C₆)alkyl, aryloxy-(C₁-C₆)alkyl, (C₁-C₆)alkylcarbonyl-(C₁-C₆)alkyl, and lower perfluoroalkyl;

wherein each aryl, heteroaryl, cycloalkyl, and heterocyclyl moiety of any R¹, R², R³, R⁵, R⁶, and Q is optionally substituted with one or more groups selected from halo, —NH₂, —OH, oxo, —CO₂H, (C₁-C₆)alkylamino, (C₁-C₆)alkoxy, (C₁-C₆)alkoxyalkyl, (C₁-C₆)alkyl, di(C₁-C₆)alkylamino, phenyl, and heterocyclyl;

and pharmaceutically acceptable derivatives thereof;

provided R¹ is not CF₃ when R² is ethoxycarbonyl, when R³ is H, when W is thiazol-4-yl and when Q is 4-pyridyl or 2-chloro-4-pyridyl; further provided Q is not 4-pyridyl, when W is thiazol-2-yl, when R¹, R³, and R² are H; further provided Q is not 2-nitro-5-furyl when W is thiazol-2-yl, when R¹ is methyl, when R³ is H, and when R² is H; further provided Q is not phenyl when W is thiazol-2-yl, when R¹ is methyl, when R³ is methyl, and when R² is H; further provided Q is not phenyl, 3,4-diacetylphenyl or 3,4-dihydroxyphenyl, when W is thiazol-2-yl, when R¹ is H, when R³ is H, and when R² is H; and further provided Q is not 3-cyano-6-methyl-2-oxo-1,2-dihydro-5-pyridyl, when W is thiazol-2-yl, when R¹ is methyl, when R³ is H, and when R² is acetyl.

The invention also relates to compounds of Formula I′ wherein Q is selected from R⁶SO₂—(C₁-C₆)alkyl-,

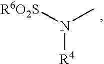

substituted phenyl, and substituted or unsubstituted 5-6 membered heteroaryl; wherein R⁴ is independently selected from H, and (C₁-C₂)alkyl; and wherein R⁶ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl- and optionally substituted heterocyclyl selected from pyridyl and thienyl; and pharmaceutically acceptable derivatives thereof; in conjunction with any of the above or below embodiments.

The invention also relates to compounds of Formula I′ wherein Q is selected from phenylsulfonylamino, N-methyl-N-(2-pyridylsulfonyl)amino, N-methyl-N-(3-pyridylsulfonyl)amino, N-methyl-N-(4-pyridylsulfonyl)amino, N-methyl-N-(2-thienylsulfonyl)amino, N-methyl-N-(phenylsulfonyl)amino, 2-pyridylsulfonylmethyl, 3-pyridylsulfonylmethyl, 4-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, 3-trifluoromethylbenzyl-sulfonylmethyl, methylsulfonylmethyl, tert-butyl-sulfonylmethyl, 4-fluorobenzylsulfonylmethyl, 4-chlorophenyl-methylsulfonylmethyl; and pharmaceutically acceptable derivatives thereof; in conjunction with any of the above or below embodiments.

The invention also relates to compounds of Formula I′ wherein Q is selected from 2-thienyl, 3-(4-chlorophenylsulfonylmethyl)-2-thienyl, phenyl substituted with one or more substituents selected from

hydroxyl, chloro, fluoro, methoxy, —O—CH₂—O—, amino, aminomethyl, methylsulfonyl, methyl, cyano, trifluoromethyl, and pyrrolyl,

unsubstituted pyridyl, and

4-pyridyl substituted with one or more substituents selected from chloro, fluoro, methyl, ethyl, —NH₂, methoxy, ethoxy, —OH, —CO₂H, phenoxyethylamino, methylamino, dimethylamino, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl; and

pharmaceutically acceptable derivatives thereof; in conjunction with any of the above or below embodiments.

The invention also relates to compounds of Formula I′ wherein W is

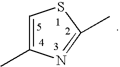

The invention also relates to compounds of Formula I′ wherein R¹ is selected from (C₁-C₆)alkyl, —(C₁-C₄)alkyl-N(R⁵)₂, —(C₁-C₄)alkyl-OR⁵, (C₃-C₅)cycloalkyl and —CF₃; wherein R⁵ is independently selected from H, C₁-C₅-alkyl, optionally substituted phenyl, optionally substituted benzyl, optionally substituted pyridyl-(C₁-C₃)-alkyl, optionally substituted thienyl-(C₁-C₃)-alkyl, optionally substituted piperazinyl-(C₁-C₃)-alkyl, 4-morpholinyl-(C₁-C₃)-alkyl, optionally substituted pyrrolidinyl-(C₁-C₃)-alkyl, optionally substituted piperidinyl-(C₁-C₃)-alkyl, optionally substituted C₃-C₆ cycloalkyl-(C₁-C₃)-alkyl, amino-(C₁-C₄)-alkyl-, benzylamino-(C₁-C₃)-alkyl-, [N—(C₁-C₃)-alkyl-N-benzylamino]-(C₁-C₃)-alkyl-, —(C₁-C₃)-alkyl-N-((C₁-C₃)-alkyl)₂, —(C₁-C₃)-alkyl-NH—(C₁-C₃)-alkyl and optionally substituted heterocyclyl selected from piperazinyl, morpholinyl, pyrrolidinyl and piperidyl; and pharmaceutically acceptable derivatives thereof; in conjunction with any of the above or below embodiments.

The invention also relates to compounds of Formula I′ wherein R¹ is selected from methyl, ethyl, propyl, isopropyl, dimethylaminomethyl, 1-pyrrolidinyltheyl, benzyloxymethyl, benzyloxyethyl, hydroxyethyl, 4-methoxy-benzyloxymethyl, methoxymethyl, cyclopropyl and —CF₃; and pharmaceutically acceptable derivatives thereof; in conjunction with any of the above or below embodiments.

The invention also relates to compounds of Formula I′ wherein R² is selected from H, halo, (C₁-C₃)alkyl, —NR⁵₂, —OR⁶, —(C₁-C₃)alkyl-OR⁵, —(C₁-C₃)alkyl-NR⁵₂—C(O)N(R⁵)₂, —CO₂R⁵, —(CH₂)₁₋₃-(5-6 membered saturated or partially unsaturated)heterocyclyl, 5-6 membered saturated or partially unsaturated heterocyclyl, —NHC(O)R⁵, and —C(O)R⁵; wherein R⁵ is independently selected from H, C₁-C₅-alkyl, optionally substituted phenyl, optionally substituted benzyl, optionally substituted pyridyl-(C₁-C₃)-alkyl, optionally substituted thienyl-(C₁-C₃)-alkyl, optionally substituted piperazinyl-(C₁-C₃)-alkyl, 4-morpholinyl-(C₁-C₃)-alkyl, optionally substituted pyrrolidinyl-(C₁-C₃)-alkyl, optionally substituted piperidinyl-(C₁-C₃)-alkyl, optionally substituted C₃-C₆ cycloalkyl-(C₁-C₃)-alkyl, amino-(C₁-C₄)-alkyl-, benzylamino-(C₁-C₃)-alkyl-, [N—(C₁-C₃)-alkyl-N-benzylamino]-(C₁-C₃)-alkyl-, —(C₁-C₃)-alkyl-N-((C₁-C₃)-alkyl)₂, —(C₁-C₃)-alkyl-NH—(C₁-C₃)-alkyl and optionally substituted heterocyclyl selected from piperazinyl, morpholinyl, pyrrolidinyl and piperidyl; and pharmaceutically acceptable derivatives thereof; in conjunction with any of the above or below embodiments.

The invention also relates to compounds of Formula I′ wherein R² is selected from H, bromo, methyl, hydroxymethyl, 1,2,5,6-tetrahydro-1-pyridylmethyl, 1-piperidylmethyl, 1-methyl-4-piperazinylmethyl, (N-diethylaminoethyl-N-methyl)aminomethyl, (N-dimethylaminoethyl-N-ethyl)aminomethyl, 4,5-dihydro-oxazol-2-yl, 5-methyl-4,5-dihydro-oxazol-2-yl, 2-furyl, amino, isobutylamino, 3-methylbutylamino, ethylcarbonyl, aminocarbonyl, 4-methoxybenzylaminocarbonyl, 2-pyridylmethylaminocarbonyl, 4-pyridylmethylaminocarbonyl, dimethylaminocarbonyl, ethylaminoethylaminocarbonyl, isopropylaminoethylaminocarbonyl, cyclopropylmethylaminocarbonyl, isobutylaminocarbonyl, ethoxycarbonyl, propoxycarbonyl, 1-methylpropoxycarbonyl, butoxycarbonyl, iso-butoxycarbonyl, tert-butoxycarbonyl, 2-thienylethoxycarbonyl, 4-morpholinylethoxycarbonyl, (4-piperidinyl)methoxycarbonyl, (1-piperazinyl)ethoxycarbonyl, (1-methyl-piperidin-3-yl)oxycarbonyl, (1-methyl-piperidin-4-yl)oxycarbonyl, (1-ethyl-piperidin-3-yl)oxycarbonyl, (1-methyl-pyrrolidin-3-yl)oxycarbonyl, 1-pyrrolidinylethoxycarbonyl, 2-oxo-pyrrolidin-1-ylethoxycarbonyl, 2-oxo-pyrrolidin-1-ylpropoxycarbonyl, 1-methyl-2-pyrrolidinylethoxycarbonyl, 1-piperidylethoxycarbonyl, diethylaminoethoxycarbonyl, di-isopropylaminoethoxycarbonyl, (N-ethyl-N-benzylamino)ethoxycarbonyl, diethylaminopropoxycarbonyl, dimethylaminoethoxycarbonyl, 2-(dimethylamino)-1-(methyl)ethoxycarbonyl, 2-(diethylamino)-1-(methyl)ethoxycarbonyl, carboxyl, methylcarbonylamino, isobutylcarbonylamino, methylaminomethylcarbonylamino, dimethylaminomethylcarbonylamino, tert-butylaminomethylcarbonylamino, (1-amino-2-methylpropyl)carbonylamino, 1-piperidinylmethylcarbonylamino, 1-piperidinylethylcarbonylamino, 1-piperidinylpropylcarbonylamino, aminomethylcarbonylamino and 1-methyl-4-piperazinylcarbonyl; and pharmaceutically acceptable derivatives thereof; in conjunction with any of the above or below embodiments.

The invention also relates to compounds of Formula I′ wherein R¹ and R² may be joined together with the pyridone ring to form optionally substituted 2-oxo-1,5,7,8-tetrahydro-2H-[1,6]naphthyridine, optionally substituted 5,6,7,8-tetrahydro-1H-[1,6]naphthyridin-2-one, optionally substituted 5,6,7,8-tetrahydro-1H-[1,7]naphthyridin-2-one, optionally substituted 5,6,7,8-tetrahydro-1H-quinolin-2-one, optionally substituted 7,8-dihydro-1H-quinolin-2-one, 7,8-dihydro-(1H,6H)-quinoline-2,5-dione or 1,5,7,8-tetrahydro-pyrano[4,3-b]pyridin-2-one; and pharmaceutically acceptable derivatives thereof; in conjunction with any of the above or below embodiments.

The invention also relates to compounds of Formula I′ wherein R¹ and R² are joined together with the pyridone ring to form 6-benzyloxycarbonyl-2-oxo-1,5,7,8-tetrahydro-2H-[1,6]naphthyridine, 5,6,7,8-tetrahydro-1H-[1,6]naphthyridin-2-one, 7-Boc-5,6,7,8-tetrahydro-1H-[1,7]naphthyridin-2-one, 7-ethyl-5,6,7,8-tetrahydro-1H-[1,7]naphthyridin-2-one, 5-methyl-7,8-dihydro-1H-quinolin-2-one, 5-propylamino-5,6,7,8-tetrahydro-1H-quinolin-2-one, 5-propylimino-5,6,7,8-tetrahydro-1H-quinolin-2-one, 7,8-dihydro-(1H,6H)-quinoline-2,5-dione or 1,5,7,8-tetrahydro-pyrano[4,3-b]pyridin-2-one; and pharmaceutically acceptable derivatives thereof; in conjunction with any of the above or below embodiments.

The invention also relates to compounds of Formula I′ wherein R³ is H; and pharmaceutically acceptable derivatives thereof; in conjunction with any of the above or below embodiments.

The invention also relates to compounds of Formula I′ wherein A is O; and pharmaceutically acceptable derivatives thereof; in conjunction with any of the above or below embodiments.

The invention also relates to compounds of Formula I′

wherein A is O;

wherein Q is selected from

N-methyl-N-(phenylsulfonyl)amino,

2-pyridylsulfonylmethyl,

2-thienylsulfonylmethyl,

phenylsulfonylmethyl,

(1-methyl)-1-(phenylsulfonyl)ethyl,

4-chlorophenyl-sulfonylmethyl,

2-furylmethylsulfonylmethyl,

methylsulfonylmethyl,

tert-butyl-sulfonylmethyl,

4-fluorobenzylsulfonylmethyl,

2-thienyl,

phenyl substituted with one or more substituents selected from chloro, fluoro, and —O—CH₂—O—,

unsubstituted pyridyl, and

4-pyridyl substituted with one or more substituents selected from chloro, fluoro, —NH₂, methoxy, ethoxy, methyl, ethyl, phenoxyethylamino, methylamino, dimethylamino, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl;

wherein R¹ is selected from methyl, ethyl, propyl, isopropyl, dimethylaminomethyl, hydroxyethyl, benzyloxymethyl, 4-methoxy-benzyloxymethyl, methoxymethyl, cyclopropyl, and —CF₃;

wherein R² is selected from H, bromo, methyl, amino, isobutylamino, hydroxymethyl, aminocarbonyl, 4-methoxybenzylaminocarbonyl, 2-pyridylmethylaminocarbonyl, ethylaminoethylaminocarbonyl, isopropylaminoethylaminocarbonyl, cyclopropylmethylaminocarbonyl, isobutylaminocarbonyl, ethoxycarbonyl, tert-butoxycarbonyl, 4-morpholinylethoxycarbonyl, 1-pyrrolidinylethoxycarbonyl, 1-piperidylethoxycarbonyl, diethylaminopropoxycarbonyl, carboxyl, 1,2,5,6-tetrahydro-1-pyridylmethyl, 1-piperidylmethyl, 1-methyl-4-piperazinylmethyl, methylcarbonylamino, isobutylcarbonylamino, and 1-methyl-4-piperazinylcarbonyl; and

wherein R³ is H.

The invention also relates to compounds of Formula II′

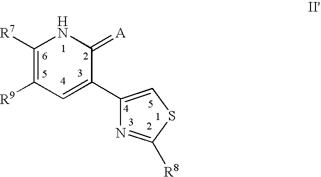

wherein R⁷ is selected from —(C₁-C₃)alkyl, —(C₁-C₃)alkyl-N(R¹⁰)₂, —(C₁-C₃)alkyl-OR¹⁰, —(C₃-C₅)cycloalkyl, and —CF₃;

wherein R⁸ is selected from R¹⁰SO₂—(C₁-C₆)alkyl-, R¹¹SO₂NH—

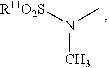

substituted phenyl, and substituted or unsubstituted 5-6 membered heteroaryl; wherein R⁹ is selected from H, halo, (C₁-C₃)alkyl, —NR¹⁰₂, —(C₁-C₃)alkyl-OR¹⁰, —C(O)N(R¹⁰)₂, —CO₂R¹⁰, (CH₂)₁₋₃-(5-6 membered saturated or partially unsaturated heterocyclyl, —NHC(O)R¹⁰, and —C(O)R¹⁰;

wherein R¹⁰ is independently selected from H, (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl- and optionally substituted heterocyclyl selected from pyridyl and thienyl; and

wherein R¹¹ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl, and optionally substituted heterocyclyl selected from pyridyl and thienyl;

and pharmaceutically acceptable derivatives thereof;

provided R⁷ is not CF₃ when R⁹ is ethoxycarbonyl and when R⁸ is 4-pyridyl or 2-chloro-4-pyridyl.

The invention also relates to compounds of Formula II′ wherein R⁷ is selected from methyl, ethyl, propyl, isopropyl, dimethylaminomethyl, 1-pyrrolidinyltheyl, benzyloxymethyl, benzyloxyethyl, hydroxyethyl, 4-methoxy-benzyloxymethyl, methoxymethyl, cyclopropyl and —CF₃; wherein R⁸ is selected from N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl, phenyl substituted with one or more substituents selected from chloro, fluoro, and —O—CH₂—O—, unsubstituted pyridyl, and

4-pyridyl substituted with one or more substituents selected from chloro, fluoro, —NH₂, methoxy, ethoxy, methyl, ethyl, phenoxyethylamino, methylamino, butylamino, isobutylamino, dimethylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl; and

wherein R⁹ is selected from H, bromo, methyl, hydroxymethyl, 1,2,5,6-tetrahydro-1-pyridylmethyl, 1-piperidylmethyl, 1-methyl-4-piperazinylmethyl, (N-diethylaminoethyl-N-methyl)aminomethyl, (N-dimethylaminoethyl-N-ethyl)aminomethyl, 4,5-dihydro-oxazol-2-yl, 5-methyl-4,5-dihydro-oxazol-2-yl, 2-furyl, amino, isobutylamino, 3-methylbutylamino, ethylcarbonyl, aminocarbonyl, 4-methoxybenzylaminocarbonyl, 2-pyridylmethylaminocarbonyl, 4-pyridylmethylaminocarbonyl, dimethylaminocarbonyl, ethylaminoethylaminocarbonyl, isopropylaminoethylaminocarbonyl, cyclopropylmethylaminocarbonyl, isobutylaminocarbonyl, ethoxycarbonyl, propoxycarbonyl, 1-methylpropoxycarbonyl, butoxycarbonyl, iso-butoxycarbonyl, tert-butoxycarbonyl, 2-thienylethoxycarbonyl, 4-morpholinylethoxycarbonyl, (4-piperidinyl)methoxycarbonyl, (1-piperidinyl)ethoxycarbonyl, (1-piperazinyl)ethoxycarbonyl, (1-methyl-piperidin-3-yl)oxycarbonyl, (1-methyl-piperidin-4-yl)oxycarbonyl, (1-ethyl-piperidin-3-yl)oxycarbonyl, (1-methyl-pyrrolidin-3-yl)oxycarbonyl, 1-pyrrolidinylethoxycarbonyl, 2-oxo-pyrrolidin-1-ylethoxycarbonyl, 2-oxo-pyrrolidin-1-ylpropoxycarbonyl, 1-methyl-2-pyrrolidinylethoxycarbonyl, 1-piperidylethoxycarbonyl, diethylaminoethoxycarbonyl, di-isopropylaminoethoxycarbonyl, (N-ethyl-N-benzylamino)ethoxycarbonyl, diethylaminopropoxycarbonyl, dimethylaminoethoxycarbonyl, 2-(dimethylamino)-1-(methyl)ethoxycarbonyl, 2-(diethylamino)-1-(methyl)ethoxycarbonyl, carboxyl, methylcarbonylamino, isobutylcarbonylamino, methylaminomethylcarbonylamino, dimethylaminomethylcarbonylamino, tert-butylaminomethylcarbonylamino, (1-amino-2-methylpropyl)carbonylamino, 1-piperidinylmethylcarbonylamino, 1-piperidinylethylcarbonylamino, 1-piperidinylpropylcarbonylamino, aminomethylcarbonylamino and 1-methyl-4-piperazinylcarbonyl; and pharmaceutically acceptable derivatives thereof.

The invention also relates to compounds of Formula II′ wherein R⁷ is selected from methyl, ethyl, propyl, and isopropyl; and pharmaceutically acceptable derivatives thereof; in conjunction with any of the above or below embodiments.

The invention also relates to compounds of Formula II′ wherein R⁸ is selected from phenylsulfonylmethyl and 4-pyridyl substituted with one or more substituents selected from chloro, fluoro, —NH₂, methoxy, ethoxy, phenoxyethylamino, methylamino, dimethylamino, methyl, ethyl, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl; and pharmaceutically acceptable derivatives thereof; in conjunction with any of the above or below embodiments.

The invention also relates to compounds of Formula II′ wherein R⁹ is selected from methyl, hydroxymethyl, 1,2,5,6-tetrahydro-1-pyridylmethyl, 1-piperidylmethyl, 1-methyl-4-piperazinylmethyl, (N-diethylaminoethyl-N-methyl)aminomethyl, (N-dimethylaminoethyl-N-ethyl)aminomethyl, 4,5-dihydro-oxazol-2-yl, 5-methyl-4,5-dihydro-oxazol-2-yl, 2-furyl, amino, isobutylamino, 3-methylbutylamino, ethylcarbonyl, aminocarbonyl, 4-methoxybenzylaminocarbonyl, 2-pyridylmethylaminocarbonyl, 4-pyridylmethylaminocarbonyl, dimethylaminocarbonyl, ethylaminoethylaminocarbonyl, isopropylaminoethylaminocarbonyl, cyclopropylmethylaminocarbonyl, isobutylaminocarbonyl, ethoxycarbonyl, propoxycarbonyl, 1-methylpropoxycarbonyl, butoxycarbonyl, iso-butoxycarbonyl, tert-butoxycarbonyl, 2-thienylethoxycarbonyl, 4-morpholinylethoxycarbonyl, (4-piperidinyl)methoxycarbonyl, (1-piperidinyl)ethoxycarbonyl, (1-piperazinyl)ethoxycarbonyl, (1-methyl-piperidin-3-yl)oxycarbonyl, (1-methyl-piperidin-4-yl)oxycarbonyl, (1-ethyl-piperidin-3-yl)oxycarbonyl, (1-methyl-pyrrolidin-3-yl)oxycarbonyl, 1-pyrrolidinylethoxycarbonyl, 2-oxo-pyrrolidin-1-ylethoxycarbonyl, 2-oxo-pyrrolidin-1-ylpropoxycarbonyl, 1-methyl-2-pyrrolidinylethoxycarbonyl, 1-piperidylethoxycarbonyl, diethylaminoethoxycarbonyl, di-isopropylaminoethoxycarbonyl, (N-ethyl-N-benzylamino)ethoxycarbonyl, diethylaminopropoxycarbonyl, dimethylaminoethoxycarbonyl, 2-(dimethylamino)-1-(methyl)ethoxycarbonyl, 2-(diethylamino)-1-(methyl)ethoxycarbonyl, carboxyl, methylcarbonylamino, isobutylcarbonylamino, methylaminomethylcarbonylamino, dimethylaminomethylcarbonylamino, tert-butylaminomethylcarbonylamino, (1-amino-2-methylpropyl)carbonylamino, 1-piperidinylmethylcarbonylamino, 1-piperidinylethylcarbonylamino, 1-piperidinylpropylcarbonylamino, aminomethylcarbonylamino and 1-methyl-4-piperazinylcarbonyl; and pharmaceutically acceptable derivatives thereof; in conjunction with any of the above or below embodiments.

The invention also relates to compounds of Formula II′ selected from:

6-Isopropyl-5-methyl-3-(2-pyrindin-4-yl-thiazol-4-yl)-1H-pyridin-2-one;

6-Ethyl-5-isopropionyl-3-(2-pyridin-4-yl-thiazol-4-yl)-1H-pyridin-2-one;

2-Isopropyl-6-oxo-5-(2-pyridin-4-yl-thiazol-4-yl)-1,6-dihydro-pyridine-3-carboxylic acid 2-(2-oxo-pyrrolidin-1-yl)-ethyl ester;

2-Isopropyl-6-oxo-5-(2-pyridin-4-yl-thiazol-4-yl)-1,6-dihydro-pyridine-3-carboxylic acid 2-diethylamino-ethyl ester;

2-Isopropyl-6-oxo-5-(2-pyridin-4-yl-thiazol-4-yl)-1,6-dihydro-pyridine-3-carboxylic acid 2-pyrrolidin-1-yl-ethyl ester;

2-Isopropyl-6-oxo-5-(2-pyridin-4-yl-thiazol-4-yl)-1,6-dihydro-pyridine-3-carboxylic acid 2-diethylamino-1-methyl-ethyl ester;

2-Isopropyl-6-oxo-5-(2-pyridin-4-yl-thiazol-4-yl)-1,6-dihydro-pyridine-3-carboxylic acid 1-ethyl-piperidin-3-yl ester;

2-Isopropyl-6-oxo-5-(2-pyridin-4-yl-thiazol-4-yl)-1,6-dihydro-pyridine-3-carboxylic acid 2-dimethylamino-ethyl ester;

2-Isopropyl-6-oxo-5-(2-pyridin-4-yl-thiazol-4-yl)-1,6-dihydro-pyridine-3-carboxylic acid 2-dimethylamino-1-methyl-ethyl ester;

2-Isopropyl-6-oxo-5-(2-pyridin-4-yl-thiazol-4-yl)-1,6-dihydro-pyridine-3-carboxylic acid 1-methyl-piperidin-3-yl ester;

2-Isopropyl-6-oxo-5-(2-pyridin-4-yl-thiazol-4-yl)-1,6-dihydro-pyridine-3-carboxylic acid 1-ethyl-pyrrolidin-3-yl ester;

5-(2-Benzenesulfonylmethyl-thiazol-4-yl)-2-isopropyl-6-oxo-1,6-pyridine-3-carboxylic acid 2-diethylamino-ethyl ester;

2-Isopropyl-6-oxo-5-(2-pyridin-4-yl-thiazol-4-yl)-1,6-dihydro-pyridine-3-carboxylic acid piperidin-4-ylmethyl ester;

5-(2-Benzenesulfonylmethyl-thiazol-4-yl)-2-isopropyl-6-oxo-1,6-pyridine-3-carboxylic acid 2-diethylamino-1-methyl-ethyl ester;

2-Isopropyl-6-oxo-5-(2-pyridin-4-yl-thiazol-4-yl)-1,6-dihydro-pyridine-3-carboxylic acid 2-(benzyl-methyl-amino)-ethyl ester;

5-(2-Benzenesulfonylmethyl-thiazol-4-yl)-2-isopropyl-6-oxo-1,6-pyridine-3-carboxylic acid 2-diethylamino-propyl ester;

5-(2-Benzenesulfonylmethyl-thiazol-4-yl)-2-isopropyl-6-oxo-1,6-pyridine-3-carboxylic acid 2-(1-methyl-pyrrolidin-2-yl)-ethyl ester;

5-[2-(2-Dimethylamino-pyridin-4-yl)-thiazol-4-yl]-2-isopropyl-6-oxo-1,6-dihydro-pyridine-3-carboxylic acid ethyl ester;

2-Isopropyl-6-oxo-5-(2-pyridin-4-yl-thiazol-4-yl)-1,6-dihydro-pyridine-3-carboxylic acid 2-piperazin-1-yl-ethyl ester;

2-Isopropyl-6-oxo-5-(2-pyridin-4-yl-thiazol-4-yl)-1,6-dihydro-pyridine-3-carboxylic acid 2-(2-oxo-pyrrolidin-1-yl)-propyl ester;

2-Isopropyl-6-oxo-5-(2-pyridin-4-yl-thiazol-4-yl)-1,6-dihydro-pyridine-3-carboxylic acid 1-methyl-pyrrolidin-3-yl ester;

3-(2-Benzenesulfonylmethyl-thiazol-4-yl)-6-isopropyl-5-methyl-1H-pyridin-2-one;

3-(2-Benzenesulfonylmethyl-thiazol-4yl)-6-ethyl-5-propionyl-1H-pyridin-2-one;

2-Isopropyl-6-oxo-5-(2-pyridin-4-yl-thiazol-4-yl)-1,6-dihydro-pyridine-3-carboxylic acid 2-morpholin-4-yl-ethyl ester;

2-Isopropyl-6-oxo-5-(2-pyridin-4-yl-thiazol-4-yl)-1,6-dihydro-pyridine-3-carboxylic acid phenethyl ester;

2-Isopropyl-6-oxo-5-(2-pyridin-4-yl-thiazol-4-yl)-1,6-dihydro-pyridine-3-carboxylic acid piperidin-4-ylmethyl ester;

2-Isopropyl-6-oxo-5-(2-pyridin-4-yl-thiazol-4-yl)-1,6-dihydro-pyridine-3-carboxylic acid 2-thiophen-2-yl-ethyl ester;

5-(4,5-Dihydro-oxazol-2-yl)-6-isopropyl-3-(2-pyridin-4-yl-thiazol-4-yl)-1H-pyridin-2-one;

5-{[(2-Dimethylamino-ethyl)-ethyl-amino]-methyl}-6-ethyl-3-(2-pyridin-4-yl-thiazol-4-yl)-1H-pyridin-2-one;

2-Isopropyl-6-oxo-5-(2-pyridin-4-yl-thiazol-4-yl)-1,6-dihydro-pyridine-3-carboxylic acid 2-piperidin-1-yl-ethyl ester;

5-{[(2-Diethylamino-ethyl)-methyl-amino]-methyl}-6-ethyl-3-(2-pyridin-4-yl-thiazol-4-yl)-1H-pyridin-2-one;

2-(2-Hydroxy-ethyl)-6-oxo-5-(2-pyridin-4-yl-thiazol-4-yl)-1,6-dihydro-pyridine-3-carboxylic acid ethyl ester;

2-Amino-N-[2-ethyl-6-oxo-5-(2-pyridin-4-yl-thiazol-4-yl)-1,6-dihydro-pyridin-3-yl]-acetamide;

2-tert-Butylamino-N-[2-ethyl-6-oxo-5-(2-pyridin-4-yl-thiazol-4-yl)-1,6-dihydro-pyridin-3-yl]-acetamide;

6-Ethyl-5-(3-methyl-butylamino)-3-(2-pyridin-4-yl-thiazol-4-yl)-1H-pyridin-2-one;

Ethyl 2-ethyl-6-oxo-5-(2-(4-pyridinyl)-1,3-thiazol-4-yl)-1,6-dihydro-pyridine-3-carboxylate;

Ethyl-2-ethyl-6-oxo-5-{2-[(thienylsulfonyl)methyl](1,3-thiazol-4-yl)}-1,6-dihydro-pyridine-3-carboxylate;

Ethyl-2-ethyl-6-oxo-5-{2-[(phenylsulfonyl)methyl](1,3-thiazol-4-yl)}-1,6-dihydro-pyridine-3-carboxylate;

Ethyl-6-oxo-5-{2-[(phenylsulfonyl)methyl](1,3-thiazol-4-yl)}-2-(trifluoromethyl)-1,6-dihydro-pyridine-3-carboxylate;

Ethyl-6-oxo-5-{2-[(2-pyridylsulfonyl)methyl](1,3-thiazol-4-yl)}-2-(trifluoromethyl)-1,6-dihydro-pyridine-3-carboxylate;

Ethyl-6-oxo-5-{2-[(2-thienylsulfonyl)methyl](1,3-thiazol-4-yl)}-2-(trifluoromethyl)-1,6-dihydro-pyridine-3-carboxylate;

Ethyl 2-isopropyl-6-oxo-5-(2-(4-pyridyl)(1,3-thiazol-4-yl)-1,6-dihydro-pyridine-3-carboxylate;

Ethyl 2-isopropyl-6-oxo-5-{2-[(thienylsulfonyl)methyl](1,3-thiazol-4-yl)}-1,6-dihydro-pyridine-3-carboxylate;

Ethyl 2-isopropyl-6-oxo-5-{2-[(phenylsulfonyl)methyl](1,3-thiazol-4-yl)}-1,6-dihydro-pyridine-3-carboxylate;

Ethyl 2-propyl-6-oxo-5-(2-(4-pyridyl)(1,3-thiazol-4-yl)-1,6-dihydro-pyridine-3-carboxylate;

Ethyl 2-propyl-6-oxo-5-{2-[(phenylsulfonyl)methyl](1,3-thiazol-4-yl)}-1,6-dihydro-pyridine-3-carboxylate;

Ethyl 2-propyl-6-oxo-5-{2-[(thienylsulfonyl)methyl](1,3-thiazol-4-yl)}-1,6-dihydro-pyridine-3-carboxylate;

Ethyl 6-oxo-2-[(phenylmethoxy)methyl]-5-(2-(4-pyridyl)(1,3-thiazol-4-yl))-1,6-dihydro-pyridine-3-carboxylate;

Ethyl 6-oxo-2-[(phenylmethoxy)methyl]-5-{2-[(phenylsulfonyl)methyl](1,3-thiazol-4-yl)}-1,6-dihydro-pyridine-3-carboxylate;

Ethyl 2-methyl-6-oxo-5-{2-[(2-thienylsulfonyl)methyl](1,3-thiazol-4-yl)}-1,6-dihydro-pyridine-3-carboxylate;

Ethyl 5-[2-({[(4-fluorophenyl)methyl]sulfonyl}methyl)(1,3-thiazol-4-yl)]-2-methyl-6-oxo-1,6-dihydro-pyridine-3-carboxylate;

Ethyl 5-[2-({[(4-fluorophenyl)methyl]sulfonyl}methyl)(1,3-thiazol-4-yl)]-2-methyl-6-oxo-1,6-dihydro-pyridine-3-carboxylate;

(Ethyl 2-methyl-6-oxo-5-{2-[(2-thienylsulfonyl)methyl]methyl](1,3-thiazol-4-yl)}-1,6-dihydro-pyridine-3-carboxylate;

Ethyl 2-methyl-6-oxo-5-{2-(phenylthiomethyl)(1,3-thiazol-4-yl)}-1,6-dihydro-pyridine-3-carboxylate;

Ethyl 5-[2-(2-chloro(4-pyridyl))(1,3-thiazol-4-yl)-2-methyl-6-oxo-1,6-dihydro-pyridine-3-carboxylate;

Ethyl 5-(2-{[(2-furylmethyl)sulfonyl]methyl}(1,3-thiazol-4-yl))-2-methyl-6-oxo-1,6-dihydro-pyridine-3-carboxylate;

Ethyl 5-(2-{[(2-furylmethyl)sulfonyl]methyl}(1,3-thiazol-4-yl))-2-methyl-6-oxo-1,6-dihydro-pyridine-3-carboxylate;

Ethyl 5-[2-(2-ethyl(4-pyridyl))(1,3-thiazol-4-yl)-2-methyl-6-oxo-1,6-dihydro-pyridine-3-carboxylate;

Ethyl 2-methyl-5-(2-(2-((2-methylpropyl)amino)-4-pyridinyl)-1,3-thiazol-4-yl)-6-oxo-1,6-dihydro-pyridine-3-carboxylate;

Ethyl 2-methyl-6-oxo-5-(2-(2-((3-pyridinylmethyl)amino)-4-pyridinyl)-1,3-thiazol-4-yl)-1,6-dihydro-pyridine-3-carboxylate;

Ethyl 2-methyl-6-oxo-5-(2-(2-((phenylmethyl)amino)-4-pyridinyl)-1,3-thiazol-4-yl)-1,6-dihydro-pyridine-3-carboxylate;

Ethyl 2-methyl-5-(2-(2-((2-((1-methylethyl)amino)ethyl)amino)-4-pyridinyl)-1,3-thiazol-4-yl)-6-oxo-1,6-dihydro-pyridine-3-carboxylate;

Ethyl 5-(2-(2-((2-(diethylamino)ethyl)amino)-4-pyridinyl)-1,3-thiazol-4-yl)-2-methyl-6-oxo-1,6-dihydro-pyridine-3-carboxylate;

Ethyl 5-(2-{2-[(fur-2-ylmethyl)-amino]-pyridin-4-yl}-thiazol-4-yl)-2-methyl-6-oxo-1,6-dihydro-pyridine-3-carboxylate;

Ethyl 5-{2-[2-(2-thien-2-yl-ethylamino)-pyridin-4-yl]-thiazol-4-yl}-2-methyl-6-oxo-1,6-dihydro-pyridine-3-carboxylate;

Ethyl 5-[2-(2-butylamino-pyridin-4-yl)-thiazol-4-yl]-2-methyl-6-oxo-1,6-dihydro-pyridine-3-carboxylate;

Ethyl 5-{2-[2-(carbamoylmethyl-amino)-pyridin-4-yl]-thiazol-4-yl}-2-methyl-6-oxo-1,6-dihydro-pyridine-3-carboxylate;

Ethyl 5-{2-[2-acetylamino-ethylamino)-pyridin-4-yl]-thiazol-4-yl}-2-methyl-6-oxo-1,6-dihydro-pyridine-3-carboxylate;

5-{2-[2-(Cyclopropylmethylamino)-pyridin-4-yl]-thiazol-4-yl}-2-methyl-6-oxo-1,6-dihydro-pyridine-3-carboxylic acid cyclopropyl-methyl amide;

Ethyl 5-{2-[2-(cyclopropylmethyl-amino)-pyridin-4-yl]-thiazol-4-yl}-2-methyl-6-oxo-1,6-dihydro-pyridine-3-carboxylate;

5-{2-[2-(Cyclopentyl)methylamino-pyridin-4-yl]-thiazol-4-yl}-2-methyl-6-oxo-1,6-dihydro-pyridine-3-carboxylate;

5-{2-[2-(4-Methoxybenzylamino)-pyridin-4-yl]-thiazol-4-yl}-2-methyl-6-oxo-1,6-dihydro-pyridine-3-carboxylic acid 4-methoxy-benzylamide;

Ethyl 2-methyl-6-oxo-5-(2-(2-amino-4-pyridinyl)-1,3-thiazol-4-yl)-1,6-dihydro-pyridine-3-carboxylate;

Ethyl 2-methyl-5-[2-(methylamino)(1,3-thiazol-4-yl)]-6-oxo-1,6-dihydro-pyridine-3-carboxylate;

6-Methyl-3-(2-(4-pyridyl)(1,3-thiazol-4-yl))-1,6-dihydro-pyridin-2-one;

Ethyl 2-methyl-5-(2-(2-(methyloxy)-4-pyridinyl)-1,3-thiazol-4-yl)-6-oxo-1,6-dihydro-pyridine-3-carboxylate;

Ethyl 2-methyl-6-oxo-5-{2-[(phenylsulfonyl)methyl](1,3-thiazol-4-yl)}-1,6-dihydro-pyridine-3-carboxylate;

Ethyl 2-methyl-6-oxo-5-(2-(4-pyridyl)(1,3-thiazol-4-yl))-1,6-dihydro-pyridine-3-carboxylate;

Ethyl 2-methyl-6-oxo-5-{2-[(2-pyridylsulfonyl)methyl](1,3-thiazol-4-yl)}-1,6-dihydro-pyridine-3-carboxylate;

Ethyl 2-methyl-5-(2-(1-methyl-1-(phenylsulfonyl)ethyl)-1,3-thiazol-4-yl)-6-oxo-1,6-dihydro-pyridine-3-carboxylate;

Ethyl 2-cyclopropyl-6-oxo-5-(2-(4-pyridinyl)-1,3-thiazol-4-yl)-1,6-dihydro-pyridine-3-carboxylate;

Ethyl 2-cyclopropyl-6-oxo-5-(2-((phenylsulfonyl)methyl)-1,3-thiazol-4-yl)-1,6-dihydro-pyridine-3-carboxylate;

5-Bromo-6-methyl-3-(2-(4-pyridinyl)-1,3-thiazol-4-yl)-2(1H)-pyridinone;

Ethyl 2-methyl-5-(2-(2-(methylamino)-4-pyridinyl)-1,3-thiazol-4-yl)-6-oxo-1,6-dihydro-pyridine-3-carboxylate;

5-Amino-6-ethyl-3-(2-(4-pyridinyl)-1,3-thiazol-4-yl)-2(1H)-pyridinone;

6-Methyl-3-(2-(2-((2-pyridinylmethyl)amino)-4-pyridinyl)-1,3-thiazol-4-yl)-2(1H)-pyridinone;

Ethyl 2-methyl-6-oxo-5-(2-(2-((2-pyridinylmethyl)amino)-4-pyridinyl)-1,3-thiazol-4-yl)-1,6-dihydro-pyridine-3-carboxylate;

Ethyl 5-[2-(methylamino-pyridin-4-yl)-thiazol-4-yl]-2-isopropyl-6-oxo-1,6-dihydro-pyridine-3-carboxylate;

1,1-Dimethylethyl 2-methyl-6-oxo-5-(2-(4-pyridinyl)-1,3-thiazol-4-yl)-1,6-dihydro-pyridine-3-carboxylate;

2-(1-Pyrrolidinyl)ethyl 2-ethyl-6-oxo-5-(2-(4-pyridinyl)-1,3-thiazol-4-yl)-1,6-dihydro-pyridine-3-carboxylate;

6-Ethyl-3-(2-pyridin-4-yl-thiazol-4-yl)-1H-pyridin-2-one;

6-Isopropyl-3-(2-pyridin-4-yl-thiazol-4-yl)-1H-pyridin-2-one;

3-(Diethylamino)propyl 2-ethyl-6-oxo-5-(2-(4-pyridinyl)-1,3-thiazol-4-yl)-1,6-dihydro-pyridine-3-carboxylate;

3-(Diethylamino)propyl 2-(1-methylethyl)-6-oxo-5-(2-(4-pyridinyl)-1,3-thiazol-4-yl)-1,6-dihydro-pyridine-3-carboxylate; and

5-Hydroxymethyl-6-methyl-3-(2-pyridin-4-yl-thiazol-4-yl)-1H-pyridin-2-one.

The invention also relates to compounds of Formula II′ selected from:

6-Isopropyl-5-methyl-3-(2-pyrindin-4-yl-thiazol-4-yl)-1H-pyridin-2-one;

3-(2-Benzenesulfonylmethyl-thiazol-4-yl)-6-isopropyl-5-methyl-1H-pyridin-2-one;

6-Ethyl-5-isopropionyl-3-(2-pyridin-4-yl-thiazol-4-yl)-1H-pyridin-2-one;

3-(2-Benzenesulfonylmethyl-thiazol-4yl)-6-ethyl-5-propionyl-1H-pyridin-2-one;

2-Isopropyl-6-oxo-5-(2-pyridin-4-yl-thiazol-4-yl)-1,6-dihydro-pyridine-3-carboxylic acid 2-pyrrolidin-1-yl-ethyl ester;

2-Isopropyl-6-oxo-5-(2-pyridin-4-yl-thiazol-4-yl)-1,6-dihydro-pyridine-3-carboxylic acid 2-(2-oxo-pyrrolidin-1-yl)-ethyl ester;

2-Isopropyl-6-oxo-5-(2-pyridin-4-yl-thiazol-4-yl)-1,6-dihydro-pyridine-3-carboxylic acid 2-diethylamino-ethyl ester;

2-Isopropyl-6-oxo-5-(2-pyridin-4-yl-thiazol-4-yl)-1,6-dihydro-pyridine-3-carboxylic acid 1-ethyl-piperidin-3-yl ester;

2-Isopropyl-6-oxo-5-(2-pyridin-4-yl-thiazol-4-yl)-1,6-dihydro-pyridine-3-carboxylic acid 1-methyl-piperidin-3-yl ester;

2-Isopropyl-6-oxo-5-(2-pyridin-4-yl-thiazol-4-yl)-1,6-dihydro-pyridine-3-carboxylic acid 2-dimethylamino-1-methyl-ethyl ester;

2-Isopropyl-6-oxo-5-(2-pyridin-4-yl-thiazol-4-yl)-1,6-dihydro-pyridine-3-carboxylic acid 2-diethylamino-1-methyl-ethyl ester;

2-Isopropyl-6-oxo-5-(2-pyridin-4-yl-thiazol-4-yl)-1,6-dihydro-pyridine-3-carboxylic acid 2-(benzyl-methyl-amino)-ethyl ester;

2-Isopropyl-6-oxo-5-(2-pyridin-4-yl-thiazol-4-yl)-1,6-dihydro-pyridine-3-carboxylic acid 1-methyl-piperidin-4-yl ester;

2-Isopropyl-6-oxo-5-(2-pyridin-4-yl-thiazol-4-yl)-1,6-dihydro-pyridine-3-carboxylic acid 2-(2-oxo-pyrrolidin-1-yl)-propyl ester;

2-Isopropyl-6-oxo-5-(2-pyridin-4-yl-thiazol-4-yl)-1,6-dihydro-pyridine-3-carboxylic acid phenethyl ester;

2-Isopropyl-6-oxo-5-(2-pyridin-4-yl-thiazol-4-yl)-1,6-dihydro-pyridine-3-carboxylic acid 2-thiophen-2-yl-ethyl ester;

5-(2-Benzenesulfonylmethyl-thiazol-4-yl)-2-isopropyl-6-oxo-1,6-dihydro-pyridine-3-carboxylic acid 2-diethylamino-ethyl ester;

5-(2-Benzenesulfonylmethyl-thiazol-4-yl)-2-isopropyl-6-oxo-1,6-dihydro-pyridine-3-carboxylic acid 2-diethylamino-1-methyl-ethyl ester;

5-(2-Benzenesulfonylmethyl-thiazol-4-yl)-2-isopropyl-6-oxo-1,6-dihydro-pyridine-3-carboxylic acid 2-diethylamino-propyl ester;

5-(2-Benzenesulfonylmethyl-thiazol-4-yl)-2-isopropyl-6-oxo-1,6-dihydro-pyridine-3-carboxylic acid 2-(1-methyl-pyrrolidin-2-yl)-ethyl ester;

2-Isopropyl-6-oxo-5-(2-pyridin-4-yl-thiazol-4-yl)-1,6-dihydro-pyridine-3-carboxylic acid methyl ester;

2-Isopropyl-6-oxo-5-(2-pyridin-4-yl-thiazol-4-yl)-1,6-dihydro-pyridine-3-carboxylic acid propyl ester;

2-Isopropyl-6-oxo-5-(2-pyridin-4-yl-thiazol-4-yl)-1,6-dihydro-pyridine-3-carboxylic acid butyl ester;

2-Isopropyl-6-oxo-5-(2-pyridin-4-yl-thiazol-4-yl)-1,6-dihydro-pyridine-3-carboxylic acid isobutyl ester;

2-Isopropyl-6-oxo-5-(2-pyridin-4-yl-thiazol-4-yl)-1,6-dihydro-pyridine-3-carboxylic acid sec-butyl ester;

5-{[(2-Diethylamino-ethyl)-methyl-amino]-methyl}-6-ethyl-3-(2-pyridin-4-yl-thiazol-4-yl)-1H-pyridin-2-one;

5-[2-(2-Dimethylamino-pyridin-4-yl)-thiazol-4-yl]-2-isopropyl-6-oxo-1,6-dihydro-pyridine-3-carboxylic acid ethyl ester;

Ethyl 2-ethyl-6-oxo-5-(2-(4-pyridinyl)-1,3-thiazol-4-yl)-1,6-dihydro-pyridine-3-carboxylate;

Ethyl 2-ethyl-6-oxo-5-{2-[(thienylsulfonyl)methyl](1,3-thiazol-4-yl)}-1,6-dihydro-pyridine-3-carboxylate;

Ethyl 2-ethyl-6-oxo-5-{2-[(phenylsulfonyl)methyl](1,3-thiazol-4-yl)}-1,6-dihydro-pyridine-3-carboxylate;

Ethyl 2-isopropyl-6-oxo-5-(2-(4-pyridyl)(1,3-thiazol-4-yl)-1,6-dihydro-pyridine-3-carboxylate;

Ethyl 2-isopropyl-6-oxo-5-{2-[(thienylsulfonyl)methyl](1,3-thiazol-4-yl)}-1,6-dihydro-pyridine-3-carboxylate;

Ethyl 2-isopropyl-6-oxo-5-{2-[(phenylsulfonyl)methyl](1,3-thiazol-4-yl)}-1,6-dihydro-pyridine-3-carboxylate;

Ethyl 2-propyl-6-oxo-5-(2-(4-pyridyl)(1,3-thiazol-4-yl)-1,6-dihydro-pyridine-3-carboxylate;

Ethyl 2-propyl-6-oxo-5-{2-[(phenylsulfonyl)methyl](1,3-thiazol-4-yl)}-1,6-dihydro-pyridine-3-carboxylate;

Ethyl 2-propyl-6-oxo-5-{2-[(thienylsulfonyl)methyl](1,3-thiazol-4-yl)}-1,6-dihydro-pyridine-3-carboxylate;

Ethyl 6-oxo-2-[(phenylmethoxy)methyl]-5-(2-(4-pyridyl)(1,3-thiazol-4-yl))-1,6-dihydro-pyridine-3-carboxylate;

Ethyl 6-oxo-2-[(phenylmethoxy)methyl]-5-{2-[(phenylsulfonyl)methyl](1,3-thiazol-4-yl)}-1,6-dihydro-pyridine-3-carboxylate;

Ethyl 2-methyl-6-oxo-5-{2-[(2-thienylsulfonyl)methyl](1,3-thiazol-4-yl)}-1,6-dihydro-pyridine-3-carboxylate;

Ethyl 5-[2-({[(4-fluorophenyl)methyl]sulfonyl}methyl)(1,3-thiazol-4-yl)]-2-methyl-6-oxo-1,6-dihydro-pyridine-3-carboxylate;

Ethyl 5-[2-({[(4-fluorophenyl)methyl]sulfonyl}methyl)(1,3-thiazol-4-yl)]-2-methyl-6-oxo-1,6-dihydro-pyridine-3-carboxylate;

Ethyl 2-methyl-6-oxo-5-{2-(phenylthiomethyl)(1,3-thiazol-4-yl)}-1,6-dihydro-pyridine-3-carboxylate;

Ethyl 5-[2-(2-ethyl(4-pyridyl))(1,3-thiazol-4-yl)-2-methyl-6-oxo-1,6-dihydro-pyridine-3-carboxylate;

Ethyl 5-[2-(2-chloro(4-pyridyl))(1,3-thiazol-4-yl)-2-methyl-6-oxo-1,6-dihydro-pyridine-3-carboxylate;

Ethyl 5-[2-(3,5-dichloro-pyridin-4-yl)-thiazol-4-yl]-2-methyl-6-oxo-1,6-dihydro-pyridine-3-carboxylate;

Ethyl 2-methyl-5-(2-(2-((2-methylpropyl)amino)-4-pyridinyl)-1,3-thiazol-4-yl)-6-oxo-1,6-dihydro-pyridine-3-carboxylate;

Ethyl 2-methyl-6-oxo-5-(2-(2-((3-pyridinylmethyl)amino)-4-pyridinyl)-1,3-thiazol-4-yl)-1,6-dihydro-pyridine-3-carboxylate;

Ethyl 2-methyl-6-oxo-5-(2-(2-((phenylmethyl)amino)-4-pyridinyl)-1,3-thiazol-4-yl)-1,6-dihydro-pyridine-3-carboxylate;

Ethyl 2-methyl-5-(2-(2-((2-((1-methylethyl)amino)ethyl)amino)-4-pyridinyl)-1,3-thiazol-4-yl)-6-oxo-1,6-dihydro-pyridine-3-carboxylate;

Ethyl 5-(2-(2-((2-(diethylamino)ethyl)amino)-4-pyridinyl)-1,3-thiazol-4-yl)-2-methyl-6-oxo-1,6-dihydro-pyridine-3-carboxylate;

Ethyl 5-(2-(2-[(fur-2-ylmethyl)-amino]-pyridin-4-yl)-thiazol-4-yl)-2-methyl-6-oxo-1,6-dihydro-pyridine-3-carboxylate;

Ethyl 5-{2-[2-(2-thien-2-yl-ethylamino)-pyridin-4-yl]-thiazol-4-yl}-2-methyl-6-oxo-1,6-dihydro-pyridine-3-carboxylate;

Ethyl 5-[2-(2-butylamino-pyridin-4-yl)-thiazol-4-yl]-2-methyl-6-oxo-1,6-dihydro-pyridine-3-carboxylate;

Ethyl 5-{2-[2-(carbamoylmethyl-amino)-pyridin-4-yl]-thiazol-4-yl}-2-methyl-6-oxo-1,6-dihydro-pyridine-3-carboxylate;

Ethyl 5-{2-[2-acetylamino-ethylamino)-pyridin-4-yl]-thiazol-4-yl}-2-methyl-6-oxo-1,6-dihydro-pyridine-3-carboxylate;

5-{2-[2-(Cyclopropylmethylamino)-pyridin-4-yl]-thiazol-4-yl}-2-methyl-6-oxo-1,6-dihydro-pyridine-3-carboxylic acid cyclopropyl-methyl amide;

Ethyl 5-{2-[2-(cyclopropylmethyl-amino)-pyridin-4-yl]-thiazol-4-yl}-2-methyl-6-oxo-1,6-dihydro-pyridine-3-carboxylate;

Ethyl 5-{2-[2-(cyclopentyl)methylamino-pyridin-4-yl]-thiazol-4-yl}-2-methyl-6-oxo-1,6-dihydro-pyridine-3-carboxylate;

Ethyl 2-methyl-6-oxo-5-(2-(2-(amino)-4-pyridinyl)-1,3-thiazol-4-yl)-1,6-dihydro-pyridine-3-carboxylate;

Ethyl 2-methyl-5-[2-(methylamino)(1,3-thiazol-4-yl)]-6-oxo-1,6-dihydro-pyridine-3-carboxylate;

Ethyl 2-methyl-6-oxo-5-{2-[(phenylsulfonyl)methyl](1,3-thiazol-4-yl)}-1,6-dihydro-pyridine-3-carboxylate;

Ethyl 2-methyl-6-oxo-5-(2-(4-pyridyl)(1,3-thiazol-4-yl))-1,6-dihydro-pyridine-3-carboxylate;

Ethyl 2-methyl-6-oxo-5-{2-[(2-pyridylsulfonyl)methyl](1,3-thiazol-4-yl)}-1,6-dihydro-pyridine-3-carboxylate;

Ethyl 2-methyl-5-(2-(1-methyl-1-(phenylsulfonyl)ethyl)-1,3-thiazol-4-yl)-6-oxo-1,6-dihydro-pyridine-3-carboxylate;

Ethyl 2-cyclopropyl-6-oxo-5-(2-((phenylsulfonyl)methyl)-1,3-thiazol-4-yl)-1,6-dihydro-pyridine-3-carboxylate;

5-Bromo-6-methyl-3-(2-(4-pyridinyl)-1,3-thiazol-4-yl)-2(1H)-pyridinone;

Ethyl 2-methyl-5-(2-(2-(methylamino)-4-pyridinyl)-1,3-thiazol-4-yl)-6-oxo-1,6-dihydro-pyridine-3-carboxylate;

2-Methyl-6-oxo-N-(2-pyridinylmethyl)-5-(2-(2-((2-pyridinylmethyl)amino)-4-pyridinyl)-1,3-thiazol-4-yl)-1,6-dihydro-pyridine-3-carboxamide;

Ethyl 2-methyl-6-oxo-5-(2-(2-((2-pyridinylmethyl)amino)-4-pyridinyl)-1,3-thiazol-4-yl)-1,6-dihydro-pyridine-3-carboxylate;

Ethyl 5-[2-(methylamino-pyridin-4-yl)-thiazol-4-yl]-2-isopropyl-6-oxo-1,6-dihydro-pyridine-3-carboxylate;

1,1-Dimethylethyl 2-methyl-6-oxo-5-(2-(4-pyridinyl)-1,3-thiazol-4-yl)-1,6-dihydro-pyridine-3-carboxylate;

2-(1-Pyrrolidinyl)ethyl 2-ethyl-6-oxo-5-(2-(4-pyridinyl)-1,3-thiazol-4-yl)-1,6-dihydro-pyridine-3-carboxylate;

6-Ethyl-3-(2-pyridin-4-yl-thiazol-4-yl)-1H-pyridin-2-one;

6-Isopropyl-3-(2-pyridin-4-yl-thiazol-4-yl)-1H-pyridin-2-one;

3-(Diethylamino)propyl 2-ethyl-6-oxo-5-(2-(4-pyridinyl)-1,3-thiazol-4-yl)-1,6-dihydro-pyridine-3-carboxylate; and

3-(Diethylamino)propyl 2-(1-methylethyl)-6-oxo-5-(2-(4-pyridinyl)-1,3-thiazol-4-yl)-1,6-dihydro-pyridine-3-carboxylate.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups from the genus. The phrase “Formula I-III” includes sub formulas such as I′ and II′.

Indications

Compounds of the present invention would be useful for, but not limited to, the treatment of cell proliferative diseases, cell death or of apoptosis.

The compounds of the invention are endowed with serine-threonine kinase inhibitory activity, such as CDK/cyclin kinase inhibitory activity.

The compounds of the invention are useful in therapy as antineoplasia agents.

Compounds of the invention would be useful for the treatment of neoplasia including cancer, including, but not limited to, carcinoma such as cancer of the bladder, breast, colon, kidney, liver, lung (including small cell lung cancer), esophagus, gall-bladder, ovary, pancreas, stomach, cervix, thyroid, prostate, and skin (including squamous cell carcinoma); hematopoietic tumors of lymphoid lineage (including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g. soft tissue and bone); tumors of the central and peripheral nervous system (including astrocytoma, neuroblastoma, glioma and schwannomas); and other tumors (including melanoma, seminoma, teratocarcinoma, osteosarcoma, xenoderoma pigmentosum, keratoctanthoma, thyroid follicular cancer and Kaposi's sarcoma).

Preferably, the compounds are useful for the treatment of neoplasia selected from lung cancer, colon cancer and breast cancer.

Due to the key role of CDKs in the regulation of cellular proliferation, these compounds are also useful in the treatment of a variety of cell proliferative disorders such as, for instance, blood vessel proliferative disorders including arthritis and restenosis; fibrotic disorders including hepatic cirrhosis and atherosclerosis; mesangial cell proliferative disorders including glomerulonephritis, diabetic nephropathy, malignant nephrosclerosis, thrombotic microangiopathy syndromes, transplant rejection and glomerulopathies; metabolic disorders including psoriasis, diabetes mellitus, chronic wound healing, inflammation, and diabetic retinopathy and other vision disorders; and others including benign prostate hyperplasia, familial adenomatosis polyposis, neuro-fibromatosis, pulmonary fibrosis, angiogenesis, metastasis, vascular smooth cell proliferation, post-surgical stenosis and hypertrophic scar formation, eczema, inflammatory bowel disease, endotoxic shock, and fungal infections.

The compounds of the invention are useful to prevent the phosphorylation of tau protein.

The compounds of the invention are useful in the treatment of neurological disorders, including neurological injuries and neurodegenerative diseases, such as, but not limited to, stroke, brain trauma, epilepsy, spinal cord injury, ischemia, multiple sclerosis, vision related disorders including but not limited to glaucoma and macular degeneration, hearing loss, AIDS-related dementia, retinitis pigmentosa, spinal muscular atrophy, cerebellar degeneration, amyotrophic lateral sclerosis, Parkinson's disease, Huntington's disease and Alzheimer's disease.

Compounds of Formula I-III, as inhibitors of the CDKs, can modulate the level of cellular RNA and DNA synthesis. These agents would therefore be useful in the treatment of viral infections, including but not limited to HIV, human papilloma virus, herpesvirus, poxvirus, Epstein-Barr virus, Sindbis virus and adenovirus.

The compounds of this invention may also act as inhibitors of other protein kinases, e.g. GSK, and thus be effective in the treatment of diseases associated with other protein kinases.

Besides being useful for human treatment, these compounds are also useful for veterinary treatment of companion animals, exotic animals and farm animals, including mammals, rodents, and the like. More preferred animals include horses, dogs, and cats.

Inhibitors of certain kinases may have utility in the treatment of diseases when the kinase is not misregulated, but is nonetheless essential for maintenance of the disease state. In this case, inhibition of the kinase activity would act either as a cure or palliative for these diseases. For example, many viruses, such as human papilloma virus, disrupt the cell cycle and drive cells into the S-phase of the cell cycle. Preventing cells from entering DNA synthesis after viral infection by inhibition of essential S-phase initiating activities such as CDK2, may disrupt the virus life cycle by preventing virus replication. This same principle may be used to protect normal cells of the body from toxicity of cycle-specific chemotherapeutic agents. Inhibition of CDK2 or CDK4 will prevent progression into the cycle in normal cells and limit the toxicity of cytotoxics which act in S-phase, G2 or mitosis. Furthermore, CDK2/cyclin E activity has also been shown to regulate NF-κB. Inhibition of CDK2 activity may have utility in cases where regulation of NF-κB plays a role in etiology of disease. A further example may be taken from fungal infections: Inhibition of the Aspergillus kinases Cdc2/CDC28 or Nim A may cause arrest or death in the fungi, improving the therapeutic outcome for patients with these infections.

The compounds of the invention are useful as modulators of apoptosis. As such they are useful in the prevention of AIDS development in HIV-infected individuals, autoimmune diseases (including but not limited to systemic lupus, erythematosus, autoimmune mediated glomerulonephritis, rheumatoid arthritis and autoimmune diabetes mellitus), myelodysplastic syndromes, aplastic anemia, ischemic injury associated with myocardial infarctions, stroke and reperfusion injury, vision related disorders including but not limited to glaucoma and macular degeneration, arrhythmia, atherosclerosis, toxin-induced or alcohol related liver diseases, hematological diseases (including but not limited to chronic anemia and aplastic anemia), degenerative diseases of the musculoskeletal system (including but not limited to osteoporosis) aspirin-sensitive rhinosinusitis, cystic fibrosis, kidney diseases and cancer pain.

Definitions

The phrase “therapeutically-effective” is intended to qualify the amount of each agent, which will achieve the goal of improvement in disorder severity and the frequency of incidence over treatment of each agent by itself, while avoiding adverse side effects typically associated with alternative therapies. For example, effective neoplastic therapeutic agents prolong the survivability of the patient, inhibit the rapidly-proliferating cell growth associated with the neoplasm, or effect a regression of the neoplasm. Alternatively, effective therapeutic agents for the treatment of neurological disorders minimize the damage from injury, improve cognitive functions, and the like.

The term “treatment” includes therapeutic treatment as well as prophylactic treatment (either preventing the onset of disorders altogether or delaying the onset of a preclinically evident stage of disorders in individuals).

The term “H” denotes a single hydrogen atom. This radical may be attached, for example, to an oxygen atom to form a hydroxyl radical.

Where the term “alkyl” is used, either alone or within other terms such as “haloalkyl”, “cyanoalkyl” and “alkylamino”, it embraces linear or branched radicals having one to about twenty carbon atoms or, preferably, one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. Even more preferred are lower alkyl radicals having one to four carbon atoms. The term “alkylenyl” embraces bridging divalent alkyl radicals such as methylenyl and ethyleneyl.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about four carbon atoms. Examples of alkenyl radicals include ethenyl, 2-propenyl, allyl, butenyl and 4-methylbutenyl. The terms “alkenyl” and “lower alkenyl”, embrace radicals having “cis” and “trans” orientations, or alternatively, “E” and “Z” orientations.

The term “alkynyl” denotes linear or branched radicals having at least one carbon-carbon triple bond and having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about four carbon atoms. Examples of such radicals include propargyl, butynyl, and the like.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals including perhaloalkyl. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Even more preferred are lower haloalkyl radicals having one to three carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl” means alkyl radicals having all hydrogen atoms replaced with fluoro atoms. Examples include trifluoromethyl and pentafluoroethyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “alkoxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. The “alkoxy” radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy, and fluoropropoxy.

The term “aryl”, alone or in combination, means a carbocyclic aromatic system containing one or two rings wherein such rings may be attached together in a pendent manner or may be fused. The term “aryl” embraces aromatic radicals such as phenyl, naphthyl, tetrahydronaphthyl, indane and biphenyl. More preferred aryl is phenyl. Said “aryl” group may have 1 to 3 substituents such as lower alkyl, hydroxyl, halo, haloalkyl, nitro, cyano, alkoxy, and lower alkylamino. Benzodioxolyl is considered aryl.

The term “heterocyclyl” embraces saturated, partially saturated and unsaturated heteroatom-containing ring radicals, where the heteroatoms may be selected from nitrogen, sulfur and oxygen. It does not include rings containing —O—O—, —O—S— or —S—S— portions. Said “heterocyclyl” group may have 1 to 3 substituents such as hydroxyl, halo, haloalkyl, cyano, lower alkyl, lower aralkyl, oxo, lower alkoxy, amino, and lower alkylamino.

Examples of saturated heterocyclic radicals include saturated 3 to 8-membered heteromonocyclic group containing 1 to 4 nitrogen atoms [e.g. pyrrolidinyl, imidazolidinyl, piperidino, piperazinyl]; saturated 3 to 8-membered heteromonocyclic group containing 1 to 2 oxygen atoms and 1 to 3 nitrogen atoms [e.g. morpholinyl]; saturated 3 to 8-membered heteromonocyclic group containing 1 to 2 sulfur atoms and 1 to 3 nitrogen atoms [e.g., thiazolidinyl]. Examples of partially saturated heterocyclyl radicals include dihydrothiophene, dihydropyran, dihydrofuran and dihydrothiazole.

Examples of unsaturated heterocyclic radicals, also termed “heteroaryl” radicals, include unsaturated 5 to 6 membered heteromonocyclyl groups containing 1 to 4 nitrogen atoms, for example, pyrrolyl, pyrrolinyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 3 to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

The term also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated condensed heterocyclic group containing 1 to 2 oxygen atoms and 1 to 3 nitrogen atoms [e.g. benzoxazolyl, benzoxadiazolyl]; unsaturated condensed heterocyclic group containing 1 to 2 sulfur atoms and 1 to 3 nitrogen atoms [e.g., benzothiazolyl, benzothiadiazolyl].

The term also includes bridged, spiro and oxo-containing heterocyclic rings, such as 1,4-dioxa-8-aza-spiro[4.5]decyl, phthalimidyl, 1,4-dioxa-8-aza-spiro[4.5]decyl, and (1-aza-bicyclo[2.2.2]oct-3-yl).

Preferred heterocyclic radicals include five to ten membered fused or unfused radicals. More preferred examples of heteroaryl radicals include quinolyl, isoquinolyl, imidazolyl, pyridyl, thienyl, thiazolyl, oxazolyl, furyl, and pyrazinyl. Even more preferred heteroaryl radicals are 5- or 6-membered heteroaryl, containing one or two heteroatoms selected from sulfur, nitrogen and oxygen, selected from thienyl, furanyl, pyrrolyl, thiazolyl, oxazolyl, imidazolyl, pyrazolyl, isoxazolyl, isothiazolyl, pyridyl, piperidinyl and pyrazinyl.

The term “sulfonyl”, whether used alone or linked to other terms such as alkylsulfonyl, denotes respectively divalent radicals —SO₂—.

The terms “sulfamyl,” “aminosulfonyl” and “sulfonamidyl,” whether alone or used with terms such as “N-alkylaminosulfonyl”, “N-arylaminosulfonyl”, “N,N-dialkylaminosulfonyl” and “N-alkyl-N-arylaminosulfonyl”, denotes a sulfonyl radical substituted with an amine radical, forming a sulfonamide (—SO₂NH₂).

The term “alkylaminosulfonyl” includes “N-alkylaminosulfonyl” and “N,N-dialkylaminosulfonyl” where sulfamyl radicals are independently substituted, respectively, with one alkyl radical, or two alkyl radicals. More preferred alkylaminosulfonyl radicals are “lower alkylaminosulfonyl” radicals having one to six carbon atoms. Even more preferred are lower alkylaminosulfonyl radicals having one to three carbon atoms. Examples of such lower alkylaminosulfonyl radicals include N-methylaminosulfonyl, N-ethylaminosulfonyl and N-methyl-N-ethylaminosulfonyl.

The terms “N-arylaminosulfonyl” and “N-alkyl-N-arylaminosulfonyl” denote sulfamyl radicals substituted, respectively, with one aryl radical, or one alkyl and one aryl radical. More preferred N-alkyl-N-arylaminosulfonyl radicals are “lower N-alkyl-N-arylsulfonyl” radicals having alkyl radicals of one to six carbon atoms. Even more preferred are lower N-alkyl-N-arylsulfonyl radicals having one to three carbon atoms. Examples of such lower N-alkyl-N-aryl-aminosulfonyl radicals include N-methyl-N-phenylaminosulfonyl and N-ethyl-N-phenylaminosulfonyl. Examples of such N-aryl-aminosulfonyl radicals include N-phenylaminosulfonyl.

The term “arylalkylaminosulfonyl” embraces aralkyl radicals as described above, attached to an aminosulfonyl radical. More preferred are lower arylalkylaminosulfonyl radicals having one to three carbon atoms.

The term “heterocyclylaminosulfonyl” embraces heterocyclyl radicals as described above, attached to an aminosulfonyl radical.

The terms “carboxy” or “carboxyl”, whether used alone or with other terms, such as “carboxyalkyl”, denotes —CO₂H.

The term “carbonyl”, whether used alone or with other terms, such as “aminocarbonyl”, denotes —(C═O)—.

The terms “alkylcarbonyl” denotes carbonyl radicals which have been substituted with an alkyl radical. More preferred are “lower alkylcarbonyl” having lower alkyl radicals as described above attached to a carbonyl radical.

The terms “arylcarbonyl” denotes carbonyl radicals substituted with an aryl radical. More preferred are “optionally substituted phenylcarbonyl” radicals.

The terms “cycloalkylcarbonyl” denotes carbonyl radicals substituted with an cycloalkyl radical. More preferred are “optionally substituted cycloalkylcarbonyl” radicals, even more preferably containing C₃₋₆ cycloalkyl.

The terms “heterocyclylcarbonyl” denotes carbonyl radicals substituted with an heterocyclyl radical. More preferred are “optionally substituted 5-6 membered heterocyclylcarbonyl” radicals.

The term “aminocarbonyl” when used by itself or with other terms such as “aminocarbonylalkyl”, “N-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl” and “N-alkyl-N-hydroxyaminocarbonylalkyl”, denotes an amide group of the formula H₂NC(═O)—.

The terms “N-alkylaminocarbonyl” and “N,N-dialkylaminocarbonyl” denote aminocarbonyl radicals which have been substituted with one alkyl radical and independently with two alkyl radicals, respectively. More preferred are “lower alkylaminocarbonyl” having lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical.

The term “aminoalkyl” embraces linear or branched alkyl radicals having one to about ten carbon atoms any one of which may be substituted with one or more amino radicals. More preferred aminoalkyl radicals are “lower aminoalkyl” radicals having one to six carbon atoms and one or more amino radicals. Examples of such radicals include aminomethyl, aminoethyl, aminopropyl, aminobutyl and aminohexyl. Even more preferred are lower aminoalkyl radicals having one to three carbon atoms.

The term “alkylaminoalkyl” embraces aminoalkyl radicals having the nitrogen atom independently substituted with an alkyl radical. More preferred alkylaminoalkyl radicals are “lower alkylaminoalkyl” radicals having alkyl radicals of one to six carbon atoms. Even more preferred are lower alkylaminoalkyl radicals having alkyl radicals of one to three carbon atoms. Suitable alkylaminoalkyl radicals may be mono or dialkyl substituted, such as N-methylaminomethyl, N,N-dimethyl-aminoethyl, N,N-diethylaminomethyl and the like.

The term “heterocyclylalkyl” embraces heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkyl radicals having alkyl portions of one to three carbon atoms. Examples include such radicals as pyridylmethyl and thienylmethyl.

The term “aralkyl” embraces aryl-substituted alkyl radicals. Preferable aralkyl radicals are “lower aralkyl” radicals having aryl radicals attached to alkyl radicals having one to six carbon atoms. Even more preferred are lower aralkyl radicals phenyl attached to alkyl portions having one to three carbon atoms. Examples of such radicals include benzyl, diphenylmethyl and phenylethyl. The aryl in said aralkyl may be additionally substituted with halo, alkyl, alkoxy, haloalkyl and haloalkoxy.

The term “arylalkenyl” embraces aryl-substituted alkenyl radicals. Preferable arylalkenyl radicals are “lower arylalkenyl” radicals having aryl radicals attached to alkenyl radicals having two to six carbon atoms. Examples of such radicals include phenylethenyl. The aryl in said arylalkenyl may be additionally substituted with halo, alkyl, alkoxy, haloalkyl and haloalkoxy.

The term “arylalkynyl” embraces aryl-substituted alkynyl radicals. Preferable arylalkynyl radicals are “lower arylalkynyl” radicals having aryl radicals attached to alkynyl radicals having two to six carbon atoms. Examples of such radicals include phenylethynyl. The aryl in said aralkyl may be additionally substituted with halo, alkyl, alkoxy, haloalkyl and haloalkoxy. The terms benzyl and phenylmethyl are interchangeable.

The term “alkylthio” embraces radicals containing a linear or branched alkyl radical, of one to ten carbon atoms, attached to a divalent sulfur atom. Even more preferred are lower alkylthio radicals having one to three carbon atoms. An example of “alkylthio” is methylthio, (CH₃S—).

The term “haloalkylthio” embraces radicals containing a haloalkyl radical, of one to ten carbon atoms, attached to a divalent sulfur atom. Even more preferred are lower haloalkylthio radicals having one to three carbon atoms. An example of “haloalkylthio” is trifluoromethylthio.

The term “alkylsulfinyl” embraces radicals containing a linear or branched alkyl radical, of one to ten carbon atoms, attached to a divalent —S(═O)— atom. More preferred are lower alkylsulfinyl radicals having one to three carbon atoms.

The term “arylsulfinyl”, embraces radicals containing an aryl radical, attached to a divalent —S(═O)— atom. Even more preferred are optionally substituted phenylsulfinyl radicals.

The term “haloalkylsulfinyl” embraces radicals containing a haloalkyl radical, of one to ten carbon atoms, attached to a divalent —S(═O)— atom. Even more preferred are lower haloalkylsulfinyl radicals having one to three carbon atoms.

The term “alkylamino” denotes amino groups which have been substituted with one alkyl radical and with two alkyl radicals, including terms “N-alkylamino” and “N,N-dialkylamino”. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable “alkylamino” may be mono or dialkylamino such as N-methylamino, N-ethylamino, N,N-dimethylamino, N,N-diethylamino and the like.

The term “arylamino” denotes amino groups which have been substituted with one or two aryl radicals, such as N-phenylamino. The “arylamino” radicals may be further substituted on the aryl ring portion of the radical.

The term “heteroarylamino” denotes amino groups which have been substituted with one or two heteroaryl radicals, such as N-thienylamino. The “heteroarylamino” radicals may be further substituted on the heteroaryl ring portion of the radical.

The term “aralkylamino” denotes amino groups which have been substituted with one or two aralkyl radicals. More preferred are phenyl-C₁-C₃-alkylamino radicals, such as N-benzylamino. The “aralkylamino” radicals may be further substituted on the aryl ring portion of the radical.

The term “alkylaminoalkylamino” denotes alkylamino groups which have been substituted with one or two alkylamino radicals. More preferred are C₁-C₃-alkylamino-C₁-C₃-alkylamino radicals.

The term “alkylaminoalkoxy” embraces alkoxy radicals substituted with alkylamino radicals. More preferred alkylaminoalkoxy radicals are “lower alkylaminoalkoxy” radicals having alkoxy radicals of one to six carbon atoms. Even more preferred are lower alkylaminoalkoxy radicals having alkyl radicals of one to three carbon atoms. Suitable alkylaminoalkoxy radicals may be mono or dialkyl substituted, such as N-methylaminoethoxy, N,N-dimethylaminoethoxy, N,N-diethylaminoethoxy and the like.

The terms “N-aralkyl-N-alkylamino” and “N-alkyl-N-arylamino” denote amino groups which have been substituted with one aralkyl and one alkyl radical, or one aryl and one alkyl radical, respectively, to an amino group.

The term “arylthio” embraces aryl radicals of six to ten carbon atoms, attached to a divalent sulfur atom. An example of “arylthio” is phenylthio.

The term “aralkylthio” embraces aralkyl radicals as described above, attached to a divalent sulfur atom. More preferred are phenyl-C₁-C₃-alkylthio radicals. An example of “aralkylthio” is benzylthio.

The term “aryloxy” embraces optionally substituted aryl radicals, as defined above, attached to an oxygen atom. Examples of such radicals include phenoxy.

The term “aralkoxy” embraces oxy-containing aralkyl radicals attached through an oxygen atom to other radicals. More preferred aralkoxy radicals are “lower aralkoxy” radicals having optionally substituted phenyl radicals attached to lower alkoxy radical as described above.

The term “heterocyclylalkoxy” embraces oxy-containing heterocyclylalkyl radicals attached through an oxygen atom to other radicals. More preferred heterocyclylalkoxy radicals are “lower heteroarylalkoxy” radicals having optionally substituted heteroaryl radicals attached to lower alkoxy radical as described above.

The term “heterocyclyloxyalkyl” embraces heteroaryl radicals attached through an ether oxygen atom to an alkyl radical. More preferred heterocyclyloxyalkyl radicals are “lower heteroaryloxyalkyl” radicals having optionally substituted heteroaryl radicals attached to an —O—C₁₋₆ alkyl radical.

The term “cycloalkyl” includes saturated carbocyclic groups. Preferred cycloalkyl groups include C₃-C₆ rings. More preferred compounds include cyclopentyl, cyclopropyl, and cyclohexyl.

The term “cycloalkenyl” includes carbocyclic groups have one or more carbon-carbon double bonds. “Cycloalkenyl” and “cycloalkyldienyl” compounds are included. Preferred cycloalkenyl groups include C₃-C₆ rings. More preferred compounds include, for example, cyclopentenyl, cyclopentadienyl, cyclohexenyl and cycloheptadienyl.

The term “comprising” is meant to be open ended, including the indicated component but not excluding other elements.

The present invention preferably includes compounds that inhibit CDK2 and/or CDK5.

The present invention also comprises the use of a compound of the invention, or pharmaceutically acceptable salt thereof, in the manufacture of a medicament for the treatment either acutely or chronically of a cell proliferation or apoptosis mediated disease state, including those described previously. The compounds of the present invention are also useful in the manufacture of an anti-cancer medicament. The compounds of the present invention are also useful in the manufacture of a medicament to attenuate or prevent disorders through inhibition of CDKs and other kinases. The compounds of the present invention are also useful in the manufacture of a medicament to treat neurological disorders.

The present invention comprises a pharmaceutical composition comprising a therapeutically-effective amount of a compound of Formulas I-III in association with at least one pharmaceutically-acceptable carrier, adjuvant or diluent.

The present invention also comprises a method of treating cell proliferative disorders, apoptosis mediated disorders, cancer, CDK mediated disorders or neurological disorders, in a subject, the method comprising treating the subject having or susceptible to such disorder with a therapeutically-effective amount of a compound of Formulas I-III.

Combinations

While the compounds of the invention can be administered as the sole active pharmaceutical agent, they can also be used in combination with one or more compounds of the invention or other agents. When administered as a combination, the therapeutic agents can be formulated as separate compositions that are administered at the same time or sequentially at different times, or the therapeutic agents can be given as a single composition.

The phrase “co-therapy” (or “combination-therapy”), in defining use of a compound of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent.

Specifically, the administration of compounds of the present invention may be in conjunction with additional therapies known to those skilled in the art in the treatment of neoplasia, such as with radiation therapy or with cytostatic or cytotoxic agents; or in the treatment of neurological disorders, such as with thrombolytic and anticoagulant agents, anti-inflammatory agents, NMDA inhibitors, anti-Parkinsonian agents, and inhibitors of lipid peroxidation.

If formulated as a fixed dose, such combination products employ the compounds of this invention within the accepted dosage ranges. Compounds of Formula I-III may also be administered sequentially with known agents when a combination formulation is inappropriate. The invention is not limited in the sequence of administration; compounds of the invention may be administered either prior to, at the same time with or after administration of the other agent.

Currently, standard treatment of primary tumors consists of surgical excision followed by either radiation or IV administered chemotherapy. The typical chemotherapy regime consists of either DNA alkylating agents, DNA intercalating agents or microtubule poisons. The chemotherapy doses used are just below the maximal tolerated dose and therefore dose limiting toxicities typically include, nausea, vomiting, diarrhea, hair loss, neutropenia and the like. Experiments performed in in vivo animal models and in in vitro cell based assays have demonstrated that combining chemotherapeutic agents with cell cycle inhibitors, such as CDK inhibitors, typically results in either decreased rate of tumor growth or, in some cases, tumor regression. Combining chemotherapy with a CDK inhibitor typically results in an increased therapeutic index and lower levels of both agents are required. This ultimately results in a decrease in toxicity and an increase in efficacy.

Schwartz et al, Clin. Can. Res., 3:1467-1472 (1997) have demonstrated that combining the CDK inhibitor flavopiridol with mitomycin-C (DNA alkylating agent) resulted in an increased rate of apoptosis in gastric and breast cancer cells. Bible et al., Cancer Res., 57:3375-3380 (1997) have also demonstrated therapeutic synergy exists between flavopiridol and paclitaxel, cytarabine, topotecan, doxorubicin, and etoposide (all standard chemotherapeutic agents) when tested in cell based assays using human non-small cell lung cancer cells. Preclinical models (cell culture) suggest that a cell cycle inhibitor potentiates the effect of a cytotoxic agent when administered after the chemotherapeutic agent. The chemotherapeutic agent will induce specific DNA/mitotic damage checkpoints in normal cells which in combination with a CDK inhibitor will cause a cell cycle arrest or cytostatic effect. In contrast, tumor cells will be driven into apoptosis or cell death when a chemotherapeutic agent and a CDK inhibitor are combined due to tumor cells attempting to activate defective DNA damage and cell cycle checkpoints. In addition, scheduling of a CDK inhibitor for clinical trials should include a rest period to allow the patients normal cells to recover and reduce the potential for cytotoxic side effects.

There are large numbers of antineoplastic agents available in commercial use, in clinical evaluation and in pre-clinical development, which would be selected for treatment of neoplasia by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents.

A first family of antineoplastic agents which may be used in combination with compounds of the present invention consists of antimetabolite-type/thymidilate synthase inhibitor antineoplastic agents. Suitable antimetabolite antineoplastic agents may be selected from but not limited to the group consisting of 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrill Dow DDFC, deazaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine protein kinase inhibitors, Taiho UFT and uricytin.

A second family of antineoplastic agents which may be used in combination with compounds of the present invention consists of alkylating-type antineoplastic agents. Suitable alkylating-type antineoplastic agents may be selected from but not limited to the group consisting of Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

A third family of antineoplastic agents which may be used in combination with compounds of the present invention consists of antibiotic-type antineoplastic agents. Suitable antibiotic-type antineoplastic agents may be selected from but not limited to the group consisting of Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thriazine, tricrozarin A, Upjohn U-73975, Kyowa Hakko UCN-10028A, Fujisawa WF-3405, Yoshitomi Y-25024 and zorubicin.

A fourth family of antineoplastic agents which may be used in combination with compounds of the present invention consists of a miscellaneous family of antineoplastic agents, including tubulin interacting agents, topoisomerase II inhibitors, topoisomerase I inhibitors and hormonal agents, HDAC inbitors, EGF inhibitors, ErbB inhibitos, Her2 inhibitors, selected from but not limited to the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B. cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, herceptin, hexadecylphosphocholine, Green Cross HO-221, homoharringtonine, hydroxyurea, BTG ICRF-187, Iressa, ilmofosine, isoglutamine, isotretinoin, Otsuka JI-36, Ramot K-477, Otsuak K-76COONa, Kureha Chemical K-AM, MECT Corp KI-8110, American Cyanamid CL-623, leukoregulin, lonidamine, Lundbeck LU-23-112, Lilly LY-186641, NCI (US) MAP, marycin, Merrel Dow MDL-27048, Medco MEDR-340, merbarone, merocyanlne derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SAHA, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides and Yamanouchi YM-534.

Alternatively, the present compounds may also be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celecoxib, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Alternatively, the present compounds may also be used in co-therapies with other anti-neoplastic agents, such as other kinase inhibitors including KDR inhibitors, p38 inhibitors, TNF inhibitors, metallomatrix proteases inhibitors (MMP), COX-2 inhibitors, NSAID's, SOD mimics or αᵥβ₃ inhibitors.

Alternatively, the present compounds may also be used in co-therapies with other treatments for neurological treatments such as thrombolytic and anticoagulant agents including tPA, urokinase and inhibitors of platelet aggregation, p38 inhibitors, IL1ra, NMDA inhibitors, anti-Parkinsonian agents including carbidopa and levodopa, and inhibitors of lipid peroxidation, for example.

The present invention comprises a process for the preparation of a compound of Formula I-III.

Compounds of the present invention can possess, in general, one or more asymmetric carbon atoms and are thus capable of existing in the form of optical isomers as well as in the form of racemic or non-racemic mixtures thereof. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using optically active starting materials. These isomers may be in the form of a free acid, a free base, an ester or a salt.

Compounds of the present invention can possess, in general, tautomeric forms, which are included in the family of compounds in Formula I-III.

Also included in the family of compounds of Formula I-III are the pharmaceutically-acceptable salts thereof. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I-III may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic carboxylic and sulfonic classes of organic acids, example of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, β-hydroxybutyric, salicylic, galactaric and galacturonic acid. Suitable pharmaceutically-acceptable base addition salts of compounds of Formula I-III include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethylmorpholine, piperazine, piperidine, triethylamine, trimethylamine. All of these salts may be prepared by conventional means from the corresponding compound of the invention by reacting, for example, the appropriate acid or base with the compound of Formula I-III.

Also, the basic nitrogen-containing groups can be quaternized with such agents as lower alkyl halides, such as methyl, ethyl, propyl, and butyl chloride, bromides and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids that may be employed to from pharmaceutically acceptable acid addition salts include such inorganic acids as HCl, H₂SO₄ and H₃PO₄ and such organic acids as oxalic acid, maleic acid, succinic acid and citric acid. Other examples include salts with alkali metals or alkaline earth metals, such as sodium, potassium, calcium or magnesium or with organic bases.

Additional examples of such salts can be found in Berge et al., J. Pharm. Sci., 66:1 (1977).

### General Synthetic Procedures

The compounds of the invention can be synthesized according to the following procedures of Schemes 1-12, wherein the substituents are as defined above, except where further noted. The following abbreviations are used:

AcOH, HOAc—acetic acid

Ac₂O—acetic anhydride

CH₃CN—acetonitrile

NH₃—ammonia

NH₄OAc—ammonium acetate

NH₄OH—ammonium hydroxide

BCl₃—boron trichloride

Br₂—bromine

BuLi—butyllithium

CDI—carbonyl diimidazole

CHCl₃—chloroform

Cu—copper

DDQ—2,3-dichloro-5,6-dicyano-1,4-benzoquinone

CH₂Cl₂—dichloromethane

Et₂O—diethyl ether

DMAP—4-(dimethylamino)pyridine

DIAD—diisopropyl azodicarboxylate

DIPEA, DIEA—diisopropylethylamine

Me₂NH—dimethylamine

dppa—diphenylphosphoryl azide

DMF—dimethylformamide

DMSO—dimethylsulfoxide

EtOAc—ethyl acetate

EtOH—ethanol

g—gram

h—hour

HCl—hydrochloric acid

H₂S—hydrogen sulfide

iPrOH—isopropanol

LDA—lithium diisopropylamide

MeOH—methanol

mL—milliliter

min—minutes

MnO₂—manganese oxide

MgSO₄—magnesium sulfate

MeI—methyl iodide

MeMgBr—methyl magnesium bromide

NBS—N-bromosuccinimide

P₂S₅—phosphorous pentasulfide

K₂CO₃—potassium carbonate

KOH—potassium hydroxide

KSCN—potassium thiocyanate

Py—pyridine

RT—room temperature

SiO₂—silica

NaHCO₃—sodium bicarbonate

NaBH₄—sodium borohydride

Na₂CO₃—sodium carbonate

NaOEt—sodium ethoxide

Na₂SO₄—sodium sulfate

NaH—sodium hydride

NaOH—sodium hydroxide

NaBH(OAc)₃—sodium triacetoxyborohydride

HBF₄—tetrafluoroboric acid

TFA—trifluoroacetic acid

THF—tetrahydrofuran

(Ph₃P)₄Pd—terakis(triphenylphosphine)palladium(0)

TEA, Et₃N—triethylamine

H₂O—water

ZnBr₂—zinc bromide

ZnCl₂—zinc chloride

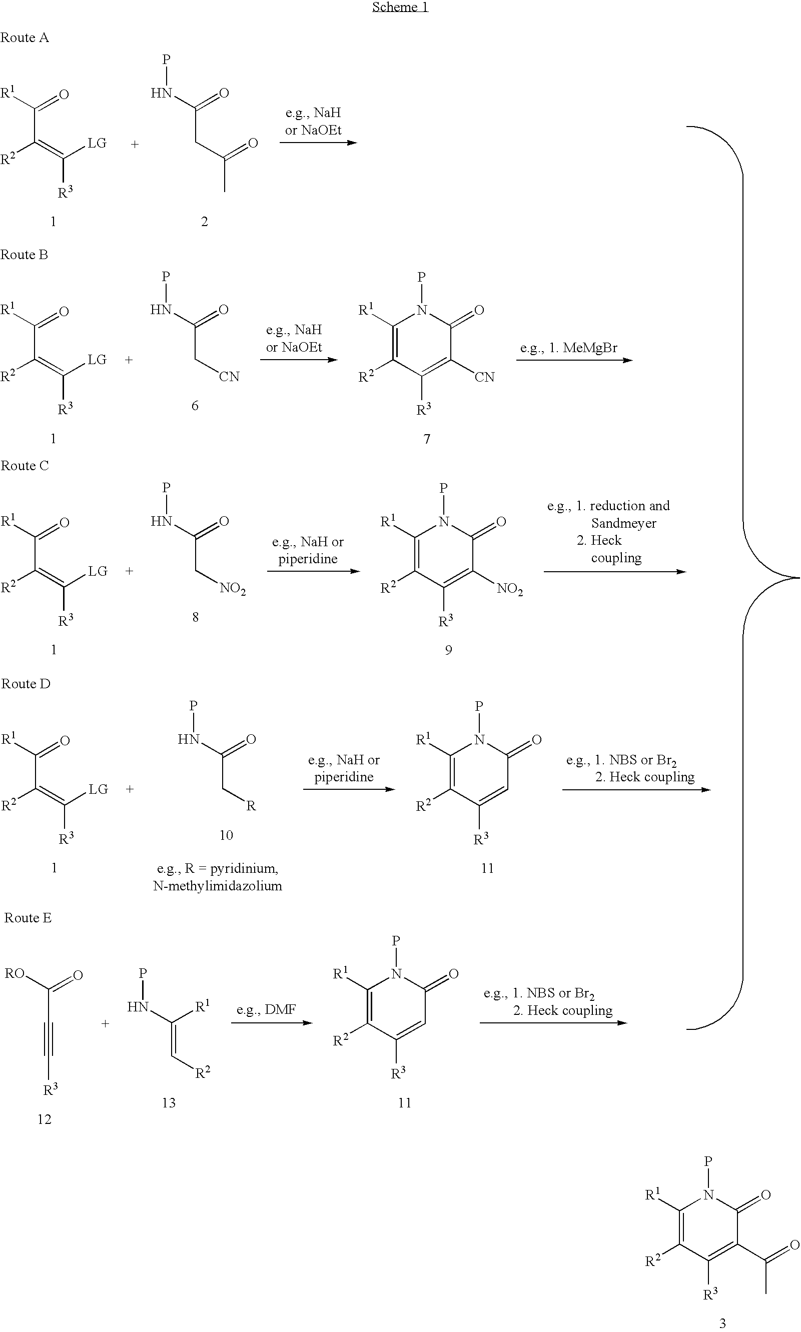

3-Acetyl-pyrid-2-one derivatives 3 can be synthesized according to the methods set out in Scheme 1 (where P is H, a protecting group, or a polymer and LG is a leaving group (e.g., —NMe₂, —OR, —ONa, —OTf, or halogen (where R is lower alkyl, allyl or benzyl, etc.)). Following Route A, acetoacetamide 1 (preferably in an excess) in a dry solvent such as THF, is reacted with base, such as NaH or NaOEt (preferably about 0.8-1.0 eq.), then with a prop-2-enoate 2 (preferably in an excess), preferably at a temperature above RT and more preferably at temperature of about 60° C. to form the 3-acetylpyrid-2-one 3. Alternately, 3-acetyl-pyrid-2-one derivatives 3 can be formed through the 5-cyanopyridone 7 (Route B), the 5-nitropyridone (Route C), or the pyridone (Routes D and E) (where R is lower alkyl) and the appropriate starting materials.

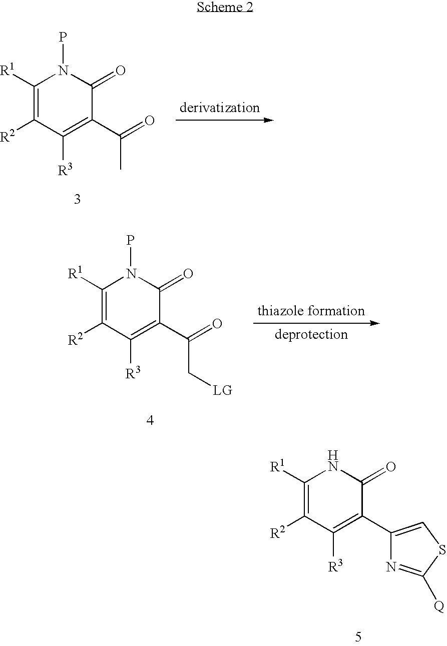

3-(2-Substituted thiazol-4-yl)pyrid-2-one derivatives 5 can be synthesized according to the methods set out in Scheme 2 (where P is H, a protecting group, or a polymer and LG is a leaving group (e.g., —NMe₂, —OR, —ONa, —OTf, halogen (where R is e.g., lower alkyl, allyl, benzyl))). Derivatization of the 3-acetylpyrid-2-one 3, such as halogenation, e.g. treatment with 5,5′-dibromobarbituric acid in a dry solvent, such as THF, preferably at a temperature above RT and more preferably at temperature of about 60° C. forms the 3-derivatized pyrid-2-one 4. The 3-(2-substituted thiazol-4-yl)pyrid-2-one 5 is formed by treatment of 3-derivatized pyrid-2-one 4 with substituted thioamides (preferably more than 1 eq.), in a solvent, such as an alcohol, preferably EtOH, such as in a microwave synthesizer, preferably at a temperature above RT, more preferably at temperature above about 100° C. and even more preferably at temperature of about 150° C.

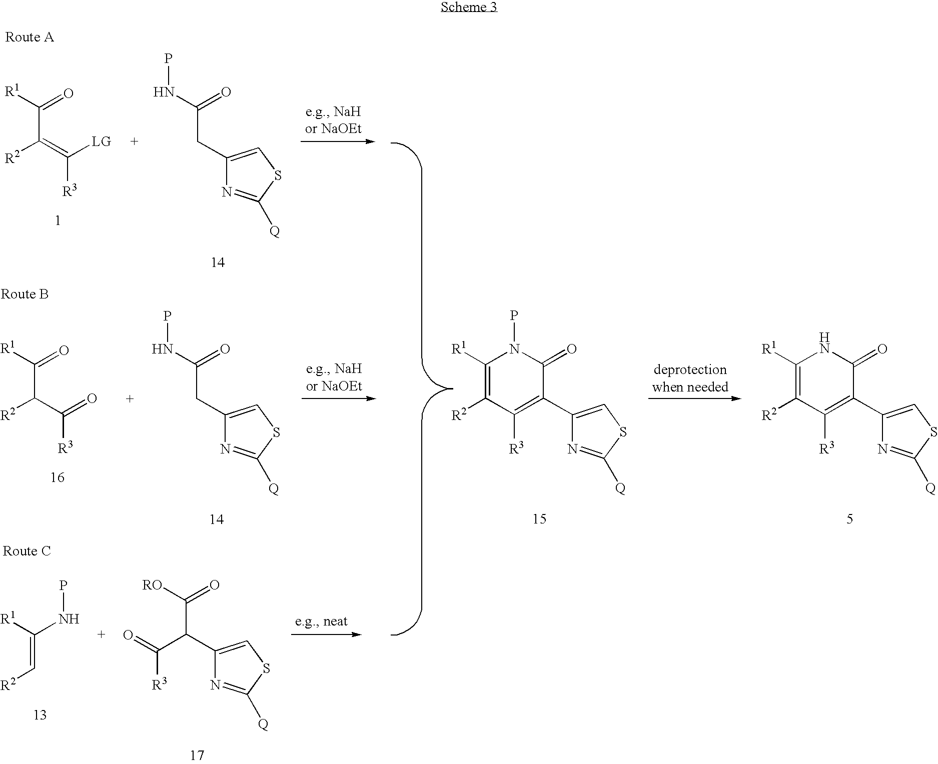

3-(2-Substituted thiazol-4-yl)pyrid-2-one derivatives 5 also can be synthesized according to the methods set out in Scheme 3 (where P is H, a protecting group, or a polymer; and LG is a leaving group (e.g., —NMe₂, —OR, —ONa, —OTf, or halogen (where R is e.g., lower alkyl, allyl, benzyl))). Following Route A, acetoacetamide 1 is reacted with substituted thiazolylmethylamides 14, and with base, such as NaH or NaOEt, to form the protected 3-thiazolylpyridone 15. Deprotection of protected 3-thiazolylpyridone 15 yields 3-(2-substituted thiazol-4-yl)pyrid-2-one derivatives 5. Alternatively, following Route B, protected 3-thiazolylpyridone 15 can be prepared from reaction of substituted thiazolylmethylamides 14 and diones 16 with base, such as NaH or NaOEt. According to Route C, 2-(thiazolyl)-3-oxo-propionic acid ester 17 (where R is lower alkyl) can be reacted with aminoalkenes 13 to form protected 3-thiazolylpyridone 15.

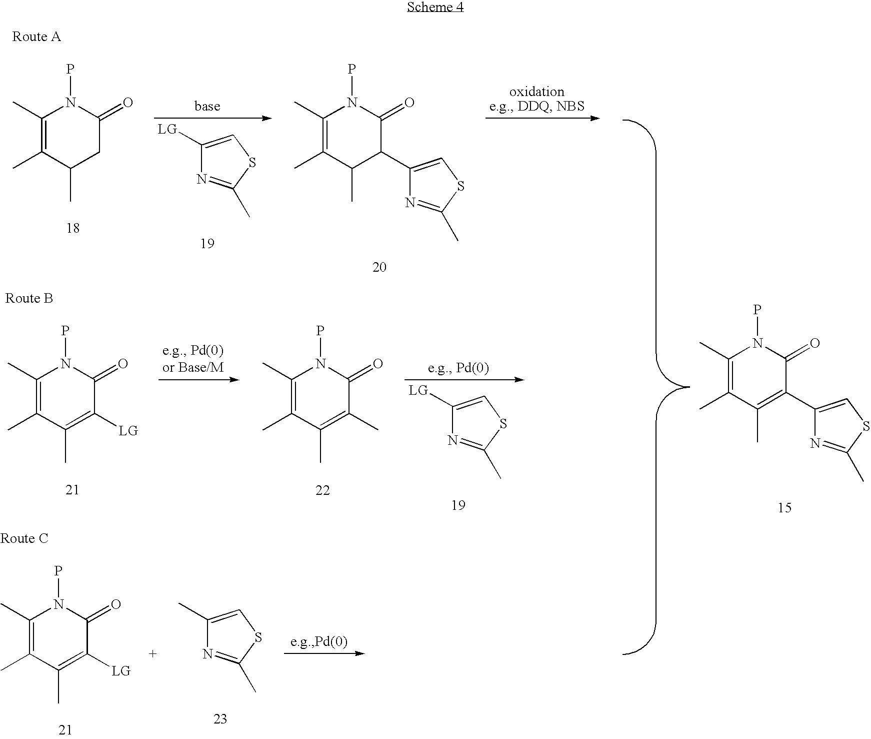

Protected 3-(2-substituted thiazol-4-yl)pyrid-2-one derivatives 15 also can be synthesized according to the methods set out in Scheme 4 (where P is H, a protecting group, or a polymer; M is for example B(OR)₂, SnR₃, ZnCl, or ZnBr; and LG is a leaving group (e.g., —NMe₂, —OR, —ONa, —OTf, or halogen (where R is e.g., lower alkyl, allyl, benzyl))). Following Route A, 3,4-dihydro-pyridones are coupled with a thiazole 19, such as with base treatment, to yield 3,4-dihydro-3-(2-substituted thiazol-4-yl)pyrid-2-one derivatives 20. The 3,4-dihydro-3-(2-substituted thiazol-4-yl)pyrid-2-one derivatives 20 are oxidized, such as in the presence of DDQ or NBS, to provide N-protected 3-(2-substituted thiazol-4-yl)pyrid-2-one derivatives 15.

Alternatively, pyrid-2-one derivatives 21 can be converted to activated pyridones 22. The activated pyridones 22 are then coupled with thiazolyl derivatives 19, such as in the presence of a Pd catalyst to yield pyrid-2-one derivatives 15.

Pyrid-2-one derivatives 15 can also be prepared directly by coupling N-protected pyrid-2-one derivatives 21 with activated thiazolyl derivatives 23, such as in the presence of a Pd catalyst.

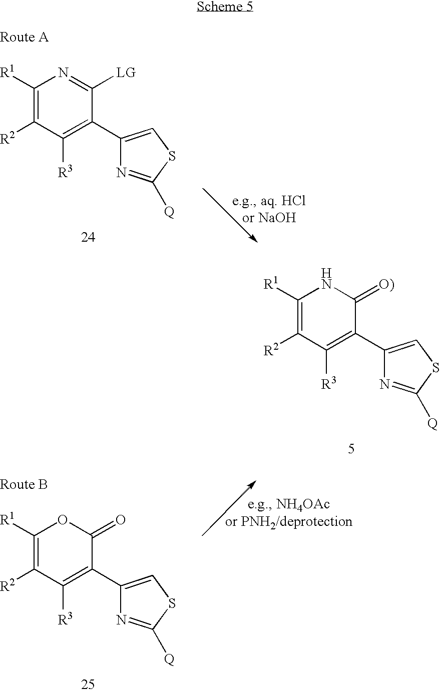

3-(2-Substituted thiazol-4-yl)pyrid-2-one derivatives 5 also can be synthesized according to the methods set out in Scheme 5 (where P is H, a protecting group, or a polymer; and where LG is a halogen, —OR (where R is e.g., lower alkyl, allyl, benzyl) or —S(O)ₙR^{a}) (where R^{a} is e.g., lower alkyl, benzyl, tosyl)). 3-(2-Substituted thiazol-4-yl)pyrid-2-one derivatives 5 can be prepared from the corresponding pyridines such as by treatment with acid or base (Route A). Alternatively, 3-(2-substituted thiazol-4-yl)pyrid-2-one derivatives 5 can be prepared by treatment of pyran-2-one 25 with ammonium acetate or with protected amines and a corresponding deprotection step.

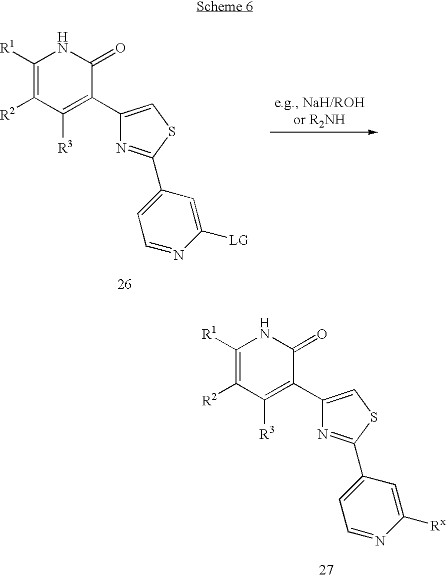

3-(2-(2-Substituted-pyridyl)-thiazol-4-yl)pyrid-2-one derivatives 27 can be synthesized according to the method set out in Scheme 6 (where LG is a halogen or —S(O)ₙR, where R^{x} is —OR, —NR₂ or heterocyclyl, and where R is e.g., optionally substituted alkyl or optionally substituted aryl) where 3-(2-(2-substituted-pyridyl)-thiazol-4-yl)pyrid-2-one derivatives 26 are treated with base and with an alcohol, or alternatively with an amine.

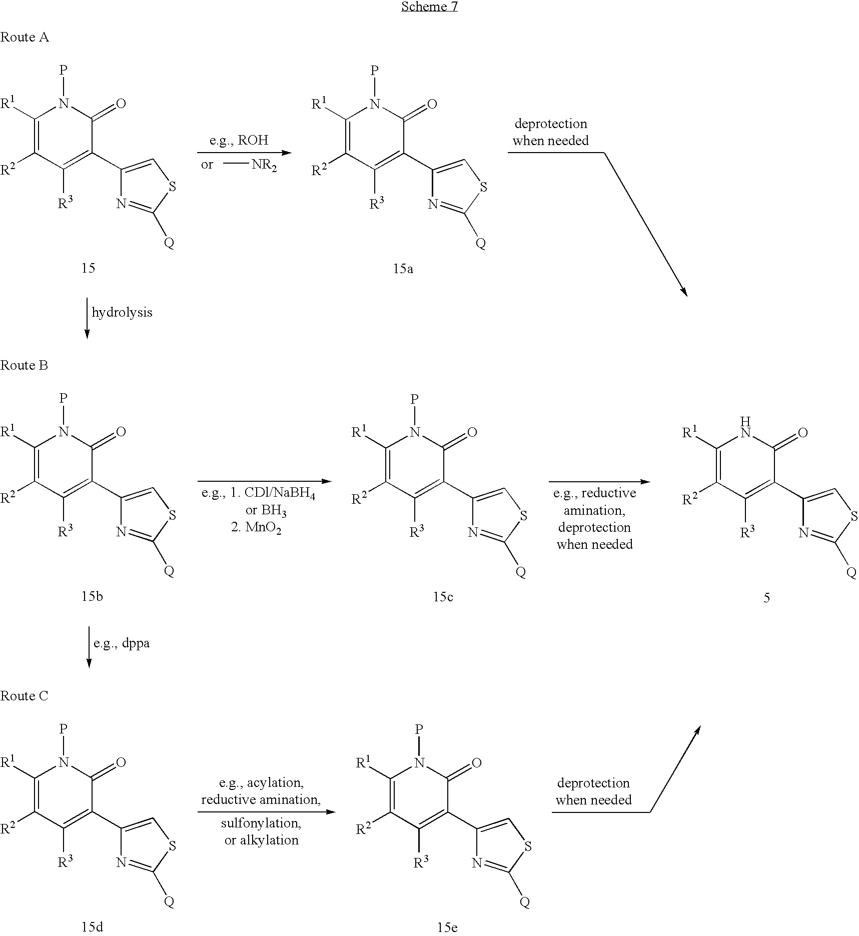

3-(2-Substituted thiazol-4-yl)pyrid-2-one derivatives 5 can be synthesized according to the methods set out in Scheme 7. Protected 3-thiazolylpyridone 15 (where P is H, a protecting group, or a polymer; and R¹, R² or R³ is an ester) is hydrolyzed to yield the corresponding acids 15b (where P is H, a protecting group, or a polymer and R¹, R² or R³ is CO₂H). The acids 15b can be reduced to the corresponding alcohol and then oxidized to the corresponding aldehydes 15c (where P is H, a protecting group, or a polymer; and R¹, R² or R³ is CHO) as shown in Route B. The acids 15b can be converted to the corresponding amines 15d (where P is H, a protecting group, or a polymer; and R¹, R² or R³ is —N(R⁵)₂ (where R⁵ is alkyl, aryl, and the like)). The amine 15d can be derivatized as shown in Route C. The protected 3-thiazolylpyridone 15 can also be converted to other esters or amides 15a (where P is H, a protecting group, or a polymer; and R¹, R² or R³ is —CO₂R⁵ or —CO₂N(R⁵)₂) as provided in Route A.

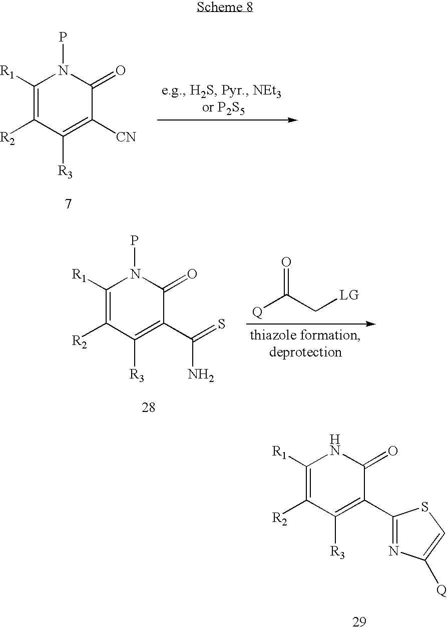

3-(4-Substituted thiazol-2-yl)pyrid-2-one derivatives 29 can be synthesized from the corresponding 3-cyanopyrid-2-ones according to the method set out in Scheme 8. Thioamides 28 are prepared from the 3-cyano-pyrid-2-one 7 (where P is H, a protecting group, or a polymer) such as by the addition of H₂S and a base, such as Et₃N, preferably an excess of base. The thioamide 28 is converted to the protected thiazole such as by the treatment with an acylating agent (where LG is a leaving group, such as halogen, —OTs, —OMs, and —OTf), such as an acyl bromide, in a solvent, such as an alcohol, preferably EtOH. A microwave synthesizer can be used in the preparation of the thiazole. Deprotection yields the 3-(4-substituted thiazol-2-yl)pyrid-2-one derivative 29.

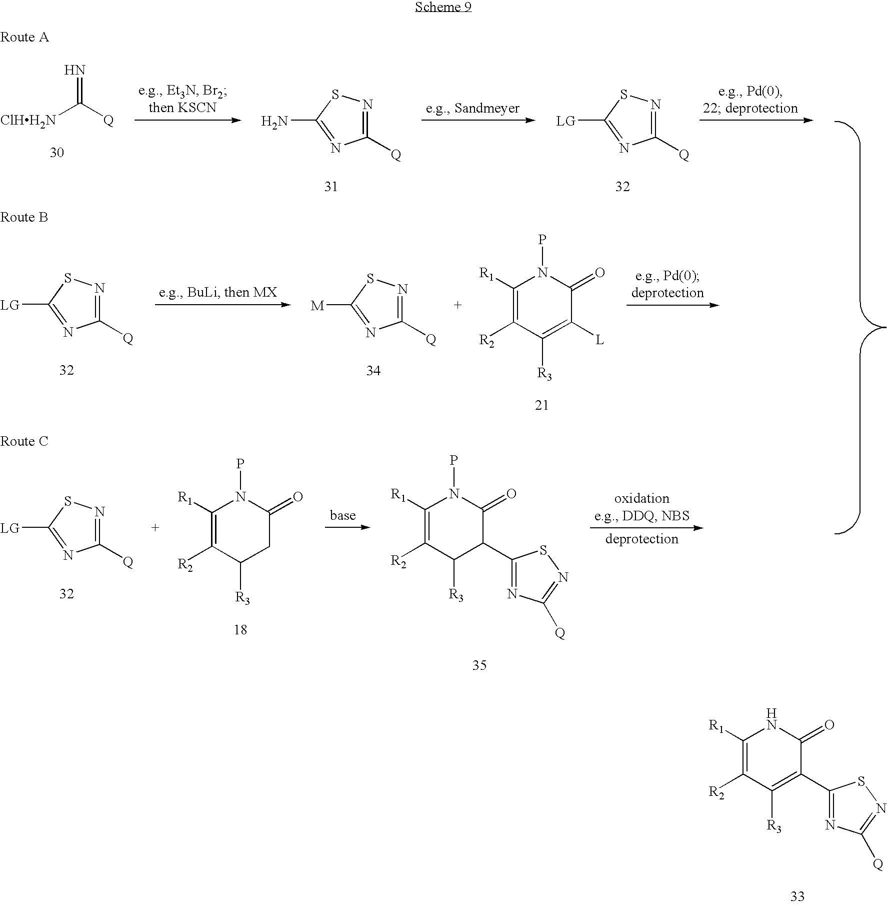

Protected 3-(3-substituted thiadiazol-5-yl)pyrid-2-one derivatives 33 can be synthesized according to the methods set out in Scheme 9 (where P is H, a protecting group, or a polymer; and LG is a leaving group (e.g., —OTf, halogen)). Following Route A, substituted 2-amino-thiadiazole 31 is formed, such as from the corresponding amidine 30, then derivatized to form the 2,4-substituted thiadiazole 32. The 2,4-substituted thiadiazole 32 is coupled with activated pyridones 22 such as in the presence of a Pd catalyst, to yield pyrid-2-one derivatives 33.

Alternatively, following Route B, 2,4-substituted thiadiazole 32 can be converted to activated thiadiazoles 34, where M is for example B(OR)₂, SnR₃, ZnCl, or ZnBr. The activated thiadiazoles 34 are then coupled with activated pyridones 22 (where L is e.g. Br, I, —OTf, etc.) such as in the presence of a Pd catalyst to yield pyrid-2-one derivatives 33.

Following Route C, pyrid-2-one derivatives 33 can also be prepared from 3,4-dihydro-3-(3-substituted thiadiazol-5-yl)pyrid-2-one derivatives 35 such as by oxidation, e.g. in the presence of DDQ or NBS. The 3,4-dihydro-(3-substituted thiadiazol-5-yl)pyrid-2-one derivatives 35 are prepared from the coupling of 3,5-substituted thiadiazole 32 and N-protected 3,4-dihydro-pyrid-2-one derivative 18, such as by base mediated coupling.

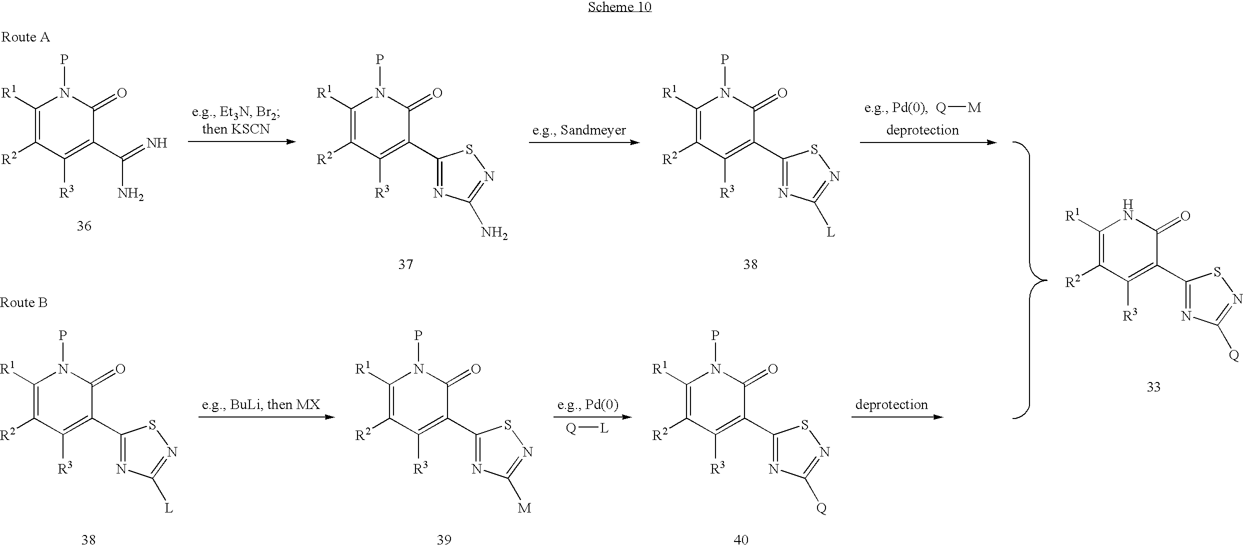

3-(3-Substituted thiadiazol-5-yl)pyrid-2-one derivatives 33 also can be synthesized according to the methods set out in Scheme 10 (where P is H, a protecting group, or a polymer; where M is for example B(OR)₂, SnR₃, ZnCl, or ZnBr; and L is a leaving group (e.g., —OTf, halogen)). Following Route A, substituted 4-amino-2-thiadiazole 37 is formed, such as from the corresponding amidine 36, then derivatized to form the (3-substituted thiadiazol-5-yl)pyrid-2-one 38. The (3-substituted thiadiazol-5-yl)pyrid-2-one 38 is coupled with Q-M, such as in the presence of a Pd catalyst, and deprotected to yield pyrid-2-one derivatives 33.

Alternatively, following Route B, (3-substituted thiadiazol-5-yl)pyrid-2-one 38 can be converted to activated (thiadiazol-5-yl)pyrid-2-one 39. The activated thiadiazoles 39 are then coupled with Q-L, such as in the presence of a Pd catalyst to yield protected pyrid-2-one derivatives 40. Deprotection provides the 3-(4-substituted thiadiazol-2-yl)pyrid-2-one derivatives 33.

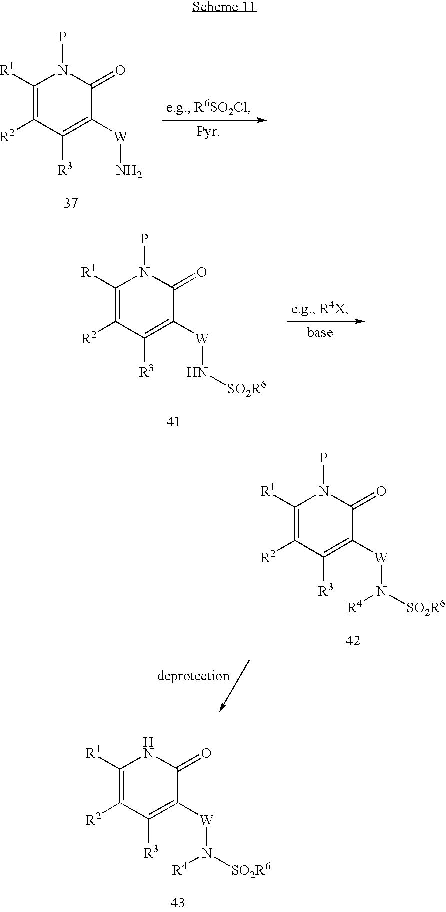

Sulfonamidyl substituted pyrid-2-one derivatives 43 (compounds of Formula I where Q is

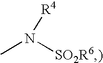

can be synthesized according to the methods set out in Scheme 11 (where P is H, a protecting group, or a polymer). Amines 37 are reacted with substituted sulfones to provide the sulfonamide 41. Disubstituted sulfonamides 42 are prepared by alkylation of sulfonamides 41. Deprotection of either disubstituted sulfonamides 42 or sulfonamides 41 provides sulfonamidyl substituted pyrid-2-one derivatives 43.

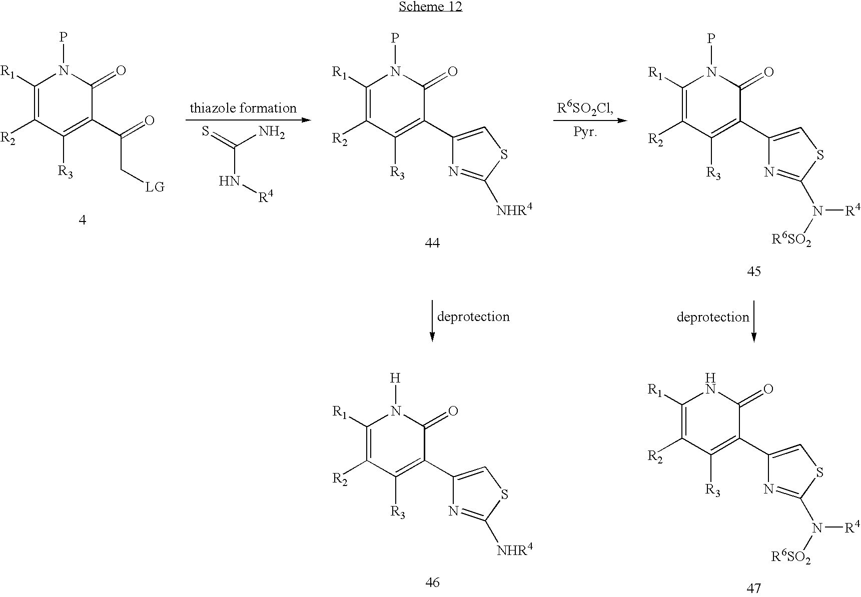

3-(2-Aminosubstituted thiazol-4-yl)pyrid-2-one derivatives 46 and 47 can be synthesized according to the methods set out in Scheme 12 (where P is H, a protecting group, or a polymer and LG is a leaving group (e.g., —OTs, —OMs, —OTf, halogen)). The protected 3-(2-substituted thiazol-4-yl)pyrid-2-one 44 is formed by treatment of 3-acetylpyrid-2-one derivative 4 with substituted thioureas. 3-(2-Substituted thiazol-4-yl)pyrid-2-one 44 can be deprotected to form the amine 46 or further treated with reagents, such as substituted sulfonyl chlorides, to form sulfonamides 47.

In the preparation of starting materials, existing functional groups, for example carboxy, hydroxy, amino, or mercapto, which do not participate in the reaction should, if necessary, be protected. Such protecting groups are those or similar to those usually used in the synthesis of peptide compounds, cephalosporins, penicillins, nucleic acid derivatives or sugars. Preferred protecting groups, their introduction and their removal are described above or in the examples.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves to ready removal, i.e. without undesired secondary reactions, typically by solvolysis, reduction, photolysis, or also by enzyme activity, for example under conditions analogous to physiological conditions, and that they are not present in the end-products. One skilled in the art knows, or can easily establish, which protecting groups are suitable with the reactions mentioned above and hereinafter.

The protection of such functional groups by such protecting groups, the protecting groups themselves, and their removal reactions are described for example in standard reference works, such as J. F. W. McOmie, “Protective Groups in Organic Chemistry”, Plenum Press, London and New York (1973); in T. W. Greene, “Protective Groups in Organic Synthesis”, Wiley, New York, 3^{rd} Edition, (1999); in “The Peptides”; Volume 3 (editors: E. Gross and J. Meienhofer), Academic Press, London and New York (1981); in “Methoden der organischen Chemie” (Methods of organic chemistry), Houben Weyl, 4th edition, Volume 15/1, Georg Thieme Verlag, Stuttgart (1974); in H.-D. Jakubke and H. Jescheit, “Aminosäuren, Peptide, Proteine” (Amino acids, peptides, proteins), Verlag Chemie, Weinheim, Deerfield Beach, and Basel (1982); and in Jochen Lehmann, “Chemie der Kohlenhydrate: Monosaccharide und Derivate” (Chemistry of carbohydrates: monosaccharides and derivatives), Georg Thieme Verlag, Stuttgart (1974).

In the additional process steps, carried out as desired, functional groups of the starting compounds which should not take part in the reaction may be present in unprotected form or may be protected for example by one or more of the protecting groups mentioned above. The protecting groups are then wholly or partly removed according to one of the methods previously described.

In certain cases, typically in hydrogenation processes, it is possible to achieve stereoselective reactions, allowing for example easier recovery of individual isomers.

The solvents from which those can be selected which are suitable for the reaction in question include, for example, water, esters, typically lower alkyl-lower alkanoates, e.g. EtOAc, ethers, typically aliphatic ethers, e.g. Et₂O, or cyclic ethers, e.g. THF, liquid aromatic hydrocarbons, typically benzene or toluene, alcohols, typically MeOH, EtOH or 1-propanol or iPrOH, nitrites, typically CH₃CN, halogenated hydrocarbons, typically CH₂Cl₂, carboxamides, typically DMF, bases, typically heterocyclic nitrogen bases, e.g. pyridine, carboxylic acids, typically lower alkanecarboxylic acids, e.g. AcOH, carboxylic acid anhydrides, typically lower alkyl acid anhydrides, e.g. Ac₂O, cyclic, linear, or branched hydrocarbons, typically cyclohexane, hexane, or isopentane, or mixtures of these solvents, e.g. aqueous solutions, unless otherwise stated in the description of the process.

The invention relates also to those forms of the process in which one starts from a compound obtainable at any stage as a transient and carries out the missing steps, or breaks off the process at any stage, or forms a starting material under the reaction conditions, or uses said starting material in the form of a reactive derivative or salt, or produces a compound obtainable by means of the process according to the invention and processes the said compound in situ. In the preferred embodiment, one starts from those starting materials which lead to the compounds described above as preferred.

The compounds of Formula I-III, including their salts, are also obtainable in the form of hydrates, or their crystals can include for example the solvent used for crystallization (present as solvates).

New starting materials and/or intermediates, as well as processes for the preparation thereof, are likewise the subject of this invention. In the preferred embodiment, such starting materials are used and reaction conditions so selected as to enable the preferred compounds to be obtained.

Starting materials of the invention, are known, are commercially available, or can be synthesized in analogy to or according to methods that are known in the art.

All remaining starting materials are known, capable of being prepared according to known processes, or commercially obtainable; in particular, they can be prepared using processes as described above or as in the examples.

The compounds of this invention may contain one or more asymmetric centers and thus occur as racemates and racemic mixtures, scalemic mixtures, single enantiomers, individual diastereomers and diastereomeric mixtures. All such isomeric forms of these compounds are expressly included in the present invention.

The compounds of this invention may also be represented in multiple tautomeric forms, for example, as illustrated below:

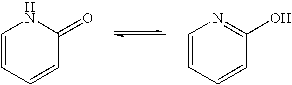

The invention expressly includes all tautomeric forms of the compounds described herein.

The compounds may also occur in cis- or trans- or E- or Z-double bond isomeric forms. All such isomeric forms of such compounds are expressly included in the present invention. All crystal forms of the compounds described herein are expressly included in the present invention.

Substituents on ring moieties (e.g., phenyl, thiazolyl, etc.) may be attached to specific atoms, whereby they are intended to be fixed to that atom, or they may be drawn unattached to a specific atom, whereby they are intended to be attached at any available atom that is not already substituted by an atom other than H (hydrogen).

The compounds of this invention may contain heterocyclic ring systems attached to another ring system. Such heterocyclic ring systems may be attached through a carbon atom or a heteroatom in the ring system.

A compound of any of the formulas delineated herein may be synthesized according to any of the processes delineated herein. In the processes delineated herein, the steps may be performed in an alternate order and may be preceded, or followed, by additional protection/deprotection steps as necessary. The processes may further comprise use of appropriate reaction conditions, including inert solvents, additional reagents, such as bases (e.g., LDA, DIEA, pyridine, K₂CO₃, and the like), catalysts, and salt forms of the above. The intermediates may be isolated or carried on in situ, with or without purification. Purification methods are known in the art and include, for example, crystallization, chromatography (liquid and gas phase), extraction, distillation, trituration, reverse phase HPLC and the like. Reactions conditions such as temperature, duration, pressure, and atmosphere (inert gas, ambient) are known in the art and may be adjusted as appropriate for the reaction. Additionally, the compounds can be produced metabolically.

As can be appreciated by one skilled in the art, the above synthetic schemes are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds. Synthetic chemistry transformations and protecting group methodologies (protection and deprotection) useful in synthesizing the inhibitor compounds described herein are known in the art and include, for example, those such as described in R. Larock, Comprehensive Organic Transformations, VCH Publishers (1989); T. Greene and P. Wuts, Protective Groups in Organic Synthesis, 3rd edition, John Wiley and Sons (1999); L. Fieser and M. Fieser, Fieser and Fieser's Reagents for Organic Synthesis, John Wiley and Sons (1994); and L. Paquette (editor), Encyclopedia of Reagents for Organic Synthesis, John Wiley and Sons (1995); P. Lopez et al., Synthesis, 2:186 (1998); A. Mikhalev, et al., Khim. Geterotsikl Soedin, 5:697 (1997); M. Fernandez, et al., Synthesis, 11:1362 (1995); P. Desos, et al., J. Med. Chem., 39:197 (1996); G. Timari, et al., Synlett, 9:1067 (1997); Y. Tagawa, et al., J. Heterocycl. Chem., 34:1677 (1997); A. Fuerstner, et al., Chem. Sci. 50:326 (1995); and A. Katritzky and A. Pozharski, Handbook of Heterocyclic Chemistry, 2^{nd} edition (2001).

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

The following examples contain detailed descriptions of the methods of preparation of compounds of Formulas I-III. These detailed descriptions fall within the scope, and serve to exemplify, the above-described General Synthetic Procedures which form part of the invention. These detailed descriptions are presented for illustrative purposes only and are not intended as a restriction on the scope of the invention.

### EXAMPLES

Unless otherwise noted, all materials were obtained from commercial suppliers and used without further purification. All parts are by weight and temperatures are in degrees centigrade unless otherwise indicated. All microwave-assisted reactions were conducted with a Smith Synthesizer from Personal Chemistry, Uppsala, Sweden. All compounds showed NMR spectra consistent with their assigned structures. Melting points were determined on a Buchi apparatus and are uncorrected. Mass spectral data was determined by electrospray ionization technique. All examples were purified to >95% purity as determined by high-performance liquid chromatography. Unless otherwise stated, reactions were run at RT.

### Example 1

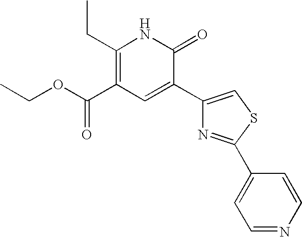

### Ethyl 2-ethyl-6-oxo-5-(2-(4-pyridinyl)-1,3-thiazol-4-yl)-1,6-dihydro-3-pyridinecarboxylate

(a) Ethyl-2-propionyl-3-(dimethylamino)prop-2-enoate. Ethyl propionylacetate (9.85 g, 68.3 mmol, Aldrich Chemical Co.) and N,N′-dimethylformamide dimethyl acetal (22.0 mL, 165.6 mmol) were combined and stirred at 110° C. for 2 h. The mixture was cooled to RT and poured into brine. The aqueous solution was extracted with EtOAc (4×). The combined EtOAc layers were washed with H₂O (2×) and brine, dried over MgSO₄, and concentrated in vacuo to give a dark-red oil. MS m/z: 200 (M+1). Calc'd for C₁₀H₁₇NO₃: 199.12.

(b) Ethyl 5-acetyl-2-ethyl-6-oxo-1,6-dihydropyridine-3-carboxylate. To a solution of acetoacetamide (5.87 g, 58.0 mmol) in dry THF (116 mL) was added NaH (60% in mineral oil, 1.88 g, 47.0 mmol) in portions over 15 min. After stirring for an additional 15 min, a solution of ethyl-2-propionyl-3-(dimethylamino)prop-2-enoate (Step a, 11.58 g, 58.1 mmol) in dry THF (116 mL) was added at a fast drip. After the addition the reaction was stirred at 60° C. overnight. The thickened material was cooled to RT and concentrated in vacuo. To the resulting yellow solid was added 250 mL of H₂O, and the solution was acidified to pH 1 with the addition of 5N HCl (aq). The resulting precipitate was filtered and dried in vacuo at 70° C. to give the title compound as a yellow solid. MS m/z: 238 (M+1).

Calc'd for C₁₂H₁₅NO₄: 237.10.

(c) Ethyl 5-(2-bromoacetyl)-2-ethyl-6-oxo-1,6-dihydropyridine-3-carboxylate. To a solution of ethyl 5-acetyl-2-ethyl-6-oxo-1,6-dihydropyridine-3-carboxylate (Step b, 1.03 g, 4.3 mmol) in 50 mL of dry THF was added 5,5′-dibromobarbituric acid (0.76 g, 2.7 mmol, Aldrich Chemical Co.). The solution was stirred at 60° C. for 3 h, then additional 5,5′-dibromobarbituric acid (90 mg) was added. After an additional 3 h the solution was cooled to RT and concentrated in vacuo. The solid was redissolved in EtOAc and the solution was washed with H₂O and brine, dried over MgSO₄, and concentrated in vacuo to give an orange solid that was used without further purification. MS m/z: 316 and 318 (M+1). Calc'd for C₁₂H₁₄BrNO₄: 315.01.

(d) Ethyl 2-ethyl-6-oxo-5-(2-(4-pyridyl)(1,3-thiazol-4-yl)-1,6-dihydro-3-pyridinecarboxylate. A solution of ethyl 5-(2-bromoacetyl)-2-ethyl-6-oxo-1,6-dihydropyridine-3-carboxylate (Step c, 100 mg, 0.3 mmol), isothionicotinamide (50 mg, 0.4 mmol, Lancaster Synthesis), and EtOH (2 mL) were heated in the microwave synthesizer at 150° C. for 5 min. The resulting solution was concentrated in vacuo and purified by flash chromatography on silica gel using 5% MeOH/CH₂Cl₂ to give a yellow solid. MS m/z: 356 (M+1).

Calc'd: 355.10. Anal. Calc'd. C₁₈H₁₇N₃O₃S: C, 60.83; H, 4.82; N, 11.82. Found: C, 60.67; H, 4.78; N, 11.69.

### Example 2

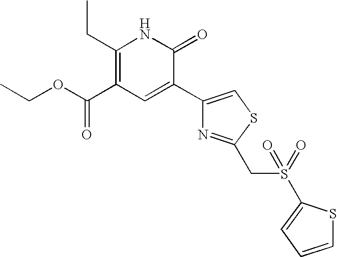

### Ethyl 2-ethyl-6-oxo-5-{2-[(thienylsulfonyl)methyl](1,3-thiazol-4-yl)}-1,6-dihydro-3-pyridinecarboxylate

This compound was prepared in a similar manner to Example 1d using ethyl 5-(2-bromoacetyl)-2-ethyl-6-oxo-1,6-dihydro-3-pyridinecarboxylate (Example 1c) (100 mg, 0.3 mmol), 2-(2-thienylsulfonyl)ethanethioamide (70 mg, 0.3 mmol, Maybridge), and 2 mL of EtOH. The resulting solution was diluted with hexanes and filtered. The solid was suspended in a minimum of EtOH and filtered to give a light pink solid. MS m/z: 439 (M+1). Calc'd 438.04. Anal. Calc'd. C₁₈H₁₈N₂O₅S₃.0.3H₂O: C, 48.70; H, 4.22; N, 6.31. Found: C, 48.37; H, 4.05; N, 6.16.

### Example 3

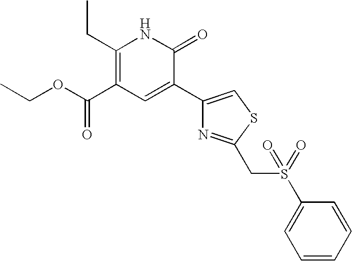

### Ethyl 2-ethyl-6-oxo-5-{2-[(phenylsulfonyl)methyl](1,3-thiazol-4-yl)}-1,6-dihydro-3-pyridinecarboxylate

This compound was prepared in a similar manner to Example 1d using ethyl 5-(2-bromoacetyl)-2-ethyl-6-oxo-1,6-dihydro-3-pyridinecarboxylate (Example 1c) (100 mg, 0.3 mmol), 2-(phenylsulfonyl)ethanethioamide (70 mg, 0.3 mmol), and 2 mL of EtOH. The resulting solution was diluted with hexanes and filtered. The solid was suspended in a minimum of EtOAc and filtered to give a brown solid. MS m/z: 433 (M+1). Calc'd Exact Mass: 432.08. Anal. Calc'd C₂₀H₂₀N₂O₅S₂•0.3H₂O: C, 54.85; H, 4.74; N, 6.40. Found: C, 54.83; H, 4.72; N, 6.50.

### Example 4

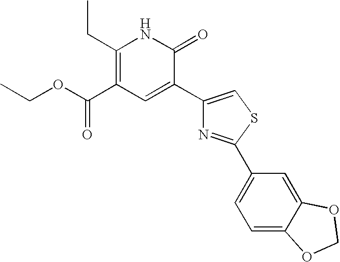

### Ethyl 2-ethyl-6-oxo-5-{2-(benzo[1,3]dioxol-5-yl)(1,3-thiazol-4-yl)}-1,6-dihydro-3-pyridinecarboxylate

This compound was prepared in a similar manner to Example 1d using ethyl 5-(2-bromoacetyl)-2-ethyl-6-oxo-1,6-dihydro-3-pyridinecarboxylate (Example 1c) (100 mg, 0.3 mmol), benzo[1,3]dioxole-5-carbothioic acid amide (60 mg, 0.3 mmol, Maybridge), and 2 mL of EtOH. The resulting solution was diluted with hexanes and filtered. The solid was suspended in a minimum of EtOAc. A small amount of dark-red solid settled to the bottom of the light-pink precipitate. The suspension solution of light-pink solid was carefully pipetted away from the dark red solid, and then filtered to give a light-pink solid. The light-pink solid was once again suspended in a minimum of EtOAc and filtered to give a light-pink solid. MS m/z: 399 (M+1).

Calc'd Exact Mass: 398.09. Anal. Calc'd C₂₀H₁₈N₂O₅S.0.1H₂O: C, 60.02; H, 4.58; N, 7.00. Found: C, 59.86; H, 4.54; N, 7.08.

### Example 5

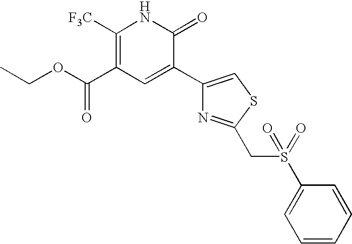

### Ethyl 6-oxo-5-{2-[(phenylsulfonyl)methyl](1,3-thiazol-4-yl)}-2-(trifluoromethyl)-1,6-dihydro-3-pyridinecarboxylate

(a) Ethyl 2-trifluoroacetyl-3-(dimethylamino)prop-2-enoate. N,N′-Dimethylformamide dimethyl acetal (65.5 mL, 493.1 mmol) was added slowly to ethyl 4,4,4-trifluoroacetoacetate (36.9 g, 200.0 mmol, Aldrich Chemical Co.). The solution was stirred at RT for 1.5 h, and at 80° C. for 1 h. The resulting solution was cooled to RT and diluted with 300 mL of brine. The aqueous solution was extracted with EtOAc (4×). The combined EtOAc layers were washed with H₂O (2×) and brine, dried over MgSO₄, and concentrated in vacuo to give a dark-red oil.

(b) Ethyl 5-acetyl-2-trifluoromethyl-6-oxo-1,6-dihydro-3-pyridinecarboxylate. To a solution of acetoacetamide (14.3 g, 141.5 mmol) in 300 mL of anhydrous THF was added NaH (60% in mineral oil, 5.0 g, 124.0 mmol) in portions over 10 min. After stirring for an additional 25 min, a solution of ethyl 2-trifluoroacetyl-3-(dimethylamino)prop-2-enoate (Step a, 33.8 g, 141.5 mmol) in 200 mL of anhydrous THF was added at a fast drip. The resulting solution was stirred at 60° C. overnight, then cooled to RT and concentrated in vacuo. The resulting residue was dissolved in 500 mL of H₂O and acidified to pH 1 with the addition of 5N HCl (aq). The aqueous solution was extracted with EtOAc (3×). The combined EtOAc layers were washed with brine, dried over MgSO₄, and concentrated in vacuo to give an oil that later solidified. Additional compound remained in the H₂O layer, but no attempt was made at further recovery. MS m/z: 278 (M+1). Calc'd for C₁₁H₁₀F₃NO₄: 277.06.

(c) Ethyl 5-(2-bromoacetyl)-2-trifluoromethyl-6-oxo-1,6-dihydro-3-pyridinecarboxylate. The compound was prepared in a similar manner to Example 1c using ethyl 5-acetyl-2-trifluoromethyl-6-oxo-1,6-dihydro-3-pyridinecarboxylate (Step b, 4.1 g, 14.7 mmol) and 5,5′-dibromobarbituric acid (2.18 g, 7.6 mmol). The crude material was semi-purified by flash chromatography on silica gel using 2% MeOH/CH₂Cl₂ to give an orange solid. This material was used without further purification. MS m/z: 356 and 358 (M+1). Calc'd for C₁₁H₉BrF₃NO₄: 354.97.

(d) Ethyl 6-oxo-5-{2-[(phenylsulfonyl)methyl](1,3-thiazol-4-yl)}-2-(trifluoromethyl)-1,6-dihydro-3-pyridinecarboxylate. The compound was prepared in a similar manner to Example 1d using ethyl 5-(2-bromoacetyl)-2-trifluoromethyl-6-oxo-1,6-dihydro-3-pyridinecarboxylate (Step c, 160 mg, 0.2 mmol), 2-(phenylsulfonyl)ethanethioamide (80 mg, 0.4 mmol, Maybridge), and 2 mL of MeOH. The resulting material was concentrated in vacuo, then suspended in EtOAc and filtered to give a brown solid. MS m/z: 473 (M+1). Calc'd Exact Mass: 472.04. Anal. Calc'd C₁₉H₁₅F₃N₂O₅S₂: C, 48.30; H, 3.20; N, 5.93. Found: C, 48.13; H, 3.28; N, 5.67.

### Example 6

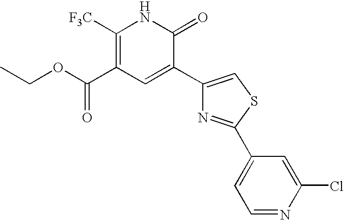

### Ethyl 2-trifluoromethyl-6-oxo-5-(2-(3-chloro-4-pyridyl)(1,3-thiazol-4-yl)-1,6-dihydro-3-pyridinecarboxylate

A solution of ethyl 5-(2-bromoacetyl)-2-trifluoromethyl-6-oxo-1,6-dihydro-3-pyridinecarboxylate (Example 5c, 160 mg, 0.2 mmol), and 3-chloro-isothionicotinamide (60 mg, 0.4 mmol), in 2 mL of MeOH was heated in a microwave synthesizer at 150° C. for 5 min. The resulting solution was filtered to give a yellow solid. MS m/z: 430 (M+1). Calc'd Exact Mass: 429.02. Anal. Calc'd C₁₇H₁₁ClN₃O₃S: C, 47.51; H, 2.58; N, 9.78. Found: C, 47.24; H, 2.71; N, 9.46.

### Example 7

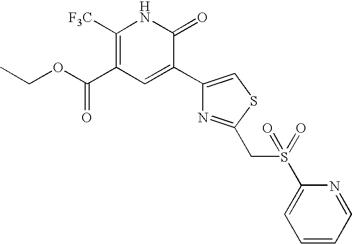

### Ethyl 6-oxo-5-{2-[(2-pyridylsulfonyl)methyl](1,3-thiazol-4-yl)}-2-(trifluoromethyl)-1,6-dihydro-3-pyridinecarboxylate

A solution of ethyl 5-(2-bromoacetyl)-2-trifluoromethyl-6-oxo-1,6-dihydro-3-pyridinecarboxylate (Example 5c, 160 mg, 0.2 mmol), and 2-(2-pyridylsulfonyl)ethane-thioamide (90 mg, 0.4 mmol, Maybridge), in 2 mL of MeOH was heated in the microwave synthesizer at 150° C. for 5 min. The resulting solution was concentrated in vacuo. The residue was suspended in a 1:1 mixture of EtOH:hexanes and filtered to give a light yellow solid. MS m/z: 474 (M+1). Calc'd Exact Mass: 473.03.

Anal. Calc'd. C₁₈H₁₄F₃N₃O₅S₂: C, 45.66; H, 2.98; N, 8.88. Found: C, 45.47; H, 3.04; N, 8.74.

### Example 8

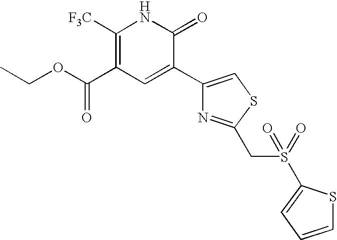

### Ethyl 6-oxo-5-{2-[(2-thienylsulfonyl)methyl](1,3-thiazol-4-yl)}-2-(trifluoromethyl)-1,6-dihydro-3-pyridinecarboxylate

A solution of ethyl 5-(2-bromoacetyl)-2-trifluoro-methyl-6-oxo-1,6-dihydro-3-pyridinecarboxylate (Example 5c) (160 mg, 0.2 mmol), and 2-(2-thienylsulfonyl)ethanethioamide (60 mg, 0.3 mmol, Maybridge), in 2 mL of MeOH was heated in the Microwave synthesizer at 150° C. for 5 min. The resulting solution was filtered and the filtrate was concentrated in vacuo. The concentrated filtrate was suspended in a 1:1 solution of EtOH:hexanes and then filtered to give an off-white solid. The solid was resuspended in a 1:1 EtOH:hexanes solution and heated. Upon cooling the precipitate was filtered to give an off-white solid. MS m/z: 479 (M+1). Calc'd Exact Mass: 477.99. Anal. Calc'd C₁₇H₁₃F₃N₂O₅S₃.0.2H₂O: C, 42.35; H, 2.80; N, 5.81. Found: C, 42.06; H, 2.78; N, 5.81.

### Example 9

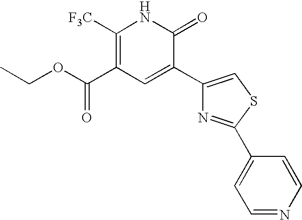

### Ethyl 2-trifluoromethyl-6-oxo-5-(2-(4-pyridyl)(1,3-thiazol-4-yl)-1,6-dihydro-3-pyridinecarboxylate

A solution of ethyl 5-(2-bromoacetyl)-2-trifluoro-methyl-6-oxo-1,6-dihydro-3-pyridinecarboxylate (Example 5c) (340 mg, 1.0 mmol), and isothionicotinamide (140 mg, 1.0 mmol) in EtOH (10 mL) was stirred at 80° C. overnight. The resulting solution was cooled to RT and filtered. The solid was washed with EtOH to give a pink solid which was suspended in 10 mL of EtOH and treated with a catalytic amount of p-toluenesulfonic acid. The solution was stirred at reflux for 3 h. The resulting solution was concentrated to ⅓ volume, filtered and washed with EtOAc to give a light pink solid. The light pink solid was suspended in 2 mL of DMSO and 8 mL of H₂O. The precipitate was filtered and washed with CH₂Cl₂ to give a light pink solid. MS m/z: 396 (M+1). HRMS Calc'd for C₁₇H₁₃F₃N₃O₃S [M+H], 396.0615, Found, 396.0624.

### Example 10

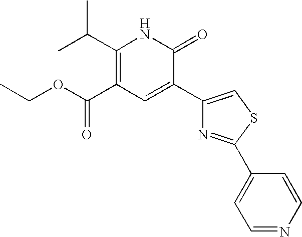

### Ethyl 2-isopropyl-6-oxo-5-(2-(4-pyridyl)(1,3-thiazol-4-yl)-1,6-dihydro-3-pyridinecarboxylate

(a) Ethyl 3-(dimethylamino)-2-(2-methylpropanoyl)prop-2-enoate. This compound was prepared in a similar manner to Example 1a using ethyl isobutyrylacetate (8.00 g, 50.6 mmol, Lancaster Synthesis) and N,N′-dimethylformamide dimethyl acetal (17.0 mL, 128.0 mmol) to give a red oil. MS m/z: 214 (M+1). Calc'd for C₁₁H₁₉NO₃: 213.14.

(b) Ethyl 5-acetyl-2-isopropyl-6-oxo-1,6-dihydropyridine-3-carboxylate. This compound was prepared in a similar manner to Example 1b using ethyl 3-(dimethylamino)-2-(2-methyl-propanoyl)prop-2-enoate (Step a, 8.91 g, 41.8 mmol), acetoacetamide (4.10 g, 40.5 mmol), and NaH (60% in mineral oil, 1.35 g, 33.8 mmol) to give a yellow solid. MS m/z: 252 (M+1). Calc'd for C₁₃H₁₇NO₄: 251.12.

(c) Ethyl 5-(2-bromoacetyl)-2-isopropyl-6-oxo-1,6-dihydropyridine-3-carboxylate. To a solution of ethyl 5-acetyl-2-isopropyl-6-oxo-1,6-dihydropyridine-3-carboxylate (Step b, 1.08 g, 4.3 mmol) in dry THF (50 mL) was added 5,5′-dibromobarbituric acid (0.89 g, 3.1 mmol). The solution was stirred at 60° C. overnight, then concentrated in vacuo to give an orange solid that was used for next step without further purification. MS m/z: 330, 332 (M+1).

Calc'd for C₁₃H₁₆BrNO₄: 329.03.

(d) Ethyl 2-isopropyl-6-oxo-5-(2-(4-pyridyl)(1,3-thiazol-4-yl)-1,6-dihydro-3-pyridinecarboxylate. A solution of ethyl 5-(2-bromoacetyl)-2-isopropyl-6-oxo-1,6-dihydropyridine-3-carboxylate (Step c, 210 mg, 0.6 mmol), and isothionicotinamide (70 mg, 0.5 mmol), in 10 mL of EtOH was stirred at reflux overnight. The resulting solution was cooled to RT and filtered to give a red solid. MS m/z: 370 (M+1). Calc'd Exact Mass: 369.11. Anal. Calc'd C₁₉H₁₉N₃O₃S.0.6HBr.1.1H₂O: C, 52.13; H, 5.02; N, 9.60. Found: C, 51.96; H, 4.76; N, 9.81.

### Example 11

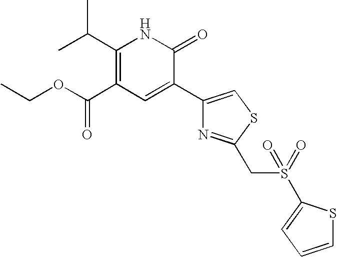

### Ethyl 2-isopropyl-6-oxo-5-{2-[(thienylsulfonyl)methyl](1,3-thiazol-4-yl)}-1,6-dihydro-3-pyridinecarboxylate

This compound was prepared in a similar manner to Example 10d using ethyl 5-(2-bromoacetyl)-2-isopropyl-6-oxo-1,6-dihydropyridine-3-carboxylate (Example 10c) (200 mg, 0.6 mmol), 2-(2-thienylsulfonyl)ethanethioamide (100 mg, 0.5 mmol), and 10 mL of EtOH to give a pink solid. MS m/z: 453 (M+1). Calc'd Exact Mass: 452.05. Anal. Calc'd C₁₉H₂₀N₂O₅S₃: C, 50.43; H, 4.45; N, 6.19. Found: C, 50.27; H, 4.44; N, 6.09.

### Example 12

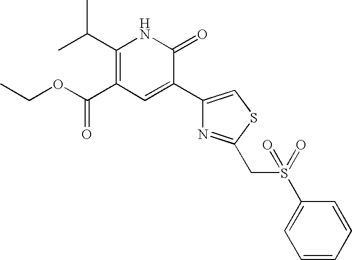

### Ethyl 2-isopropyl-6-oxo-5-{2-[(phenylsulfonyl)methyl](1,3-thiazol-4-yl)}-1,6-dihydro-3-pyridinecarboxylate

This compound was prepared in a similar manner to Example 10d using ethyl 5-(2-bromoacetyl)-2-isopropyl-6-oxo-1,6-dihydropyridine-3-carboxylate (Example 10c) (190 mg, 0.6 mmol), 2-(phenylsulfonyl)ethanethioamide (90 mg, 0.4 mmol), and 10 mL of EtOH to give a brown solid. MS m/z: 447 (M+1). Calc'd Exact Mass: 446.10. Anal. Calc'd C₂₁H₂₂N₂O₅S₂: C, 56.49; H, 4.97; N, 6.27. Found: C, 56.45; H, 4.94; N, 6.41.

### Example 13

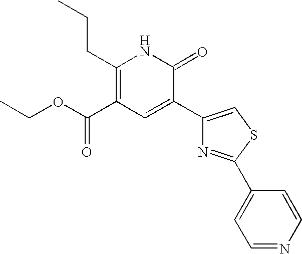

### Ethyl 2-propyl-6-oxo-5-(2-(4-pyridyl)(1,3-thiazol-4-yl)-1,6-dihydro-3-pyridinecarboxylate

(a) Ethyl 2-propyl-3-(dimethylamino)prop-2-enoate. This compound was prepared in a similar manner to Example 1a using ethyl butyrylacetate (5.01 g, 31.7 mmol, Lancaster Synthesis) and N,N′-dimethylformamide dimethyl acetal (11.0 mL, 82.8 mmol) to give a dark red oil. MS m/z: 214 (M+1).

Calc'd for C₁₀H₁₉NO₂: 185.14.

(b) Ethyl 5-acetyl-2-propyl-6-oxo-1,6-dihydropyridine-3-carboxylate. This compound was prepared in a similar manner to Example 1b using ethyl 2-propyl-3-(dimethylamino)prop-2-enoate (Step a, 6.17 g, 28.9 mmol), acetoacetamide (2.91 g, 28.8 mmol), and NaH (60% in mineral oil, 0.94 g, 23.5 mmol) to give a yellow solid. MS m/z: 252 (M+1). Calc'd for C₁₃H₁₇NO₄: 251.12.

(c) Ethyl 5-(2-bromoacetyl)-2-propyl-6-oxo-1,6-dihydropyridine-3-carboxylate. This compound was prepared in a similar manner to Example 10c using ethyl 5-acetyl-2-propyl-6-oxo-1,6-dihydropyridine-3-carboxylate (Step b, 1.08 g, 4.3 mmol), 5,5′-dibromobarbituric acid (0.89 g, 3.1 mmol), and 50 mL of dry THF to give an orange solid that was used for next step without further purification. MS m/z: 330, 332 (M+1). Calc'd for C₁₃H₁₆BrNO₄: 329.03.

(d) Ethyl 2-propyl-6-oxo-5-(2-(4-pyridyl)(1,3-thiazol-4-yl)-1,6-dihydro-3-pyridinecarboxylate. This compound was prepared in a similar manner to Example 9 using ethyl 5-(2-bromoacetyl)-2-propyl-6-oxo-1,6-dihydropyridine-3-carboxylate (Step c, 210 mg, 0.6 mmol), isothionicotinamide (80 mg, 0.6 mmol), and 8 mL of EtOH to give a red solid. The solid was purified by flash chromatography on silica gel using 2% MeOH/CH₂Cl₂ to give a white solid. MS m/z: 370 (M+1). Calc'd Exact Mass: 369.11. Anal. Calc'd. C₁₉H₁₉N₃O₃S: C, 61.77; H, 5.18; N, 11.37. Found: C, 61.92; H, 5.46; N, 11.32.

### Example 14

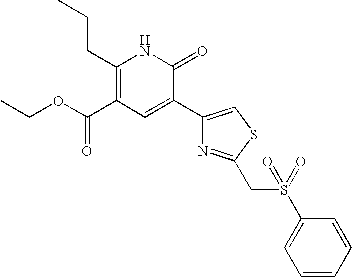

### Ethyl 2-propyl-6-oxo-5-{2-[(phenylsulfonyl)methyl](1,3-thiazol-4-yl)}-1,6-dihydro-3-pyridinecarboxylate

This compound was prepared in a similar manner to Example 10d using ethyl 5-(2-bromoacetyl)-2-propyl-6-oxo-1,6-dihydropyridine-3-carboxylate (Example 13c) (200 mg, 0.6 mmol), 2-(phenylsulfonyl)-ethanethioamide (90 mg, 0.4 mmol), and 8 mL of EtOH to give a brown solid. MS m/z: 447 (M+1). Calc'd Exact Mass: 446.10. Anal. Calc'd. C₂₁H₂₂N₂O₅S₂.0.1H₂O: C, 56.26; H, 4.99; N, 6.25. Found: C, 55.97; H, 4.90; N, 6.37.

### Example 15

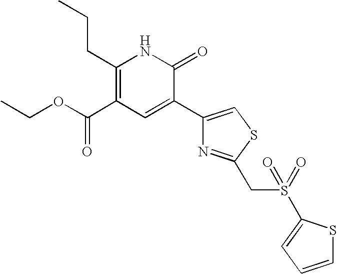

### Ethyl 2-propyl-6-oxo-5-{2-[(thienylsulfonyl)methyl](1,3-thiazol-4-yl)}-1,6-dihydro-3-pyridinecarboxylate

This compound was prepared in a similar manner to Example 10d using ethyl 5-(2-bromoacetyl)-2-propyl-6-oxo-1,6-dihydropyridine-3-carboxylate (Example 13c) (200 mg, 0.6 mmol), 2-(2-thienylsulfonyl)ethanethioamide (100 mg, 0.5 mmol), and 7 mL of EtOH to give a pink solid. MS m/z: 453 (M+1). Calcld Exact Mass: 452.05. Anal. Calc'd. C₁₉H₂₀N₂O₅S₃.0.7H₂O: C, 49.06; H, 4.64; N, 6.02. Found: C, 48.77; H, 4.30; N, 5.99.

### Example 16

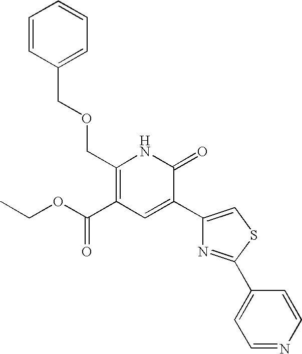

### Ethyl 6-oxo-2-[(phenylmethoxy)methyl]-5-(2-(4-pyridyl)(1,3-thiazol-4-yl))-1,6-dihydro-3-pyridinecarboxylate

(a) Ethyl 3-oxo-4-(phenylmethoxy)butanoate. To a solution of ethyl chloroacetoacetate (21.0 mL, 155.4 mmol, Aldrich Chemical Co.) in dry toluene (300 mL) was added NaH (60% in mineral oil, 13.77 g, 344.3 mmol) in portions over 0.5 h. After the addition was complete the solution was stirred for 0.5 h, and benzyl alcohol (31.0 mL, 299.6 mmol, Aldrich Chemical Co.) was added dropwise over 0.5 h. The resulting mixture was stirred at RT overnight before slowly quenched with H₂O, and neutralized with 1N HCl (aq). The organic layer was separated, washed with brine, dried over MgSO₄, and concentrated in vacuo. The resulting oil was purified by flash chromatography on silica gel using 9:1 CH₂Cl₂:EtOAc to give an oil that contained the title compound and benzyl alcohol. MS m/z: 259 (M+Na). Calc'd for C₁₃H₁₆O₄: 236.10.

(b) Ethyl 3-(dimethylamino)-2-[2-(phenylmethoxy)acetyl]prop-2-enoate. This compound was prepared in a manner similar to Example 1a using crude ethyl 3-oxo-4-(phenylmethoxy)butanoate (Step a, 2.04 g) and N,N′-dimethylformamide dimethyl acetal (3.00 mL, 22.6 mmol) to give a red oil that contained both the title compound and benzyl alcohol. MS m/z: 292 (M+1). Calc'd for C₁₆H₂₁NO₄: 291.15.

(c) Ethyl 5-acetyl-6-oxo-2-[(phenylmethoxy)methyl]-1,6-dihydropyridine-3-carboxylate. To a solution of acetoacetamide (6.92 g, 68.4 mmol) in 200 mL of anhydrous THF was added NaH (60% in mineral oil, 2.20 g, 55.0 mmol) in portions over 10 min. The solution was stirred at RT for 15 min, and a solution of crude ethyl 3-(dimethylamino)-2-[2-(phenylmethoxy)acetyl]prop-2-enoate (Step b, 19.97 g, 68.6 mmol) in anhydrous THF (200 mL) was added at a fast drip to the reaction. After the addition was completed the reaction was stirred at 60° C. for 3 days. The solution was cooled to RT, and concentrated in vacuo. The resulting material was suspended in 400 mL of H₂O and acidified with the addition of 5N HCl (aq). The solution was carefully decanted through a fritted filter keeping most of the solid residue in the flask. The remaining residue was suspended in Et₂O, filtered, and washed with MeOH to give a yellow solid. MS m/z: 330 (M+1). Calc'd for C₁₈H₁₉NO₅: 329.13.

(d) Ethyl 5-(2-bromoacetyl)-6-oxo-2-[(phenylmethoxy) methyl]-1,6-dihydropyridine-3-carboxylate. To a solution of ethyl 5-acetyl-6-oxo-2-[(phenylmethoxy)methyl]-1,6-dihydropyridine-3-carboxylate (Step c, 1.65 g, 5.0 mmol) in anhydrous THF (50 mL) was added 5,5′-dibromobarbituric acid (0.87 g, 3.0 mmol). The reaction was stirred at 60° C. After 4 h, additional 5,5′-dibromobarbituric acid (90 mg, 0.3 mmol) was added. After an additional 6 h the reaction was cooled to RT and stirred overnight. The solution was concentrated in vacuo. The resulting residue was dissolved in EtOAc, washed with H₂O and brine, and concentrated in vacuo. The residue was purified by flash chromatography on silica gel using 5% MeOH:CH₂Cl₂ to give a light-yellow oil which solidified upon standing. MS m/z: 408, 410 (M+1).

Calc'd for C₁₈H₁₈BrNO₅: 407.04.

(e) Ethyl 6-oxo-2-[(phenylmethoxy)methyl]-5-(2-(4-pyridyl)(1,3-thiazol-4-yl))-1,6-dihydropyridine-3-carboxylate. A solution of ethyl 5-(2-bromoacetyl)-6-oxo-2-[(phenylmethoxy)methyl]-1,6-dihydropyridine-3-carboxylate (Step d, 90 mg, 0.2 mmol), and isothionicotinamide (34 mg, 0.3 mmol, Lancaster Synthesis), in 15 mL of MeOH was stirred at reflux overnight. The resulting solution was concentrated in vacuo, absorbed onto silica gel, and purified by flash chromatography on silica gel using 10% EtOAc:CH₂Cl₂ to give a light-yellow solid. The solid was suspended in a 1:1 solution of CH₂Cl₂:Et₂O and filtered to give another solid. This was repeated one more time to give a light-yellow solid. MS m/z: 448 (M+1). Calc'd Exact Mass: 447.13. Anal. Calc'd C₂₄H₂₁N₃O₄S.0.2H₂O: C, 63.90; H, 4.78; N, 9.32. Found: C, 63.72; H, 4.73; N, 9.24.

### Example 17

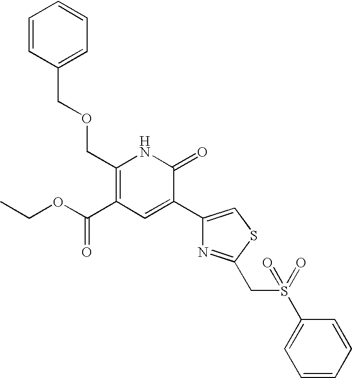

### Ethyl 6-oxo-2-[(phenylmethoxy)methyl]-5-{2-[(phenylsulfonyl)methyl](1,3-thiazol-4-yl)}-1,6-dihydro-3-pyridinecarboxylate

A solution of ethyl 5-(2-bromoacetyl)-6-oxo-2-[(phenylmethoxy)methyl]-1,6-dihydropyridine-3-carboxylate (Example 13c, 200 mg, 0.5 mmol), and 2-(phenylsulfonyl) ethanethioamide (130 mg, 0.6 mmol), in 2 mL of MeOH was heated by microwave at 150° C. for 500 sec. The resulting solution was concentrated in vacuo and purified by flash chromatography on silica gel using 5% EtOAc:CH₂Cl₂ to give an oil that solidified upon standing. The solid was suspended in a minimum of CH₂Cl₂ and filtered to give a light-yellow solid. MS m/z: 525 (M+1). Calc'd Exact Mass: 524.11. Anal. Calc'd. C₂₆H₂₄N₂O₆S₂: C, 59.53; H, 4.61; N, 5.34. Found: C, 59.40; H, 4.62; N, 5.21.

### Example 18

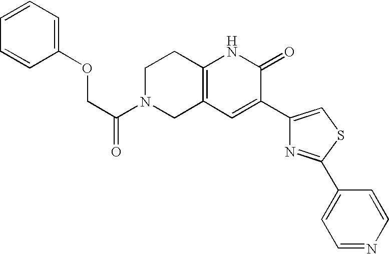

### Phenylmethyl 2-oxo-3-(2-(4-pyridyl)(1,3-thiazol-4-yl))-1,5,6,7,8-pentahydropyridino[3,2-c]pyridine-6-carboxylate

(a) Phenylmethyl 3-[(dimethylamino)methylene]-4-oxoazaperhydroine carboxylate. Benzyl 4-oxo-1-piperidinecarboxylate (5.01 g, 21.5 mmol, Aldrich) and N,N′-dimethylformamide dimethyl acetal (7.2 mL, 54.2 mmol) were combined and heated neat to 100° C. for 4 h. The solution was concentrated to a constant weight. MS m/z: 288.8 (M+1).

(b) 2-(2-Pyridin-4-yl-thiazol-4-yl)-acetamide. Eight 5 mL microwave reaction tubes each containing isothionicotinamide (Pfaltz-Bauer) (505 mg, 3.6 mmol), methyl 4-chloroaceto-acetate (Aldrich) (0.38 mL, 496 mg, 3.3 mmol) and 3 mL MeOH were heated to 150° C. for 6 min in a Microwave synthesizer. The reaction mixtures were combined and the solvent was removed in vacuo. The oily residue was dissolved in 1,4-dioxane, 80 mL concentrated NH₄OH was added and the reaction was stirred at RT. After 39 h the solvent was removed in vacuo and the residue was dissolved in MeOH. The solution was evaporated onto SiO₂ and the material was purified by flash column chromatography eluting with MeOH:CH₂Cl₂ (0:1 to 1:9) to give a tan amorphous solid. MS m/z: 220 (M+1); 218 (M−1). Calc'd for C₁₀H₇N₂O₂S Exact Mass: 219.02.

(c) Benzyl 2-oxo-3-(2-(4-pyridyl)(1,3-thiazol-4-yl))-1,5,6,7,8-pentahydropyridino[3,2-c]pyridine-6-carboxylate. To a solution of phenylmethyl 3-[(dimethylamino)methylene]-4-oxoazaperhydroinecarboxylate (Step a, 1.29 g, 4.5 mmol) and 2-(2-(4-pyridyl)-1,3-thiazol-4-yl)acetamide (Step b, 1.00 g, 4.6 mmol), in 100 mL of anhydrous DMF was added NaH (60% in mineral oil, 0.40 g, 10 mmol) in two portions over 3 min. The solution was stirred at 70° C. for 4 h, then cooled to RT and diluted with H₂O. The aqueous solution was acidified with 5N HCl (aq). The resulting precipitate was filtered and washed with H₂O and hexanes. The solid was stirred in hexanes for 4 h, filtered and dried in funnel to give a brown solid. MS m/z: 445 (M+1). Calc'd for C₂₄H₂₀N₄O₃S Exact Mass: 444.13.

### Example 19

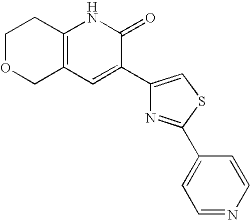

### 3-(2-(4-Pyridyl)-1,3-thiazol-4-yl)-1,7,8-trihydro-5H-pyrano[4,3-b]pyridin-2-one

(a) 3-[(Dimethylamino)methylene]-2H-5,6-dihydropyran-4-one. A mixture of tetrahydro-4H-pyran-4-one (1.77 g, 17.7 mmol) and N,N′-dimethylformamide dimethyl acetal (2.35 mL, 17.7 mmol) was heated at 100° C. for 1.5 h. The resulting solution was concentrated in vacuo to a constant weight. MS: m/z 156 (M+1). Calc'd for C₈H₁₃NO₂ Exact Mass: 155.09.

(b) 3-(2-(4-Pyridyl)-1,3-thiazol-4-yl)-1,7,8-trihydro-5H-pyrano[4,3-b]pyridin-2-one. To a solution of 3-[(dimethylamino)methylene]-2H-5,6-dihydropyran-4-one (Step a, 0.84 g, 3.5 mmol), 2-(2-(4-pyridyl)-1,3-thiazol-4-yl)acetamide (Example 18b) (0.78 g, 3.6 mmol), and 20 mL of anhydrous DMF was added NaH (60% in mineral oil, 0.30 g, 7.5 mmol) in one portion. The resulting solution was stirred at RT overnight, diluted with H₂O and acidified with 2N HCl (aq) to pH ˜4. The resulting precipitate was filtered, dissolved in 10% MeOH/CH₂Cl₂, washed with H₂O and saturated NaHCO₃, dried over MgSO₄, and concentrated in vacuo to give a yellow solid. The solid was stirred in 150 mL of hexanes for 2 h, then filtered to give a light-brown solid. MS m/z: 312 (M+1). HRMS Calc'd for C₁₆H₁₄N₃O₂S [M+H], 312.0801, Found: 312.0797.

### Example 20

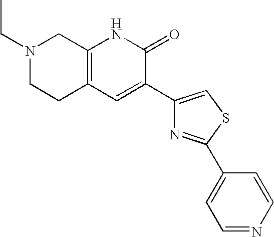

### 7-Ethyl-3-(2-(4-pyridyl)(1,3-thiazol-4-yl))-1,5,6,7,8-pentahydropyridino[3,2-c]pyridin-2-one

(a) 4-[(Dimethylamino)methylene]-1-ethylazaperhydroin-3-one. 1-Ethyl-3-piperidone HCl salt was dissolved in 5% MeOH/CH₂Cl₂ and washed with saturated NaHCO₃. The aqueous solution was extracted with 5% MeOH/CH₂Cl₂ (2×). The combined organic layers were dried over MgSO₄ and concentrated in vacuo to give 0.40 g (3.1 mmol) of a golden oil. N,N′-Dimethylformamide dimethyl acetal (0.40 mL, 3.0 mmol) was added to the oil and the solution was heated neat at 100° C. for 1.25 h. The resulting solution was concentrated in vacuo to give a black oil. MS: m/z 183 (M+1).

(b) 7-Ethyl-3-(2-(4-pyridyl)(1,3-thiazol-4-yl))-1,5,6,7,8-pentahydropyridino[3,2-c]pyridin-2-one. To a solution of 4-[(dimethylamino)methylene]-1-ethylazaperhydroin-3-one (Step a, 0.51 g), 2-(2-(4-pyridyl)-1,3-thiazol-4-yl)acetamide (Example 18b) (0.61 g, 2.8 mmol), and 25 mL of anhydrous DMF was added NaH (60% in mineral oil, 0.24 g, 6.0 mmol) in one portion. The resulting solution was stirred at RT overnight, diluted with H₂O and acidified with 2N HCl (aq) to pH ˜4. The aqueous solution was extracted with EtOAc (4×). The EtOAc layers were concentrated in vacuo. The resulting solid was suspended between EtOAc/H₂O and filtered to give a tan solid. MS m/z: 339.2 (M+1).

Calc'd Exact Mass: 338.12. Anal. Calc'd. C₁₈H₁₈N₄OS: C, 62.55; H, 5.48; N, 16.21. Found: C, 62.37; H, 5.31; N, 16.03.

### Example 21

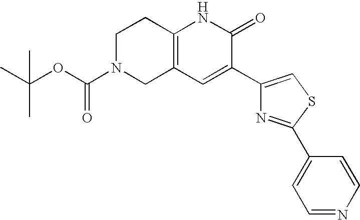

### tert-Butyl 2-oxo-3-(2-(4-pyridyl)(1,3-thiazol-4-yl))-1,5,6,7,8-pentahydropyridino[3,2-c]pyridine-6-carboxylate

tert-Butyl 4-oxo-1-piperidinecarboxylate (0.98 g, 4.9 mmol) and N,N′-dimethylformamide dimethyl acetal (0.65 mL, 4.9 mmol) were suspended in toluene and stirred at 100° C. for 2.5 h. The resulting solution was concentrated in vacuo to a constant weight. MS: m/z 256 (M+2). To this oil was added 2-(2-(4-pyridyl)-1,3-thiazol-4-yl)acetamide (Example 18b) (1.10 g, 5.0 mmol), 20 mL of anhydrous DMF, and finally NaH (60% in mineral oil, 0.34 g, 8.5 mmol) in one portion. The resulting solution was stirred at RT over the weekend. The solution was diluted with H₂O and acidified to pH ˜4. The resulting precipitate was filtered and washed with H₂O. The solid was stirred in 150 mL of hexanes and filtered to give a tan solid. MS m/z: 411 (M+1). Calc'd for C₂₁H₂₂N₄O₃S Exact Mass: 410.14.

### Example 22

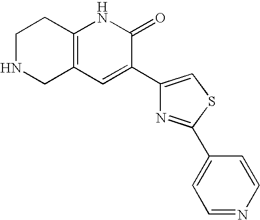

### 3-(2-(4-Pyridyl)-1,3-thiazol-4-yl)-1,5,6,7,8-pentahydropyridino[3,2-c]pyridin-2-one

tert-Butyl 2-oxo-3-(2-(4-pyridyl)(1,3-thiazol-4-yl))-1,5,6,7,8-pentahydropyridino[3,2-c]pyridine-6-carboxylate (Example 21) (0.63 g, 1.53 mmol) was suspended in 20 mL of dioxane and 4M HCl (in dioxane, 3 mL, 12 mmol, Aldrich) was added. The mixture was stirred at RT. After 6.5 h, 4M HCl (in dioxane, 1.5 mL, 6 mmol) was added and stirring continued overnight. The solution was filtered to give the HCl salt as a rust colored solid. MS m/z: 311 (M+1). HRMS

Calc'd for C₁₆H₁₄N₄OS [M+H], 311.0961, Found: 311.0938.

### Example 23

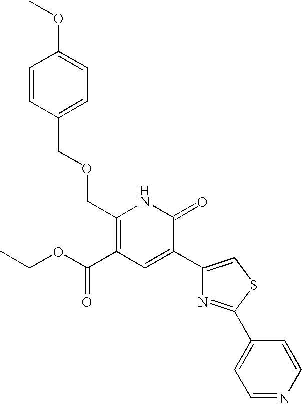

### Ethyl 2-{[(4-methoxyphenyl)methoxy]methyl}-6-oxo-5-(2-(4-pyridyl)(1,3-thiazol-4-yl))-1,6-dihydro-3-pyridinecarboxylate

(a) Ethyl 4-[(4-methoxyphenyl)methoxy]-3-oxobutanoate. To a suspension of NaH (60% in mineral oil, 4.52 g, 113.0 mmol) in anhydrous toluene was added 4-methoxybenzyl alcohol (15.0 mL, 108.6 mmol, Avocado Research Chemicals) dropwise over 20 min. After stirring for 1 h, ethyl chloroacetoacetate (7.4 mL, 54.76 mmol) was added dropwise over 15 min. After the addition was complete the reaction was stirred at RT overnight. The reaction was slowly quenched with 2N HCl (aq). The aqueous layer was separated and extracted with toluene (2×). The combined toluene layers were dried over MgSO₄ and concentrated in vacuo. The resulting red oil was stirred with heptane (2×20 mL) and the heptane layer was separated away. The oil was concentrated in vacuo to remove any residual heptane. The oil was purified by flash chromatography on silica gel using a gradient of pure hexanes to 8% EtOAc/hexanes to give a light-yellow oil. MS: m/z 265 (M−1). Calc'd for C₁₄H₁₈O₅: 266.12.

(b) Ethyl 3-(dimethylamino)-2-{2-[(4-methoxyphenyl)methoxy]-acetyl}prop-2-enoate. Ethyl 4-[(4-methoxyphenyl)methoxy]-3-oxobutanoate (Step a, 5.25 g, 19.7 mmol) and N,N′-dimethylformamide dimethyl acetal (5.00 mL, 37.6 mmol) were heated neat at 100° C. for 2 h. The resulting solution was concentrated in vacuo to give a dark red oil. MS: m/z 322 (M+1). Calc'd for C₁₇H₂₃NO₅: 321.16.

(c) Ethyl 5-acetyl-2-{[(4-methoxyphenyl)methoxy]methyl}-6-oxo-1,6-dihydropyridine-3-carboxylate. To a solution of acetoacetamide (1.97 g, 19.5 mmol) in 150 mL of anhydrous THF was added NaH (60% in mineral oil, 0.64 g, 16.0 mmol) in portions over 5 min. The solution was stirred at RT for 15 min, then a solution of ethyl 3-(dimethylamino)-2-{2-[(4-methoxyphenyl}methoxy]acetyl)prop-2-enoate (Step b, 6.28 g, 19.5 mmol) in 60 mL of anhydrous THF was added at a fast drip to the reaction. After the addition was completed the reaction was stirred at 60° C. overnight. The solution was cooled to RT and concentrated in vacuo. The resulting material was suspended in 200 mL of H₂O and acidified with 5N HCl (aq) to pH ˜2. The aqueous solution was extracted with EtOAc (3×). The combined EtOAc layers were washed with brine, dried over MgSO₄, and concentrated in vacuo to give an oil. The oil was treated with Et₂O and the resulting precipitate was filtered to give a light-yellow solid. MS m/z: 360 (M+1). Calc'd for C₁₉H₂₁NO₆: 359.14.

(d) Ethyl 5-(2-bromoacetyl)-2-{[(4-methoxyphenyl)methoxy]methyl}-6-oxo-1,6-dihydropyridine-3-carboxylate. To a solution of ethyl 5-acetyl-2-{[(4-methoxyphenyl)methoxy]methyl}-6-oxo-1,6-dihydropyridine-3-carboxylate (Step c, 1.06 g, 3.0 mmol) in 50 mL of anhydrous THF was added 5,5′-dibromobarbituric acid (0.60 g, 3.0 mmol). The reaction was stirred at 60° C. overnight. The solution was concentrated in vacuo and the resulting residue treated with Et₂O. The precipitate was filtered to give a light-orange solid that was used without further purification. MS m/z: 438, 440 (M+1). Calc'd for C₁₉H₂₀BrNO₆: 437.05.

(e) Ethyl 2-{[(4-methoxyphenyl)methoxy]methyl}-6-oxo-5-(2-(4-pyridyl)(1,3-thiazol-4-yl))-1,6-dihydro-3-pyridinecarboxylate. A solution of ethyl 5-(2-bromoacetyl)-2-([(4-methoxyphenyl)-methoxy]methyl)-6-oxo-1,6-dihydropyridine-3-carboxylate (1.0 g, 2.3 mmol), and isothionicotinamide (0.23 mg, 1.7 mmol) in 25 mL of EtOH was stirred at reflux overnight. The resulting solution was cooled to RT and an orange-brown solid was filtered. The solid was coated onto silica gel and purified on an ISCO flash chromatography instrument using a gradient of 1% MeOH/CH₂Cl₂ to 3% MeOH/CH₂Cl₂ to give a light-yellow solid that was suspended in a minimum of EtOH and filtered to give an off-white solid. MS m/z: 478 (M+1). Calc'd for C₂₅H₂₃N₃O₅S Exact Mass: 477.14.

### Example 24

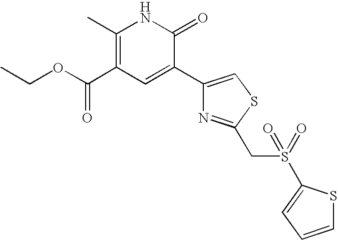

### Ethyl 2-methyl-6-oxo-5-{2-[(2-thienylsulfonyl)methyl](1,3-thiazol-4-yl)}-1,6-dihydro-3-pyridinecarboxylate

(a) Ethyl 2-acetyl-3-(dimethylamino)prop-2-enoate. Ethyl acetoacetate (25.0 mL, 196.1 mmol, Aldrich Chemical Co.) and N,N′-dimethylformamide dimethyl acetal (65.0 mL, 489.3 mmol, Aldrich Chemical Co.) were combined and stirred at 110° C. for 2 h. The mixture was cooled to RT, then poured into 400 mL of brine. The aqueous solution was extracted with EtOAc (4×). The combined EtOAc layers were washed with H₂O (2×) and brine, dried over MgSO₄, and concentrated in vacuo to give a dark red oil. MS m/z: 186 (M+1). Calc'd for C₉H₁₅NO₃: 185.11.

(b) Ethyl 5-acetyl-2-methyl-6-oxo-1,6-dihydropyridine-3-carboxylate. To a solution of acetoacetamide (10.52 g, 104 mmol) in dry THF (200 mL) was added NaH (60% in mineral oil, 3.60 g, 90.0 mmol) in portions over 15 min. After stirring for an additional 15 min, a solution of ethyl 2-acetyl-3-(dimethylamino)prop-2-enoate (Step a, 19.27 g, 104 mmol) in dry THF (200 mL) was added at a fast drip. After the addition the reaction was stirred at 60° C. overnight. The thickened material was cooled to RT and concentrated in vacuo. To the resulting yellow solid was added 500 mL of H₂O, and the solution was acidified to pH 1 with the addition of 5N HCl (aq). The resulting precipitate was filtered and dried in vacuo at 70° C. to give a yellow solid. MS m/z: 224 (M+1). Calc'd for C₁₁H₁₃NO₄: 223.08.

(c) 5-(2-Bromo-acetyl)-2-methyl-6-oxo-1,6-dihydropyridine-3-carboxylic acid ethyl ester. To a stirred, cooled (0° C.) mixture of ethyl 5-acetyl-2-methyl-6-oxo-1,6-dihydropyridine-3-carboxylate (Step b, 5.0 g, 22.4 mmol) and HBF₄ (4.74 g, 29.12 nmol) in anhydrous CH₃CN (120 mL) was added NBS (8.0 g, 44.8 mmol). The reaction mixture was stirred at RT for 24 h, concentrated, taken up in H₂O, extracted with CH₂Cl₂ (3×). The combined extracts were dried over MgSO₄, concentrated, and purified by flash column chromatography (50% EtOAc/Hexane) to give a tan solid.

(d) Ethyl 2-methyl-6-oxo-5-{2-[(2-thienylsulfonyl)methyl](1,3-thiazol-4-yl)}-1,6-dihydro-3-pyridinecarboxylate. A mixture of 5-(2-bromo-acetyl)-2-methyl-6-oxo-1,6-dihydropyridine-3-carboxylic acid ethyl ester (Step c, 0.10 g, 0.33 mmol) and 2-(thiophene-2-sulfonyl)-thioacetamide (0.1 g, 0.43 mmol) in EtOH (3 mL) was heated at 150° C. by microwave for 7 min. The solid was filtered and triturated with MeOH, filtered and dried by air to give an off white solid. MS (M+1): 425.4. Calc'd for C₁₇H₁₆N₂O₅S₃ Exact Mass: 424.02. MP: 300° C. (dec).

### Example 25

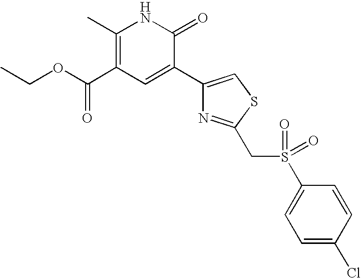

### Ethyl 5-[2-({[(4-chlorophenyl)methyl]sulfonyl}methyl)(1,3-thiazol-4-yl)]-2-methyl-6-oxo-1,6-dihydro-3-pyridinecarboxylate

A mixture of ethyl 5-(2-bromo-acetyl)-2-methyl-6-oxo-1,6-dihydro-pyridine-3-carboxylate (Example 24c) (0.10 g, 0.33 mmol) and 2-(4-chloro-benzenesulfonyl)-thioacetamide (0.11 g, 0.43 mmol) in EtOH (3 mL) was heated at 150° C. by microwave for 7 min. The solid was filtered and triturated with MeOH, filtered and dried by air to give an off white solid. MS (M+1): 453.4. Calc'd for C₁₉H₁₇ClN₂O₅S₂ Exact Mass: 452.03. MP: 300° C. (dec).

### Example 26

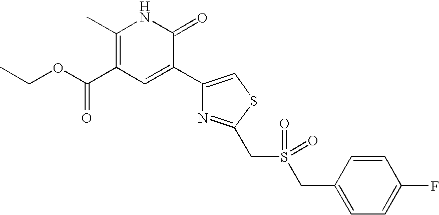

### Ethyl 5-[2-({[(4-fluorophenyl)methyl]sulfonyl}m thyl)(1,3-thiazol-4-yl)]-2-methyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 2-(4-fluoro-phenylmethanesulfonyl)-thioacetamide in EtOH (3 mL) was heated at 150° C. by microwave for 7 min. The solid was filtered and triturated with MeOH, filtered and dried by air to give an off-white solid. MS (M+1): 451.4. Calc'd for C₂₀H₁₉FN₂O₅S₂ Exact Mass: 450.07. MP: 300° C. (dec).

### Example 27

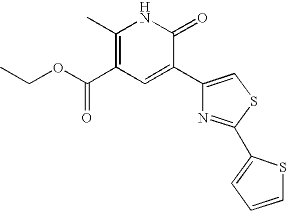

### Ethyl 2-methyl-6-oxo-5-{2-[2-thienyl(1,3-thiazol-4-yl)}-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 2-thienylthioamide (0.06 g, 0.43 mmol) in EtOH (3 mL) was heated at 150° C. by microwave for 7 min. The solid was filtered and triturated with MeOH, filtered and dried by air to give a tan solid. MS (M+1): 347.4. Calc'd for C₁₆H₁₄N₂O₃S₂ Exact Mass: 346.04. MP: 230° C. (dec).

### Example 28

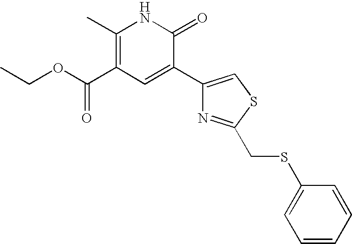

### Ethyl 2-methyl-6-oxo-5-{2-(phenylthiomethyl)(1,3-thiazol-4-yl)}-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 2-phenylsulfanyl-thioacetamide (0.07 g, 0.43 mmol) in EtOH (3 mL) was heated at 150° C. by microwave for 7 min. The solid was filtered and triturated with MeOH, filtered and dried by air to give an off white solid. MS (M+1): 387.4. Calc'd for C₁₉H₁₈N₂O₃S₂ Exact Mass: 386.08. MP: 260° C. (dec).

### Example 29

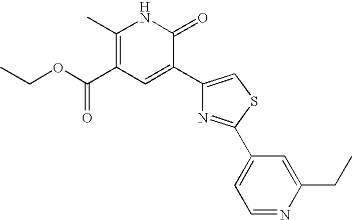

### Ethyl 5-[2-(2-ethyl(4-pyridyl))(1,3-thiazol-4-yl)-2-methyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 4-(2-ethylpyridinyl)thioamide (0.07 g, 0.43 mmol) in EtOH (3 mL) was heated at 150° C. by microwave for 7 min. The solid was filtered and triturated with MeOH, filtered and dried by air to give a tan solid. MS (M+1): 370.4. Calc'd for C₁₉H₁₉N₃O₃S Exact Mass: 369.11. MP: 270° C. (dec).

### Example 30

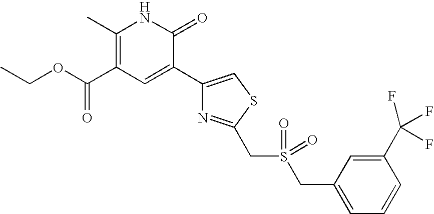

### Ethyl 2-methyl-6-oxo-5-{2-[({[3-trifluromethyl)phenyl]methyl}-sulfonyl)methyl]](1,3-thiazol-4-yl)}-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and (3-trifluoromethylbenzylsulfonyl)-ethanethioamide (0.09 g, 0.43 mmol) in EtOH (3 mL) was heated at 150° C. by microwave for 7 min. The solid was filtered and triturated with MeOH, filtered and dried by air to give an off-white solid. MS (M+1): 501.4. Calc'd for C₂₁H₁₉F₃N₂O₅S₂ Exact Mass: 500.07. MP: 300° C. (dec).

### Example 31

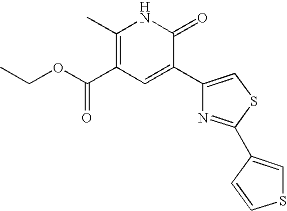

### Ethyl 2-methyl-6-oxo-5-{2-[3-thienyl](1,3-thiazol-4-yl)}-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 3-thienylthioamide (0.06 g, 0.43 mmol) in EtOH (3 mL) was heated at 150° C. by microwave for 7 min. The solid was filtered and triturated with MeOH, filtered and dried by air to give an off-white solid. MS (M+1): 347.4. Calc'd for C₁₆H₁₄N₂O₃S₂ Exact Mass: 346.04. MP: 230° C. (dec).

### Example 32

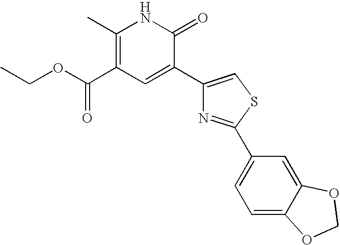

### Ethyl 5-(2-(2H-benzo[d]1,3-dioxolan-5-yl)(1,3-thiazol-4-yl))-2-m thyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and benzo[1,3]dioxole-5-carbothioic acid amide (0.06 g, 0.43 mmol) in EtOH (3 mL) was heated at 150° C. by microwave for 7 min. The solid was filtered and triturated with MeOH, filtered and dried by air to give a tan solid. MS (M+1): 385.4. Calc'd for C₁₉H₁₆N₂O₅S Exact Mass: 384.08. MP: 230° C. (dec).

### Example 33

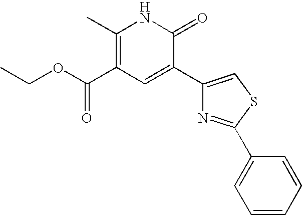

### Ethyl 2-methyl-6-oxo-5-{2-phenyl(1,3-thiazol-4-yl)}-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and thiobenzamide (0.07 g, 0.43 mmol) in EtOH (3 mL) was heated at 150° C. by microwave for 7 min. The solid was filtered and triturated with MeOH, filtered and dried by air to give an off-white solid. MS (M+1): 341.4. Calc'd for C₁₈H₁₆N₂O₃S Exact Mass: 340.09. MP: 260° C. (dec).

### Example 34

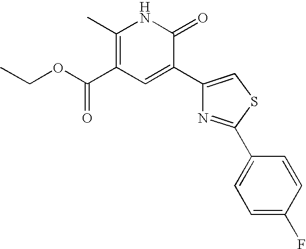

### Ethyl 2-methyl-6-oxo-5-{2-[4-fluorophenyl](1,3-thiazol-4-yl)}-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 4-fluoro-thiobenzamide (0.09 g, 0.43 mmol) in EtOH (3 mL) was heated at 150° C. by microwave for 7 min. The solid was filtered and triturated with MeOH, filtered and dried by air to give an off-white solid. MS (M+1): 359.

Calc'd for C₁₈H₁₅FN₂O₃S. MP: 260° C. (dec).

### Example 35

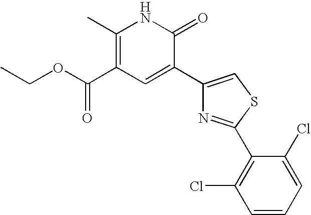

### Ethyl 5-[2-(2,6-dichlorophenyl)(1,3-thiazol-4-yl)]2-methyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 2,6-dichloro-thiobenzamide (0.08 g, 0.43 mmol) in EtOH (3 mL) was heated at 150° C. by microwave for 7 min. The solid was filtered and triturated with MeOH, filtered and dried by air to give a white solid. MS (M+1): 409.4. Calc'd for C₁₈H₁₄Cl₂N₂O₃S Exact Mass: 408.01. MP: 260° C. (dec).

### Example 36

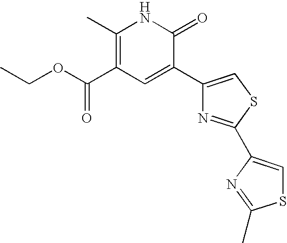

### Ethyl 2-methyl-5-[2-(2-methyl)(1,3-thiazol-4-yl))(1,3-thiazol-4-yl)]-6-oxo-1,6-dihydro-3-pyridinecarboxylate

A mixture of 5-(2-bromo-acetyl)-2-methyl-6-oxo-1,6-dihydro-pyridine-3-carboxylic acid ethyl ester (Example 24c) (0.10 g, 0.33 mmol) and 2-methyl-thiazole-4-carbothioic acid amide (0.07 g, 0.43 mmol) in EtOH (3 mL) was heated at 150° C. by microwave for 7 min. The solid was filtered and triturated with MeOH, filtered and dried by air to give a tan solid. MS (M+1): 362.1. Calc'd for C₁₆H₁₅N₃O₃S₂ Exact Mass: 361.06. MP: 195° C. (dec).

### Example 37

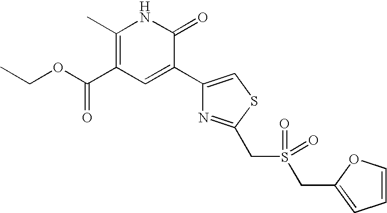

### Ethyl 5-(2-{[(2-furylmethyl)sulfonyl]methyl}(1,3-thiazol-4-yl))-2-methyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 2-(furan-2-ylmethanesulfonyl)-thioacetamide (0.08 g, 0.43 mmol) in EtOH (3 mL) was heated at 150° C. by microwave for 7 min. The solid was filtered and triturated with MeOH, filtered and dried by air to give an off-white solid. MS (M+1): 423.1. Calc'd for C₁₈H₁₈N₂O₆S₂ Exact Mass: 422.06. MP: 290° C. (dec).

### Example 38

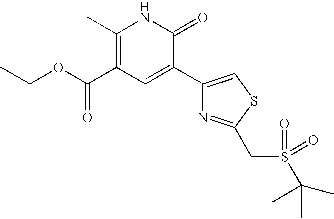

### Ethyl 5-(2-{[(tert-butyl)sulfonyl]methyl}(1,3-thiazol-4-yl))-2-methyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and tert-butylsulfonyl-thioacetamide (0.08 g, 0.43 mmol) in EtOH (3 mL) was heated at 150° C. by microwave for 7 min. The solid was filtered and triturated with MeOH, filtered and dried by air to give an off-white solid. MS (M+1): 399.1. Calc'd for C₁₇H₂₂N₂O₅S₂ Exact Mass: 398.10. MP: 250° C. (dec).

### Example 39

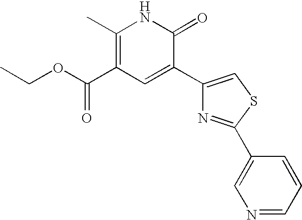

### Ethyl 2-methyl-6-oxo-5-2-(3-pyridyl)(1,3-thiazol-4-yl))-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 3-pyridinylthioacetamide (0.08 g, 0.43 mmol) in EtOH (3 mL) was heated at 150° C. by microwave for 7 min. The solid was filtered and triturated with MeOH, filtered and dried by air to give a tan solid. MS (M+1): 342.4.

Calc'd for C₁₇H₁₅N₃O₃S Exact Mass: 341.08. MP: 230° C. (dec).

### Example 40

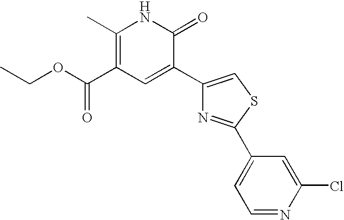

### Ethyl 5-[2-(2-chloro-(4-pyridyl))(1,3-thiazol-4-yl)-2-methyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.20 g, 0.66 mmol) and 2-chloroisothionicotinamide (0.18 g, 0.86 mmol) in EtOH (6 mL) was heated at 150° C. by microwave for 7 min. The solid was filtered and triturated with MeOH, filtered and dried by air to give a light yellow solid. MS (m+2): 377.4. Calc'd for C₁₇H₁₄ClN₃O₃S Exact Mass: 375.04. MP: 250° C. (dec).

### Example 41

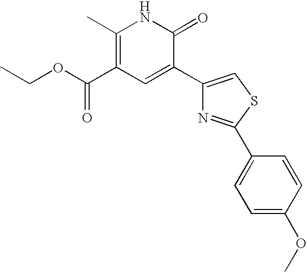

### Ethyl 2-methyl-6-oxo-5-{2-[4-methoxyphenyl](1,3-thiazol-4-yl)}-1,6-dihydro-3-pyridinecarboxylate

A mixture of ethyl 5-(2-bromo-acetyl)-2-methyl-6-oxo-1,6-dihydropyridine-3-carboxylate (Example 24c) (0.10 g, 0.33 mmol) and 4-methoxyphenyl-thioacetamide (0.09 g, 0.43 mmol) in EtOH (3 mL) was heated at 150° C. by microwave for 7 min. The solid was filtered and triturated with MeOH, filtered and dried by air to give a light yellow solid. MS (M+1): 371.1. Calc'd for C₁₉H₁₈N₂O₄S Exact Mass: 370.10. MP: 240° C. (dec).

### Example 42

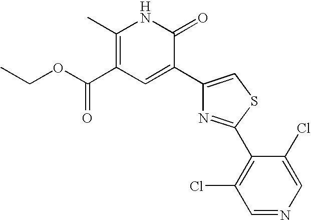

### Ethyl 5-[2-(3,5-dichloro-pyridyl-4-yl)-thiazol-4-yl]-2-methyl-6-oxo-1,6-dihydro-3-pyridinecarboxylate

A mixture of ethyl 5-(2-bromoacetyl)-2-methyl-6-oxo-1,6-dihydropyridine-3-carboxylate (Example 24c) (0.10 g, 0.33 mmol) and 2,6-dichloroisothionicontinamide (0.09 g, 0.43 mmol) in EtOH (3 mL) was heated at 150° C. by microwave for 7 min. The solid was filtered and triturated with MeOH, filtered and dried by air to give a light-yellow solid. MS (m+4): 414.1. Calc'd for C₁₇H₁₃Cl₂N₃O₃S Exact Mass: 409.01. MP: 290° C. (dec).

### Example 43

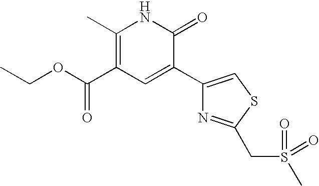

### Ethyl 5-(2-{[(methyl)sulfonyl]methyl}(1,3-thiazol-4-yl))-2-methyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.05 g, 0.13 mmol) and methylsulfonyl-thioacetamide (0.07 g, 0.43 mmol) in EtOH (3 mL) was heated at 150° C. for 7 min by microwave. The mixture was cooled and concentrated, taken up in H₂O, stirred, and filtered. The solid was purified by HPLC to give an off-white solid. MS (M+1): 357.1. Calc'd for C₁₄H₁₆N₂O₅S₂ Exact Mass: 356.05. MP: 230° C. (dec).

### Example 44

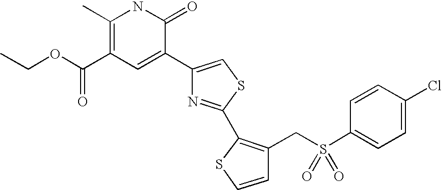

### Ethyl 5-[2-(3-{[4-chlorophenyl)sulfonyl]methyl)(2-thienyl))(1,3-thiazol-4-yl)]-2-methyl-6-oxo-1,6-dihydro-3-pyridinecarboxylate

A mixture of ethyl 5-(2-bromo-acetyl)-2-methyl-6-oxo-1,6-dihydropyridine-3-carboxylate (Example 24c) (0.05 g, 0.13 mmol) and 3-(4-chloro-benzenesulfonylmethyl)-thiophene-2-carbothioic acid amide (0.17 g, 0.43 mmol) in EtOH (3 mL) was heated at 150° C. for 7 min by microwave. The mixture was cooled and concentrated, taken up in H₂O, stirred, and filtered. The solid was purified by HPLC to give a tan solid. MS (m+2): 537.1. Calc'd for C₂₃H₁₉ClN₂O₅S₃ Exact Mass: 534.01. MP: 300° C. (dec).

### Example 45

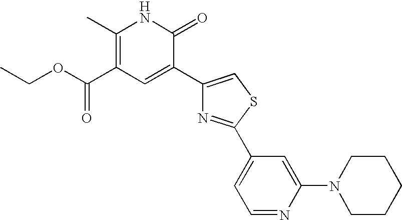

### Ethyl 2-methyl-6-oxo-5-(2-(2-(1-piperidinyl)-4-pyridinyl)-1,3-thiazol-4-yl)-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and piperidine (1 mL) was heated at 150° C. for 20 min. by microwave. The mixture was cooled, concentrated, and purified by flash column chromatography (3% MeOH/CH₂Cl₂) to give a light-yellow solid. MS (M+1): 425.1. Calc'd for C₂₂H₂₄N₄O₃S Exact Mass: 424.16. MP: 260° C. (dec).

### Example 46

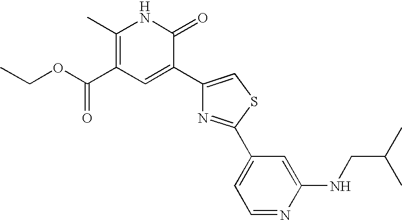

### Ethyl 2-methyl-5-(2-(2-((2-methylpropyl)amino)-4-pyridinyl)-1,3-thiazol-4-yl)-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and isobutylamine (1 mL) was heated at 160° C. for 1 h. The mixture was cooled, concentrated, and purified by flash column chromatography (3% MeOH/CH₂Cl₂) to give a tan solid. MS (M+1): 413.1. Calc'd for C₂₁H₂₄N₄O₃S Exact Mass: 412.16. MP: 260° C. (dec).

### Example 47

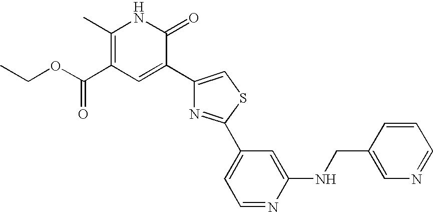

### Ethyl 2-methyl-6-oxo-5-(2-(2-((3-pyridinylmethyl)amino)-4-pyridinyl)-1,3-thiazol-4-yl)-1,6-dihydro-3-pyridin carboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and 3-pyridylmethylamine (1 mL) was heated at 160° C. for 1 h. The mixture was cooled, concentrated, and purified by flash column chromatography (7% MeOH/CH₂Cl₂) to give an off white solid. MS (M+1): 448.1. Calc'd for C₂₃H₂₁N₅O₃S Exact Mass: 447.14. MP: 280° C. (dec).

### Example 48

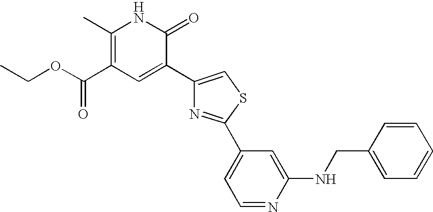

### Ethyl 2-methyl-6-oxo-5-(2-(2-((phenylmethyl)amino)-4-pyridinyl)-1,3-thiazol-4-yl)-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and benzylamine (1 mL) was heated at 160° C. for 1 h. The mixture was cooled, concentrated, and purified by flash column chromatography (3% MeOH/CH₂Cl₂) to give an off white solid. MS (M+1): 447.1. Calc'd for C₂₄H₂₂N₄O₃S Exact Mass: 446.14. MP: 290° C. (dec).

### Example 49

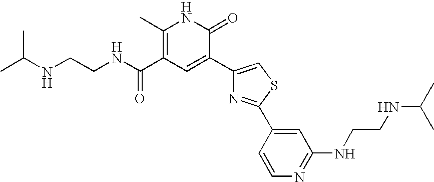

### 2-Methyl-N-(2-((1-methylethyl)amino)ethyl)-5-(2-(2-((2-((1-methylethyl)amino)ethyl)amino)-4-pyridinyl)-1,3-thiazol-4-yl)-6-oxo-1,6-dihydro-3-pyridinecarboxylate

### Example 50

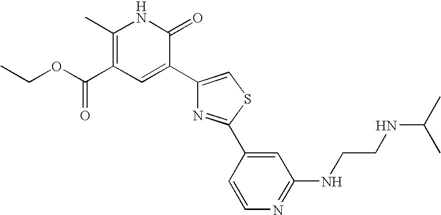

### Ethyl 2-methyl-5-(2-(2-((2-((1-methylethyl)amino)ethyl)amino)-4-pyridinyl)-1,3-thiazol-4-yl)-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and 2-isopropylamino-ethylamine (0.11 g, 0.8 mmol) and Cu powder (0.09 g, 0.14 mmol) in 2,4,6-collidine (3 mL) was heated at 160° C. for 16 h. The mixture was cooled, concentrated, and purified by flash column chromatography (3% MeOH/CH₂Cl₂) to give Example 49 and Example 50 which were isolated as tan solid. Example 49: MS (M+1): 498.2. Calc'd for C₂₅H₃₅N₇O₂S: 497.26. MP: 260° C. (dec). Example 50: MS (M+1): 442.1.

Calc'd for C₂₂H₂₇N₅O₃S: 441.18. MP: 260° C. (dec).

### Example 51

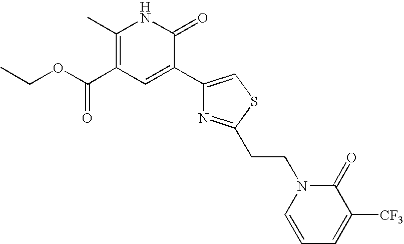

### Ethyl 2-methyl-6-oxo-5-(2-(2-(2-oxo-3-(trifluoromethyl)-1(2H)-pyridinyl)ethyl)-1,3-thiazol-4-yl)-1,6-dihydro-3-pyridinecarboxylate

A mixture of ethyl 5-(2-bromoacetyl)-2-methyl-6-oxo-1,6-dihydro-pyridine-3-carboxylate (Example 24c) (0.06 g, 0.16 mmol) and 3-(2-oxo-3-trifluoromethyl-2H-pyridin-1-yl)-thiopropionamide (0.05 g, 0.21 mmol) in EtOH (3 mL) was heated at 170° C. for 7 min by microwave. The mixture was cooled and concentrated, taken up in H₂O, stirred, and filtered. The solid was purified by HPLC to give a yellow solid. MS (M+1): 454.1. Calc'd for C₂₀H₁₈F₃N₃O₄S Exact Mass: 453.10. MP: 250° C. (dec).

### Example 52

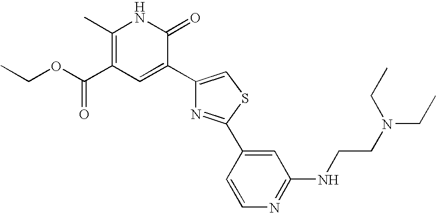

### Ethyl 5-(2-(2-((2-(diethylamino)ethyl)amino)-4-pyridinyl)-1,3-thiazol-4-yl)-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and 2-isopropylamino-ethylamine (1 mL) was heated at 160° C. for 1 h. The mixture was cooled, concentrated, and purified by flash column chromatography (10% MeOH/CH₂Cl₂) to give a tan solid. MS (M+1): 456.2. Calc'd for C₂₃H₂₉N₅O₃S Exact Mass: 455.20. MP: 250° C. (dec).

### Example 53

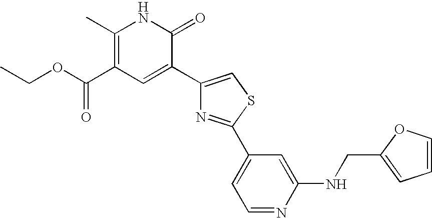

### Ethyl 5-(2-(2-[(fur-2-ylmethyl)-amino]-pyridin-4-yl}-thiazol-4-yl)-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and 2-furan-2-yl-methylamine (0.11 g, 0.8 mmol) and Cu powder (0.09 g, 0.14 mmol) in 2,4,6-collidine (3 mL) was heated at 160° C. for 16 h. The mixture was cooled, concentrated, and purified by flash column chromatography (3% MeOH/CH₂Cl₂) to give a tan solid. The solid was dissolved in warm 1,4-dioxane and treated with 1M HCl in ether. The HCl salt was filtered and dried by air. MS (M+1): 437.4. Calc'd for C₂₂H₂₀N₄O₄S. MP: 260° C. (dec).

### Example 54

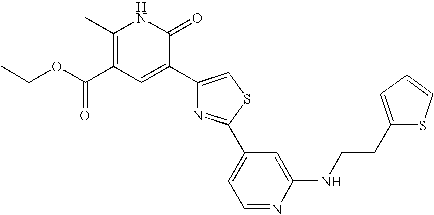

### Ethyl 5-{2-[2-(2-thien-2-yl-ethylamino)-pyridin-4-yl]-thiazol-4-yl}-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and 2-thiophene-2-yl-ethylamine (0.11 g, 0.8 mmol) and Cu powder (0.09 g, 0.14 mmol) in 2,4,6-collidine (3 mL) was heated at 160° C. for 16 h. The mixture was cooled, concentrated, and purified by flash column chromatography (3% MeOH/CH₂Cl₂) to give a tan solid. The solid was dissolved in warm 1,4-dioxane and treated with 1M HCl in ether. The HCl salt was filtered and dried by air. MS(M+1): 437.4. Calc'd for C₂₂H₂₀N₄O₄S. MP: 280° C. (dec).

### Example 55

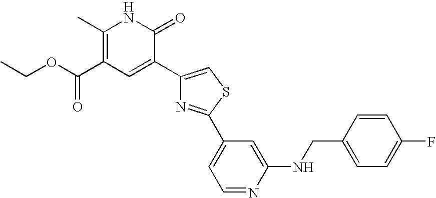

### Ethyl 5-{2-[2-(4-fluoro-benzylamino)-pyridin-4-yl]-thiazol-4-yl}-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and 4-fluorobenzylamine (0.07 g, 0.8 mmol) and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a light yellow solid. The solid was dissolved in warm 1,4-dioxane and treated with 1 M HCl in ether. The HCl salt was filtered and dried by air. MS(M+1): 465.1. Calc'd for C₂₄H₂₁FN₄O₃S Exact Mass: 464.13. MP: 280° C. (dec).

### Example 56

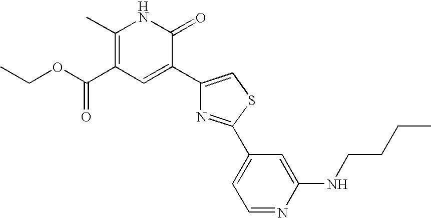

### Ethyl 5-[2-(2-butylamino-pyridin-4-yl)-thiazol-4-yl]-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol), n-butylamine (0.09 g, 1.33 mmol), and Cu powder (0.09 g, 0.14 mmol) in 2,4,6-collidine (3 mL) was heated at 160° C. for 16 h. The mixture was cooled, concentrated, and purified by flash column chromatography (3% MeOH/CH₂Cl₂) to give a tan solid which was dissolved in warm 1,4-dioxane and treated with 1M HCl in Et₂O (0.12 mL). The precipitated HCl salt was filtered and dried by air. MS(M+1): 413.1. Calc'd for C₂₁H₂₄N₄O₃S Exact Mass: 412.16. MP: 230° C. (dec).

### Example 57

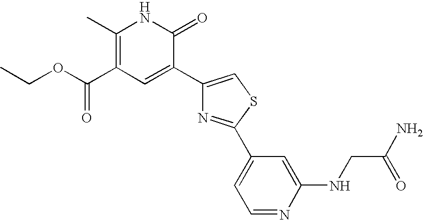

### Ethyl 5-{2-[2-(carbamoylmethyl-amino)-pyridin-4-yl]-thiazol-4-yl}-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol), K₂CO₃ (0.09 g, 0.81 mmol), 2-aminoacetamide hydrochloride (0.09 g, 0.81 mmol), and Cu powder (0.09 g, 0.14 mmol) in 2,4,6-collidine and DMSO (1:1, 4 mL) was heated at 160° C. for 16 h. The mixture was cooled, concentrated, and purified by flash column chromatography (5% MeOH/CH₂Cl₂) to give a tan solid which was dissolved in warm 1,4-dioxane and treated with 1M HCl in ether (0.12 mL). The precipitated HCl salt was filtered and dried by air. MS(M+1): 414.1. Calc'd for C₁₉H₁₉N₅O₄S Exact Mass: 413.12. MP: 270° C. (dec).

### Example 58

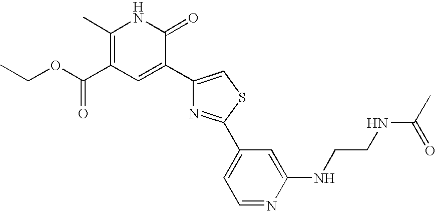

### Ethyl 5-{2-[2-acetylamino-ethylamino)-pyridin-4-yl]-thiazol-4-yl}-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydro-3-pyridinecarboxylate (Example 40) (0.10 g, 0.27 mmol), K₂CO₃ (0.18 g, 1.33 mmol), N-(2-amino-ethyl)-acetamide (0.11 g, 0.8 mmol), and Cu powder (0.09 g, 0.14 mmol) in 2,4,6-collidine and DMSO (1:1, 4 mL) was heated at 160° C. for 16 h. The mixture was cooled, concentrated, and purified by flash column chromatography (5% MeOH/CH₂Cl₂) to give a tan solid. The solid was dissolved in warm 1,4-dioxane and treated with 1M HCl in ether. The HCl salt was filtered and dried by air. MP: 270° C. (dec). MS (M+1): 442.4.

Calc'd for C₂₁H₂₃N₅O₄S.

### Example 59

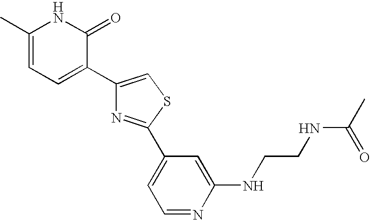

### N-(2-{4-[4-(6-Methyl-2-oxo-1,6-dihydropyridin-3-yl)-thiazol-2-yl]-pyridin-2-ylamino}-ethyl)-acetamide

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol), K₂CO₃ (0.18 g, 1.33 mmol), N-(2-aminoethyl)-acetamide (0.11 g, 0.8 mmol), and Cu powder (0.09 g, 0.14 mmol) in 2,4,6-collidine and DMSO (1:1, 4 mL) was heated at 160° C. for 16 h. The mixture was cooled, concentrated, and purified by flash column chromatography (5% MeOH/CH₂Cl₂) to give a tan solid. MS (M+1): 370.1. Calc'd for C₁₈H₁₉N₅O₂S Exact Mass: 369.13. MP: 230° C. (dec).

### Example 60

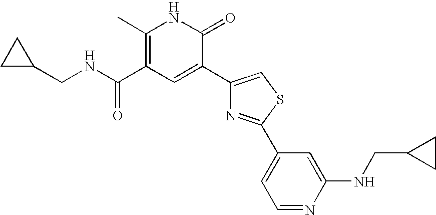

### N-(Cyclopropylmethyl)-5-(2-(2-((cyclopropylmethyl)amino)-4-pyridinyl)-1,3-thiazol-4-yl)-2-methyl-6-oxo-1,6-dihydro-3-pyridinecarboxamide

A mixture of ethyl 5-(2-(2-chloro-4-pyridinyl)-1,3-thiazol-4-yl)-2-methyl-6-oxo-1,6-dihydro-3-pyridinecarboxylate (Example 40) (0.10 g, 0.27 mmol), cyclopropylmethylamine (0.07 g, 0.54 mmol), and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a light yellow solid. The solid was dissolved in warm 1,4-dioxane and treated with 1M HCl in ether. The HCl salt was filtered and dried by air. MS (M+1): 436. Calc'd for C₂₃H₂₅N₅O₂S Exact Mass: 435.17. MP: >260° C.

### Example 61

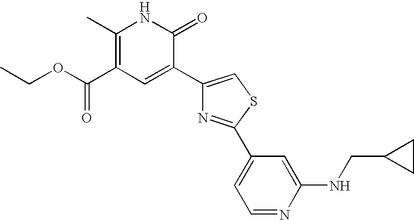

### Ethyl 5-{2-[2-(cyclopropylmethyl-amino)-pyridin-4-yl]-thiazol-4-yl}-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol), cycloppropylmethylamine (0.07 g, 0.54 mmol) and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a light yellow solid. The solid was dissolved in warm 1,4-dioxane and treated with 1M HCl in ether. The HCl salt was filtered and dried by air. MS (M+1): 411.1. Calc'd for C₂₁H₂₂N₄O₃S Exact Mass: 410.14. MP: >260° C.

### Example 62

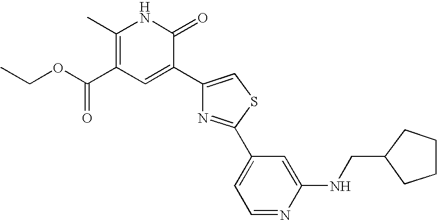

### Ethyl 5-{2-[2-(Cyclopentyl)methylamino-pyridin-4-yl]-thiazol-4-yl}-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydro-3-pyridine-carboxylate (Example 40) (0.10 g, 0.27 mmol), cyclopentyl-methylamine (0.07 g, 0.54 mmol), and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a light yellow solid. The solid was dissolved in warm 1,4-dioxane and treated with 1 M HCl in ether. The HCl salt was filtered and dried by air. MS (M+1): 439.1. Calc'd for C₂₃H₂₆N₄O₃S Exact Mass: 438.17. MP: 260° C. (dec).

### Example 63

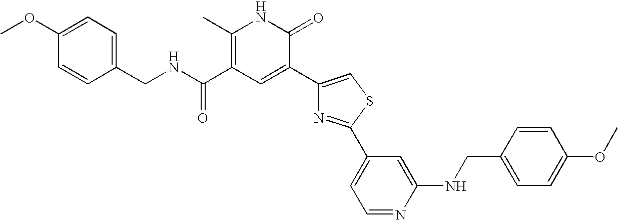

### 5-{2-[2-(4-Methoxy-benzyamino)-pyridin-4-yl]-thiazol-4-yl}-2-methyl-6-oxo-1,6-dihydropyridine-3-carboxylic acid 4-methoxy-benzylamide

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol), 4-methoxybenzylamine (0.07 g, 0.54 mmol) and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a light yellow solid. The solid was dissolved in warm 1,4-dioxane and treated with 1M HCl in ether. The HCl salt was filtered and dried by air. MS (M+1): 568.1. Calc'd for C₃₁H₂₉N₅O₄S Exact Mass: 567.19. MP: 280° C. (dec).

### Example 64

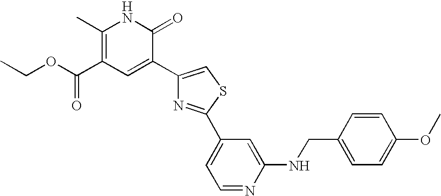

### Ehtyl 5-{2-[2-(4-Methoxy-benzyamino)-pyridin-4-yl]-thiazol-4-yl}-2-methyl-6-oxo-1,6-dihydro-3-pyridinecarboxylate

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol) and 4-methoxybenzylamine (0.07 g, 0.54 mmol) and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a light yellow solid. MS (M+1): 477.1. Calc'd for C₂₅H₂₄N₄O₄S Exact Mass: 476.15. MP: >260° C.

### Example 65

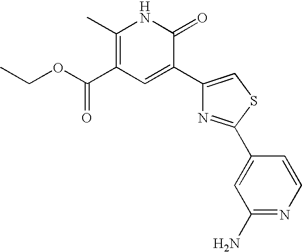

### Ethyl 2-methyl-6-oxo-5-(2-(2-(amino)-4-pyridinyl)-1,3-thiazol-4-yl)-1,6-dihydro-3-pyridinecarboxylate

A mixture of 5-{2-[2-(4-methoxy-benzylamino)-pyridin-4-yl]-thiazol-4-yl}-2-methyl-6-oxo-1,6-dihydropyridine-3-carboxylic acid ethyl ester (Example 64) (0.030 g, 0.07 mmol) and TFA (0.2 mL) in CH₂Cl₂ (2 mL) was stirred at RT for 16 h. The mixture was concentrated and triturated in MeOH to give a tan solid. MS (M+1): 357.1. Calc'd for C₁₇H₁₆N₄O₃S Exact Mass: 356.09. MP: >260° C.

### Example 66

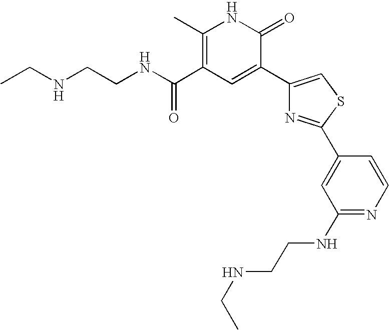

### 2-Methyl-N-(2-((1-methylethyl)amino)ethyl)-5-(2-(2-((2-((1-methylethyl)amino)ethyl)amino)-4-pyridinyl)-1,3-thiazol-4-yl)-6-oxo-1,6-dihydro-3-pyridinecarboxamide

A mixture of ethyl 5-(2-(2-chloro-4-pyridinyl)-1,3-thiazol-4-yl)-2-methyl-6-oxo-1,6-dihydropyridine-3-carboxylate (Example 40) (0.10 g, 0.27 mmol), 3-aminomethylpyridine (0.11 g, 0.8 mmol), and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a white solid. MS (M+1): 470.4. Calc'd for C₂₃H₃₁N₇O₂S. MP: >260° C.

### Example 67

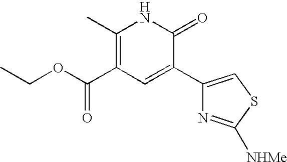

### Ethyl 2-methyl-5-[2-(methylamino)(1,3-thiazol-4-yl)]-6-oxo-1,6-dihydro-3-pyridinecarboxylate

A mixture of ethyl 5-(2-bromoacetyl)-2-methyl-6-oxo-1,6-dihydropyridine-3-carboxylate (Example 75a) (513 mg, 1.7 mmol) and N-methyl thiourea (Aldrich) (171 mg, 1.9 mmol) in EtOH (4 mL) was heated at 140° C. in the microwave for 5 min. The solids were filtered, washed with EtOH and dried in vacuo to give an off-white amorphous solid. MS m/z: 294 (M+1); 292 (M−1). Calc'd Exact Mass: 293.08.

Anal. Calc'd for C₁₃H₁₅N₃O₃S.HBr.H₂O: C, 39.80; H, 4.63; N, 10.71; Br, 20.37. Found: C, 39.88; H, 4.58; N, 10.82; Br, 20.44.

### Example 68

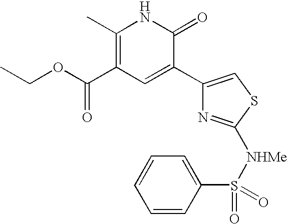

### Ethyl 2-methyl-5-{2-[methyl(phenylsulfonyl)amino](1,3-thiazol-4-yl)}-6-oxo-1,6-dihydro-3-pyridinecarboxylate

A mixture of ethyl 2-methyl-5-[2-(methylamino)(1,3-thiazol-4-yl)]-6-oxo-1,6-dihydropyridine-3-carboxylate (Example 67) (296 mg, 0.5 mmol), benzenesulfonyl chloride (0.14 mL, 1.1 mmol) and DMAP (13 mg, 0.1 mmol) in pyridine (4 mL) was heated at 50° C. After 9 h the reaction was cooled to RT and the solvent was removed in vacuo. The residue was stirred over CH₂Cl₂ and the precipitate was filtered, washed with CH₂Cl₂ and dried in vacuo to give a white amorphous solid. Mp: 231-234° C. MS m/z: 434 (M+1); 432 (M−1). Calc'd Exact Mass: 433.08. Anal. Calc'd for C₁₉H₁₉FN₃O₅S_{2.}0.5 HCl: C, 50.52; H, 4.35; N, 9.30. Found: C, 50.16; H, 4.22; N, 9.28.

### Example 69

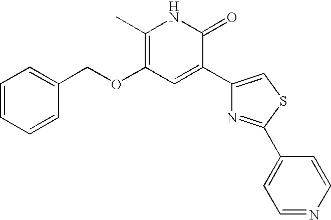

### 5-((Phenylmethyl)oxy)-3-(2-(4-pyridinyl)-1,3-thiazol-4-yl)-2(1H)-pyridinone

To a mixture of 2-(2-(4-pyridyl)-1,3-thiazol-4-yl)acetamide (148 mg, 0.7 mmol) (Example 18b) and 3-(dimethylamino)-2-(phenylmethoxy)prop-2-enal (made as described in WO98/50384)(189 mg, 0.9 mmol) in DMF (3 mL) was added 60% NaH (52 mg, 1.3 mmol) at RT. Gas evolution occurred. The reaction was heated at 70° C. After 19 h, the reaction was cooled to RT and diluted with MeOH. The mixture was purified by reverse phase preparatory HPLC to yield a yellow amorphous solid. MS m/z: 362 (M+1); 360 (M−1).

Calc'd for C₂₀H₁₅N₃O₂S: 362.0958. Found: 362.0957.

### Example 70

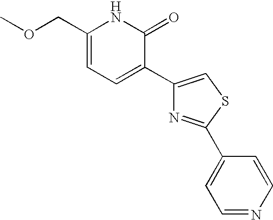

### 6-(Methoxymethyl)-3-(2-(4-pyridinyl)-1,3-thiazol-4-yl)-2(1H)-pyridinone

To a mixture of 2-(2-(4-pyridyl)-1,3-thiazol-4-yl)acetamide (Example 18b) (266 mg, 1.2 mmol) and 4-(dimethylamino)-1-methoxybut-3-en-2-one (199 mg, 1.4 mmol) in DMF (3 mL) was added 60% NaH (95 mg, 2.4 mmol) at RT. Gas evolution occurred. The reaction was heated at 70° C. After 19 h, the reaction was cooled to 0° C. and acidified with 5N HCl. The mixture was poured into H₂O and the solids were filtered and washed with H₂O and hexanes. The solid residue was dried in vacuo to give a tan amorphous solid. Mp: 249-254° C. MS m/z: 300 (M+1); 298 (M−1). Calc'd Exact Mass: 299.07. Anal. Calc'd for C₁₅H₁₃₁N₃O₂S.0.5H₂O: C, 58.42; H, 4.58; N, 13.63. Found: C, 58.22; H, 4.36; N, 13.95.

### Example 71

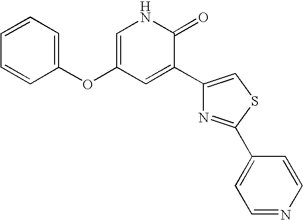

### 5-Phenoxy-3-(2-(4-pyridinyl)-1,3-thiazol-4-yl)-2(1H)-pyridinone

To a mixture of 2-(2-(4-pyridyl)-1,3-thiazol-4-yl)acetamide (Example 18b) (219 mg, 1.0 mmol) and 3-(dimethylamino)-2-phenoxyprop-2-enal (Maybridge) (233 mg, 1.2 mmol) in DMF (3 mL) was added 60% NaH (82 mg, 2.0 mmol) at RT. Gas evolution occurred. The reaction was heated at 70° C. After 19 h, the reaction was cooled to 0° C. and acidified with 5N HCl. The mixture was poured into H₂O and the solids were filtered and washed with H₂O and hexanes. The solid residue was dried in vacuo to give a yellow amorphous solid. Mp: 243-245° C. MS m/z: 348 (M+1); 346 (M−1).

Calc'd Exact Mass: 347.07. Anal. Calc'd for C₁₉H₁₃N₃O₂S.0.33 HCl.0.66H₂O: C, 61.94; H, 3.92; N, 11.41; Cl, 3.18. Found: C, 61.68; H, 3.78; N, 11.49; Cl, 2.92.

### Example 72

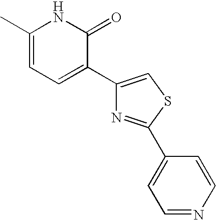

### 6-Methyl-3-(2-(4-pyridyl)(1,3-thiazol-4-yl))-(1H)-pyridin-2-one

To a mixture of 2-(2-(4-pyridyl)-1,3-thiazol-4-yl)acetamide (Example 18b) (300 mg, 1.4 mmol) and 4-(dimethylamino)but-3-en-2-one (Aldrich)(0.19 mL, 1.6 mmol) in DMF (4 mL) was added 60% NaH (118 mg, 3.0 mmol) at RT. Gas evolution occurred. The reaction was heated at 70° C. After 45 h, the reaction was allowed to cooled to 0° C. and acidified with 5N HCl. The mixture was poured into H₂O and the solids were filtered and washed with H₂O and hexanes. The solid residue was dried in vacuo to give a tan amorphous solid. MS m/z: 270 (M+1); 268 (M−1). Calc'd Exact Mass: 269.06. Anal. Calc'd for C₁₄H₁₁N₃OS.0.25H₂O: C, 61.40; H, 4.23; N, 15.35. Found: C, 61.64; H, 4.17; N, 15.00.

### Example 73

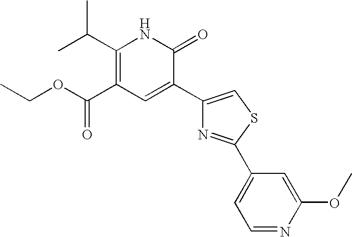

### Ethyl 2-(1-methylethyl)-5-(2-(2-methoxy-4-pyridinyl)-1,3-thiazol-4-yl)-6-oxo-1,6-dihydro-3-pyridinecarboxylate

(a) 2-Methoxylthioisonicotinamide. To a stirred mixture of 2-methoxyl-4-isonicotinonitrile (0.55 g, 4.1 mmol) and pyridine (1.62 g, 20.5 mmol) in TEA (10 mL) was bubbled with H₂S in 10 min. The resulting reaction was stirred at RT in 24 h, concentrated, stirred in H₂O, and the yellow solid was filtered and dried by air.

(b) Ethyl 5-[2-(methoxypyridin-4-yl)-thiazol-4-yl]-2-isopropyl-6-oxo-1,6-dihydro-3-pyridinecarboxylate. A mixture of ethyl 5-(2-bromoacetyl)-2-isopropyl-6-oxo-1,6-dihydropyridine-3-carboxylate (Example 10(c)) (0.10 g, 0.31 mmol) and 2-methoxylthioisonicotinamide (Step a, 0.08 g, 0.45 mmol) in EtOH (3 mL) was heated at 150° C. for 7 min by microwave. The mixture was cooled, concentrated, and purified by flash column chromatography (2% MeOH/CH₂Cl₂) to give a brown solid. MS (M+1): 400.2. Calc'd for C₂₀H₂₁N₃O₄S Exact Mass: 399.13.

### Example 74

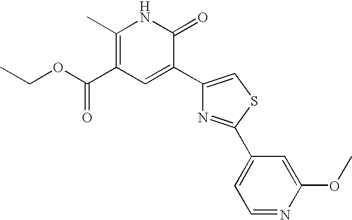

### Ethyl 2-methyl-5-(2-(2-(methoxy)-4-pyridinyl)-1,3-thiazol-4-yl)-6-oxo-1,6-dihydro-3-pyridinecarboxylate

A mixture of 5-(2-bromo-acetyl)-2-methyl-6-oxo-1,6-dihydropyridine-3-carboxylic acid ethyl ester (Example 24c) (0.10 g, 0.31 mmol) and 2-methoxythioisonicotinamide (Example 73a, 0.07 g, 0.43 mmol) in EtOH (3 mL) was heated at 150° C. for 7 min by microwave. The mixture was cooled, concentrated, and purified by flash column chromatography (2% MeOH/CH₂Cl₂) to give an off white solid. MS (M+1): 372.2. Calc'd for C₁₈H₁₇N₃O₄S Exact Mass: 371.09.

### Example 75

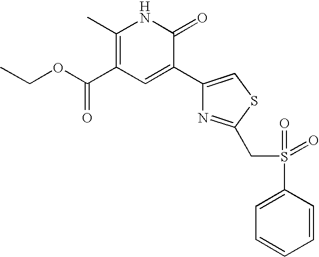

### Ethyl 2-methyl-6-oxo-5-{2-[(phenylsulfonyl)methyl](1,3-thiazol-4-yl)}-1,6-dihydro-3-pyridinecarboxylate

(a) Ethyl 5-(2-bromoacetyl)-2-methyl-6-oxo-1,6-dihydropyridine-3-carboxylate. A mixture of ethyl 5-(2-acetyl)-2-methyl-6-oxo-1,6-dihydropyridine-3-carboxylate (Bionet, 1.0 g, 4.48 mmol) and 5,5-dibromobarbituric acid (0.77 g, 2.69 mmol, Aldrich) in 50 mL of anhydrous THF was heated at reflux for 3 h. Another portion of 5,5-dibromobarbituric acid (0.1 g, 0.35 mmol) was added. Reaction was monitored by analytical HPLC until all starting materials were gone. The solvent was evaporated under reduced vacuum. The residue was partitioned between 100 mL of EtOAc and 100 mL of saturated aqueous NaHCO₃. The organic layer was separated, dried (Na₂SO₄), and concentrated to yield a yellow solid which was used directly in the next step. MS m/z: 301.9, 303.9 (M+1, equal intensity). Calc'd for C₁₁H₁₃BrNO₄: 302.00.

(b) Ethyl 2-methyl-6-oxo-5-{2-[(phenylsulfonyl)methyl]-(1,3-thiazol-4-yl)}-1,6-dihydro-3-pyridinecarboxylate. A mixture of ethyl 5-(2-bromoacetyl)-2-methyl-6-oxo-1,6-dihydropyridine-3-carboxylate (Step (a), 200 mg) and 2-phenylsulfonyl-ethanethioamide (Maybridge, 110 mg, 0.51 mmol) in 35 mL of anhydrous MeOH was heated at reflux for 6 h. A brown solution was obtained. The reaction mixture was cooled to RT and precipitates formed. The precipitates were filtered, washed carefully with CH₂Cl₂ and recrystallized from MeOH to afford the title compound as a pink solid. MS m/z: 419.2 (M+1). Calc'd for C₁₉H₁₈N₂O₅S₂ Exact Mass: 418.07.

### Example 76

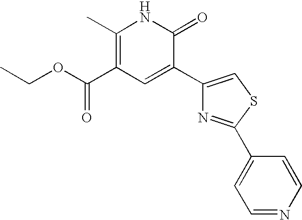

### Ethyl 2-methyl-6-oxo-5-(2-(4-pyridyl)(1,3-thiazol-4-yl))-1,6-dihydro-3-pyridinecarboxylate

A mixture of ethyl 5-(2-bromoacetyl)-2-methyl-6-oxo-1,6-dihydropyridine-3-carboxylate (Example 75(a), 270 mg) and isothionicotinamde (Lancaster, 70 mg, 0.51 mmol) in 5 mL of anhydrous MeOH was heated at 140° C. for 5 min with a microwave. The solution was cooled to RT and precipitates formed. The precipitates were filtered, washed carefully with CH₂Cl₂ and recrystallized from MeOH to afford the title compound as a yellow solid. MS m/z: 342.3 (M+1). Calc'd for C₁₇H₁₅N₃O₃S Exact Mass: 341.08.

### Example 77

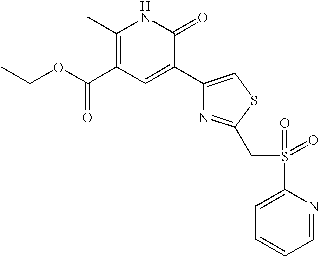

### Ethyl 2-methyl-6-oxo-5-{2-[(2-pyridylsulfonyl)methyl](1,3-thiazol-4-yl)}-1,6-dihydro-3-pyridinecarboxylate

A mixture of ethyl 5-(2-bromoacetyl)-2-methyl-6-oxo-1,6-dihydropyridine-3-carboxylate (Example 75(a), 270 mg) and 2-(2-pyridylsulfonyl)-ethanethioamide (Maybridge, 200 mg, 0.93 mmol) in 20 mL of anhydrous MeOH was heated at reflux for 6 h. The solvent was evaporated under vacuum to give a residue which was washed by 5 mL of MeOH. Crude material was collected by filtration, dissolved in minimal amount of 5% MeOH in CH₂Cl₂, and purified by prep TLC (5% MeOH in CH₂Cl₂) to afford the title compound as a light yellow solid. MS m/z: 420.1 (M+1). Calc'd for C₁₈H₁₇N₃O₅S₂ Exact Mass: 419.06.

### Example 78

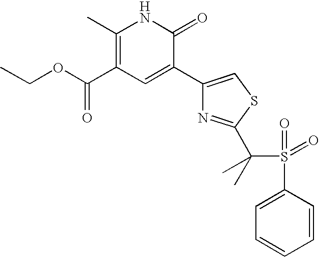

### Ethyl 2-methyl-5-(2-(1-methyl-1-(phenylsulfonyl)ethyl)-1,3-thiazol-4-yl)-6-oxo-1,6-dihydro-3-pyridinecarboxylate

(a) 2-Benzenesulfonyl-2-methyl-propionitrile. To a solution of 2-(phenylsulfonyl)acetonitrile (Aldrich-Sigma Company, 2.70 g, 15.0 mmol) in 20 mL of CH₂Cl₂ were added 10 mL of 5N NaOH, tetra-n-butylammonium iodide (0.75 g, 2.1 mmol), and 5.0 mL of MeI. The resulting mixture was stirred vigorously at RT for 1 h. Diluted with 40 mL of CH₂Cl₂ and the layers were carefully separated to avoid emulsion. The organic layer was washed with 50 mL of H₂O (2×), dried (Na₂SO₄), and concentrated to provide the title compound as a white solid. MS m/z: 231.9 (M+23). Calc'd for C₁₀H₁₁NO₂S: 209.05.

(b) 2-Amino-1,1-dimethyl-1-(phenylsulfonyl)ethane-2-thione. A solution of 2-methyl-2-(phenylsulfonyl)propanenitrile (Step a, 3.0 g, 14.4 mmol) in 20 mL of pyridine and 4 mL of TEA was purged with H₂S gas for 3 h. The resulting mixture was stirred at RT overnight. Solvents were removed under vacuum and the oily residue was azeotroped with 3×50 mL of toluene. A stock solution in 25 mL of anhydrous MeOH was then prepared and used in next steps. MS m/z: 242.2 (M−1).

Calc'd for C₁₀H₁₃NO₂S₂: 243.04.

(c) Ethyl 2-methyl-5-(2-(1-methyl-1-(phenylsulfonyl)ethyl)-1,3-thiazol-4-yl)-6-oxo-1,6-dihydro-3-pyridinecarboxylate. A mixture of ethyl 5-(2-bromoacetyl)-2-methyl-6-oxo-1,6-dihydropyridine-3-carboxylate (Example 75(a), 300 mg) and 2-amino-1,1-dimethyl-1-(phenylsulfonyl)ethane-2-thione (Step b, 1.7 mL, 1.0 mmol) in 3.5 mL of anhydrous MeOH was heated at 120° C. for 2×5 min by microwave. The reaction mixture was cooled to RT. The precipitates were collected by filtration and washed with MeOH and CH₂Cl₂ to provide the title compound as an off-white solid. MS m/z: 447.1 (M+1). Calc'd for C₂₁H₂₂N₂O₅S₂ Exact Mass: 446.10.

### Example 79

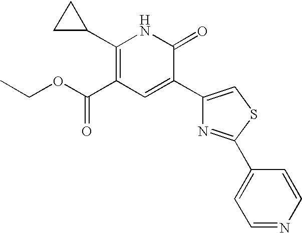

### Ethyl 2-cyclopropyl-6-oxo-5-(2-(4-pyridinyl)-1,3-thiazol-4-yl)-1,6-dihydro-3-pyridinecarboxylate

(a) 3-Cyclopropyl-3-oxo-propionic acid ethyl ester. To a solution of diethyl carbonate (10.65 g, 90.2 mmol, Aldrich Chemical Co.) and 50 mL of anhydrous THF was added (60% NaH in mineral oil, 4.87 g, 121.8 mmol) portion-wise. After stirring for 15 min, a solution of cyclopropyl methyl ketone (8.90 mL, 89.8 mmol, Aldrich Chemical Co.) in 20 mL of anhydrous THF was added dropwise to the reaction. After addition was complete the reaction was stirred at reflux for 1.5 h, cooled to RT and concentrated in vacuo. The residue was treated with cold H₂O (65 mL), followed by 1N HCl (50 mL). The resulting aqueous solution was extracted with Et₂O (3×). The combined Et₂O layers were dried over MgSO₄ and concentrated in vacuo to give a golden oil. MS m/z: 157 (M+1). Calc'd for C₈H₁₂O₃: 156.08.

(b) 2-Cyclopropanecarbonyl-3-dimethylamino-acrylic acid ethyl ester. The compound was prepared in a similar manner to Example 1a using 3-cyclopropyl-3-oxo-propionic acid ethyl ester (Step a, 9.83 g, 62.9 mmol) and N,N′-dimethylformamide dimethyl acetal (17.0 mL, 128.0 mmol) to give a reddish-brown oil. MS m/z: 212 (M+1). Calc'd for C₁₁H₁₇NO₃: 211.12.

(c) Ethyl 5-acetyl-2-cyclopropyl-6-oxo-1,6-dihydropyridine-3-carboxylate. The compound was prepared in a similar manner to Example 1b using 2-cyclopropanecarbonyl-3-dimethylamino-acrylic acid ethyl ester (Step b, 10.7 g, 50.7 mmol), acetoacetamide (5.15 g, 50.9 mmol), and NaH (60% in mineral oil, 1.61 g, 40.3 mmol) to give a yellow solid. MS m/z: 250 (M+1). Calc'd for C₁₃H₁₅NO₄: 249.10.

(d) Ethyl 5-(2-bromoacetyl)-2-cyclopropyl-6-oxo-1,6-dihydropyridine-3-carboxylate. To a solution of ethyl 5-acetyl-2-cyclopropyl-6-oxo-1,6-dihydropyridine-3-carboxylate (1.40 g, 5.6 mmol) and 80 mL of dry THF was added 5,5′-dibromobarbituric acid (1.12 g, 3.9 mmol). The solution was stirred at 60° C. overnight, cooled to RT and concentrated in vacuo to give an orange solid that was used without further purification. MS m/z: 327 and 329 (M+1).

Calc'd for C₁₃H₁₄BrNO₄: 327.01.

(e) Ethyl 2-cyclopropyl-6-oxo-5-(2-(4-pyridyl)(1,3-thiazol-4-yl)-1,6-dihydro-3-pyridinecarboxylate. A solution of crude ethyl 5-(2-bromoacetyl)-2-cyclopropyl-6-oxo-1,6-dihydropyridine-3-carboxylate (330 mg, 1.0 mmol), and isothionicotinamide (100 mg, 0.8 mmol) in 8 mL of EtOH was stirred at reflux 72 h. The resulting solution was cooled to RT and the precipitate filtered and washed with 2M NH₃ in MeOH. The precipitate was absorbed onto silica gel and purified by flash chromatography on silica gel using 97:3 CH₂Cl₂:MeOH as the eluant to give a yellow solid. The yellow solid was suspended in warm EtOH and filtered to give a yellow solid. MS m/z: 368 (M+1). HRMS Calc'd for C₁₉H₁₇N₃O₃S [M+H], 368.1063, Found: 368.1051.

### Example 80

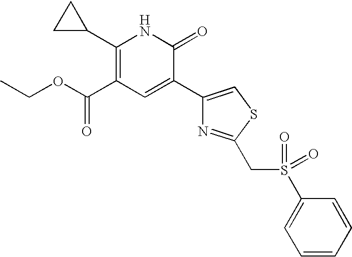

### Ethyl 2-cyclopropyl-6-oxo-5-(2-((phenylsulfonyl)methyl)-1,3-thiazol-4-yl)-1,6-dihydro-3-pyridinecarboxylate

A solution of crude ethyl 5-(2-bromoacetyl)-2-cyclopropyl-6-oxo-1,6-dihydropyridine-3-carboxylate (Example 79d, 90 mg, 0.6 mmol), 2-(phenylsulfonyl)-ethanethioamide (90 mg, 0.4 mmol), and 8 mL of EtOH were stirred at reflux for 4 h. The resulting solution was cooled to RT and the precipitate filtered and washed with ether to give a gray solid. MS m/z: 445 (M+1). HRMS Calc'd for C₂₁H₂₀N₂O₅S₂ [M+H], 445.0886, Found: 445.0877.

### Example 81

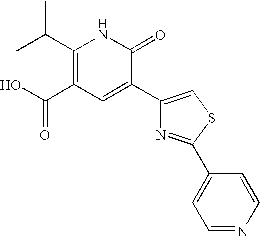

### 2-(Isopropyl)-6-oxo-5-(2-(4-pyridinyl)-1,3-thiazol-4-yl)-1,6-dihydro-3-pyridinecarboxylic acid

Ethyl 2-isopropyl-6-oxo-5-(2-(4-pyridyl)(1,3-thiazol-4-yl)-1,6-dihydropyridine-3-carboxylate (0.65 g, 1.8 mmol, Example 10d) and solid KOH (0.78g, 13.9 mmol) were suspended in 3 mL EtOH and 2 mL H₂O and heated in a microwave synthesizer for 10 min at 120° C. The resulting dark red solution was concentrated in vacuo and then diluted with H₂O. The solution was acidified to pH 1 and filtered to give a reddish solid that was dried under high vacuum at 60° C. to give the titled compound. MS m/z: 342 (M+1). Calc'd for C₁₇H₁₅N₃O₃S: 341.08.

### Example 82

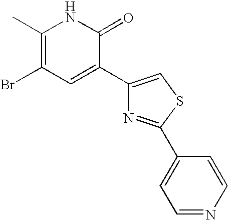

### 5-Bromo-6-methyl-3-(2-(4-pyridinyl)-1,3-thiazol-4-yl)-2(1H)-pyridinone

(a) 5-Acetyl-2-methyl-6-oxo-1,6-dihydropyridine. To a solution of trans-4-methoxy-3-butene-2-one (2.0 mL, 19.6 mmol) in 20 mL of anhydrous THF was added NaH (60% in mineral oil, 0.15 g, 3.8 mmol). After stirring for 15 min a solution of acetoacetamide in 20 mL of anhydrous THF was added dropwise. After the addition was complete the solution was stirred at 60° C. overnight. The reaction was cooled to RT, then acidified to pH 4 using 2N HCl (aq). The precipitate was filtered off and washed with hexane to give a yellow solid. MS m/z: 152 (M+1). Calc'd for C₈H₉NO₂: 151.06.

(b) 5-Acetyl-3-bromo-2-methyl-6-oxo-1,6-dihydropyridine. To a solution of 5-acetyl-2-methyl-6-oxo-1,6-dihydropyridine (Step a, 1.74 g, 11.5 mmol) in 50 mL of DMF was added NBS (2.47 g, 13.9 mmol). The solution was stirred at RT for 1.5 h, and diluted with H₂O. The resulting precipitate was filtered and the filtrate was extracted with EtOAc (3×). The combined EtOAc layers were washed with H₂O, brine, dried over MgSO₄, and concentrated in vacuo to give a tan solid. The precipitate and tan solid were shown to be equivalent by TLC and therefore combined. MS m/z: 230 and 232 (M+1). Calc'd for C₈H₈BrNO₂: 228.97.

(c) 5-(2-Bromoacetyl)-3-bromo-2-methyl-6-oxo-1,6-dihydropyridine. To a solution of 5-acetyl-3-bromo-2-methyl-6-oxo-1,6-dihydropyridine (Step b, 1.85 g, 8.0 mmol) and 100 mL anhydrous THF was added 5,5′-dibromobarbituric acid (1.61 g, 5.6 mmol). The solution was stirred at 70° C. overnight. The reaction was cooled to RT and concentrated in vacuo. The residue was suspended in ether and the precipitate filtered. The filtrate was concentrated in vacuo to give crude product that was used without further purification.

(d) 3-Bromo-2-methyl-6-oxo-5-{2-[(phenylsulfonyl)methyl]-(1,3-thiazol-4-yl)}-1,6-dihydropyridine. To a solution of crude 5-(2-bromoacetyl)-3-bromo-2-methyl-6-oxo-1,6-dihydropyridine (Step c, 1.8 g) in 25 mL of EtOH was added isothionicotinamide (0.78 g, 5.6 mmol) and the reaction stirred at reflux overnight. The reaction was cooled to RT and the solid filtered. The solid was purified by flash chromatography on silica gel using a gradient of 2% MeOH:CH₂Cl₂ to 5% MeOH:CH₂Cl₂ (in 1% increments) to give a solid. The solid was suspended in 9:1 CH₂Cl₂:MeOH and filtered to give a tan solid. MS m/z: 347 and 349 (M+1).

Calc'd for C₁₄H₁₀BrN₃OS Exact Mass: 346.97.

### Example 83

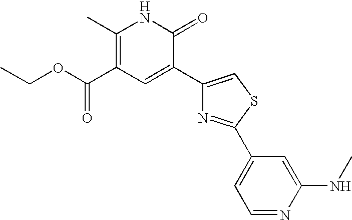

### Ethyl 2-methyl-5-(2-(2-(methylamino)-4-pyridinyl)-1,3-thiazol-4-yl)-6-oxo-1,6-dihydro-3-pyridinecarboxylate

A mixture of 5-(2-bromo-acetyl)-2-methyl-6-oxo-1,6-dihydro-pyridine-3-carboxylic acid ethyl ester (Example 24c) (0.10 g, 0.31 mmol) and 2-methylamino thioisonicotinamide (0.07 g, 0.43 mmol) in EtOH (3 mL) was heated at 150° C. for 7 min by microwave. The mixture was cooled, concentrated, and purified by flash column chromatography (3% MeOH/CH₂Cl₂) to give an off white solid. MS (M+1): 371.4. Calc'd for C₁₈H₁₈N₄O₃S Exact Mass: 370.11. MP: 270° C. (dec).

### Example 84

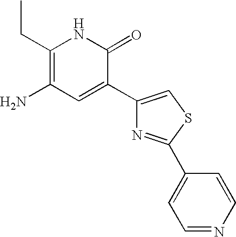

### 5-Amino-6-ethyl-3-(2-(4-pyridinyl)-1,3-thiazol-4-yl)-2(1H)-pyridinone

(a) N-(4-Methoxybenzyl)acetoacetamide. To an ice-bath cooled solution of 4-methoxybenzyl amine (17.2 g, 125.4 mmol) in 200 mL of anhydrous THF was added diketene dropwise over 0.5 h. The reaction was stirred at RT overnight. The reaction was concentrated in vacuo and the orange residue taken up in 200 mL of EtOAc and washed with H₂O, saturated NaHCO₃, dried over MgSO₄, and concentrated in vacuo to give an orange oil. The orange oil was suspended in 200 mL of Et₂O and filtered to give a yellow solid. MS m/z: 222 (M+1). Calc'd for C₁₂H₁₅NO₃: 221.11.

(b) Ethyl 5-acetyl-2-ethyl-1-(4-methoxybenzyl)-6-oxo-1,6-dihydropyridine-3-carboxylate. To a solution of N-(4-methoxybenzyl)acetoacetamide (Step a, 10.70 g, 48.4 mmol) and 150 mL of anhydrous THF was added 60% NaH (in mineral oil, 1.52 g, 38.0 mmol) portion-wise. After stirring for 15 min a solution of ethyl 2-propionyl-3-(dimethylamino)prop-2-enoate (9.62 g, 48.3 mmol, Example 1a) in 150 mL of anhydrous THF was added dropwise. After the addition was complete the reaction was stirred at 60° C. overnight. The reaction was cooled to RT and concentrated in vacuo. The resulting residue was diluted with 200 mL of H₂O and acidified to pH 3 using 1N HCl (aq). The aqueous solution was extracted with EtOAc (3×) and the combined EtOAc layers were washed with brine, dried over MgSO₄, and concentrated in vacuo to give a reddish oil. The oil was purified by flash chromatography on silica gel using 0.5% EtOAc:CH₂Cl₂ to give a reddish solid. MS m/z: 358 (M+1).

Calc'd for C₂₀H₂₃NO₅: 357.16.

(c) Ethyl 5-(2-bromoacetyl)-2-ethyl-1-(4-methoxybenzyl)-6-oxo-1,6-dihydropyridine-3-carboxylate. This compound was prepared in a similar manner to Example 1c using ethyl 5-acetyl-2-ethyl-1-(4-methoxybenzyl)-6-oxo-1,6-dihydropyridine-3-carboxylate (Step b, 6.78 g, 19.0 mmol), 5,5′-dibromobarbituric acid (4.03 g, 14.1 mmol), and 150 mL of anhydrous THF. The resulting orange solid was carried on without further purification.

(d) Ethyl 2-ethyl-1-(4-methoxybenzyl)-6-oxo-5-(2-(4-pyridyl)(1,3-thiazol-4-yl))-1,6-dihydro-3-pyridinecarboxylate. To a solution of crude ethyl 5-(2-bromoacetyl)-2-ethyl-1-(4-methoxybenzyl)-6-oxo-1,6-dihydropyridine-3-carboxylate (Step c) and 200 mL of EtOH was added isothionicotinamide (2.60 g, 18.8 mmol). The solution was stirred at reflux overnight. The reaction was cooled to RT and the precipitate was filtered and washed with EtOH to give a rust colored solid. MS m/z: 476 (M+1).

Calc'd for C₂₆H₂₅N₃O₄S: 475.16.

(e) 2-Ethyl-1-(4-methoxybenzyl)-6-oxo-5-(2-(4-pyridyl)(1,3-thiazol-4-yl))-1,6-dihydro-3-pyridinecarboxylate. To a solution of ethyl 2-ethyl-1-(4-methoxybenzyl)-6-oxo-5-(2-(4-pyridyl)(1,3-thiazol-4-yl))-1,6-dihydropyridine-3-carboxylate (0.30 g, 0.6 mmol, Step d) and 15 mL of THF was added 1N NaOH (1.3 mL, 1.3 mmol). After 2 h, an additional amount of 1N NaOH (1.3 mL, 1.3 mmol) was added. After an additional 2 h, the reaction was heated to 60° C. and stirred for 3 days. The reaction was concentrated in vacuo and the aqueous solution was acidified to pH 3 using 1N HCl (aq). The precipitate was filtered to give a yellow solid after drying in high vacuum. MS m/z: 448.1 (M+1). Calc'd for C₂₄H₂₁N₃O₄S: 447.50.

(f) [2-Ethyl-1-(4-methoxybenzyl)-6-oxo-5-(2-(4-pyridyl)(1,3-thiazol-4-yl))-1,6-dihydropyridin-3-yl]-carbamic acid tert-butyl ester. To a suspension of 2-ethyl-1-(4-methoxybenzyl)-6-oxo-5-(2-(4-pyridyl)(1,3-thiazol-4-yl))-1,6-dihydropyridine-3-carboxylate (1.89 g, 4.2 mmol, Step e) and 20 mL of anhydrous toluene/20 mL of anhydrous 2-methyl-2-propanol was added DIPEA (1.1 mL, 6.3 mmol). After stirring for 15 min, dppa (0.28 mL, 1.3 mmol) was added dropwise and the solution was stirred at 80° C. overnight. The reaction was cooled to RT and filtered. The precipitate was washed with 9:1 CH₂Cl₂:MeOH. The filtrate was concentrated in vacuo, redissolved in EtOAc (150 mL) and washed with 1N NaOH, brine, dried over MgSO₄, and concentrated in vacuo. The residue was absorbed onto silica gel and purified with an ISCO silica gel flash chromatography instrument using 3% MeOH:CH₂Cl₂ to give a yellow solid. MS m/z: 519 (M+1). Calc'd for C₂₈H₃₀N₄O₄S: 518.20.

(g) 5-Amino-6-ethyl-1-(4-methoxybenzyl)-3-(2-(4-pyridyl)(1,3-thiazol-4-yl))-1H-pyridin-2-one. To a suspension of [2-ethyl-1-(4-methoxybenzyl)-6-oxo-5-(2-(4-pyridyl)(1,3-thiazol-4-yl))-1,6-dihydropyridine]-3-carbamic acid tert-butyl ester (Step f, 1.02 g, 2.0 mmol) in 40 mL of dioxane/25 mL of MeOH was added 4M HCl (in dioxane, 6.0 mL, 24 mmol). After stirring for 8 h at RT, additional 4M HCl (in dioxane, 1.0 mL, 4 mmol) was added and the reaction was stirred overnight. The precipitate was filtered off and washed with Et₂O to give a yellow solid. MS m/z: 419 (M+1). Calc'd for C₂₃H₂₂N₄O₂S: 418.15.

(h) 5-Amino-6-ethyl-3-(2-(4-pyridyl)(1,3-thiazol-4-yl)-1H-pyridin-2-one. To a suspension of 5-amino-6-ethyl-1-(4-methoxybenzyl)-3-(2-(4-pyridyl)(1,3-thiazol-4-yl)-1H-pyridin-2-one (Step g, 0.13 g, 0.3 mmol) in 10 mL of CH₂Cl₂ was added 3-methoxybenzene thiol (0.10 mL, 0.8 mmol) and TFA (3.0 mL). The solution was stirred at 35° C. for 3 h, then cooled and concentrated in vacuo to a residue. The residue was suspended in CH₂Cl₂ and filtered to give a rust colored solid. The solid was dissolved in 9:1 CH₂Cl₂:MeOH and washed with saturated NaHCO₃. The aqueous layer was extraced with 9:1 CH₂Cl₂:MeOH (5×). The organic layers were concentrated in vacuo. The solid was purified by flash chromatography using 5% MeOH:CH₂Cl₂ to give a yellow solid. MS m/z: 299 (M+1). Calc'd for C₁₅H₁₄N₄OS Exact Mass: 298.09.

### Example 85

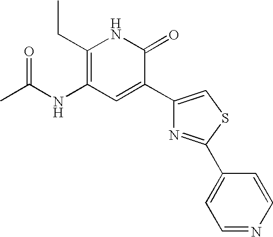

### N-[2-Ethyl-6-oxo-5-(2-(4-pyridyl)(1,3-thiazol-4-yl)-1,6-dihydropyridin-3-yl]-acetamide

To an ice-bath cooled suspension of 5-amino-6-ethyl-3-(2-(4-pyridyl)(1,3-thiazol-4-yl)-1H-pyridin-2-one (30 mg, 0.1 mmol, Example 84(h)) in 5 mL of CH₂Cl₂ was added acetyl chloride (0.007 mL, 0.1 mmol, Aldrich Chemical Co.). The solution was slowly warmed to RT. After 4 h, an additional amount of acetyl chloride (0.02 mL, 0.3 mmol) was added and the reaction was stirred overnight. The reaction was filtered and the solid washed with CH₂Cl₂. The solid was purified by flash chromatography on silica gel using 5% MeOH:CH₂Cl₂ (2×500 mL), then 10% MeOH:CH₂Cl₂ (3×500 mL) to give an off-white solid. MS m/z: 340.8 (M+1). HRMS Calc'd for C₁₇H₁₆N₄O₂S [M+H], 341.1067, Found: 341.1087.

### Example 86

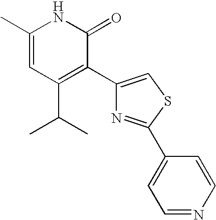

### 4-Dimethylamino-6-methyl-3-(2-pyridin-4-yl-thiazol-4-yl)-1H-pyridin-2-one

To a solution of trans-4-(dimethylamino)-3-buten-2-one (Aldrich) (4.2 g, 37 mmol) in 40 mL CH₂Cl₂ was added Br₂ (2.1 mL, 41 mmol) dropwise over a period of 20 min. After 1 h the reaction was diluted with 25 mL Et₂O and Et₃N was added dropwise. After 1 h the reaction was filtered and solids washed with Et₂O. The filtrate was concentrated in vacuo gave a brown solid that was used without further purification. A portion of this residue (209 mg, 1.0 mmol) and 2-(2-pyridin-4-yl-thiazol-4-yl)-acetamide (209 mmol, 1.1 mmol) was stirred in 5 mL DMF. To this solution was added 60% NaH (100 mg, 2.5 mmol) resulting in gas evolution and the reaction mixture was heated to 70° C. After 1.5 h the reaction was cooled to 0° C. and quenched with 1N HCl. The solution was evaporated onto silica gel and purified by flash column chromatography eluting with 2M NH₃ in MeOH/CH₂Cl₂ (0:1 1:9) to give a tan amorphous solid. MS m/z: 313 (M+1). HPLC purity: 96%. Exact mass Calc'd for C₁₆H₁₆N₄OS: 313.1118. Found: 313.1092.

### Example 87

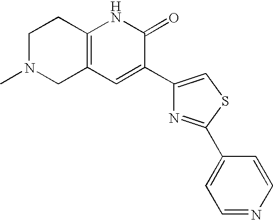

### 6-Methyl-3-(2-pyridin-4-yl-thiazol-4-yl)-5,6,7,8-tetrahydro-1H-[1,6]naphthyridin-2-one

A mixture of 1-methyl-4-piperidone (Aldrich) (5 mL, 41 mmol) and N,N′-dimethylformamide dimethyl acetal (6 mL, 45 mmol) was heated to 100° C. for 16 h. The reaction was cooled to RT and the volatiles were removed in vacuo. A portion of this residue (220 mg, 1.3 mmol) and 2-(2-pyridin-4-yl-thiazol-4-yl)-acetamide (202 mmol, 0.9 mmol) was stirred in 5 mL DMF. To this suspension was added 60% NaH (98 mg, 2.5 mmol) resulting in gas evolution. After 4 h the reaction was cooled to 0° C. and quenched with 5N HCl. The mixture was poured into water and the solvent was removed in vacuo. The residue was dissolved in MeOH, evaporated onto SiO₂ and purified by flash column chromatography eluting with 2M NH₃ in MeOH/CH₂Cl₂ (0:1 1:9) to give a yellow amorphous solid. MS m/z: 325 (M+1); 323 (M−1). Exact mass: Calc'd 325.1118. Found: 325.1114.

### Example 88

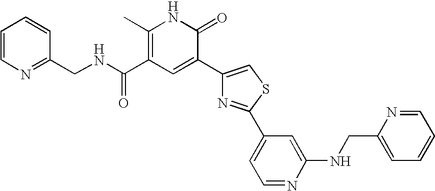

### 2-Methyl-6-oxo-N-(2-pyridinylmethyl)-5-(2-(2-((2-pyridinylmethyl)amino)-4-pyridinyl)-1,3-thiazol-4-yl)-1,6-dihydro-3-pyridinecarboxamide

A mixture of ethyl 5-(2-(2-chloro-4-pyridinyl)-1,3-thiazol-4-yl)-2-methyl-6-oxo-1,6-dihydro-3-pyridinecarboxylate (Example 40) (0.10 g, 0.27 mmol), 2-aminomethylpyridine (0.11 g, 0.8 mmol) and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a white solid. MS (M+1): 510.17. Calc'd for C₂₇H₂₃N₇O₂S Exact Mass: 509.16. MP: >260° C.

### Example 89

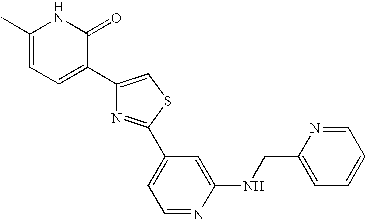

### 6-Methyl-3-(2-(2-((2-pyridinylmethyl)amino)-4-pyridinyl)-1,3-thiazol-4-yl)-2(1H)-pyridinone

A mixture of ethyl 5-(2-(2-chloro-4-pyridinyl)-1,3-thiazol-4-yl)-2-methyl-6-oxo-1,6-dihydro-3-pyridinecarboxylate (Example 40) (0.10 g, 0.27 mmol), 2-aminomethylpyridine (0.11 g, 0.8 mmol) and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a white solid. MS (M+1): 376.4. Calc'd for C₂₀H₁₇N₅OS Exact Mass: 375.12.

### Example 90

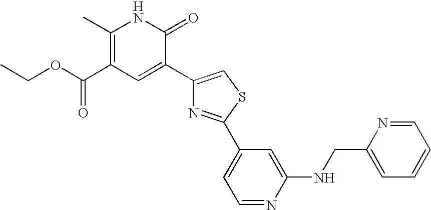

### Ethyl 2-methyl-6-oxo-5-(2-(2-((2-pyridinylmethyl)amino)-4-pyridinyl)-1,3-thiazol-4-yl)-1,6-dihydro-3-pyridinecarboxylate

A mixture of ethyl 5-(2-(2-chloro-4-pyridinyl)-1,3-thiazol-4-yl)-2-methyl-6-oxo-1,6-dihydro-3-pyridinecarboxylate (Example 40) (0.10 g, 0.27 mmol), 2-aminomethylpyridine (0.11 g, 0.8 mmol), and Cu powder (0.09 g, 0.14 mmol) in 2,4,6-collidine (3 mL) was heated at 160° C. for 16 h. The mixture was cooled, concentrated, and purified by flash column chromatography (5% MeOH/CH₂Cl₂) to give a white solid. MS (M+1): 448.4. Calc'd for C₂₃H₂₁N₅O₃S Exact Mass: 447.14. MP: 270° C. (dec).

### Example 91

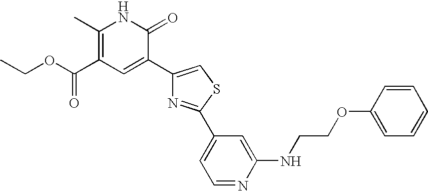

### Ethyl 2-methyl-6-oxo-5-(2-(2-((2-(phenyloxy)ethyl)amino)-4-pyridinyl)-1,3-thiazol-4-yl)-1,6-dihydro-3-pyridinecarboxylate

A mixture of ethyl 5-(2-(2-chloro-4-pyridinyl)-1,3-thiazol-4-yl)-2-methyl-6-oxo-1,6-dihydro-3-pyridinecarboxylate (Example 40) (0.10 g, 0.27 mmol) and phenoxyethylamine (0.11 g, 0.8 mmol) in EtOH (3 mL) was heated at 150° C. by microwave for 7 min. The mixture was cooled, concentrated, and purified by flash column chromatography (3% MeOH/CH₂Cl₂) to give a white solid. MS (M+1): 477.4. Calc'd for C₂₅H₂₄N₄O₄S Exact Mass: 476.15. MP: 270° C. (dec).

### Example 92

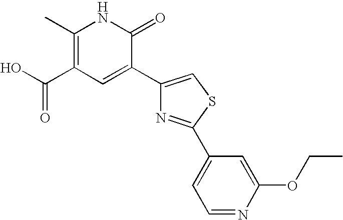

### 5-(2-(2-(Ethoxy)-4-pyridinyl)-1,3-thiazol-4-yl)-2-methyl-6-oxo-1,6-dihydropyridine-3-carboxylic acid

A mixture of 5-[2-(2-chloro-pyridin-4-yl)-thiazol-4-yl]-2-methyl-6-oxo-1,6-dihydropyridine-3-carboxylic acid (0.10 g, 0.31 mmol) and 2-methoxythioisonicotinamide (0.07 g, 0.43 mmol) in EtOH (3 mL) was heated at 150° C. for 7 min by microwave. The mixture was cooled, concentrated, and purified by flash column chromatography (2% MeOH/CH₂Cl₂) to give an off white solid. MS (M+1): 413.4. Calc'd for C₂₁H₂₄N₄O₃S. MP: 290° C. (dec).

### Example 93

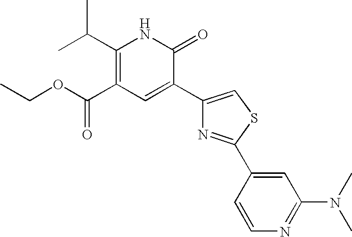

### Ethyl 5-[2-(dimethylamino-pyridin-4-yl)-thiazol-4-yl]-2-isopropyl-6-oxo-1,6-dihydro-3-pyridinecarboxylate

a) 2-Dimethylamino-4-isonicotinonitrile. A mixture of 2-chloro-4-cyanopyridine (2.0 g, 14.43 mmol) and Me₂NH (40% Wt. in H₂O, 5 mL) in THF (20 mL) was stirred at RT in a sealed tube for 18 h. The mixture was concentrated, stirred in H₂O, filtered to provide a white solid that was dried by air, and used in the next step without further purification.

b) 2-Dimethylaminothioisonicotinamide. To a stirred mixture of 2-dimethylamino-4-isonicotinonitrile (Step a, 1.5 g, 10.61 mmol) and pyridine (2.5 g, 31.82 mmol) in TEA (20 mL) was bubbled with H₂S for 10 min. The resulting reaction was stirred at RT for 24 h, concentrated, stirred in H₂O, and the dark tan solid was filtered and dried by air.

c) Ethyl 5-[2-(dimethylamino-pyridin-4-yl)-thiazol-4-yl]-2-isopropyl-6-oxo-1,6-dihydro-3-pyridinecarboxylate hydrochloride. A mixture of 5-(2-bromoacetyl)-2-isopropyl-6-oxo-1,6-dihydropyridine-3-carboxylate (Example 10(c)) (0.20 g, 0.61 mmol) and 2-dimethylaminothioisonicotinamide (Step b, 0.14 g, 0.79 mmol) in EtOH (10 mL) was heated at reflux for 24 h. The mixture was cooled, concentrated, and purified by flash column chromatography (3% MeOH/CH₂Cl₂) to give an off white solid which was dissolved in warm 1,4-dioxane and treated with 1.0M HCl in Et₂O (0.35 mL, 1.1 mmol). The off-white solid was filtered, and dried. MS (M+1): 413.2. Calc'd for C₂₁H₂₄N₄O₃S Exact Mass: 412.16. MP: >230° C.

### Example 94

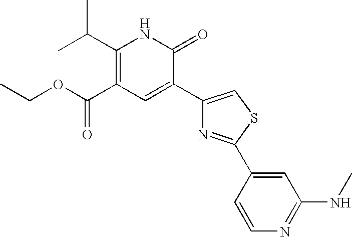

### Ethyl 5-[2-(methylamino-pyridin-4-yl)-thiazol-4-yl]-2-isopropyl-6-oxo-1,6-dihydro-3-pyridinecarboxylate

a) 2-Methylamino-4-isonicotinonitrile. A mixture of 2-chloro-4-cyanopyridine (2.0 g, 14.43 mmol) and methylamine (40% Wt. in H₂O, 5 mL) in THF (20 mL) was stirred at RT in a sealed tube for 18 h. The mixture was concentrated, stirred in H₂O, filtered to give an off white solid after drying by air, and used in the next step without further purification.

b) 2-Methylaminothioisonicotinamide. To a stirred mixture of 2-methylamino-4-isonicotinonitrile (Step a, 0.40 g, 3.01 mmol) and pyridine (1.18 g, 15.03 mmol) in TEA (10 mL) was bubbled with H₂S for 10 min. The resulting reaction was stirred at RT in 24 h, concentrated, stirred in H₂O, and the dark tan solid was filtered and dried by air.

c) Ethyl 5-[2-(methylamino-pyridin-4-yl)-thiazol-4-yl]-2-isopropyl-6-oxo-1,6-dihydro-3-pyridinecarboxylate. A mixture of ethyl 5-(2-bromoacetyl)-2-isopropyl-6-oxo-1,6-dihydropyridine-3-carboxylate (Example 10(c)) (0.10 g, 0.31 mmol) and 2-methylaminothio-isonicotinamide (Step b, 0.08 g, 0.45 mmol) in EtOH (3 mL) was heated at 150° C. for 7 min using a microwave synthesizer. The mixture was cooled, concentrated, and purified by flash column chromatography (3% MeOH/CH₂Cl₂) to give a tan solid which was dissolved in warm 1,4-dioxane and treated with 1M HCl in Et₂O (0.3 mL, 1.1 mmol) to give the HCL salt as an off-white solid after filtration and drying by air. MS (M+1): 399.5. Calc'd for C₂₀H₂₂N₄O₃S Exact Mass: 398.14. MP: >230° C.

### Example 95

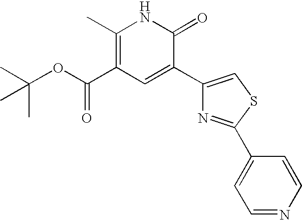

### 1,1-Dimethylethyl 2-methyl-6-oxo-5-(2-(4-pyridinyl)-1,3-thiazol-4-yl)-1,6-dihydro-3-pyridinecarboxylate

(a) 2-Dimethylaminomethylene-3-oxo-butyric acid tert-butyl ester. A mixture of ethyl acetoacetate (26.6 mL, 97%, 156 mmol, Aldrich Chemical Co.) and N,N-dimethylformamide dimethyl acetal (55.0 mL, 94%, 389 mmol) was heated at 95° C. for 2 h. A red solution resulted. Excess reagents were removed in vacuum to give quantitative yield of a dark-red oil which was used directly in the next step.

(b) 5-Acetyl-2-methyl-6-oxo-1,6-dihydro-pyridine-3-carboxylic acid tert-butyl ester. This compound was prepared in a similar manner to Example 1b using 2-dimethylaminomethylene-3-oxo-butyric acid tert-butyl ester (Step a, 34.50 g, 155.0 mmol), acetoacetamide (15.67 g, 155 mmol), and NaH (60% in mineral oil, 5.01 g, 125 mmol) to give a yellow solid. MS m/z: 252 (M+1). Calc'd for C₁₃H₁₇NO₄: 251.12.

(c) 5-(2-Bromo-acetyl)-2-methyl-6-oxo-1,6-dihydro-pyridine-3-carboxylic acid tert-butyl ester. A mixture of 5-acetyl-2-methyl-6-oxo-1,6-dihydro-pyridine-3-carboxylic acid tert-butyl ester (Step b, 10.0 g, 40 mmol) and 5,5-dibromobarbituric acid (Aldrich, 6.85 g, 23.9 mmol) in 200 mL of anhydrous THF was heated at reflux for 4 h. Reaction was monitored by analytical HPLC until all starting materials were gone. The solvent was evaporated under reduced vacuum to give a solid residue that was used directly in the next step.

(d) 2-Methyl-6-oxo-5-(2-pyridin-4-yl-thiazol-4-yl)-1,6-dihydro-pyridine-3-carboxylic acid tert-butyl ester. A mixture of 5-(2-bromo-acetyl)-2-methyl-6-oxo-1,6-dihydro-pyridine-3-carboxylic acid tert-butyl ester (crude, Step c) and isothionicotinamde (Lancaster, 5.5 g, 40 mmol) in 300 mL of anhydrous MeOH was heated at reflux for 6 h. The solution was cooled to RT. Precipitates were filtered, washed with copious amount of MeOH, CH₂Cl₂ and hexanes. This furnished the title compound as a yellow solid. MS m/z: 370.1 (M+1). This material (100 mg) was further purified by Gilson preparative HPLC. Desired fractions were combined, dried, and neutralized with NH₄OH followed by azeotroping with 3×25 mL of toluene to provide product as a white solid. MS m/z: 370.1 (M+1). Calc'd for C₁₉H₁₉N₃O₃S Exact Mass: 369.11.

### Example 96

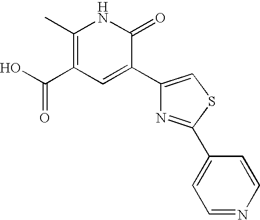

### 2-Methyl-6-oxo-5-(2-pyridin-4-yl-thiazol-4-yl)-1,6-dihydro-pyridine-3-carboxylic acid

2-Methyl-6-oxo-5-(2-pyridin-4-yl-thiazol-4-yl)-1,6-dihydro-pyridine-3-carboxylic acid tert-butyl ester (Example 95d, 1.0 g, 2.7 mmol) was treated with 5 mL of TFA:CH₂Cl₂ (1:1) at RT for 1 h. HPLC analysis indicated a complete reaction. The solvents were removed under vacuum and the brown residue was azeotroped with 3×25 mL of toluene to afford the product as a TFA salt. This material (100 mg) was purified by Gilson preparative HPLC. Desired fractions were combined, dried, and azeotroped with 3×15 mL of toluene to provide the title compound as a yellow solid. MS m/z: 314.2 (M+1). Calc'd for C₁₅H₁₁N₃O₃S Exact Mass: 313.05.

### Example 97

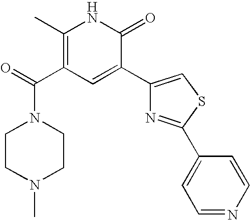

### 6-Methyl-5-((4-methyl-1-piperazinyl)carbonyl)-3-(2-(4-pyridinyl)-1,3-thiazol-4-yl)-2(1H)-pyridinone

2-Methyl-6-oxo-5-(2-pyridin-4-yl-thiazol-4-yl)-1,6-dihydro-pyridine-3-carboxylic acid (Example 96, 100 mg, 0.32 mmol) in 20 mL of anhydrous CH₂Cl₂ was treated with 0.5 mL of DIPEA and 0.5 mL of pivloyl chloride at RT for 5 h to bring about a homogeneous solution. Upon this time, 1.0 mL of 1-methylpeperizine was added and the mixture was stirred for additional 2 h. Precipitates formed. Filtration and Gilson preparative HPLC purification, followed by solvent removal, neutralization with NH₄OH, and azeotroping with 3×10 mL of toluene, afforded the title compound as an off-white solid. MS m/z: 396.1 (M+1). Calc'd for C₂₀H₂₁N₅O₂S Exact Mass: 395.14.

### Example 98

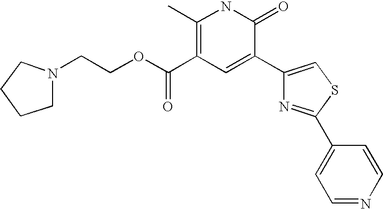

### 2-(1-Pyrrolidinyl)ethyl 2-methyl-6-oxo-5-(2-(4-pyridinyl)-1,3-thiazol-4-yl)-1,6-dihydro-3-pyridinecarboxylate

(a) 3-Oxo-butyric acid 2-pyrrolidin-1-yl-ethyl ester. To a solution of 1-(2-hydroxyethyl)-pyrrolidine (2.4 mL, 20 mmol) in 50 mL of anhydrous CH₂Cl₂ in a water bath was added dropwise 1.6 mL of diketene (20 mmol, Aldrich). The resulting mixture was stirred for 1 h at RT. The solvent was removed under vacuum and the residue was dried under high vacuum overnight to provide an oil. MS m/z: 200.2 (M+1). Calc'd for C₁₀H₁₇NO₃: 199.12.

(b) 2-Dimethylaminomethylene-3-oxo-butyric acid 2-pyrrolidin-1-yl-ethyl ester. A mixture of 3-oxo-butyric acid 2-pyrrolidin-1-yl-ethyl ester (4.0 g, Step a) and N,N-dimethylformamide dimethyl acetal (7.07 mL, 94%, 50 mmol) was heated at 95° C. for 2 h. A red solution resulted. Excess reagents were removed in vacuum to give a dark-red oil which was used directly in the next step. MS m/z: 255.3 (M+1). Calc'd for C₁₃H₂₂N₂O₃: 254.16.

(c) 2-Methyl-6-oxo-5-(2-pyridin-4-yl-thiazol-4-yl)-1,6-dihydro-pyridine-3-carboxylic acid 2-pyrrolidin-1-yl-ethyl ester. A solution of 2-dimethylaminomethylene-3-oxo-butyric acid 2-pyrrolidin-1-yl-ethyl ester (258 mg, 1.0 mmol, Step b) and 2-(2-pyridin-4-yl-thiazol-4-yl)-acetamide (250 mg, 1.2 mmol, Example 18(b)) in 35 mL of anhydrous DMF was treated with NaH (80 mg, 60% in mineral oil, 2.0 mmol). The resulting mixture was heated at 70° C. for 3 h. The reaction was cooled down to RT and quenched by addition of 50 mL of CH₂Cl₂ and 50 mL of saturated aqueous NaHCO₃. The mixture was stirred vigorously for 10 min. The CH₂Cl₂ layer was separated, washed with saturated aqueous NaHCO₃, dried (Na₂SO₄), and concentrated to yield an oil. Gilson HPLC purification followed by basic aqueous extraction (CH₂Cl₂ and saturated aqueous NaHCO₃) and drying, provided the title compound as a yellowish glassy solid. MS m/z: 411.4 (M+1).

Calc'd for C₂₁H₂₂N₄O₃S Exact Mass: 410.14.

### Example 99

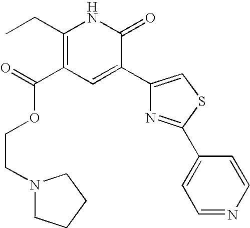

### 2-(1-Pyrrolidinyl)ethyl 2-ethyl-6-oxo-5-(2-(4-pyridinyl)-1,3-thiazol-4-yl)-1,6-dihydro-3-pyridinecarboxylate

A mixture of ethyl 2-ethyl-6-oxo-5-(2-(4-pyridyl)(1,3-thiazol-4-yl)-1,6-dihydropyridine-3-carboxylate (100 mg, 0.28 mmol, Example 1(d)), 1-(2-hydroxyethyl)-pyrrolidine (5.0 mL, 41.7 mmol), and 100 mg of Cu powder was heated at 180° C. overnight. The reaction was cooled down to RT, diluted with 50 mL of CH₂Cl₂, washed with 2×50 mL of saturated aqueous NaHCO₃. The CH₂Cl₂ layer was separated, dried (Na₂SO₄), and concentrated to yield an oil. Gilson HPLC purification followed by basic aqueous extraction (CH₂Cl₂ and saturated aqueous NaHCO₃) and drying, provided the title compound as a light yellow solid. MS m/z: 425.3 (M+1). Calc'd for C₂₂H₂₄N₄O₃S: 424.16.

### Example 100

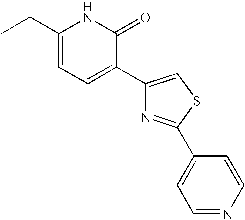

### 6-Ethyl-3-(2-pyridin-4-yl-thiazol-4-yl)-1H-pyridin-2-one

A mixture of ethyl 2-ethyl-6-oxo-5-(2-(4-pyridyl)(1,3-thiazol-4-yl)-1,6-dihydropyridine-3-carboxylate (100 mg, 0.28 mmol, Example 1(d)), 1-(2-hydroxyethyl)-pyrrolidine (5.0 mL, 41.7 mmol), and 100 mg of Cu powder was heated at 180° C. overnight. The reaction was cooled down to RT, diluted with 50 mL of CH₂Cl₂, washed with 2×50 mL of saturated aqueous NaHCO₃. The CH₂Cl₂ layer was separated, dried (Na₂SO₄), and concentrated to yield an oil. Gilson HPLC purification followed by basic aqueous extraction (CH₂Cl₂ and saturated aqueous NaHCO₃) and drying, provided the title compound as a light tan solid. MS m/z: 284.0 (M+1). Calc'd for C₁₅H₁₃N₃OS: 283.08.

### Example 101

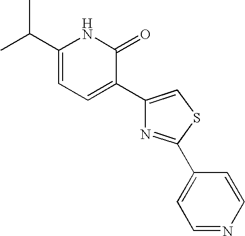

### 6-Isopropyl-3-(2-pyridin-4-yl-thiazol-4-yl)-1H-pyridin-2-one

A mixture of ethyl 2-isopropyl-6-oxo-5-(2-(4-pyridyl)(1,3-thiazol-4-yl)-1,6-dihydropyridine-3-carboxylate (80 mg, 0.22 mmol, example 10(d)), 1-(2-hydroxyethyl)-pyrrolidine (5.0 mL, 41.7 mmol), and 100 mg of Cu powder was heated at 180° C. overnight. The reaction was cooled down to RT, diluted with 50 mL of CH₂Cl₂, washed with 2×50 mL of saturated aqueous NaHCO₃. The CH₂Cl₂ layer was separated, dried (Na₂SO₄), and concentrated to yield an oil. Gilson HPLC purification, followed by basic aqueous extraction (CH₂Cl₂ and saturated aqueous NaHCO₃) and drying, provided the title compound as a light tan solid. MS m/z: 298.1 (M+1). Calc'd for C₁₆H₁₅N₃OS: 297.09.

### Example 102

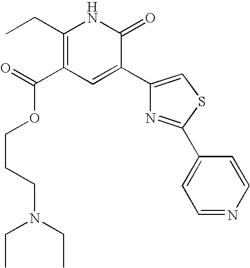

### 3-(Diethylamino)propyl 2-ethyl-6-oxo-5-(2-(4-pyridinyl)-1,3-thiazol-4-yl)-1,6-dihydro-3-pyridinecarboxylate

A mixture of ethyl 2-ethyl-6-oxo-5-(2-(4-pyridyl) (1,3-thiazol-4-yl)-1,6-dihydropyridine-3-carboxylate (100 mg, 0.28 mmol, Example 1(d)), 3-diethylamino-propan-1-ol (5.0 mL), and 100 mg of Cu powder was heated at 180° C. overnight. The reaction was cooled down to RT, diluted with 50 mL of CH₂Cl₂, washed with 2×50 mL of saturated aqueous NaHCO₃. The CH₂Cl₂ layer was separated, dried (Na₂SO₄), and concentrated to yield an oil. Gilson HPLC purification followed by basic aqueous extraction (CH₂Cl₂ and saturated aqueous NaHCO₃) and drying, provided the title compound as a light yellow solid. MS m/z: 441.1 (M+1). Calc'd for C₂₃H₂₈N₄O₃S: 440.19.

### Example 103

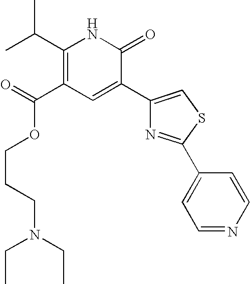

### 3-(Diethylamino)propyl 2-(1-methylethyl)-6-oxo-5-(2-(4-pyridinyl)-1,3-thiazol-4-yl)-1,6-dihydro-3-pyridinecarboxylate

A mixture of ethyl 2-isopropyl-6-oxo-5-(2-(4-pyridyl)(1,3-thiazol-4-yl)-1,6-dihydropyridine-3-carboxylate (80 mg, 0.22 mmol, Example 10(d)), 3-diethylamino-propan-1-ol (5.0 mL), and 100 mg of Cu powder was heated at 180° C. overnight. The reaction was cooled down to RT, diluted with 50 mL of CH₂Cl₂, washed with 2×50 mL of saturated aqueous NaHCO₃. The CH₂Cl₂ layer was separated, dried (Na₂SO₄), and concentrated to yield an oil. Gilson HPLC purification, followed by basic aqueous extraction (CH₂Cl₂ and saturated aqueous NaHCO₃) and drying, provided the title compound as a light yellow solid. MS m/z: 455.3 (M+1). Calc'd for C₂₄H₃₀N₄O₃S: 454.20.

### Example 104

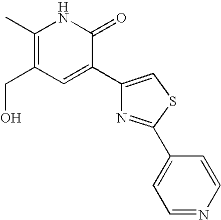

### 5-Hydroxymethyl-6-methyl-3-(2-pyridin-4-yl-thiazol-4-yl)-1H-pyridin-2-one

(a) 5-(Imidazole-1-carbonyl)-6-methyl-3-(2-pyridin-4-yl-thiazol-4-yl)-1H-pyridin-2-one. A suspension of 2-methyl-6-oxo-5-(2-pyridin-4-yl-thiazol-4-yl)-1,6-hydropyridine-3-carboxylic acid (4.0 g, 12.7 mmol, Example 98) in 100 mL of CH₂Cl₂ and 200 mL of DMF was treated with CDI (4.2 mg, 25.9 mmol, Aldrich) and DIPEA (10.0 mL, Aldrich) at RT for 3 days. Precipitates formed. Filtration, followed by washing with CH₂Cl₂, afforded the title compound as a yellowish solid. MS m/z: 364.2 (M+1). Calc'd for C₁₈H₁₃N₅O₂S: 363.08.

(b) 5-Hydroxymethyl-6-methyl-3-(2-pyridin-4-yl-thiazol-4-yl)-1H-pyridin-2-one. A suspension of 5-(imidazole-1-carbonyl)-6-methyl-3-(2-pyridin-4-yl-thiazol-4-yl)-1H-pyridin-2-one (110 mg, 0.30 mmol, Step a) in 50 mL of iPrOH and 20 mL of CHCl₃ was treated with NaBH₄ (100 mg, 2.65 mmol, Aldrich) at RT for 6 h. The reaction mixture was acidified carefully to pH 2 with 1N HCl. A clear yellow solution resulted. All solvents were removed under vacuum. Residue was purified by Gilson HPLC to provide the title compound as a yellow solid. MS m/z: 300.2 (M+1). Calc'd for C₁₅H₁₃N₃O₂S: 299.07.

### Example 105

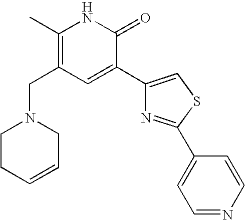

### 5-(3,6-Dihydro-2H-pyridin-1-ylmethyl)-6-methyl-3-(2-pyridin-4-yl-thiazol-4-yl)-1H-pyridin-2-one

A mixture of 5-hydroxymethyl-6-methyl-3-(2-pyridin-4-yl-thiazol-4-yl)-1H-pyridin-2-one (300 mg, 1.0 mmol, Example 104(b)) in 15 mL of pyridine was treated with methanesulfonyl chloride (0.3 mL, 3.88, Aldrich) at 0° C. The reaction was warmed slowly to RT during 4 h. The resulting mixture was concentrated to give a residue which was azeotroped with 25 mL of toluene. This solid material was dissolved in 50 mL of iPrOH and treated with 500 mg of NaBH₄ at RT for 1 h. The solvent was removed under vacuum. Gilson HPLC purification followed by basic aqueous extraction (CH₂Cl₂ and saturated aqueous NaHCO₃) and drying afforded the title compound as a yellow solid. MS m/z: 365 (M+1). Calc'd for C₂₀H₂₀N₄OS: 364.14.

### Example 106

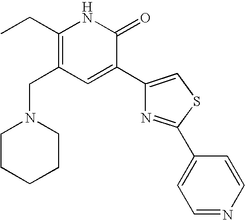

### 6-Ethyl-5-pip ridin-1-ylmethyl-3-(2-pyridin-4-yl-thiazol-4-yl)-1H-pyridin-2-one

(a) 6-Ethyl-5-hydroxymethyl-1-(4-methoxy-benzyl)-3-(2-pyridin-4-yl-thiazol-4-yl)-1H-pyridin-2-one. A mixture of 2-ethyl-1-(4-methoxybenzyl)-6-oxo-5-(2-(4-pyridyl)(1,3-thiazol-4-yl)-1,6-dihydropyridine-3-carboxylate (220 mg, 0.49 mmol, Example 84(e)) in 10 mL of CH₂Cl₂ and 2 mL of DMF was treated with CDI (260 mg, 1.6 mmol, Aldrich) at RT for 3 days. 15 mL of iPrOH was added followed by 300 mg of NaBH₄ The resulting mixture was stirred at RT for 1 h and quenched with 0.2N HCl until no bubbles were generated. After stirring vigorously for 15 min, the mixture was basicified to pH 8 with 1N NaOH and 10 mL of saturated aqueous NaHCO₃ was added. The mixture was extracted with 3×30 mL of CH₂Cl₂. The organic layers were combined, dried (Na₂SO₄), and concentrated to provide the title compound as an off-white solid which was used directly in the next step without further purification. MS m/z: 434.0 (M+1). Calc'd for C₂₄H₂₃N₃O₃S: 433.15.

(b) 6-Ethyl-1-(4-methoxy-benzyl)-5-piperidin-1-ylmethyl-3-(2-pyridin-4-yl-thiazol-4-yl)-1H-pyridin-2-one. A solution of 6-ethyl-5-hydroxymethyl-1-(4-methoxy-benzyl)-3-(2-pyridin-4-yl-thiazol-4-yl)-1H-pyridin-2-one (30 mg, 0.07 mmol, Step a) in 15 mL of CH₂Cl₂ was treated with 0.2 g of MnO₂ at RT for 2 h. HPLC indicated total conversion to aldehyde (MS m/z: 432.3 (M+1)). MnO₂ was filtered off through a Celite® pad. The filtrate was treated with 0.1 mL of piperidine, 0.05 mL of HOAc, and 0.05 mL of trimethoxyorthoformate. After stirring at RT for 30 min, 0.15 g of resin-bounded cyanoborohydride (Argonaut Technologies) was added and stirring was continued for 24 h. The resin was filtered off and solvents were removed under vacuum to give a solid which was used directly in the next step. MS m/z: 501.4 (M+1). Calc'd for C₂₉H₃₂N₄O₂S: 500.22.

(c) 6-Ethyl-5-piperidin-1-ylmethyl-3-(2-pyridin-4-yl-thiazol-4-yl)-1H-pyridin-2-one hydrochloric salt. A solution of 6-ethyl-1-(4-methoxy-benzyl)-5-piperidin-1-ylmethyl-3-(2-pyridin-4-yl-thiazol-4-yl)-1H-pyridin-2-one (Step b) in 1 mL of TFA:CH₂Cl₂ (1:1) was treated with 3-methoxybenzenethiol at 42° C. for 1 h. The reaction mixture was concentrated and the residue was dissolved in H₂O. The aqueous solution was extracted with 15 mL of CH₂Cl₂ and 2×15 mL of EtOAc. The aqueous layer was treated with 1N NaOH and 10 mL of saturated NaHCO₃, extracted with 3×10 mL of CH₂Cl₂. The organic layers were combined, dried, and concentrated to give a white solid. Gilson HPLC purification followed by basic aqueous extraction (CH₂Cl₂ and saturated aqueous NaHCO₃) and drying provided a white solid. Treatment of the solid in MeOH with excess 1N HCl in ether furnished the HCl salt as a yellow solid. MS m/z: 381.1 (M+1). Calc'd for C₂₁H₂₄N₄OS: 380.17.

### Example 107

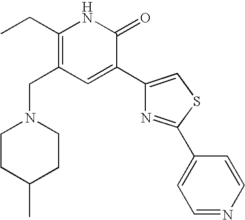

### 6-Ethyl-5-(4-methyl-piperazin-1-ylmethyl)-3-(2-pyridin-4-yl-thiazol-4-yl)-1H-pyridin-2-one

(a) 6-Ethyl-1-(4-m thoxy-benzyl)-5-(4-methyl-pip razin-1-ylmethyl)-3-(2-pyridin-4-yl-thiazol-4-yl)-1H-pyridin-2-one. The compound was prepared in a similar manner to Example 108(b) using 6-ethyl-5-hydroxymethyl-1-(4-methoxy-benzyl)-3-(2-pyridin-4-yl-thiazol-4-yl)-1H-pyridin-2-one (65 mg, 0.15 mmol, Example 106(a)). After reductive amination reaction, the resins were filtered off and the filtrate was concentrated. The resulting residue was treated with 20 mL of saturated aqueous NaHCO₃, extracted with 3×20 mL of CH₂Cl₂. The organic layers were combined, dried (Na₂SO₄), and concentrated to give a white solid without further purification. MS m/z: 516.2 (M+1). Calc'd for C₂₉H₃₃N₅O₂S: 515.24.

(b) 6-Ethyl-5-(4-methyl-piperazin-1-ylmethyl)-3-(2-pyridin-4-yl-thiazol-4-yl)-1H-pyridin-2-one hydrochloride salt. The compound was prepared in a similar manner to Example 106(c) using 6-ethyl-1-(4-methoxy-benzyl)-5-(4-methyl-piperazin-1-ylmethyl)-3-(2-pyridin-4-yl-thiazol-4-yl)-1H-pyridin-2-one (Step a). The HCl salt was isolated as a yellow solid. MS m/z: 396.2 (M+1). Calc'd for C₂₁H₂₅N₅OS: 395.18.

### Example 108

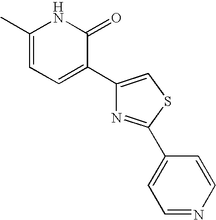

### 6-Methyl-3-(4-pyridin-4-yl-thiazol-2-yl)-1H-pyridin-2-one

To a solution of 3-cyano-6-methyl-2(1H)-pyridinone (Aldrich) (2.0 g, 15 mmol) and Et₃N (30 mL, 215 mmol) in 80 mL pyridine was bubbled H₂S gas for 5.5 h. The flask was capped and stirred overnight at RT. H₂S gas was bubbled for another 18 h and the mixture was filtered. The solid was washed with pyridine and dried in vacuo. A portion of this crude material (166 mg, 1 mmol) and 4-(bromoacetyl)pyridine hydrobromide (prepared by the method described in Aust. J. Chem., 42:1735 (1989); 299 g, 1.1 mmol) in 3 mL EtOH was heated at 150° C. for 5 min in the microwave synthesizer. The resulting solid was filtered, washed with EtOH, and dried in vacuo. The crude material was washed with a minimal amount of DMSO followed by water and dried in vacuo to give an orange amorphous solid. Mp: >300° C. MS m/z: 270 (M+1); 268 (M−1). Calc'd for C₁₄H₁₁N₃OS: 269.06.

The following compounds can be made by procedures similar to those previously described above:

a) 3-(4-(4-pyridinyl)-1,3-thiazol-2-yl)-5,6,7,8-tetrahydro-2(1H)-quinolinone;

b) 5-methyl-3-(4-(4-pyridinyl)-1,3-thiazol-2-yl)-7,8-dihydro-2(1H)-quinolinone;

c) 5-propylamino-3-(4-(4-pyridinyl)-1,3-thiazol-2-yl)-5,6,7,8-tetrahydro-2(1H)-quinolinone;

d) (5E)-5-propylimino-3-(4-(4-pyridinyl)-1,3-thiazol-2-yl)-5,6,7,8-tetrahydro-2(1H)-quinolinone; and

e) 3-(4-(4-pyridinyl)-1,3-thiazol-2-yl)-7,8-dihydro-2,5(1H,6H)-quinolinedione.

Other compounds included in this invention are set forth in Tables 1-2 below.

TABLE 1

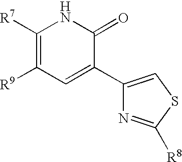

| # | R⁸ | R⁷ | R⁹ |
|---|---|---|---|
| 109. | 4-pyridyl | dimethylaminomethyl | H |
| 110. | 4-pyridyl | isopropyl | (Et)₂N(CH₂)₃—OC(O)— |
| 111. | 2-(Et)₂N(CH₂)₂—NH— | methyl | EtOC(O)— |
| | 4-pyridyl | | |
| 112. | 2-(2-furyl)CH₂—NH— | methyl | EtOC(O)— |
| | 4-pyridyl | | |
| 113. | 2-(2-thienyl)-(CH₂)₂—NH— | methyl | EtOC(O)— |
| | 4-pyridyl | | |
| 114. | 2-(4-F-phenyl)CH₂—NH— | methyl | EtOC(O)— |
| | 4-pyridyl | | |
| 115. | 2-(butyl-NH)-4-pyridyl | methyl | EtOC(O)— |
| 116. | 2-(NH₂—C(O)—CH₂—NH)— | methyl | EtOC(O)— |
| | 4-pyridyl | | |
| 117. | 2-(CH₃—C(O)NH—(CH₂)₂— | methyl | EtOC(O)— |
| | NH)-4-pyridyl | | |
| 118. | 2-(CH₃—C(O)NH—(CH₂)₂— | methyl | H |
| | NH)-4-pyridyl | | |
| 119. | 2-(4-CH₃O-phenyl)CH₂— | methyl | EtOC(O)— |
| | NH-4-pyridyl | | |
| 120. | 2-(4-CH₃O-phenyl)CH₂— | methyl | 4-CH₃O-benzyl- |
| | NH-4-pyridyl | | NHC(O)— |
| 121. | 2-(cyclopropyl-(CH₂)— | methyl | EtOC(O)— |
| | NH)-4-pyridyl | | |
| 122. | 2-(cyclopropyl-(CH₂)— | methyl | cyclopropyl- |
| | NH)-4-pyridyl | | (CH₂)—NH(C(O)— |
| 123. | 2-(cyclopentyl-(CH₂)— | methyl | EtOC(O)— |
| | NH)-4-pyridyl | | |
| 124. | 2-amino-4-pyridyl | methyl | EtOC(O)— |
| 125. | 2-(EtNHEtNH)— | methyl | (EtNHEtNH)—C(O)— |
| | 4-pyridyl | | |
| 126. | 4-pyridyl | 4-CH₃O-benzyloxy-CH₂— | EtOC(O)— |
| 127. | 4-pyridyl | methyl | HOCH₂— |

TABLE 2

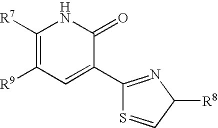

| # | R⁸ | R⁷ | R⁹ |
|---|---|---|---|
| 128. | 4-pyridyl | methyl | EtOC(O)— |
| 129. | 4-pyridyl | isopropyl | H |
| 130. | 4-pyridyl | ethyl | H |
| 131. | (2-thienyl)-SO₂CH₂— | isopropyl | H |
| 132. | (2-thienyl)-SO₂CH₂— | methyl | H |
| 133. | phenylSO₂CH₂— | isopropyl | H |
| 134. | phenylSO₂CH₂— | methyl | H |
| 135. | (2-pyridyl)-SO₂CH₂— | isopropyl | H |
| 136. | (4-pyridyl)-SO₂CH₂— | methyl | H |
| 137. | 4-pyridyl | H | H |

### Example 138

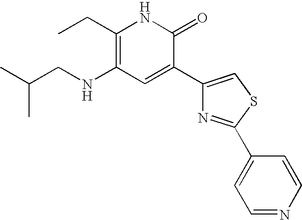

### 6-Ethyl-5-isobutylamino-3-(2-pyridin-4-yl-thiazol-4-yl)-1H-pyridin-2-one

(a) N-(4-Methoxybenzyl)acetoacetamide. To an ice-bath cooled solution of 4-methoxybenzyl amine (17.2 g, 125.4 mmol) in 200 mL of anhydrous THF was added diketene dropwise over 30 min. The reaction was stirred at RT overnight. The mixture was concentrated in vacuo and the orange residue was taken up in 200 mL of EtOAc, washed with H₂O, saturated NaHCO₃, dried over MgSO₄, and concentrated in vacuo to give an orange oil. The orange oil was suspended in 200 mL of EtₓO and filtered to give a yellow solid. MS m/z: 222 (M+1). Calc'd for C₁₂H₁₅NO₃: 221.11.

(b) Ethyl 5-acetyl-2-ethyl-1-(4-methoxybenzyl)-6-oxo-hydropyridine-3-carboxylate. To a solution of N-(4-methoxy-benzyl)acetoacetamide (Step a, 10.70 g, 48.4 mmol) and 150 mL of anhydrous THF was added 60% NaH (in mineral oil, 1.52 g, 38.0 mmol) portion-wise. After stirring for 15 min, a solution of ethyl (2Z)-2-propionyl-3-(dimethylamino)prop-2-enoate (9.62 g, 48.3 mmol, Example 1(a) in 150 mL of anhydrous THF was added dropwise. After the addition was complete the reaction was stirred at 60° C. overnight. The reaction was cooled to RT and concentrated in vacuo. The resulting residue was diluted with 200 mL of H₂O and acidified to pH 3 using 1N HCl (aq). The aqueous solution was extracted with EtOAc (3×). The combined EtOAc layers were washed with brine, dried over MgSO₄, and concentrated in vacuo to give a reddish oil. The oil was purified by flash chromatography on silica gel using 0.5% EtOAc:CH₂Cl₂ to give a reddish solid. MS m/z: 358 (M+1). Calc'd for C₂₀H₂₃NO₅ to 357.

(c) Ethyl 5-(2-bromoacetyl)-2-ethyl-1-(4-methoxybenzyl)-6-oxo-hydropyridine-3-carboxylate. This compound was prepared in a similar manner to Example 1c using ethyl 5-acetyl-2-ethyl-1-(4-methoxybenzyl)-6-oxohydropyridine-3-carboxylate (Step b, 6.78 g, 19.0 mmol), 5,5′-dibromobarbaturic acid (4.03 g, 14.1 mmol), and 150 mL of anhydrous THF. The resulting orange solid was carried on without further purification.

(d) Ethyl 2-ethyl-1-(4-methoxybenzyl)-6-oxo-5-(2-(4-pyridyl)(1,3-thiazol-4-yl)hydropyridine)-3-carboxylate. To a solution of crude ethyl 5-(2-bromoacetyl)-2-ethyl-1-(4-methoxybenzyl)-6-oxohydropyridine-3-carboxylate (Step c) and 200 mL of EtOH was added isothionicotinamide (2.60 g, 18.8 mmol). The solution was stirred at reflux overnight. The residue was cooled to RT, the precipitate was filtered and washed with EtOH to give a rust colored solid. MS m/z: 476 (M+1). Calc'd for C₂₆H₂₅N₃O₄S: 475.16.

(e) 2-Ethyl-1-(4-methoxybenzyl)-6-oxo-5-(2-(4-pyridyl)(1,3-thiazol-4-yl)hydropyridine-3-carboxylic acid. To a solution of ethyl 2-ethyl-1-(4-methoxybenzyl)-6-oxo-5-(2-(4-pyridyl) (1,3-thiazol-4-yl)hydropyridine-3-carboxylate (Step d, 0.30 g, 0.6 mmol) and 15 mL of THF was added 1N NaOH (1.3 mL, 1.3 mmol). After 2 h, an additional amount of 1N NaOH (1.3 mL, 1.3 mmol) was added. After an additional 2 h, the reaction was heated to 60° C. and stirred over the weekend. The reaction was concentrated in vacuo and the aqueous solution was acidified to pH 3 using 1N HCl (aq). The precipitate was filtered to give a yellow solid after drying in high vacuum. MS m/z: 448 (M+1). Calc'd for C₂₄H₂₁N₃O₄S: 447.13.

(f) [2-Ethyl-1-(4-methoxybenzyl)-6-oxo-5-(2-(4-pyridyl)(1,3-thiazol-4-yl)hydropyridin-3-yl]-carbamic acid tert-butyl ester. To a suspension of 2-ethyl-1-(4-methoxybenzyl)-6-oxo-5-(2-(4-pyridyl)(1,3-thiazol-4-yl)hydropyridine-3-carboxylic acid (Step e, 1.89 g, 4.2 mmol) and 20 mL of anhydrous toluene/20 mL of anhydrous 2-methyl-2-propanol was added DIEA (1.1 mL, 6.3 mmol). After stirring for 15 min, dppa (0.28 mL, 1.3 mmol) was added dropwise and the solution was stirred at 80° C. overnight. The reaction was cooled to RT and filtered. The resulting precipitate was washed with 9:1 CH₂Cl₂:MeOH. The filtrate was concentrated in vacuo, redissolved in EtOAc (150 mL) and washed with 1N NaOH, brine, dried over MgSO₄, and concentrated in vacuo. The residue was absorbed onto silica gel and purified by silica gel (ISCO flash chromatography instrument) using 3% MeOH:CH₂Cl₂ to give a yellow solid. MS m/z: 519 (M+1) Calc'd for C₂₈H₃₀N₄O₄S: 518.20.

(g) 6-Ethyl-5-isobutylamino-1-(4-methoxy-benzyl)-3-(2-pyridin-4-yl-thiazol-4-yl)-1H-pyridin-2-one. To a solution of [2-ethyl-1-(4-methoxybenzyl)-6-oxo-5-(2-(4-pyridyl)(1,3-thiazol-4-yl)hydropyridin-3-yl]-carbamic acid tert-butyl ester (Step f, 0.16 g, 0.31 mmol) in 5 mL of anhydrous DMF was added NaH (60% in mineral oil, 25 mg, 0.63 mmol). After stirring for 10 min, isobutyl bromide (0.05 mL, 0.46 mmol, Aldrich Chemical Co.) was added dropwise and stirred at RT overnight. The reaction was quenched with H₂O and concentrated in vacuo. The resulting residue was taken up in CH₂Cl₂:MeOH and 1 mL of 4M HCl in dioxane was added. After stirring for 2 h at RT, the mixture was neutralized with sat'd NaHCO₃. The organic layer was washed with brine, dried over MgSO₄, and concentrated in vacuo. The material was purified on the ISCO silica gel flash chromatography instrument using a gradient of 100% CH₂Cl₂ to 6% MeOH/CH₂Cl₂ to give a material that was carried on to the next step, without further purification. MS m/z: 475.1 (M+1). Calc'd for C₂₇H₃₀N₄O₂S: 474.21.

(h) 6-Ethyl-5-isobutylamino-3-(2-pyridin-4-yl-thiazol-4-yl)-1H-pyridin-2-one. This compound was prepared according to the method described in Example 84 by employing 6-ethyl-5-isobutylamino-1-(4-methoxy-benzyl)-3-(2-pyridin-4-yl-thiazol-4-yl)-1H-pyridin-2-one (Step g). MS m/z: 355.0 (M+1). Calc'd for C₁₉H₂₂N₄OS: 354.15.

### Example 139

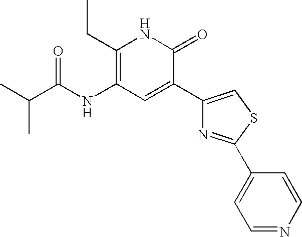

### N-[2-Ethyl-6-oxo-5-(2-pyridin-4-yl-thiazol-4-yl)-1,6-dihydo-pyridin-3-yl]-isobutyramide

(a) 5-Amino-6-ethyl-1-(4-methoxybenzyl)-3-(2-(4-pyridyl) (1,3-thiazol-4-yl))-1H-pyridin-2-one. To a suspension of [2-ethyl-1-(4-methoxybenzyl)-6-oxo-5-(2-(4-pyridyl) (1,3-thiazol-4-yl)hydropyridine]-3-carbamic acid tert-butyl ester (Example 138, Step f, 1.02 g, 2.0 mmol) in 40 mL of dioxane/25 mL of MeOH was added 4M HCl (in dioxane, 6.0 mL, 24 mmol). After stirring for 8 h at RT, an additional amount of 4M HCl (in dioxane, 1.0 mL, 4 mmol) was added and the reaction was stirred overnight. The resulting precipitate was filtered off and washed with ether to give a yellow solid. MS m/z: 419 (M+1). Calc'd for C₂₃H₂₂N₄O₂S: 418.15.

b) N-[2-Ethyl-1-(4-methoxy-benzyl)-6-oxo-5-(2-pyridin-4-yl-thiazol-4-yl)-1,6-dihydo-pyridin-3-yl]-isobutyramide. To a solution of 5-amino-6-ethyl-1-(4-methoxybenzyl)-3-(2-pyridin-4-yl-thiazol-4-yl)-1H-pyridin-2-one (Step a, 0.10 g, 0.24 mmol) in 5 mL of CH₂Cl₂ was added DIEA (0.04 mL, 0.24 mmol). After stirring for 5 min. the homogenous solution was placed in an ice bath and cooled. Isobutyryl chloride was added and stirring continued for 1 h. The yellow solution was filtered and washed with CH₂Cl₂ to give a solid. MS m/z: 489.0 (M+1). Calc'd for C₂₇H₂₈N₄O₃S: 488.19.

c) N-[2-Ethyl-6-oxo-5-(2-pyridin-4-yl-thiazol-4-yl)-1,6-dihydo-pyridin-3-yl]-isobutyramide. To a suspension of N-[2-ethyl-1-(4-methoxy-benzyl)-6-oxo-5-(2-pyridin-4-yl-thiazol-4-yl)-1,6-dihydo-pyridin-3-yl]-isobutyramide (Step b, 0.09 g, 0.18 mmol) in 9 mL of CH₂Cl₂ was added 3-methoxybenzenethiol (6 drops, Aldrich Chemical Co.) and TFA (3 mL) and the reaction was stirred at 40° C. overnight. The reaction was cooled to RT, diluted with CH₂Cl₂ and washed with sat'd NaHCO₃. An emulsion that developed between the organic and aqueous layers was filtered, dissolved in CH₂Cl₂:MeOH (9:1) and concentrated to dryness to give a yellow solid. MS m/z: 368.8 (M+1). Calc'd for C₁₉H₂₀N₄O₂S: 368.13.

### Example 140

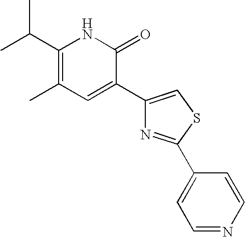

### 6-Isopropyl-5-methyl-3-(2-pyrindin-4-yl-thiazol-4-yl)-1H-pyridin-2-one

(a) 1-Dimethylamino-2,4-dimethylpent-1-en-3-one. To a microwave vial was added 2-methylpentan-3-one (2.0 mL, 16.19 mmol, Aldrich Chemical Co.) and N,N-dimethylformamide dimethyl acetal (3.0 mL, 22.58 mmol). The vial was heated by microwave for 7 min at 100° C. The temperature was elevated to 225° C. and continued for 130 min. The mixture was poured into 100 mL of brine and extracted with EtOAc (2×). The combined EtOAc layers were washed with H₂O, brine, dried over MgSO₄, and concentrated in vacuo to give a dark orange oil, which was used without further purification. MS m/z: 156.2 (M+1). Calc'd for C₉H₁₇NO: 155.13.

(b) 3-acetyl-6-isopropyl-5-methyl-1H-pyridin-2-one. To a solution of acetoacetamide (0.64 g, 6.33 mmol) in 20 mL of anhydrous THF was added NaH (60% in mineral oil, 0.19 g, 4.75 mmol) in portions. After 15 min, a solution of 1-dimethylamino-2,4-dimethylpent-1-en-3-one (Step a, 0.99 g, 6.38 mmol) in 10 mL of anhydrous THF was added dropwise. Upon completion of the addition the reaction was stirred at 60° C. overnight. The reaction was concentrated in vacuo and taken up in H₂O. The aqueous solution was acidified with 1N HCl to pH 3. The resulting precipitate was filtered and washed with hexane. The solid was purified with an ISCO silica gel flash chromatography instrument using a gradient of 20%→40% EtOAc:Hexanes over 20 min to give a yellow solid. MS m/z: 194.1 (M+1). Calc'd for C₁₁H₁₅NO₂: 193.11.

(c) 6-Isopropyl-5-methyl-3-(2-pyrindin-4-yl-thiazol-4-yl)-1H-pyridin-2-one. To a solution of 3-acetyl-6-isopropyl-5-methyl-1H-pyridin-2-one (Step b, 0.40 g, 2.07 mmol) and 40 mL of THF was added 5,5′-dibromobarbaturic acid (0.33 g, 1.15 mmol) and the reaction was heated to 60° C. for 5 h. The reaction was concentrated in vacuo and the residue was suspended in EtOAc. A tan solid was filtered, both filtrate and solid contained mono-bromination and di-bromination products. The filtrate was concentrated in vacuo and 10 mL of EtOH and isothionicotinamide (0.13 g, 0.94 mmol) were added. The solution was stirred at 80° C. overnight. The mixture was concentrated in vacuo and taken up in CH₂Cl₂. The solution was washed with sat'd NaHCO₃, H₂O, dried over MgSO₄, and concentrated in vacuo. The material was purified on an ISCO silica gel flash chromatography instrument using a gradient of CH₂Cl₂ to 3% MeOH/CH₂Cl₂ over 25 min to give a yellow solid. The solid was suspended in ether and filtered to give a yellow solid. MS m/z: 311.7 (M+1). Calc'd for C₁₇H₁₇N₃OS: 311.11.

### Example 141

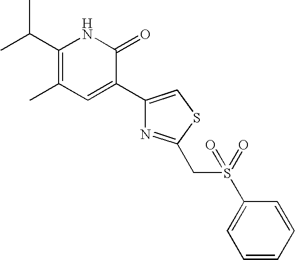

### 3-(2-Benzenesulfonylmethyl-thiazol-4-yl)-6-isopropyl-5-methyl-1H-pyridin-2-one

A solution of 3-(2-bromoacetyl)-6-isopropyl-5-methyl-1H-pyridin-2-one (0.18 g, solid from Example 140(c) containing both mono and di-brominated material), 2-(phenylsulfonyl)-ethanethioamide (0.13 g, 0.60 mmol), and 10 mL of EtOH was stirred at reflux for 4.5 h and filtered while hot. The solid was washed with hot EtOH, then hot EtOAc to give a tan solid. MS m/z: 389.3 (M+1). Calc'd for C₁₉H₂₀N₂O₃S₂: 388.09.

### Example 142

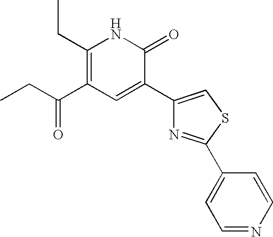

### 6-Ethyl-5-isopropionyl-3-(2-pyridin-4-yl-thiazol-4-yl)-1H-pyridin-2-one

(a) 4-Dimethylaminomethylene-heptane-3,5-dione. The compound was prepared according to the method described in Example 140(a) employing heptane-3,5-dione (2.0 mL, 14.76 mmol, Aldrich Chemical Co.) and N,N-dimethylformamide dimethyl acetal (3.0 mL, 22.58 mmol) to give a yellow oil. MS m/z: 184.3 (M+1). Calc'd for C₁₀H₁₇NO₂: 183.13.

(b) 3-Acetyl-6-ethyl-5-propionyl-1H-pyridin-2-one. This compound was prepared according to the method described in Example 140(b) employing 4-dimethylaminomethylene-heptane-3,5-dione (Step a, 1.60 g, 8.73 mmol), acetoacetamide (0.88 g, 8.70 mmol), and NaH (0.25 g, 6.25 mmol) to give a light yellow solid. MS m/z: 221.9 (M+1). Calc'd for C₁₂H₁₅NO₃: 221.11.

(c) 3-(2-Bromoacetyl)-6-ethyl-5-propionyl-1H-pyridin-2-one. To a solution of 3-acetyl-6-ethyl-5-propionyl-1H-pyridin-2-one (Step b, 0.65 g, 2.94 mmol) in 30 mL of THF was added 5,5′-dibromobarbaturic acid (0.43 g, 1.50 mmol) and stirred at 60° C. overnight. Additional 5,5′-dibromobarbaturic acid (0.08 g, 0.28 mmol) was added and the reaction was stirred for 1.5 h, at which time the starting material had been consumed. The reaction was concentrated in vacuo and the residue suspended in EtOAc and filtered to give a crude orange solid that was used without further purification. MS m/z: 300.0 and 302.0 (M+1). Calc'd for C₁₂H₁₄BrNO₃: 299.02.

(d) 6-Ethyl-5-propionyl-3-(2-pyridin-4-yl-thiazol-4-yl)-1H-pyridin-2-one. This compound was prepared according to the method described in Example 140 by employing crude 3-(2-bromoacetyl)-6-ethyl-5-propionyl-1H-pyridin-2-one (Step c, 0.30 g, 0.50 mmol), isothionicotinamide (0.11 g, 0.80 mmol) and 8 mL of EtOH to give a white solid. MS m/z: 340.2 (M+1). Calc'd for C₁₈H₁₇N₃O₂S: 339.10.

### Example 143

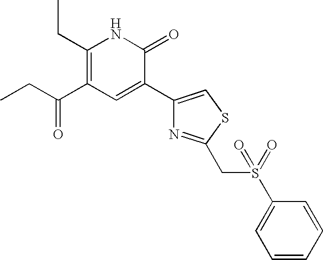

### 3-(2-Benzenesulfonylmethyl-thiazol-4yl)-6-ethyl-5-propionyl-1H-pyridin-2-one

This compound was prepared according to the method described in Example 141 by employing crude 3-(2-bromoacetyl)-6-ethyl-5-propionyl-1H-pyridin-2-one (Example 142, Step c, 0.30 g, 0.50 mmol), 2-(phenylsulfonyl) ethanethioamide (0.16 g, 0.74 mmol) and 8 mL of EtOH to give an off-white solid. MS m/z: 416.9 (M+1). Calc'd for C₂₀H₂₀N₂O₄S₂: 416.09.

### Example 144

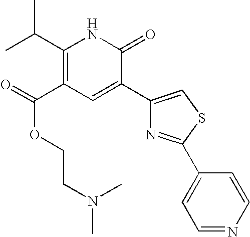

### 2-Isopropyl-6-oxo-5-(2-pyridin-4-yl-thiazol-4-yl)-1,6-dihydro-pyridine-3-carboxylic acid 2-dimethylamino-ethyl ester

(a) 5-(Imidazole-1-carbonyl)-6-isopropyl-3-(2-pyridin-4-yl-thiazol-4-yl)-1H-pyridin-2-one. To a suspension of 2-isopropyl-6-oxo-5-(2-pyridin-4-yl-thiazol-4-yl)-1,6-dihydro-pyridine-3-carboxylic acid (Example 81, 5.62 g, 16.46 mmol) and CDI (5.62 g, 34.66 mmol, Aldrich Chemical Co.) in 100 mL of CH₂Cl₂/30 mL of DMF was added DIEA (5.8 mL, 33.30 mmol). The reaction was stirred at RT overnight, filtered and the resulting solids were washed with CH₂Cl₂ to give an off-white solid. More solid was isolated by concentrating the filtrate and suspending the resulting material in CH₂Cl₂ to an off-white solid. The solids were combined to give the compound. MS m/z: 392.1 (M+1). Calc'd for C₂₀H₁₇N₅O₂S: 391.11.

(b) 2-Isopropyl-6-oxo-5-(2-pyridin-4-yl-thiazol-4-yl)-1,6-dihydro-pyridine-3-carboxylic acid 2-dimethylamino-ethyl ester. To a microwave tube was added 5-(imidazole-1-carbonyl)-6-isopropyl-3-(2-pyridin-4-yl-thiazol-4-yl)-1H-pyridin-2-one (Step a, 0.22 g, 0.56 mmol) and 2-dimethylaminoethanol (1 mL, Aldrich Chemical Co.). The solution was treated in the Smith Synthesizer for 10 min at 150° C. The reaction was diluted with 30 mL of CH₂Cl₂, washed with sat'd NaHCO₃ (2×), brine, dried over MgSO₄, and concentrated in vacuo to give an off-white solid. MS m/z: 413.0 (M+1). Calc'd for C₂₁H₂₄N₄O₃S: 412.16.

### Example 145

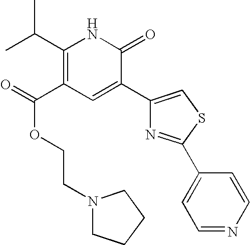

### 2-Isopropyl-6-oxo-5-(2-pyridin-4-yl-thiazol-4-yl)-1,6-dihydro-pyridine-3-carboxylic acid 2-pyrrolidin-1-yl-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 2-pyrrolidin-1-yl-ethanol (1 mL, Aldrich Chemical Co.) to give an off-white solid. MS m/z: 439.2 (M+1). Calc'd for C₂₃H₂₆N₄O₃S: 438.17.

### Example 146

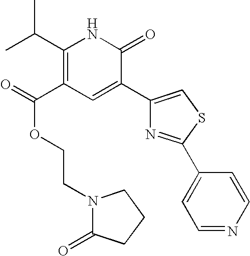

### 2-Isopropyl-6-oxo-5-(2-pyridin-4-yl-thiazol-4-yl)-1,6-dihydro-pyridine-3-carboxylic acid 2-(2-oxo-pyrrolidin-1-yl)-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 2-(2-oxo-pyrrolidin-1-yl)-ethanol (1 mL, Aldrich Chemical Co.) to give a white solid. MS m/z: 453.4 (M+1). Calc'd for C₂₃H₂₄N₄O₄S: 452.15.

### Example 147

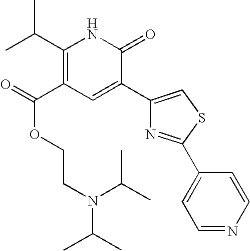

### 2-Isopropyl-6-oxo-5-(2-pyridin-4-yl-thiazol-4-yl)-1,6-dihydro-pyridine-3-carboxylic acid 2-diisopropylamino-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 2-diisopropylaminoethanol (1 mL, Aldrich Chemical Co.) to give a light pink solid. MS m/z: 469.2 (M+1). Calc'd for C₂₅H₃₂N₄O₃S: 468.22.

### Example 148

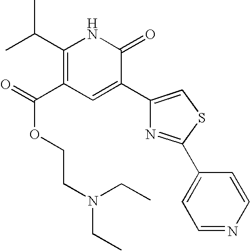

### 2-Isopropyl-6-oxo-5-(2-pyridin-4-yl-thiazol-4-yl)-1,6-dihydro-pyridine-3-carboxylic acid 2-diethylamino-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 2-diethylaminoethanol (0.5 mL, Aldrich Chemical Co.) to give a light pink solid. MS m/z: 441.1 (M+1). Calc'd for C₂₃H₂₈N₄O₃S: 440.19.

### Example 149

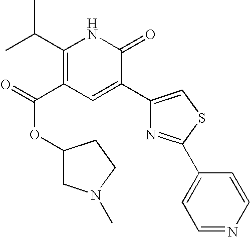

### 2-Isopropyl-6-oxo-5-(2-pyridin-4-yl-thiazol-4-yl)-1,6-dihydro-pyridine-3-carboxylic acid 1-methyl-pyrrolidin-3-yl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 1-methyl-pyrrolidin-3-ol (1 mL, Aldrich Chemical Co.) to give a white solid. MS m/z: 425.3 (M+1). Calc'd for C₂₂H₂₄N₄O₃S: 424.16.

### Example 150

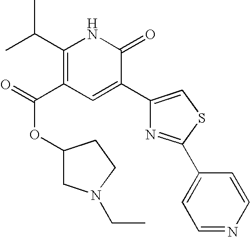

### 2-Isopropyl-6-oxo-5-(2-pyridin-4-yl-thiazol-4-yl)-1,6-dihydro-pyridine-3-carboxylic acid 1-ethyl-pyrrolidin-3-yl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 1-ethyl-pyrrolidin-3-ol (0.5 mL, Aldrich Chemical Co.) to give a light pink solid. MS m/z: 439.0 (M+1). Calc'd for C₂₃H₂₆N₄O₃S: 438.17.

### Example 151

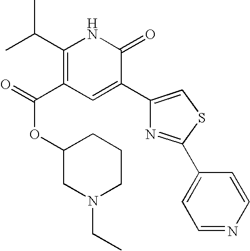

### 2-Isopropyl-6-oxo-5-(2-pyridin-4-yl-thiazol-4-yl)-1,6-dihydro-pyridine-3-carboxylic acid 1-ethyl-piperidin-3-yl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 1-ethyl-piperidin-3-ol (0.5 mL, Aldrich Chemical Co.) to give a white solid. MS m/z: 453.1 (M+1). Calc'd for C₂₄H₂₈N₄O₃S: 452.19.

### Example 152

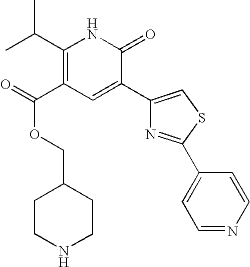

### 2-Isopropyl-6-oxo-5-(2-pyridin-4-yl-thiazol-4-yl)-1,6-dihydro-pyridine-3-carboxylic acid piperidin-4-ylmethyl ester

(a) 2-Isopropyl-6-oxo-5-(2-pyridin-4-yl-thiazol-4-yl)-1,6-dihydro-pyridine-3-carboxylic acid 1-tert-butoxycarbonyl-piperidin-4-yl-methyl ester. This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a 0.15 g, 0.38 mmol), 4-hydroxymethylpiperidine-1-carboxylic acid tert-butyl ester (0.14 g, 0.65 mmol), and DMF (2.5 mL) to give a white solid. MS m/z: 539.3 (M+1). Calc' for C₂₈H₃₄N₄O₅S: 538.22.

(b) 2-Isopropyl-6-oxo-5-(2-pyridin-4-yl-thiazol-4-yl)-1,6-dihydro-pyridine-3-carboxylic acid piperidin-4-ylmethyl ester. To a solution of 2-isopropyl-6-oxo-5-(2-pyridin-4-yl-thiazol-4-yl)-1,6-dihydro-pyridine-3-carboxylic acid 1-tert-butoxycarbonyl-piperidin-4-ylmethyl ester (Example 152, 65 mg, 0.12 mmol) in 15 mL of CH₂Cl₂ was added 4M HCl (in dioxane, 0.40 mL, 1.60 mmol). After stirring overnight the reaction was diluted with CH₂Cl₂ (50 mL) and washed with sat'd NaHCO₃. The aqueous layer was back extracted with CH₂Cl₂:MeOH (9:1). The combined organic layers were washed with brine, dried over MgSO₄, and concentrated in vacuo to give a white solid. MS m/z: 439.2 (M+1). Calc'd for C₂₃H₂₆N₄O₃S: 438.17.

### Example 153

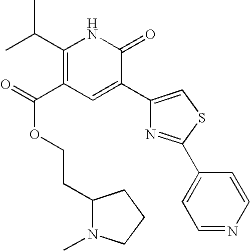

### 2-Isopropyl-6-oxo-5-(2-pyridin-4-yl-thiazol-4-yl)-1,6-dihydro-pyridine-3-carboxylic acid 2-(1-methyl-pyrrolidin-2-yl)-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 2-(1-methyl-pyrrolidin-2-yl)-ethanol (0.75 mL, TCI) to give a tan solid. MS m/z: 453.2 (M+1). Calc'd for C₂₄H₂₈N₄O₃S: 452.19.

### Example 154

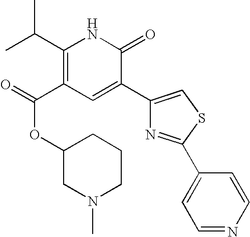

### 2-Isopropyl-6-oxo-5-(2-pyridin-4-yl-thiazol-4-yl)-1,6-dihydro-pyridine-3-carboxylic acid 1-methyl-piperidin-3-yl ester

This compound was prepared according to the method described in Example 144(b) by employing 5-(imidazole-1-carbonyl)-6-isopropyl-3-(2-pyridin-4-yl-thiazol-4-yl)-1H-yridin-2-one (Example 144, Step a, 0.12 g, 0.31 mmol) and 1-methyl-piperidin-3-ol (1 mL, Aldrich Chemical Co.) to give an off-white solid. MS m/z: 439.1 (M+1). Calc'd for C₂₃H₂₆N₄O₃S.

### Example 155

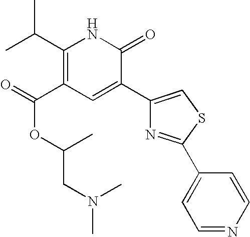

### 2-Isopropyl-6-oxo-5-(2-pyridin-4-yl-thiazol-4-yl)-1,6-dihydro-pyridine-3-carboxylic acid 2-dimethylamino-1-methyl-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 1-dimethylamino-propan-2-ol (0.75 mL, Aldrich Chemical Co.) to give an off-white solid. MS m/z: 427.3 (M+1). Calc'd for C₂₂H₂₆N₄O₃S: 426.17.

### Example 156

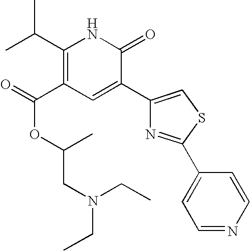

### 2-Isopropyl-6-oxo-5-(2-pyridin-4-yl-thiazol-4-yl)-1,6-dihydro-pyridine-3-carboxylic acid 2-diethylamino-1-methyl-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and l-diethylamino-propan-2-ol (0.75 mL, Aldrich Chemical Co.) to give a white solid. MS m/z: 455.1 (M+1). Calc'd for C₂₄H₃₀N₄O₃S: 454.20.

### Example 157

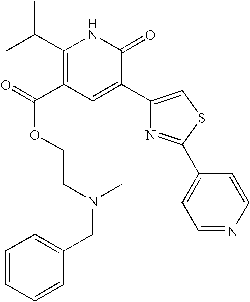

### 2-Isopropyl-6-oxo-5-(2-pyridin-4-yl-thiazol-4-yl)-1,6-dihydro-pyridine-3-carboxylic acid 2-(benzyl-methyl-amino)-thyl ester

This compound was prepared according to the method described in Example 144, Step b, by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 1-diethylamino-propan-2-ol (0.75 mL, Aldrich Chemical Co.) to give a white solid. MS m/z: 489.2 (M+1). Calc'd for C₂₇H₂₈N₄O₃S: 488.19.

### Example 158

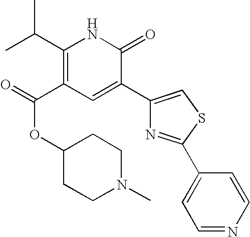

### 2-Isopropyl-6-oxo-5-(2-pyridin-4-yl-thiazol-4-yl)-1,6-dihydro-pyridine-3-carboxylic acid 1-methyl-piperidin-4-yl ester

This compound was prepared according to the method described in Example 144, Step b, by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 1-methyl-piperidin-4-ol (1.0 g, Aldrich Chemical Co.) to give an off-white solid. MS m/z: 439.3 (M+1). Calc'd for C₂₃H₂₆N₄O₃S: 438.17.

### Example 159

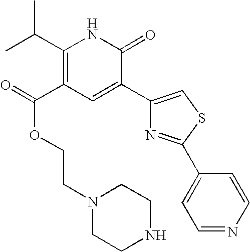

### 2-Isopropyl-6-oxo-5-(2-pyridin-4-yl-thiazol-4-yl)-1,6-dihydro-pyridine-3-carboxylic acid 2-piperazin-1-yl-ethyl ester

This compound was prepared according to the method described in Example 144, Step b, by employing 5-(imidazole-1-carbonyl)-6-isopropyl-3-(2-pyridin-4-yl-thiazol-4-yl)-1H-pyridin-2-one (Example 144, Step a, 0.19 g, 0.49 mmol) and 4-(2-hydroxyethyl)-piperazine-1-carboylic acid tert-butyl ester (0.42 g, 1.82 mmol) to give a white solid. To a solution of this solid in CH₂Cl₂ was added 4M HCl (in dioxane, 0.5 mL, 2.0 mmol). After stirring overnight the solution was concentrated to half volume and washed with sat'd NaHCO₃ (2×), H₂O, and brine. The resulting organic layer was concentrated in vacuo and the resulting solid suspended in ether and filtered to give a solid that was further purified on an ISCO silica gel flash chromatography instrument using a gradient of 5%-15% MeOH/CH₂Cl₂ to give an off-white solid. MS m/z: 454.1 (M+1). Calc'd for C₂₃H₂₇N₅O₃S: 453.18.

### Example 160

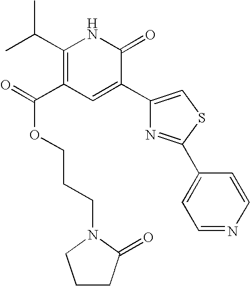

### 2-Isopropyl-6-oxo-5-(2-pyridin-4-yl-thiazol-4-yl)-1,6-dihydro-pyridine-3-carboxylic acid 2-(2-oxo-pyrrolidin-1-yl)-propyl ester

This compound was prepared according to the method described in Example 144, Step b, by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 3-(2-oxo-pyrrolidin-1-yl)-propanol (0.75 mL, Aldrich Chemical Co.) to give a light pink solid. MS m/z: 467.0 (M+1). Calc'd for C₂₄H₂₆N₄O⁴S: 466.17.

### Example 161

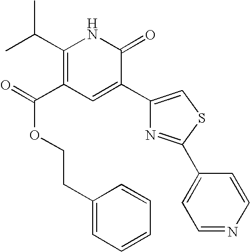

### 2-Isopropyl-6-oxo-5-(2-pyridin-4-yl-thiazol-4-yl)-1,6-dihydro-pyridine-3-carboxylic acid ph nethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 2-phenyl-ethanol (0.75 mL, Acros) to give a white solid. MS m/z: 446.2 (M+1). Calc'd for C₂₅H₂₃N₃O₃S: 445.15.

### Example 162

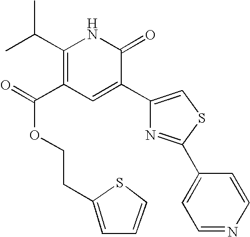

### 2-Isopropyl-6-oxo-5-(2-pyridin-4-yl-thiazol-4-yl)-1,6-dihydro-pyridine-3-carboxylic acid 2-thiophen-2-yl-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 0.12 g, 0.31 mmol) and 2-thiophen-2-yl-ethanol (0.75 mL, Aldrich Chemical Co.) to give an off-white solid. MS m/z: 452.0 (M+1). Calc'd for C₂₃H₂₁N₃O₃S₂: 451.10.

### Example 163

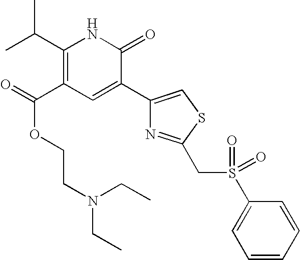

### 5-(2-Benzenesulfonylmethyl-thiazol-4-yl)-2-isopropyl-6-oxo-1,6-pyridine-3-carboxylic acid 2-diethylamino-ethyl ester

(a) 5-(2-Benzenesulfonylmethyl-thiazol-4-yl)-2-isopropyl-6-oxo-1,6-dihydro-pyridine carboxylic acid. To a solution of ethyl 5-(2-benzenesulfonylmethyl-thiazol-4-yl)-2-isopropyl-6-oxo-1,6-dihydro-pyridine carboxylate (Example 12, 1.8 g, 4.0 mmol) in 125 mL of a 3:1:1 mixture of THF:MeOH:H₂O was added 10 mL of 1M LiOH and 6 pellets of NaOH. After stirring overnight the solution was concentrated in vacuo to an aqueous solution and washed with CH₂Cl₂. The aqueous solution was acidified to pH 2 with 2N HCl and the resulting solids filtered. The solids suspended in toluene and concentrated in vacuo. This was repeated 4× to give a tan solid. MS m/z: 419.0 (M+1).

(b) 3-(2-Benzenesulfonylmethyl-thiazol-4-yl)-5-(imidazole-1-carbonyl)-6-isopropyl-1H-pyridin-2-one. This compound was prepared according to the method described in Example 144(a) by employing 5-(2-benzenesulfonylmethyl-thiazol-4-yl)-2-isopropyl-6-oxo-1,6-dihydro-pyridine-3-carboxylic acid (step a, 1.8 g, 4.30 mmol), CDI (1.36 g, 8.39 mmol), and DIEA (0.75 mL, 4.30 mmol) to give a solid. MS m/z: 469.1 (M+1).

Calc'd for C₂₂H₂₀N₄O₄S₂: 468.09.

(c) 5-(2-Benzenesulfonylm thyl-thiazol-4-yl)-2-isopropyl-6-oxo-1,6-pyridine-3-carboxylic acid 2-diethylamino-ethyl ester. This compound was prepared according to the method described in Example 144(b) by employing 3-(2-benzenesulfonyl-methyl-thiazol-4-yl)-5-(imidazole-1-carbonyl)-6-isopropyl-1H-pyridin-2-one (Step a, 0.13 g, 0.28 mmol) and 2-diethylaminoethanol (0.75 mL) to give a light yellow solid. MS m/z: 518.2 (M+1). Calc'd for C₂₅H₃₁N₃O₅S₂: 517.17.

### Example 164

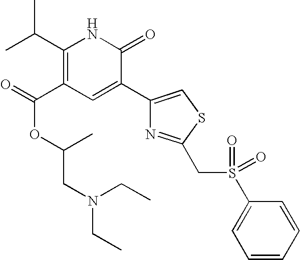

### 5-(2-Benzenesulfonylmethyl-thiazol-4-yl)-2-isopropyl-6-oxo-1,6-pyridine-3-carboxylic acid 2-diethylamino-1-methyl-ethyl ester

This compound was prepared according to the method described in Example 144(b) by employing 3-(2-benzenesulfonyl-methyl-thiazol-4-yl)-5-(imidazole-1-carbonyl)-6-isopropyl-1H-pyridin-2-one (Example 164, Step a, 0.13 g, 0.28 mmol) and 1-diethylamino-propan-2-ol (0.75 mL) to give a yellow solid. MS m/z: 532.2 (M+1). Calc'd for C₂₆H₃₃N₃O₅S₂: 531.19.

### Example 165

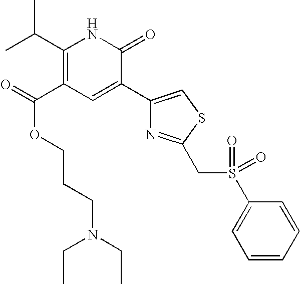

### 5-(2-Benzenesulfonylmethyl-thiazol-4-yl)-2-isopropyl-6-oxo-1,6-pyridine-3-carboxylic acid 2-diethylamino-propyl ester

This compound was prepared according to the method described in Example 144(b) by employing 3-(2-benzenesulfonyl-methyl-thiazol-4-yl)-5-(imidazole-1-carbonyl)-6-isopropyl-1H-pyridin-2-one (Example 164, Step a, 0.13 g, 0.28 mmol) and 3-diethylamino-propan-1-ol (0.75 mL) to give a tan solid. MS m/z: 532.2 (M+1). Calc'd for C₂₆H₃₃N₃O₅S₂: 531.19.

### Example 166

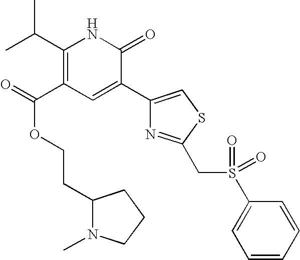

### 5-(2-Benzenesulfonylmethyl-thiazol-4-yl)-2-isopropyl-6-oxo-1,6-pyridine-3-carboxylic acid 2-(1-methyl-pyrrolidin-2-yl)-ethyl ester

This compound was prepared according to the method described in Example 144(b) by employing 3-(2-benzenesulfonyl-methyl-thiazol-4-yl)-5-(imidazole-1-carbonyl)-6-isopropyl-1H-pyridin-2-one (Example 164, Step a, 0.13 g, 0.28 mmol) and 2-(1-methyl-pyrrolidin-2-yl)-ethanol (0.75 mL) to give a light yellow solid. MS m/z: 530.5 (M+1). Calc'd for C₂₆H₃₁N₃O₅S₂: 529.17.

### Example 167

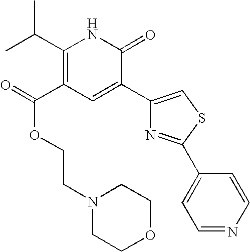

### 2-Isopropyl-6-oxo-5-(2-pyridin-4-yl-thiazol-4-yl)-1,6-dihydro-pyridine-3-carboxylic acid 2-morpholin-4-yl-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 75 mg, 0.19 mmol) and 2-morpholin-4-yl-ethanol (1.0 mL, Aldrich Chemical Co.) to give a white solid. MS m/z: 455.2 (M+1). Calc'd for C₂₃H₂₆N₄O₄S: 454.17.

### Example 168

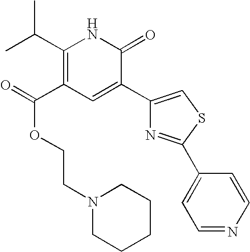

### 2-Isopropyl-6-oxo-5-(2-pyridin-4-yl-thiazol-4-yl)-1,6-dihydro-pyridine-3-carboxylic acid 2-piperidin-1-yl-ethyl ester

This compound was prepared according to the method described in Example 144(b) by employing 5-(imidazole-1-carbonyl)-6-isopropyl-3-(2-pyridin-4-yl-thiazol-4-yl)-1H-pyridin-2-one (Example 144, Step a, 120 mg, 0.31 mmol) and 2-piperidin-1-yl-ethanol (1.0 mL, Aldrich Chemical Co.) to give an off-white solid. MS m/z: 453.2 (M+1). Calc'd for C₂₄H₂₈N₄O₃S: 452.19.

### Example 169

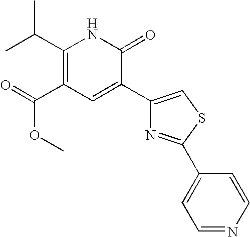

### 2-Isopropyl-6-oxo-5-(2-pyridin-4-yl-thiazol-4-yl)-1,6-dihydro-pyridine-3-carboxylic acid methyl ester

This compound was prepared by heating the mixture of 5-(imidazole-1-carbonyl)-6-isopropyl-3-(2-pyridin-4-yl-thiazol-4-yl)-1H-pyridin-2-one (Example 144, Step a, 55 mg, 0.14 mmol) and anhydrous methanol (3.0 mL, Aldrich Chemical Co.) in the microwave smithsynthesizer at 120° C. for 10 min to obtain a yellow solid, which was further purified by HPLC to provide the TFA salt. MS m/z: 356.2 (M+1). Calc'd for C₁₈H₁₇N₃O₃S: 355.10.

### Example 170

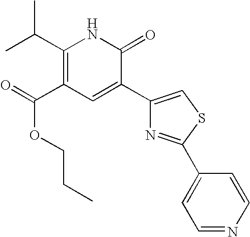

### 2-Isopropyl-6-oxo-5-(2-pyridin-4-yl-thiazol-4-yl)-1,6-dihydro-pyridine-3-carboxylic acid propyl ester

This compound was prepared by heating the mixture of 5-(imidazole-1-carbonyl)-6-isopropyl-3-(2-pyridin-4-yl-thiazol-4-yl)-1H-pyridin-2-one (Example 144, Step a, 50 mg, 0.14 mmol) and anhydrous 1-propanol (3.0 mL, Aldrich Chemical Co.) in the microwave smithsynthesizer at 150° C. for 2×10 min to obtain crude product, which was further purified by HPLC to provide the TFA salt as a yellow solid. MS m/z: 384.1 (M+1). Calc'd for C₂₀H₂₁N₃O₃S: 383.13.

### Example 171

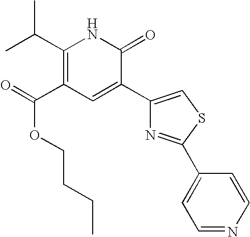

### 2-Isopropyl-6-oxo-5-(2-pyridin-4-yl-thiazol-4-yl)-1,6-dihydro-pyridine-3-carboxylic acid butyl ester

This compound was prepared by heating the mixture of 5-(imidazole-1-carbonyl)-6-isopropyl-3-(2-pyridin-4-yl-thiazol-4-yl)-1H-pyridin-2-one (Example 144, Step a, 50 mg, 0.14 mmol) and anhydrous 1-butanol (3.0 mL, Aldrich Chemical Co.) in the microwave smithsynthesizer at 150° C. for 2×10 min to obtain crude product, which was further purified by HPLC to provide the TFA salt as a yellow solid. MS m/z: 398.2 (M+1). Calc'd for C₂₁H₂₃N₃O₃S: 397.15.

### Example 172

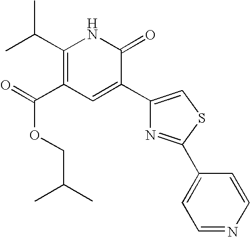

### 2-Isopropyl-6-oxo-5-(2-pyridin-4-yl-thiazol-4-yl)-1,6-dihydro-pyridine-3-carboxylic acid isobutyl ester

This compound was prepared by heating the mixture of 5-(imidazole-1-carbonyl)-6-isopropyl-3-(2-pyridin-4-yl-thiazol-4-yl)-1H-pyridin-2-one (Example 144, Step a, 50 mg, 0.14 mmol) and anhydrous iso-butanol (3.0 mL, Aldrich Chemical Co.) in the microwave smithsynthesizer at 150° C. for 2×10 min to obtain crude product, which was further purified by HPLC to provide the TFA salt as a yellow solid. MS m/z: 398.3 (M+1). Calc'd for C₂₁H₂₃N₃O₃S: 397.15.

### Example 173

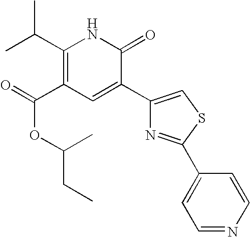

### 2-Isopropyl-6-oxo-5-(2-pyridin-4-yl-thiazol-4-yl)-1,6-dihydro-pyridine-3-carboxylic acid sec-butyl ester

This compound was prepared by heating the mixture of 5-(imidazole-1-carbonyl)-6-isopropyl-3-(2-pyridin-4-yl-thiazol-4-yl)-1H-pyridin-2-one (Example 164, Step a, 50 mg, 0.14 mmol) and anhydrous sec-butanol (3.0 mL, Aldrich Chemical Co.) in the microwave smithsynthesizer at 150° C. for 2×10 min to obtain crude product, which was further purified by HPLC to provide the TFA salt as a yellow solid. MS m/z: 398.2 (M+1). Calc'd for C₂₁H₂₃N₃O₃S: 397.15.

### Example 174

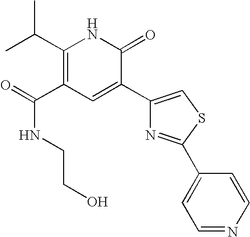

### 2-Isopropyl-6-oxo-5-(2-pyridin-4-yl-thiazol-4-yl)-1,6-dihydro-pyridine-3-carboxylic acid (2-hydroxy-ethyl)-amide

A mixture of 5-(imidazole-1-carbonyl)-6-isopropyl-3-(2-pyridin-4-yl-thiazol-4-yl)-1H-pyridin-2-one (Example 144, Step a, 300 mg, 0.77 mmol), 2-hydroxy-ethylamine (1.0 mL, Aldrich Chemical Co.), and DIEA (0.5 mL, Aldrich Chemical Co.) in 20 mL of anhydrous CH₂Cl₂ was stirred at RT for 3 days. Precipitate was collected by filtration and washed by CH₂Cl₂:hexanes (1:1) to give the title compound as an off-white solid. MS m/z: 385.1 (M+1). Calc'd for C₁₉H₂₀N₄O₃S: 384.13.

### Example 175

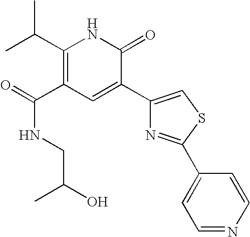

### 2-Isopropyl-6-oxo-5-(2-pyridin-4-yl-thiazol-4-yl)-1,6-dihydro-pyridine-3-carboxylic acid (2-hydroxy-propyl)-amide

A mixture of 5-(imidazole-1-carbonyl)-6-isopropyl-3-(2-pyridin-4-yl-thiazol-4-yl)-1H-pyridin-2-one (Example 144, Step a, 300 mg, 0.77 mmol), 2-hydroxy-propylamine (1.0 mL, Aldrich Chemical Co.), and DIEA (0.5 mL, Aldrich Chemical Co.) in 20 mL of anhydrous CH₂Cl₂ was stirred at RT for 3 days. Precipitate was collected by filtration and washed by CH₂Cl₂:hexanes (1:1) to give the title compound as an off-white solid. MS m/z: 399.4 (M+1). Calc'd for C₂₀H₂₂N₄O₃S: 398.14.

### Example 176

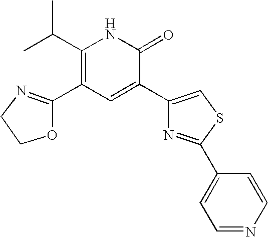

### 5-(4,5-Dihydro-oxazol-2-yl)-6-isopropyl-3-(2-pyridin-4-yl-thiazol-4-yl)-1H-pyridin-2-one

A mixture of 2-isopropyl-6-oxo-5-(2-pyridin-4-yl-thiazol-4-yl)-1,6-dihydro-pyridine-3-carboxylic acid (2-hydroxy-ethyl)-amide (Example 175, 150 mg, 0.39 mmol), PPh₃ (260 mg, 1.0 mmol, Aldrich Chemical Co.), and DIAD (0.15 mL, 0.76 mmol, Aldrich Chemical Co.) in 25 mL of anhydrous CH₂Cl₂ was stirred at RT overnight. Precipitate was collected by filtration and washed by CH₂Cl₂ to give the title compound as a white solid. MS m/z: 367.0 (M+1).

Calc'd for C₁₉H₁₈N₄O₂S: 366.12.

### Example 177

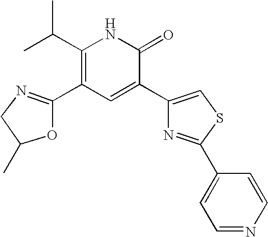

### 6-Isopropyl-5-(5-methyl-4,5-dihydro-oxazol-2-yl)-3-(2-pyridin-4-yl-thiazol-4-yl)-1H-pyridin-2-one

A mixture of 2-isopropyl-6-oxo-5-(2-pyridin-4-yl-thiazol-4-yl)-1,6-dihydro-pyridine-3-carboxylic acid (2-hydroxy-propyl)-amide (Example 176, 150 mg, 0.38 mmol), PPh₃ (260 mg, 1.0 mmol, Aldrich Chemical Co.), and DIAD (0.15 mL, 0.76 mmol, Aldrich Chemical Co.) in 25 mL of anhydrous CH₂Cl₂ was stirred at RT overnight. The reaction mixture was concentrated and the residue was purified twice by Prep-TLC using MeOH:CH₂Cl₂ (5:95) as eluent to give the title compound as an off-white solid. MS m/z: 381.0 (M+1).

Calc'd for C₂₀H₂₀N₄O₂S: 380.13.

### Example 178

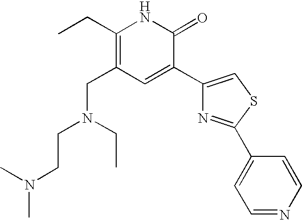

### 5-{[(2-Dimethylamino-ethyl)-ethyl-amino]-methyl}-6-ethyl-3-(2-pyridin-4-yl-thiazol-4-yl)-1H-pyridin-2-one

(a) 5-{[(2-Dimethylamino-ethyl)-ethyl-amino]-methyl}-6-ethyl-1-(4-methoxy-benzyl)-3-(2-pyridin-4-yl-thiazol-4-yl)-1H-pyridin-2-one. The compound was prepared in a similar anner to Example 108(b) using 6-ethyl-5-hydroxymethyl-1-(4-methoxy-benzyl)-3-(2-pyridin-4-yl-thiazol-4-yl)-1H-pyridin-2-one (100 mg, 0.23 mmol, Example 108(a)), N′-ethyl-N,N-dimethyl-ethane-1,2-diamine (0.5 mL, Aldrich), and NaBH(OAc)₃ (250 mg, 1.18 mmol, Aldrich) in 30 mL of CH₂Cl₂. After reductive amination reaction, the mixture was treated with 20 mL of saturated aqueous NaHCO₃ and the layers were separated. The organic layer was washed again with 20 mL of saturated aqueous NaHCO₃. The organic layer was separated, dried (Na₂SO₄), and concentrated to give an oil without further purification. MS m/z: 532.3 (M+1). Calc'd for C₃₀H₃₇N₅O₂S: 531.27.

(b) 5-{[(2-Dimethylamino-ethyl)-ethyl-amino]-methyl}-6-ethyl-3-(2-pyridin-4-yl-thiazol-4-yl)-1H-pyridin-2-one. The compound was prepared in a similar manner to Example 108(c) using 5-{[(2-dimethylamino-ethyl)-ethyl-amino]-methyl}-6-ethyl-1-(4-methoxy-benzyl)-3-(2-pyridin-4-yl-thiazol-4-yl)-1H-pyridin-2-one (Example 178(a)) and purified by Prep-TLC using MeOH:CH₂Cl₂ (10:90) to afford a white solid. MS m/z: 412.3 (M+1). Calc'd for C₂₂H₂₉N₅OS: 411.21.

### Example 179

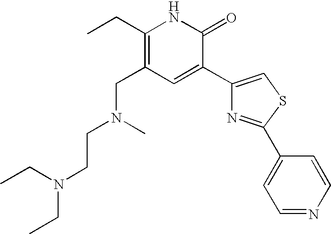

### 5-{[(2-Diethylamino-ethyl)-m thyl-amino]-methyl}-6-ethyl-3-(2-pyridin-4-yl-thiazol-4-yl)-1H-pyridin-2-one

(a) 5-([(2-Diethylamino-ethyl)-methyl-amino]-methyl)-6-ethyl-1-(4-methoxy-benzyl)-3-(2-pyridin-4-yl-thiazol-4-yl)-1H-pyridin-2-one. The compound was prepared in a similar manner to Example 178(a) using 6-ethyl-5-hydroxymethyl-1-(4-methoxy-benzyl)-3-(2-pyridin-4-yl-thiazol-4-yl)-1H-pyridin-2-one (100 mg, 0.23 mmol, Example 108(a)), N,N-diethyl-N′-methyl-ethane-1,2-diamine (0.5 mL, Aldrich) and NaBH(OAc)₃ (250 mg, 1.18 mmol, Aldrich) in 30 mL of CH₂Cl₂. After reductive amination reaction, the mixture was treated with 20 mL of saturated aqueous NaHCO₃ and the layers were separated. The organic layer was washed again with 20 mL of saturated aqueous NaHCO₃. The organic layer was separated, dried (Na₂SO₄), and concentrated to give an oil without further purification. MS m/z: 546.4 (M+1). Calc'd for C₃₁H₃₉N₅O₂S: 545.28.

(b) 5-{[(2-Dimethylamino-ethyl)-ethyl-amino]-methyl}-6-ethyl-3-(2-pyridin-4-yl-thiazol-4-yl)-1H-pyridin-2-one. The compound was prepared in a similar manner to Example 108(c) using 5-{[(2-diethylamino-ethyl)-methyl-amino)-methyl}-6-ethyl-1-(4-methoxy-benzyl)-3-(2-pyridin-4-yl-thiazol-4-yl)-1H-pyridin-2-one (Example 179(a)) and purified by Prep-TLC using MeOH:CH₂Cl₂ (10:90) to afford a white solid. MS m/z: 426.4 (M+1). Calc'd for C₂₃H₃₁N₅OS: 425.22.

### Example 180

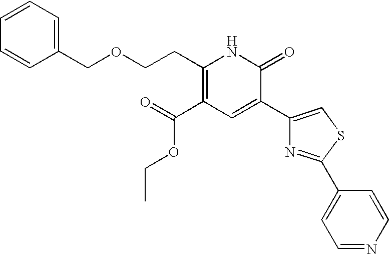

### 2-(2-Benzyloxy-ethyl)-6-oxo-5-(2-pyridin-4-yl-thiazol-4-yl)-1,6-dihydro-pyridine-3-carboxylic acid ethyl ester

(a) 5-Benzyloxy-2-dimethylaminomethylene-3-oxo-pentanoic acid ethyl ester. A mixture of N,N-dimethylformamide dimethyl acetal (8.0 mL, 60.0 mmol) and 5-benzyloxy-3-oxo-pentanoic acid ethyl ester (10.0 g, 40 mmol, prepared by following a literature procedure, Claffey, et al., J. Org. Chem., 64:8267 (1999) was heated at 95° C. for 2 h. The resulting red solution was concentrated to constant weight to provide a dark red oil. MS m/z: 306.3 (M+1). Calc'd for C₁₇H₂₃NO₄: 305.16.

(b) 5-Acetyl-2-(2-benzyloxy-ethyl)-6-oxo-1,6-dihydro-pyridine-3-carboxylic acid ethyl ester. This compound was prepared in a similar manner to Example 1(b) using 5-benzyloxy-2-dimethylaminomethylene-3-oxo-pentanoic acid ethyl ester (12.08 g, 39.56 mmol), acetoacetamide (4.03 g, 39.86 mmol), and NaH (60% in mineral oil, 1.24 g, 31.0 mmol) to give a yellow solid. MS m/z: 344.4 (M+1). Calc'd for C₁₉H₂₁NO₅: 343.14.

(c) 2-(2-Benzyloxy-ethyl)-5-(2-bromo-acetyl)-6-oxo-1,6-dihydro-pyridine-3-carboxylic acid ethyl ester. This compound was prepared in a similar manner to Example 1(c) using 5-acetyl-2-(2-benzyloxy-ethyl)-6-oxo-1,6-dihydro-pyridine-3-carboxylic acid ethyl ester (2.18 g, 6.36 mmol, Step b) and 5,5-dibromobarbituric acid (1.1 g, 3.82 mmol) to provide a yellow solid which was used directly in the next step without further purification.

(d) 2-(2-Benzyloxy-ethyl)-6-oxo-5-(2-pyridin-4-yl-thiazol-4-yl)-1,6-dihydro-pyridine-3-carboxylic acid ethyl ester. This compound was prepared in a similar manner to Example 1(d) using 2-(2-benzyloxy-ethyl)-5-(2-bromo-acetyl)-6-oxo-1,6-dihydro-pyridine-3-carboxylic acid ethyl ester (Step c) and isothionicotinamide (0.89 g, 6.4 mmol) to provide a pink solid. Crude material (50 mg) was purified by Prep-TLC with MeOH:CH₂Cl₂ (5:95) to afford the title compound as an off-white solid. MS m/z: 462.1 (M+1). Calc'd for C₂₅H₂₃N₃O₄S: 461.14.

### Example 181

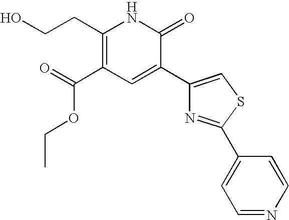

### 2-(2-Hydroxy-ethyl)-6-oxo-5-(2-pyridin-4-yl-thiazol-4-yl)-1,6-dihydro-pyridine-3-carboxylic acid ethyl ester

A suspension of 2-(2-benzyloxy-ethyl)-6-oxo-5-(2-pyridin-4-yl-thiazol-4-yl)-1,6-dihydro-pyridine-3-carboxylic acid ethyl ester (75 mg, 0.16 mmol, Example 180(d)) in 25 mL of CH₂Cl₂ was treated with BCl₃ (1.0 M, 0.5 mL) in CH₂Cl₂ at RT overnight. The reaction was quenched by addition of 10 mL of 1M HCl. A few min later, saturated aqueous NaHCO₃ was added to adjust the pH to 8. Layers were separated after vigorous mixing. The aqueous layer was extracted again with 30 mL of CH₂Cl₂. The organic layers were combined, concentrated to give a residue, which was re-suspended in CH₂Cl₂ and filtered to provide the title compound as a pink solid. MS m/z: 372.1 (M+1). C₁₈H₁₇N₃O₄S: 371.09.

### Example 182

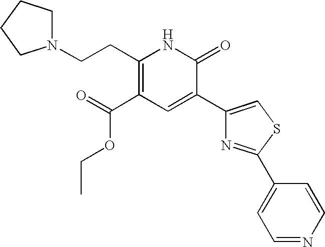

### 6-Oxo-5-(2-pyridin-4-yl-thiazol-4-yl)-2-(2-pyrrolidin-1-yl-ethyl)-1,6-dihydro-pyridine-3-carboxylic acid ethyl ester

A solution of 2-(2-hydroxyethyl)-6-oxo-5-(2-pyridin-4-yl-thiazol-4-yl)-1,6-dihydro-pyridine-3-carboxylic acid ethyl ester (50 mg, 0.14 mmol, Example 181) in 5 mL of anhydrous CH₂Cl₂ and 5 mL of pyridine was treated with mesyl chloride (0.15 mL). After stirring for 15 min, solvents were removed and the residue was azeotroped with 2×10 mL of toluene. This crude material was used in the next step without further purification. MS m/z: 450.0 (M+1). Calc'd for C₁₉H₁₉N₃O₆S₂: 449.07. The residue from above containing the mesylate was treated with 1.5 mL of pyrrolidine at RT for 3 min followed by heating at 60° C. for 5 min. Pyrrolidine was then removed. The residue was partitioned between 35 mL of CH₂Cl₂ and 20 mL of 1M HCl. The aqueous layer was separated, basicified with saturated aqueous NaHCO₃, and extracted with 3×20 mL of CH₂Cl₂. The organic layers were combined, dried (Na₂SO₄), and concentrated to give a residue, which was purified by Prep-TLC using MeOH:CH₂Cl₂ (10:90) to afford the title compound as an off-white solid. MS m/z: 425.1 (M+1). Calc'd for C₂₂H₂₄N₄O₃S: 424.16.

### Example 183

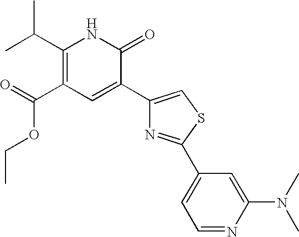

### 5-[2-(2-Dimethylamino-pyridin-4-yl)-thiazol-4-yl]-2-isopropyl-6-oxo-1,6-dihydro-pyridine-3-carboxylic acid ethyl ester

A mixture of 5-(2-bromoacetyl)-2-isopropyl-6-oxo-1,6-dihydro-pyridine-3-carboxylic acid ethyl ester (Example 10c, 0.20 g, 0.61 mmol) and 2-dimethylamino-thioisonicotinamide (0.14 g, 0.79 mmol) in EtOH (10 mL) was heated at reflux for 24 h. The mixture was cooled, concentrated, and purified by flash column chromatography (3% MeOH/CH₂Cl₂) to give an off-white solid. MS (m/z, M+1): 413.4. Calc'd for C₂₁H₂₄N₄O₃S: 412.16.

### Example 184

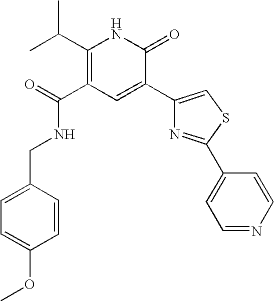

### 2-(1-Isopropyl)-N-(4-methoxybenzyl)-6-oxo-5-(2-(4-pyridinyl)-1,3-thiazol-4-yl)-1,6-dihydro-3-pyridinecarboxamide

A mixture of 2-isopropyl-6-oxo-5-(2-pyridin-4-yl)thiazol-4-yl)-1,6-dihydo-pyridine-3-carboxylic acid (Example 81, 0.15 g, 0.44 mmol), HOAt (0.08 g, 0.53 mmol), DIEA (0.28 g, 2.2 mmol), p-methoxylbenzylamine (0.073 g, 0.53 mmol), and EDC (0.17 g, 0.88 mmol) in DMF (10 mL) was stirred at RT for 24 h. The mixture was concentrated, and taken up in H₂O. The tan solid was filtered, and air-dried. MS (m/z, M+1): 461.4. Calc'd for C₂₅H₂₄N₄O₃S: 460.16.

### Example 185

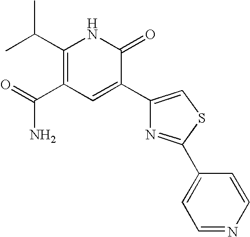

### 2-Isopropyl-6-oxo-5-(2-pyridin-4-yl)thiazol-4-yl)-1,6-dihydo-pyridine-3-carboxylic acid amide

A mixture of 2-(1-isopropyl)-N-(4-methoxybenzyl)-6-oxo-5-(2-(4-pyridinyl)-1,3-thiazol-4-yl)-1,6-dihydro-3-pyridinecarboxamide (Example 184, 0.09 g, 0.20 mmol), TFA (5 mL), and p-anisole (10 mL) was heated at 120° C. for 36 h. The mixture was cooled, concentrated, and taken up in H₂O. The yellow solid was filtered, and triturated in EtOH to give a light yellow solid. MS (m/z, M+1): 341.4. Calc'd for C₁₇H₁₆N₄O₂S: 340.10.

### Example 186

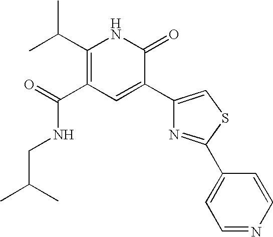

### 2-Isopropyl-6-oxo-5-(2-pyridin-4-yl)thiazol-4-yl)-1,6-dihydo-pyridine-3-carboxylic acid isobutylamide

This compound was prepared in a similar manner to Example 184 using 2-isopropyl-6-oxo-5-(2-pyridin-4-yl)thiazol-4-yl)-1,6-dihydo-pyridine-3-carboxylic acid (Example 81) and isobutylamine to give the title product as an off-white solid. MS (m/z, M+1): 397.4. Calc'd for C₂₁H₂₄N₄O₂S: 396.16.

### Example 187

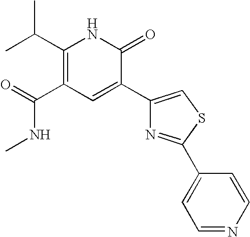

### 2-Isopropyl-6-oxo-5-(2-pyridin-4-yl)thiazol-4-yl)-1,6-dihydo-pyridine-3-carboxylic acid methylamide

This compound was prepared in a similar manner to Example 184 using 2-isopropyl-6-oxo-5-(2-pyridin-4-yl)thiazol-4-yl)-1,6-dihydo-pyridine-3-carboxylic acid (Example 81) and methylamine to give the title product as an off-white solid. MS (m/z, M+1): 355.4. Calc'd for C₁₈H₁₈N₄O₂S: 354.12.

### Example 188

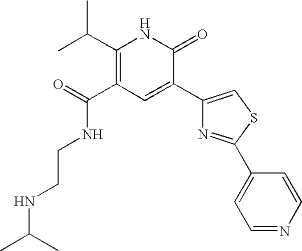

### 2-Isopropyl-6-oxo-5-(2-pyridin-4-yl)thiazol-4-yl)-1,6-dihydo-pyridine-3-carboxylic acid (2-isopropylamino-ethyl)-amide

This compound was prepared in a similar manner to Example 184 using 2-isopropyl-6-oxo-5-(2-pyridin-4-yl)thiazol-4-yl)-1,6-dihydo-pyridine-3-carboxylic acid (Example 81) and 2-isopropylamino-ethylamine to give the title product as a light yellow solid. MS (m/z, M+1): 426.4.

Calc'd for C₂₂H₂₇N₅O₂S: 425.19.

### Example 189

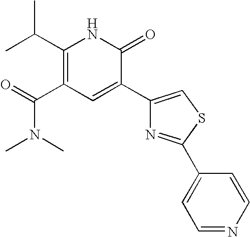

### 2-Isopropyl-6-oxo-5-(2-pyridin-4-yl)thiazol-4-yl)-1,6-dihydo-pyridine-3-carboxylic acid dimethylamide

This compound was prepared in a similar manner to Example 184 using 2-isopropyl-6-oxo-5-(2-pyridin-4-yl)thiazol-4-yl)-1,6-dihydo-pyridine-3-carboxylic acid (Example 81) and dimethylamine to give the title product as a tan solid. MS (m/z, M+1): 369.4. Calc'd for C₁₉H₂₀N₄O₂S: 368.13.

### Example 190

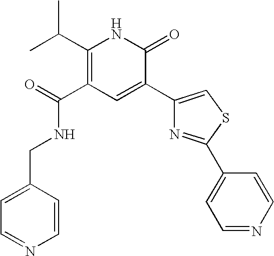

### 2-Isopropyl-6-oxo-5-(2-pyridin-4-yl)thiazol-4-yl)-1,6-dihydo-pyridine-3-carboxylic acid (pyridine-4-ylmethyl)-amide

This compound was prepared in a similar manner to Example 184 using 2-isopropyl-6-oxo-5-(2-pyridin-4-yl)thiazol-4-yl)-1,6-dihydo-pyridine-3-carboxylic acid (Example 81) and pyridin-4-yl-methylamine to give the title product as an off-white solid. MS (m/z, M+1): 432.4. Calc'd for C₂₃H₂₁N₅O₂S: 431.14.

### Example 191

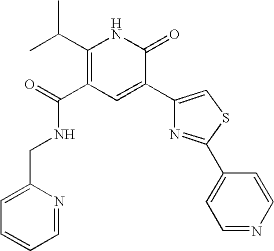

### 2-Isopropyl-6-oxo-5-(2-pyridin-4-yl)thiazol-4-yl)-1,6-dihydo-pyridine-3-carboxylic acid (pyridine-2-ylmethyl)-amide

This compound was prepared in a similar manner to Example 184 using 2-isopropyl-6-oxo-5-(2-pyridin-4-yl)thiazol-4-yl)-1,6-dihydo-pyridine-3-carboxylic acid (Example 81) and pyridin-2-yl-methylamine to give the title product as an off white solid. MS (m/z, M+1): 432.4. Calc'd for C₂₃H₂₁N₅O₂S: 431.14.

### Example 192

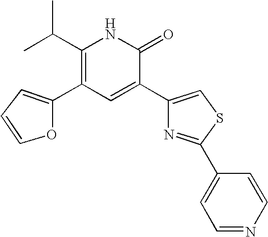

### 5-Furan-2-yl-6-isopropyl-3-(2-pyridin-4-ylthiazol-4-yl)-1H-pyridin-2-one

(a) 3-Acetyl-5-bromo-6-isopropyl-1H-pyridin-2-one. A mixture of 3-acetyl-6-isopropyl-1H-pyridin-2-one (1.28 g, 7.14 mmol) and NBS (1.53 g, 8.57 mmol) in CCl₄ (20 mL) was stirred at RT overnight. The mixture was concentrated, taken up in H₂O, extracted with EtOAc (3×), dried over MgSO₄, concentrated and purified with an ISCO silica gel flash chromatography instrument (30% EtOAc/Hexane) to give an off-white solid. MS (m/z, M+1): 258.4. Calc'd for C₁₀H₁₂BrNO₂: 257.01.

(b) 3-Acetyl-5-furan-2-yl-6-isopropyl-1H-pyridin-2-one. A mixture of 3-acetyl-5-bromo-6-isopropyl-1H-pyridin-2-one (step a, 0.30 g, 1.22 mmol), 2-furanylboronic acid (0.13 g, 1.59 mmol), (Ph₃P)₄Pd, and 2M Na₂CO₃ in toluene/EtOH (1:1, 6 mL) was heated at 150° C. for 20 min. using a microwave smithsynthesizer. The mixture was cooled and the layers were separated. The organic layer was dried over MgSO₄, purified with an ISCO silica gel flash chromatography instrument (30% EtOAc/Hexane) to give a light yellow solid. MS (m/z, M+1): 246.4. Calc'd for C₁₄H₁₅NO₃: 245.11.

(c) 3-(2-Bromo-acetyl)-5-furan-2-yl-6-isopropyl-1H-pyridin-2-one. A mixture of 3-acetyl-5-furan-2-yl-6-isopropyl-1H-pyridin-2-one (step b, 58 mg, 0.24 mmol), 5,5-dibromobarbituric acid (44 mg, 0.154 mmol) in THF (2 mL) was stirred at 70° C. for 36 h. The mixture was cooled, concentrated, taken up in H₂O, extracted with EtOAc (3×), dried over MgSO₄, concentrated to give a brown oil. MS (m/z, M+1): 324.4. Calc'd for C₁₄H₁₄BrNO₃.

(d) 5-Furan-2-yl-6-isopropyl-3-(2-pyridin-4-ylthiazol-4-yl)-1H-pyridin-2-one. A mixture of 3-(2-bromo-acetyl)-5-furan-2-yl-6-isopropyl-1H-pyridin-2-one (Step c, 60 mg, 0.19 mmol) and thioisonicotinamide (51 mg, 0.37 mmol) in EtOH (2 mL) was heated at 160° C. for 12 min using a microwave smithsynthesizer. The mixture was concentrated to give a residue, which was purified with an ISCO silica gel flash chromatography instrument (2% MeOH/CH₂Cl₂) to provide a light yellow solid. MS m/z 364.4). Calc'd for C₂₀H₁₇N₃O₂S: 363.10.

### Example 193

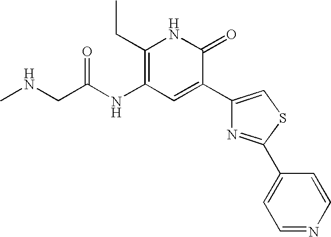

### N-[2-Ethyl-6-oxo-5-(2-pyridin-4-yl-thiazol-4-yl)-1,6-dihydro-pyridin-3-yl]-2-methylamino-acetamide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and (tert-butoxycarbonyl-methyl-amino)-acetic acid in the first step (under suitable standard amide bond forming conditions) followed by deprotection with 3-methoxybenzenethiol and TFA at 40° C. overnight to form an amorphous solid. MS m/z: 370.0 (M+1).

Calc'd for C₁₈H₁₉N₅O₂S: 369.13.

### Example 194

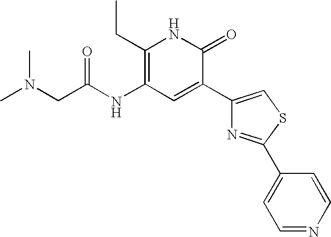

### 2-Dimethylamino-N-[2-ethyl-6-oxo-5-(2-pyridin-4-yl-thiazol-4-yl)-1,6-dihydro-pyridin-3-yl]-acetamide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and dimethylamino acetic acid in the first step (under standard amide bond forming conditions) followed by deprotection with 3-methoxybenzenethiol and TFA at 40° C. overnight to form an amorphous solid. MS m/z: 384.0 (M+1). Calc'd for C₁₉H₂₁NsO₂S: 383.14.

### Example 195

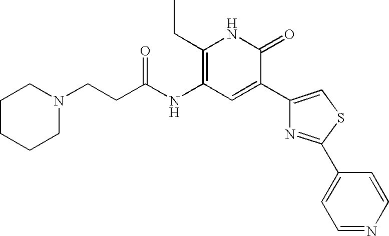

### N-[2-Ethyl-6-oxo-5-(2-pyridin-4-yl-thiazol-4-yl)-1,6-dihydro-pyridin-3-yl]-3-piperidin-1-yl-propionamide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and 3-piperidin-1-yl-propionic acid in the first step (under standard amide bond forming conditions) followed by deprotection with 3-methoxybenzenethiol and TFA at 40° C. overnight to yield an amorphous solid. MS m/z: 438.1 (M+1). Calc'd for C₂₃H₂₇N₅O₂S: 437.19.

### Example 196

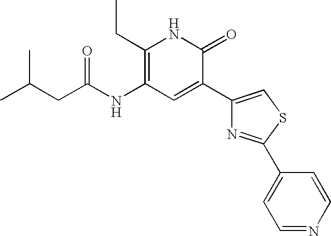

### N-[2-Ethyl-6-oxo-5-(2-pyridin-4-yl-thiazol-4-yl)-1,6-dihydro-pyridin-3-yl]-3-methyl-butyramide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and 3-methyl-butyric acid in the first step (under standard amide bond forming conditions) followed by deprotection with 3-methoxybenzenethiol and TFA at 40° C. overnight to yield an amorphous solid. MS m/z: 383.1 Calc'd for C₂₀H₂₂N₄O₂S: 382.15.

### Example 197

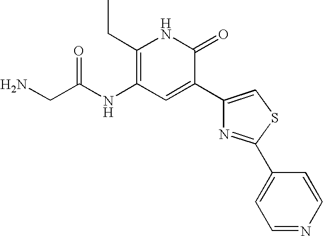

### 2-Amino-N-[2-ethyl-6-oxo-5-(2-pyridin-4-yl-thiazol-4-yl)-1,6-dihydro-pyridin-3-yl]-acetamide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and tert-butoxycarbonylglycine in the first step (under standard amide bond forming conditions) followed by deprotection with 3-methoxybenzenethiol and TFA at 40° C. overnight to form an amorphous solid. MS m/z: 356.2. Calc'd for C₁₇H₁₇N₅O₂S: 355.11.

### Example 198

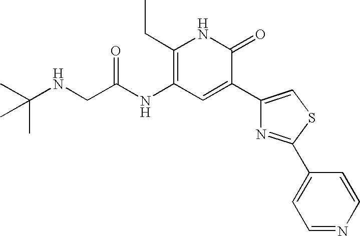

### 2-tert-Butylamino-N-[2-ethyl-6-oxo-5-(2-pyridin-4-yl-thiazol-4-yl)-1,6-dihydro-pyridin-3-yl]-acetamide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and tert-butylamino acetic acid (readily available from methyl bromoacetate and tert-butylamine via a amination and hydrolysis sequence) in the first step (under standard amide bond forming conditions) followed by deprotection with 3-methoxybenzenethiol and TFA at 40° C. overnight to form an amorphous solid. MS m/z: 412.1. Calc'd for C₂₁H₂₅N₅O₂S: 411.17.

### Example 199

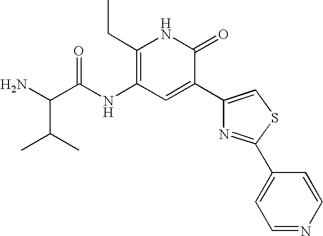

### 2-Amino-N-[2-ethyl-6-oxo-5-(2-pyridin-4-yl-thiazol-4-yl)-1,6-dihydro-pyridin-3-yl]-3-methyl-butyramide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and tert-butoxycarbonylvaline in the first step (under standard amide bond forming conditions) followed by deprotection with 3-methoxybenzenethiol and TFA at 40° C. overnight to yield an amorphous solid. MS m/z: 398.2. Calc'd for C₂₀H₂₃N₅O₂S: 397.16.

### Example 200

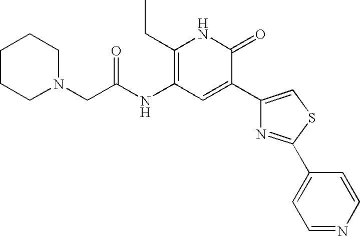

### N-[2-Ethyl-6-oxo-5-(2-pyridin-4-yl-thiazol-4-yl)-1,6-dihydro-pyridin-3-yl]-2-piperidin-1-yl-acetamide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and piperidin-1-yl-acetic acid (readily available from piperidin-1-yl-acetic acid ethyl ester via hydrolysis) in the first step (under suitable standard amide bond forming conditions) followed by deprotection with 3-methoxybenzenethiol and TFA at 40° C. overnight to provide an amorphous solid. MS m/z: 424.3. Calc'd for C₂₂H₂₅N₅O₂S: 423.17.

### Example 201

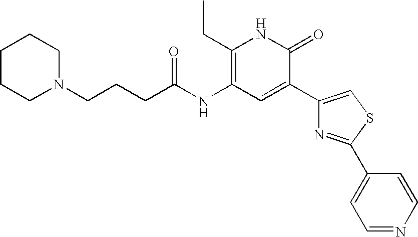

### N-[2-Ethyl-6-oxo-5-(2-pyridin-4-yl-thiazol-4-yl)-1,6-dihydro-pyridin-3-yl]-4-piperidin-1-yl-butyramide

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and 4-piperidin-1-yl-butyric acid (readily available from ethyl 4-bromobutyrate and piperidine via a amination and hydrolysis sequence) in the first step (under standard amide bond forming conditions) followed by deprotection with 3-methoxybenzenethiol and TFA at 40° C. overnight to yield an amorphous solid. MS m/z: 452.4. Calc'd for C₂₄H₂₉N₅O₂S: 451.20.

### Example 202

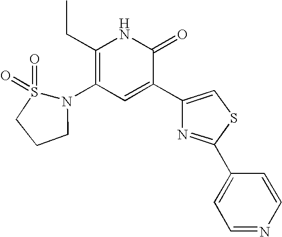

### 5-(1,1-dioxido-2-isothiazolidinyl)-6-ethyl-3-(2-(4-pyridinyl)-1,3-thiazol-4-yl)-2(1H)-pyridinone

This compound was prepared in a similar manner to that described in Example 139 using 5-amino-6-ethyl-1-(4-methoxybenzyl)-3-(2-pyridin-4-yl-thiazol-4-yl)-1H-pyridin-2-one (Example 139, Step a) and 3-chloro-propane-1-sulfonyl chloride in the first step followed by deprotection with 3-methoxybenzenethiol and TFA at 40° C. overnight to provide an amorphous solid. MS m/z: 403.2. Calc'd for C₁₈H₁₈N₄O₃S₂: 402.08.

The pharmacological properties of the compounds of this invention may be confirmed by a number of pharmacological assays. The exemplified pharmacological assays which follow have been carried out with the compounds according to the invention and their salts. The compounds of invention exhibited more than 10% CDK5/p25 or CDK2/cyclin inhibition at 10 μM.

### Biological Evaluation

Protocols for Cyclin E2/CDK2

Cloning of CDK2 and Cyclin 2/Generation of CDK2 and Cyclin 2 Recombinant Baculovirus

The following oligonucleotide primers flanking the coding sequence of the human CDK2 cDNA clone were used to amplify the gene and place EcoRI and HindIII restriction sites at the 5′ and 3′ ends of the gene respectively. [5′ oligo-5′-AAGCGCGCGGAATTCATAAATATGGAGAACTTCCAAAAGGTGGAA-3′ (SEQ ID NO: 1); 3′ oligo-5′-CTCGACAAGCTTATTAGAGTCGAAGATGGGGTAC-3′ (SEQ ID NO: 2)]

The following oligonucleotide primers flanking the coding sequence of the human CycE2 cDNA clone were used to amplify the gene and place XhoI and SphI restriction sites at the 5′ and 3′ ends of the gene respectively. A His tag was also placed at the N-terminus of the CycE2 protein. [5′ oligo-5′-CCCGGGATCTCGAGATAAATATGCATCATCATCATCATTCAAGACGAAGTAGCCGTTTACAA-3′ (SEQ ID NO: 3); 3′ oligo-5′-CCCGGTACCGCATGCTTAGTGTTTTCCTGGTGGTTTTTC-3′ (SEQ ID NO: 4)]

CycE-2 and CDK2 PCR fragments were subcloned into the vector pFastBacDual (Gibco/LifeTechnologies) using the restriction sites indicated above. Recombinant virus was made following protocols supplied by the manufacturer.

Expression of Cyclin 2/CDK2 in Insect Cells

Hi5 cells were grown to a cell density of 1×10⁶ cells per mL in 800 mL of Excell 405 media (JRH). Cells were infected with virus at a multiplicity of 1. Infected cultures were incubated with shaking at 28° C. Cells were harvested by centrifugation.

Cloning of CDK5 and p25/Generation of CDK5 and p25 Recombinant Baculovirus

Based on the reported sequences of human CDK5 and p35, GenBank accession numbers X66364 and X80343 respectively, oligonucleotide primers flanking the coding sequence of each gene were used to amplify CDK5 (5′-GCGATGCAGAAATACGAGAAACT-3′ (SEQ ID NO: 5); 5′-CCCCACTGTCTCACCCTCTCAA-3′ (SEQ ID NO: 6)) and p35 (5′-CGGTGAGCGGTTTTATCCC-TCC-3′ (SEQ ID NO: 7); 5′-GCATTGAATCCTTGAGCCATGACG-3′ (SEQ ID NO: 8)) from a human fetal brain cDNA library (Clontech). p25, a C-terminal proteolytic fragment corresponding to amino acids 99-307 of full-length p35 (Lew et. al), was PCR subcloned from the p35 sequence using oligonucleotide primers (5′-CGGGATCCATGGCCCAGCCCCCACCGGCCCA-3′ (SEQ ID NO: 9); 5′-CCAAGCTTTCACCGATCCAGGCCTAG-3′ (SEQ ID NO: 10)). The p25 PCR product (629 bp) was cloned into the pFastBacHTb baculovirus expression vector (Gibco BRL) using BamHI and HindIII. CDK5 was PCR subcloned using oligonucleotide primers (5′-CGGGATCC-GCCACCATGCAGAAATACGAGAAACTGG-3′ (SEQ ID NO: 11); 5′-GGACTAGTCTAGGGCGGAC-AGAAGTCG-3′ (SEQ ID NO: 12)). The CDK5 PCR product (879 bp) was cloned into the pFastBacl baculovirus expression vector (Gibco BRL) using BamHI and SpeI. Recombinant baculovirus expressing human CDK5 and N-terminally six histidine tagged p25 were generated using the Bac-to-Bac system (Gibco BRL).

Expression of P25/CDK5 in Insect Cells

Coinfections of Hi5 cells by recombinant baculovirus containing the P25 gene and another containing the CDK5 gene were done at a multiplicity of infection of 5 (each virus). The Hi5 cultures were set to a cell concentration of 1×10⁶ cells per ml in 800 mL of Excell media by JRH. The cultures were grown in 2.6 L fernbach flasks with shaking (110 rpm) at 27° C. for 60 h. The cells were harvested by centrifugation.

Purification of Complexes

All steps were performed at 4° C. Insect cells expressing either cyclin E2/CDK2 or p25/CDK5 were lysed using a microfluidizer (Microfluidics Corporation.) The lysis buffer contained 10 mM Hepes, 150 mM NaCl, 20 mM MgCl₂, 20 mm imidazole, 0.5 mM EDTA, 10% glycerol, 25 μg/mL Aprotinin, 25 μg/ml Leupeptin, 1 mM Pefabloc, pH 7.5). Total protein was determined on the resulting lysate using the Bradford method with a BSA standard curve. Protamine sulfate was added to the lysate to give a final 30:1 protein:protamine sulfate, incubated for 15-20 min and centrifuged at 14000×g for 30 min to remove insoluble material. Ni-NTA superflow resin (Qiagen Inc) was equilibrated in lysis buffer and incubated with the centrifugation supernatant for 1 h while rotating. The slurry was packed in a glass column and washed until a stable UV baseline was reached. Proteins were eluted with a linear gradient of 20-300 mM imidazole over 15 column volumes. Fractions were analyzed by SDS-PAGE and Western blot. Appropriate fractions were pooled, total protein determined, and submitted for kinase assay.

CDK2 Kinase Assay

CDK2 kinase assays were carried out with inhibitor (dissolved in DMSO) in a total volume of 50 μL with 1 nM enzyme (His-tagged cyclin 2/CDK2), 1 μM Histone-H1 (Gibco), 25 μM ATP, 20 μCi/mL ³³P-ATP (Amersham; 2500 Ci/mmol) in kinase buffer (50 mM Tris-HCl₁, pH 7.5, 5 mM MgCl₂, 1 mM EGTA, 5 mM DTT, 200 μg/mL BSA and 20 mM β-glycerophosphate for 60 min at 25° C. Reactions were stopped by the addition of an equal volume of 30% trichloroacetic acid (Sigma). Precipitates were formed by incubation at 4° C. for 60 min then collected by filtration on Millipore® filter plates (MAFC NOB10). MicroScint-20 (40 μL, Packard) was added, and counted on a Packard TopCount®. Raw cpms were analyzed with a four-parameter logistic fit using the Levenburg Marquardt algorithm (Xlfit software IDBS LTD). Kinetic parameters were calculated by non-linear regression analysis using Grafit (Erithacus Software LTD). Riscovitine (BIOMOL Research Labs Inc., Plymouth Meeting, Pa.) and staurosporin (Sigma, St. Louis Mo.) were used as standards.

CDK5 Kinase Assay

CDK5 kinase assays were carried out with inhibitor (dissolved in DMSO) in a total volume of 50 μL with 1 nM enzyme (His-tagged p25/CDK5), 1 μM Histone-H1 (Gibco), 25 μM ATP, 20 μCi/mL ³³P-ATP (Amersham; 2500 Ci/mmol) in kinase buffer (50 mM Tris-HCl, pH 7.5, 5 mM MgCl₂, 1 mM EGTA, 5 mM DTT, 200 μg/mL BSA and 20 mM β-glycerophosphate) for 60 min at 25° C. Reactions were stopped by the addition of an equal volume of 30% trichloroacetic acid (Sigma). Precipitates were formed by incubation at 4° C. for 60 min then collected by filtration on Millipore® filter plates (MAFC NOB10). MicroScint-20 (40 μL, Packard) was added, and counted on a Packard TopCount®. Raw cpms were analyzed with a four-parameter logistic fit using the Levenburg Marquardt algorithm (Xlfit software IDBS LTD). Kinetic parameters were calculated by non-linear regression analysis using Grafit (Erithacus Software LTD). Riscovitine (BIOMOL Research Labs Inc., Plymouth Meeting, Pa.) and staurosporine (Sigma) were used as standards.

Examples 1-3, 10-17, 24-26, 28-29, 40, 42, 46-48, 50, 52-54, 56-58, 60-62, 65, 67, 75-78, 80, 82-83, 88, 90, 94-95, and 99-103 exhibited CDK2/cyclin kinase activity with IC₅₀ values less than 0.5 μM. The compounds of examples 1-3, 5, 7-8, 10-19, 24-29, 37, 40, 46-48, 50, 52-54, 56-58, 60-63, 65, 67, 72, 74-80, 82-83, 84, 89-90, 94-95, and 99-104 exhibited CDK5/p25 kinase activity with IC₅₀ values less than 0.5 μM.

### Cell Proliferation Assay

Cell proliferation was measured using a colorimetric immunoassay (B/M Roche #164 7229), based on the measurement of pyrimidine analog BrdU incorporation during DNA synthesis in proliferating cells. Cells, e.g., human PC-3 prostate carconima cells, huFSF normal human foreskin fibroblast cells, HCT 116 human colon carcinoma cells or HT 29 human colon carcinoma cells, were cultured in a 96-well plate for 24 h, until a cell count of 3×10³ to 6×10³ cells per well in duplicate wells were achieved, in a well volume of 200 μL. The media was changed and 1 μL of 200× control inhibitors or compounds was added to each well. Cells are incubated for 48 h at 37° C. The cells were labeled with BrdU for 4 h at 37° C. The labeling medium was removed and in one step, the cells were fixed and the DNA was denatured (30 min at RT). Anti-BrdU-POD antibody was added to bind to the BrdU incorporated in newly synthesized cellular DNA (60-90 min at RT). The cells were washed 3× with washing buffer, substrate (100 μL) was added and the cells were incubated for 10 min at RT. The substrate reaction was stopped by adding 25 μL of 1M H₂SO₄. The amount of BrdU incorporated was quantified by measuring the absorbance at 450 nm using ELISA reader. IC₅₀'s were calculated using GraFit (Sigma). The compounds of examples 1-3, 12, 24, 47 and 50 inhibited proliferation with IC₅₀ values less than 1.0 μM.

### Ischemic Stroke Model: Middle Cerebral Artery Occlusion (MCAO) In Vivo

The compounds' effect on treating stroke was measured in a MCAO rat model. (L. Belayev et al., Stroke, 27:1616-23 (1996). Male Sprague-Dawley rats (300-330 g body weight) were anesthetized with halothane and MCAO was induced by inserting a poly-L-lysine coated monofilament suture to the beginning of the middle cerebral artery (MCA). After various time points (60, 90 or 120 min), the intraluminal suture was carefully removed to start reperfusion. Physiological conditions (blood O₂, CO₂, pH, glucose, blood pressure) were monitored and kept stable during the surgery. The compound was dissolved in 20% Captisol in phosphate buffered saline and administered (orally, IV or IP) 90 min after ischemia onset, at the beginning of reperfusion. Further dosing occurred at 4-8 h and twice a day thereafter.

The use of behavioral tests was directly analogous to the clinical neurological examination for assessing ischemic deficits and rates of behavioral recovery. The battery consisted of four tests: (1) postural reflex test, (2) forelimb placing test (J B Bederson et al., Stroke, 17:472-476 (1986) (L. Belayev et al., Stroke, 26:2313-2320 (1995), (3) contralateral foot fault index (A. Tamura et al., J. Cereb Blood Flow Metab., 1:53-60 (1981) (D. M. Freeney, Science, 217:855-857 (1982), and (4) cylinder asymmetry (T. A. Jones and T. Schallert, J. Neurosci., 14:2140-2152 (1994). Tests were performed once a day for three days and then once a week for a period of 30 days. These tests are useful in assessing neurological deficits for short-term studies; the cylinder asymmetry test appeared to be the most useful for long-term experiments.

At the end of the experiment, the infarct volume was measured (J. B. Bederson et al., Stroke, 17:1304-1308 (1986) (K. A. Osborne et al, J. Neurol Neurosurg. Psychiatry, 50:402 (1987) (R. A. Swanson et al., J. Cereb. Blood Flow Metab., 10:290-293 (1990). The brains were removed and sliced coronally at 1 mm thickness. The brain slices were stained with 2% (w/vol) 2,3,5-triphenyltetrazolium chloride (TTC) which stains the infarcted areas of the brain in white and allows for the measurement of infarct volume by an image-analysis system. Edema volume that contributes to infarct volume was subtracted by comparison with the total volume of the contralateral hemisphere.

Formulations

Also embraced within this invention is a class of pharmaceutical compositions comprising the active compounds of Formula I-III in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg, preferably from about 1 to 500 mg, more preferably from about 5 to 150 mg. A suitable daily dose for a human or other mammal may vary widely depending on the condition of the patient and other factors, but, once again, can be determined using routine methods.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A daily dose of about 0.01 to 500 mg/kg body weight, preferably between about 0.5 and about 50 mg/kg body weight and most preferably between about 0.1 to 20 mg/kg body weight, may be appropriate. The daily dose can be administered in one to four doses per day.

For therapeutic purposes, the active compounds of this invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

In the case of psoriasis and other skin conditions, it may be preferable to apply a topical preparation of compounds of this invention to the affected area two to four times a day.

Formulations suitable for topical administration include liquid or semi-liquid preparations suitable for penetration through the skin (e.g., liniments, lotions, ointments, creams, or pastes) and drops suitable for administration to the eye, ear, or nose. A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

When formulated in an ointment, the active ingredients may be employed with either paraffinic or a water-miscible ointment base. Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs.

The compounds of this invention can also be administered by a transdermal device. Preferably transdermal administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The active ingredients are preferably present in such formulations in a concentration of 0.5 to 20%, advantageously 0.5 to 10% and particularly about 1.5% w/w.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (ie. Captisol), cosolvent solubilization (ie. propylene glycol) or micellar solubilization (ie. tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

For pulmonary administration, the pharmaceutical composition may be administered in the form of an aerosol or with an inhaler including dry powder aerosol.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

No unacceptable toxological effects are expected when compounds of the present invention are administered in accordance with the present invention.

All mentioned references, patents, applications and publications, are hereby incorporated by reference in their entirety, as if here written.

What is claimed is:

1. A compound of Formula I 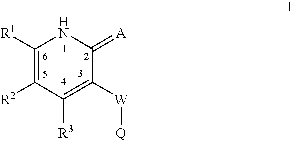
wherein A is O or S;
wherein Q is selected from —N(R⁵)₂, —NR⁵C(O)R⁵, —(C₁-C₈)alkyl-OR⁵, —(C₁-C₈)alkyl-S(O)ₙR⁶, 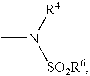
substituted aryl, an unsubstituted or substituted monocyclic or bicyclic, non-aromatic carbocyclic ring, an unsubstituted or substituted monocyclic or bicyclic, heteroaryl ring, and an unsubstituted or substituted monocyclic or bicyclic, non-aromatic heterocyclic ring,
wherein a ring is unsubstituted or substituted with one or more groups selected from halo, (C₁-C₈)alkyl, (C₂-C₈)alkynyl, (C₂-C₈)alkenyl, —OR⁵, —O—(CH₂)₁₋₂—O—, —N(R⁵)₂, —(C₁-C₈)alkyl-N(R⁵) 2, (C₁-C₈)haloalkyl, lower cyanoalkyl, —(C₁-C₈)alkyl-OR⁵, lower alkylaminoalkoxy, lower aminoalkoxyalkyl, —(C₁-C₈)alkyl-S(O)ₙR⁵, —N(R⁵)—(C₁-C₈)alkyl-N(R⁵)₂, —N(R⁵)—(C₁-C₈)alkyl-OR⁵, —N(R⁵)—(C₁-C₈)alkyl-NHC(O)R⁵, —N(R⁵)—(C₁-C₈)alkyl-C(O)N(R⁵)₂, lower alkoxyalkyl, —S(O)ₙR⁵, —SO₂NR⁵R⁵, —NR⁵S(O)ₙR⁵, cyano, nitro, optionally substituted (C₃-C₁₀)cycloalkyl, optionally substituted aryl, optionally substituted 4-7 membered heterocyclyl, optionally substituted phenoxyalkyl, optionally substituted heterocyclyloxyalkyl, —C(O)N(R⁵)₂, —CO₂R⁵, —CO₂N(R⁵)₂, —SO₂NHC(O)R⁵, optionally substituted phenylalkyl, optionally substituted heterocyclylalkyl, —NR⁵C(O)N(R⁵)₂, —NR⁵C(O)R⁵, —NR⁵CO₂R⁵ and —C(O)R⁵;
wherein W is selected from 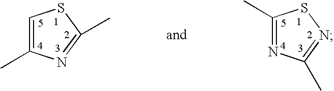
wherein n is 0, 1 or 2;
wherein R¹ is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵) 2, —CO₂R⁵, —(CR⁵₂)₁₋₈aryl, —(CR⁵₂)₁₋₈heterocyclyl, —NR⁵C(O)N(R⁵)₂, —NR⁵C(O)R⁵, —NR⁵CO₂R⁵, and —C(O)R⁵; wherein R¹ and R² may be joined to form a 5-10 membered saturated or partially unsaturated carbocyclic or heterocyclic ring;
wherein R² is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵)₂, —CO₂R⁵, —(CR⁵₂)₁₋₈aryl, —(CR⁵₂)₁₋₈heterocyclyl, NR⁵C(O)N(R⁵)₂, —NR⁵C(O)R⁵, —NR⁵CO₂R⁵, and —C(O)R⁵;
wherein R³ is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵)₂, —CO₂R⁵, —(CR⁵₂)₁₋₈aryl, —(CR⁵₂)₁₋₈heterocyclyl, NR⁵C(O)N(R⁵)₂, —NR⁵C(O)R⁵, —NR⁵CO₂R⁵, and —C(O)R⁵; wherein R² and R³ may be joined to form a 5-10 membered saturated or partially unsaturated carbocyclic or heterocyclic ring;
wherein R⁴ is independently selected from H, and (C₁-C₆)alkyl;
wherein R⁵ is independently selected from H, lower alkyl, optionally substituted aryl, optionally substituted aralkyl, optionally substituted heterocyclyl, optionally substituted heterocyclylalkyl, optionally substituted C₃-C₆ cycloalkyl, optionally substituted C₃-C₆ cycloalkyl-alkyl, lower alkylamino-lower alkyl, aryloxyalkyl, alkylcarbonylalkyl, and lower perfluoroalkyl; and
wherein R⁶ is independently selected from lower alkyl, optionally substituted aryl, optionally substituted aralkyl, optionally substituted heterocyclyl, optionally substituted heterocyclylalkyl, optionally substituted C₃-C₆ cycloalkyl, optionally substituted C₃-C₆ cycloalkyl-alkyl, lower alkylamino-lower alkyl, aryloxyalkyl, alkylcarbonylalkyl, and lower perfluoroalkyl;
wherein each aryl, heteroaryl, cycloalkyl, and heterocyclyl moiety of any R¹, R², R³, R⁵, R⁶, and Q is optionally substituted with one or more groups selected from halo, —NH₂, —OH, —CO₂H, (C₁-C₆)alkylamino, (C₁-C₆)alkoxy, (C₁-C₆)alkoxyalkyl, (C₁-C₆)alkyl, di(C₁-C₆)alkylamino, phenyl, and heterocyclyl;
and pharmaceutically acceptable derivatives thereof;
provided R¹ is not CF₃ when R² is ethoxycarbonyl, when R³ is H, when W is thiazol-4-yl and when Q is 4-pyridyl or 2-chloro-4-pyridyl; further provided Q is not 4-pyridyl, when W is thiazol-2-yl, when R¹, R³, and R² are H; further provided Q is not 2-nitro-5-furyl when W is thiazol-2-yl, when R¹ is methyl, when R³ is H, and when R² is H; further provided Q is not phenyl when W is thiazol-2-yl, when R¹ is methyl, when R³ is methyl, and when R² is H; further provided Q is not phenyl, 3,4-diacetylphenyl or 3,4-dihydroxyphenyl, when W is thiazol-2-yl, when R¹ is H, when R³ is H, and when R² is H; and further provided Q is not 3-cyano-6-methyl-2-oxo-1,2-dihydro-5-pyridyl, when W is thiazol-2-yl, when R¹ is methyl, when R³ is H, and when R² is acetyl.

2. Compound of claim 1 wherein Q is selected from R⁶SO₂—(C₁-C₆)alkyl-, 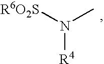
substituted phenyl, and substituted or unsubstituted 5-6 membered heteroaryl;
wherein R⁴ is independently selected from H, and (C₁-C₂)alkyl; and
wherein R⁶ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl- and optionally substituted heterocyclyl selected from pyridyl and thienyl; and pharmaceutically acceptable derivatives thereof.

3. Compound of claim 2 wherein Q is selected from phenylsulfonylamino, N-methyl-N-(2-pyridylsulfonyl)amino, N-methyl-N-(3-pyridylsulfonyl)amino, N-methyl-N-(4-pyridylsulfonyl)amino, N-methyl-N-(2-thienylsulfonyl)amino, N-methyl-N-(phenylsulfonyl)amino, 2-pyridylsulfonylmethyl, 3-pyridylsulfonylmethyl, 4-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, 3-trifluoromethylbenzyl-sulfonylmethyl, methylsulfonylmethyl, tert-butyl-sulfonylmethyl, 4-fluorobenzylsulfonylmethyl, 4-chlorophenyl-methylsulfonylmethyl, 2-thienyl, 3-(4-chlorophenylsulfonylmethyl)-2-thienyl, phenyl substituted
with one or more substituents selected from hydroxyl, chloro, fluoro, methoxy, —O—CH₂—O—, amino, aminomethyl, methylsulfonyl, methyl, cyano, trifluoromethyl, and pyrrolyl,
unsubstituted pyridyl, and
4-pyridyl substituted with one or more substituents selected from chloro, fluoro, methyl, ethyl, —NH₂, methoxy, ethoxy, —OH, —CO₂H, phenoxyethylamino, methylamino, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl; and
pharmaceutically acceptable derivatives thereof.

4. Compound of claim 1, and pharmaceutically acceptable derivatives thereof, wherein W is thiazol-4-yl.

5. Compound of claim 1 wherein R¹ is selected from (C₁-C₆)alkyl, —(C₁-C₄)alkyl-N(R⁵)₂, —(C₁-C₄)alkyl-OR⁵, —(C₃-C₅)cycloalkyl, and —CF₃;
wherein R² is selected from H, halo, (C₁-C₃)alkyl, —NR⁵₂, —OR⁶, —(C₁-C₃)alkyl-OR⁵, —C(O)N(R⁵)₂, —CO₂R⁵, —(CH₂)₁₋₃-(5-6 membered saturated or partially unsaturated) heterocyclyl, —NHC(O)R⁵, and —C(O)R⁵;
wherein R¹ and R² may be joined together with the pyridone ring to form optionally substituted 2-oxo-1,5,7,8-tetrahydro-2H-[1,6]naphthyridine, optionally substituted 5,6,7,8-tetrahydro-1H-[1,6]naphthyridin-2-one, optionally substituted 5,6,7,8-tetrahydro-1H-[1,7]naphthyridin-2-one, optionally substituted 5,6,7,8-tetrahydro-1H-quinolin-2-one, optionally substituted 7,8-dihydro-1H-quinolin-2-one, 7,8-dihydro-(1H,6H)-quinoline-2,5-dione or 1,5,7,8-tetrahydro-pyrano[4,3-b]pyridin-2-one;
wherein R³ is H;
wherein R⁵ is independently selected from H, C₁-C₄-alkyl, optionally substituted phenyl, optionally substituted benzyl, optionally substituted heterocyclyl selected from piperazinyl, morpholinyl, pyrrolidinyl, and piperidinyl, optionally substituted pyridyl-(C₁-C₃)-alkyl, optionally substituted piperazinyl-(C₁-C₃)-alkyl, 4-morpholinyl-(C₁-C₃)-alkyl, pyrrolidinyl-(C₁-C₃)-alkyl, 1-piperidinyl-(C₁-C₃)-alkyl, optionally substituted C₃-C₆ cycloalkyl-(C₁-C₃)-alkyl, —(C₁-C₃)-alkyl-N-((C₁-C₃)-alkyl) 2 and —(C₁-C₃)-alkyl-NH—(C₁-C₃)-alkyl;
and pharmaceutically acceptable derivatives thereof.

6. Compound of claim 5 wherein R¹ is selected from methyl, ethyl, propyl, isopropyl, hydroxyethyl, dimethylaminomethyl, benzyloxymethyl, 4-methoxy-benzyloxymethyl, methoxymethyl, cyclopropyl, and —CF₃;
wherein R² is selected from H, bromo, methyl, amino, isobutylamino, hydroxymethyl, aminocarbonyl, 4-methoxybenzylaminocarbonyl, 2-pyridylmethylaminocarbonyl, ethylaminoethylaminocarbonyl, isopropylaminoethylaminocarbonyl, cyclopropylmethylaminocarbonyl, isobutylaminocarbonyl, ethoxycarbonyl, tert-butoxycarbonyl, 4-morpholinylethoxycarbonyl, 1-pyrrolidinylethoxycarbonyl, 1-piperidinylethoxycarbonyl, diethylaminopropoxycarbonyl, carboxyl, 1,2,5,6-tetrahydro-1-pyridylmethyl, 1-piperidinylmethyl, 1-methyl-4-piperazinylmethyl, methylcarbonylamino, isobutylcarbonylamino, and 1-methyl-4-piperazinylcarbonyl;
wherein R¹ and R² may be joined together with the pyridone ring to form 6-benzyloxycarbonyl-2-oxo-1,5,7,8-tetrahydro-2H-[1,6]naphthyridine, 5,6,7,8-tetrahydro-1H-[1,6]naphthyridin-2-one, 7-Boc-5,6,7,8-tetrahydro-1H-[1,7]naphthyridin-2-one, 7-ethyl-5,6,7,8-tetrahydro-1H-[1,7]naphthyridin-2-one, 5-methyl-7,8-dihydro-1H-quinolin-2-one, 5-propylamino-5,6,7,8-tetrahydro-1H-quinolin-2-one, 5-propylimino-5,6,7,8-tetrahydro-1H-quinolin-2-one, 7,8-dihydro-(1H,6H)-quinoline-2,5-dione or 1,5,7,8-tetrahydro-pyrano[4,3-b]pyridin-2-one; and pharmaceutically acceptable derivatives thereof.

7. Compound of claim 4, and pharmaceutically acceptable derivatives thereof, wherein A is O; wherein Q is selected from N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl, phenyl substituted
with one or more substituents selected from chloro, fluoro, and —O—CH₂—O—, unsubstituted pyridyl, and
4-pyridyl substituted with one or more substituents selected from chloro, fluoro, —NH₂, methoxy, ethoxy, phenoxyethylamino, methylamino, methyl, ethyl, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl;
wherein R¹ is selected from methyl, ethyl, propyl, isopropyl, dimethylaminomethyl, hydroxyethyl, benzyloxymethyl, 4-methoxy-benzyloxymethyl, methoxymethyl, cyclopropyl, and —CF₃;
wherein R² is selected from H, bromo, methyl, amino, isobutylamino, hydroxymethyl, aminocarbonyl, 4-methoxybenzylaminocarbonyl, 2-pyridylmethylaminocarbonyl, ethylaminoethylaminocarbonyl, isopropylaminoethylaminocarbonyl, cyclopropylmethylaminocarbonyl, isobutylaminocarbonyl, ethoxycarbonyl, tert-butoxycarbonyl, 4-morpholinylethoxycarbonyl, 1-pyrrolidinylethoxycarbonyl, 1-piperidinylethoxycarbonyl, diethylaminopropoxycarbonyl, carboxyl, 1,2,5,6-tetrahydro-1-pyridylmethyl, 1-piperidinylmethyl, 1-methyl-4-piperazinylmethyl, methylcarbonylamino, isobutylcarbonylamino, and 1-methyl-4-piperazinylcarbonyl;
wherein R¹ and R² may be joined together with the pyridone ring to form 6-benzyloxycarbonyl-2-oxo-1,5,7,8-tetrahydro-2H-[1,6]naphthyridine, 5,6,7,8-tetrahydro-1H-[1,6]naphthyridin-2-one, 7-Boc-5,6,7,8-tetrahydro-1H-[1,7]naphthyridin-2-one, 7-ethyl-5,6,7,8-tetrahydro-1H-[1,7]naphthyridin-2-one, 5-methyl-7,8-dihydro-1H-quinolin-2-one, 5-propylamino-5,6,7,8-tetrahydro-1H-quinolin-2-one, 5-propylimino-5,6,7,8-tetrahydro-1H-quinolin-2-one, 7,8-dihydro-(1H,6H)-quinoline-2,5-dione or 1,5,7,8-tetrahydro-pyrano[4,3-b]pyridin-2-one; and
wherein R³ is H.

8. Compound of claim 1 wherein A is O; and
pharmaceutically acceptable derivatives thereof.

9. A compound of claim 1 having Formula II 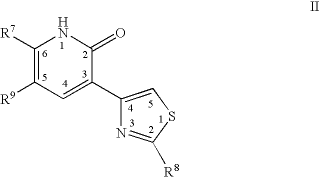
wherein R⁷ is selected from —(C₁-C₃)alkyl, —(C₁-C₃)alkyl-N(R¹⁰)₂, —(C₁-C₃)alkyl-OR¹⁰, —(C₃-C₅)cycloalkyl, and —CF₃;
wherein R⁸ is selected from R¹⁰SO₂—(C₁-C₆)alkyl-, R¹¹SO₂NH— 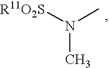
substituted phenyl, and substituted or unsubstituted 5-6 membered heteroaryl;
wherein R⁹ is selected from H, halo, (C₁-C₃)alkyl, —NR¹⁰₂, —(C₁-C₃)alkyl-OR¹⁰, —C(O)N(R¹⁰)₂, —CO₂R¹⁰, (CH₂)₁₋₃-(5-6 membered saturated or partially unsaturated heterocyclyl, —NHC(O)R¹⁰, and —C(O)R¹⁰;
wherein R¹⁰ is independently selected from H, (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl- and optionally substituted heterocyclyl selected from pyridyl and thienyl; and
wherein R¹¹ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl, and optionally substituted heterocyclyl selected from pyridyl and thienyl;
and pharmaceutically acceptable derivatives thereof;
provided R⁷ is not CF₃ when R⁹ is ethoxycarbonyl and when R⁸ is 4-pyridyl or 2-chloro-4-pyridyl.

10. Compound of claim 9 wherein R⁷ is selected from methyl, ethyl, propyl, isopropyl, dimethylaminomethyl, benzyloxymethyl, hydroxyethyl, 4-methoxy-benzyloxymethyl, methoxymethyl, cyclopropyl, and —CF₃; wherein R⁸ is selected from N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl, phenyl substituted
with one or more substituents selected from chloro, fluoro, and —O—CH₂—O—, unsubstituted pyridyl, and
4-pyridyl substituted with one or more substituents selected from chloro, fluoro, —NH₂, methoxy, ethoxy, phenoxyethylamino, methylamino, methyl, ethyl, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl; and
wherein R⁹ is selected from H, bromo, methyl, amino, isobutylamino, hydroxymethyl, aminocarbonyl, 4-methoxybenzylaminocarbonyl, 2-pyridylmethylaminocarbonyl, ethylaminoethylaminocarbonyl, isopropylaminoethylaminocarbonyl, cyclopropylmethylaminocarbonyl, isobutylaminocarbonyl, ethoxycarbonyl, tert-butoxycarbonyl, 4-morpholinylethoxycarbonyl, 1-pyrrolidinylethoxycarbonyl, 1-piperidinylethoxycarbonyl, diethylaminopropoxycarbonyl, carboxyl, 1,2,5,6-tetrahydro-1-pyridylmethyl, 1-piperidinylmethyl, 1-methyl-4-piperazinylmethyl, methylcarbonylamino, isobutylcarbonylamino, and 1-methyl-4-piperazinylcarbonyl; and pharmaceutically acceptable derivatives thereof.

11. A compound of claim 1 having Formula III 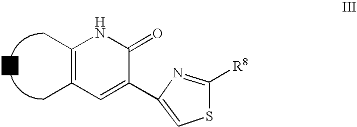
wherein R⁸ is selected from R¹¹SO₂—(C₁-C₆)alkyl-, R¹¹SO₂NH— 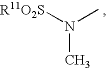
substituted phenyl, and substituted or unsubstituted 5-6 membered heteroaryl;
wherein ring A together with the pyridone ring forms optionally substituted 2-oxo-1,5,7,8-tetrahydro-2H-[1,6]naphthyridine, optionally substituted 5,6,7,8-tetrahydro-1H-[1,6]naphthyridin-2-one, optionally substituted 5,6,7,8-tetrahydro-1H-quinolin-2-one, optionally substituted 5,6,7,8-tetrahydro-1H-[1,7]naphthyridin-2-one, or 1,5,7,8-tetrahydro-pyrano[4,3-b]pyridin-2-one; and
wherein R¹¹ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl, (C₁-C₂)alkylcarbonyl-(C₁-C₂)alkyl, and optionally substituted heterocyclyl selected from pyridyl and thienyl;
and pharmaceutically acceptable derivatives thereof.

12. Compound of claim 11 wherein R⁸ is selected from N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl,
phenyl substituted with one or more substituents selected from chloro, fluoro, and —O—CH₂—O—,
unsubstituted pyridyl, and
4-pyridyl substituted with one or more substituents selected from chloro, fluoro, —NH₂, methoxy, ethoxy, phenoxyethylamino, methylamino, methyl, ethyl, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl;
and pharmaceutically acceptable derivatives thereof.

13. Compound of claim 12 and pharmaceutically acceptable derivatives thereof selected from:
Phenylmethyl 2-oxo-3-(2-(4-pyridyl)(1,3-thiazol-4-yl))-1,5,6,7,8-pentahydropyridino[3,2-c]pyridine-6-carboxylate;
3-(2-(4-Pyridyl)-1,3-thiazol-4-yl)-1,7,8-trihydro-5H-pyrano[4,3-b]pyridin-2-one;
7-Ethyl-3-(2-(4-pyridyl)(1,3-thiazol-4-yl))-1,5,6,7,8-pentahydropyridino[3,2-c]pyridin-2-one;
tert-Butyl 2-oxo-3-(2-(4-pyridyl)(1,3-thiazol-4-yl))-1,5,6,7,8-pentahydropyridino[3,2-c]pyridine-6-carboxylate;
3-(2-(4-Pyridyl)-1,3-thiazol-4-yl)-1,5,6,7,8-pentahydropyridino[3,2-c]pyridin-2-one, dihydrochloride; and
6-Methyl-3-(2-pyridin-4-yl-thiazol-4-yl)-5,6,7,8-tetrahydro-1H-[1,6]naphthyridin-2-one.

14. A compound of Formula I′ 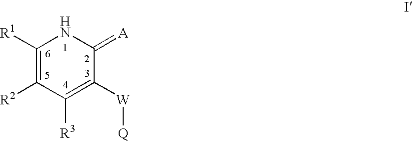
wherein A is O or S;
wherein Q is selected from —N(R⁵)₂, —NR⁵C(O)R⁵, —(C₁-C₈)alkyl-OR⁵, —(C₁-C₈)alkyl-S(O)ₙR⁶, 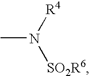
substituted aryl, an unsubstituted or substituted monocyclic or bicyclic, non-aromatic carbocyclic ring, an unsubstituted or substituted monocyclic or bicyclic, heteroaryl ring, and an unsubstituted or substituted monocyclic or bicyclic, non-aromatic heterocyclic ring,
wherein a ring is unsubstituted or substituted with one or more groups selected from halo, (C₁-C₈)alkyl, (C₂-C₈)alkynyl, (C₂-C₈)alkenyl, —OR⁵, —O—(CH₂)₁₂—O—, —N(R⁵)₂, —(C₁-C₈)alkyl-N(R⁵)₂, (C₁-C₈)haloalkyl, lower cyanoalkyl, —(C₁-C₈)alkyl-OR⁵, lower alkylaminoalkoxy, lower aminoalkoxyalkyl, —(C₁-C₈)alkyl-S(O)ₙR⁵, —N(R⁵)—(C₁-C₈)alkyl-N(R⁵)₂, —N(R⁵)—(C₁-C₈)alkyl-OR⁵, —N(R⁵)—(C₁-C₈)alkyl-NHC(O)R⁵, —N(R⁵)—(C₁-C₈)alkyl-C(O)N(R⁵)₂, lower alkoxyalkyl, —S(O)ₙR⁵, —SO₂NR⁵R⁵, —NR⁵S(O)ₙR⁵ cyano, nitro, optionally substituted (C₃-C₁₀)cycloalkyl, optionally substituted aryl, optionally substituted 4-7 membered heterocyclyl, optionally substituted phenoxyalkyl, optionally substituted heterocyclyloxyalkyl, —C(O)N(R⁵)₂, —CO₂R⁵, —CO₂N(R⁵)₂, —SO₂NHC(O)R⁵, optionally substituted phenylalkyl, optionally substituted heterocyclylalkyl, —NR⁵C(O)N(R⁵)₂, —NR⁵C(O)R⁵, —NR⁵CO₂R⁵ and —C(O)R⁵;
wherein W is selected from 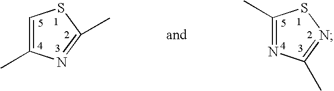
wherein n is 0, 1 or 2;
wherein R¹ is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵)₂, —CO₂R⁵, —(CR⁵₂)₁₋₈aryl, —(CR⁵₂)₁₋₈heterocyclyl, —NR⁵C(O)N(R⁵)₂, —NR⁵C(O)R⁵, —NR⁵CO₂R⁵, and —C(O)R⁵; wherein R¹ and R² may be joined to form a 5-10 membered saturated or partially unsaturated carbocyclic or heterocyclic ring;
wherein R is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵) 2, —CO₂R⁵, —(CR⁵₂)₁₋₈aryl, —(CR⁵₂)₁₋₈heterocyclyl, NR⁵C(O)N(R⁵)₂, —NR⁵C(O)R⁵, —NR⁵CO₂R⁵, and —C(O)R⁵;
wherein R³ is selected from H, —OR⁶, halo, aryl, (C₁-C₈)alkyl, (C₂-C₈)alkenyl, (C₂-C₈)alkynyl, (C₁-C₈)perfluoroalkyl, —NR⁵₂, —(C₁-C₈)alkyl-NR⁵₂, —(C₁-C₈)alkyl-OR⁵, —S(O)ₙ-alkyl, —S(O)ₙ-aryl, —S(O)ₙ-heteroaryl, (C₃-C₁₀)cycloalkyl, nitro, heterocyclyl, —NR⁵SO₂R⁵, —C(O)N(R⁵) 2, —CO₂R⁵, —(CR⁵₂)₁₋₈aryl, —(CR⁵₂)₁₋₈heterocyclyl, NR⁵C(O)N(R⁵)₂, —NR⁵C(O)R⁵, —NR⁵CO₂R⁵, and —C(O)R⁵; wherein R² and R³ may be joined to form a 5-10 membered saturated or partially unsaturated carbocyclic or heterocyclic ring;
wherein R⁴ is independently selected from H, and (C₁-C₆)alkyl;
wherein R⁵ is independently selected from H, lower alkyl, optionally substituted aryl, optionally substituted aralkyl, optionally substituted heterocyclyl, optionally substituted heterocyclylalkyl, optionally substituted C₃-C₆ cycloalkyl, optionally substituted C₃-C₆ cycloalkyl-alkyl, lower aminoalkyl, aryl-(C₁-C₆)alkylamino-(C₁-C₆)alkyl, (C₁-C₆)alkylamino-(C₁-C₆)alkyl, aryloxyalkyl, alkylcarbonylalkyl, and lower perfluoroalkyl; and
wherein R⁶ is independently selected from lower alkyl, optionally substituted aryl, optionally substituted aryl-(C₁-C₆)alkyl, optionally substituted heterocyclyl, optionally substituted heterocyclyl-(C₁-C₆)alkyl, optionally substituted C₃-C₆ cycloalkyl, optionally substituted C₃-C₆ cycloalkyl-(C₁-C₆)alkyl, (C₁-C₆)alkylamino-(C₁-C₆)alkyl, aryloxy-(C₁-C₆)alkyl, (C₁-C₆)alkylcarbonyl-(C₁-C₆)alkyl, and lower perfluoroalkyl;
wherein each aryl, heteroaryl, cycloalkyl, and heterocyclyl moiety of any R¹, R², R³, R⁵, R⁶, and Q is optionally substituted with one or more groups selected from halo, —NH₂, —OH, oxo, —CO₂H, (C₁-C₆)alkylamino, (C₁-C₆)alkoxy, (C₁-C₆)alkoxyalkyl, (C₁-C₆)alkyl, di(C₁-C₆)alkylamino, phenyl, and heterocyclyl;
and pharmaceutically acceptable derivatives thereof;
provided R¹ is not CF₃ when R² is ethoxycarbonyl, when R³ is H, when W is thiazol-4-yl and when Q is 4-pyridyl or 2-chloro-4-pyridyl; further provided Q is not 4-pyridyl, when W is thiazol-2-yl, when R¹, R³, and R² are H; further provided Q is not 2-nitro-5-furyl when W is thiazol-2-yl, when R¹ is methyl, when R³ is H, and when R² is H; further provided Q is not phenyl when W is thiazol-2-yl, when R¹ is methyl, when R³ is methyl, and when R² is H; further provided Q is not phenyl, 3,4-diacetylphenyl or 3,4-dihydroxyphenyl, when W is thiazol-2-yl, when R¹ is H, when R³ is H, and when R² is H; and further provided Q is not 3-cyano-6-methyl-2-oxo-1,2-dihydro-5-pyridyl, when W is thiazol-2-yl, when R¹ is methyl, when R³ is H, and when R² is acetyl.

15. Compound of claim 14 wherein Q is selected from R⁶SO₂—(C₁-C₆)alkyl-, 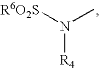
substituted phenyl, and substituted or unsubstituted 5-6 membered heteroaryl;
wherein R⁴ is independently selected from H, and (C₁-C₂)alkyl; and
wherein R⁶ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl- and optionally substituted heterocyclyl selected from pyridyl and thienyl; and pharmaceutically acceptable derivatives thereof.

16. Compound of claim 15 wherein Q is selected from phenylsulfonylamino, N-methyl-N-(2-pyridylsulfonyl)amino, N-methyl-N-(3-pyridylsulfonyl)amino, N-methyl-N-(4-pyridylsulfonyl)amino, N-methyl-N-(2-thienylsulfonyl)amino, N-methyl-N-(phenylsulfonyl)amino, 2-pyridylsulfonylmethyl, 3-pyridylsulfonylmethyl, 4-pyridylsulfonylmethyl, 2-thienylsulfonylmethyl, 3-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, 3-trifluoromethylbenzyl-sulfonylmethyl, methylsulfonylmethyl, tert-butyl-sulfonylmethyl, 4-fluorobenzylsulfonylmethyl, 4-chlorophenyl-methylsulfonylmethyl, 2-thienyl, 3-(4-chlorophenylsulfonylmethyl)-2-thienyl, phenyl substituted
with one or more substituents selected from hydroxyl, chloro, fluoro, methoxy, —O—CH₂—O—, amino, aminomethyl, methylsulfonyl, methyl, cyano, trifluoromethyl, and pyrrolyl,
unsubstituted pyridyl, and
4-pyridyl substituted with one or more substituents selected from chloro, fluoro, methyl, ethyl, —NH₂, methoxy, ethoxy, —OH, —CO₂H, phenoxyethylamino, methylamino, dimethylamino, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl; and
pharmaceutically acceptable derivatives thereof.

17. Compound of claim 14, and pharmaceutically acceptable derivatives thereof, wherein W is thiazol-4-yl.

18. Compound of claim 14 wherein R¹ is selected from (C₁-C₆)alkyl, —(C₁-C₄)alkyl-N(R⁵)₂, —(C₁-C₄)alkyl-OR⁵, (C₃-C₅)cycloalkyl and —CF₃; wherein R⁵ is independently selected from H, C₁-C₅-alkyl, optionally substituted phenyl, optionally substituted benzyl, optionally substituted pyridyl-(C₁-C₃)-alkyl, optionally substituted thienyl-(C₁-C₃)-alkyl, optionally substituted piperazinyl-(C₁-C₃)-alkyl, 4-morpholinyl-(C₁-C₃)-alkyl, optionally substituted pyrrolidinyl-(C₁-C₃)-alkyl, optionally substituted piperidinyl-(C₁-C₃)-alkyl, optionally substituted C₃-C₆ cycloalkyl-(C₁-C₃)-alkyl, amino-(C₁-C₄)-alkyl-, benzylamino-(C₁-C₃)-alkyl-, [N—(C₁-C₃)-alkyl-N-benzylamino]-(C₁-C₃)-alkyl-, —(C₁-C₃)-alkyl-N-((C₁-C₃)-alkyl)₂, —(C₁-C₃)-alkyl-NH—(C₁-C₃)-alkyl and optionally substituted heterocyclyl selected from piperazinyl, morpholinyl, pyrrolidinyl and piperidinyl; and pharmaceutically acceptable derivatives thereof.

19. Compound of claim 18 wherein R¹ is selected from methyl, ethyl, propyl, isopropyl, dimethylaminomethyl, 1-pyrrolidinyltheyl, benzyloxymethyl, benzyloxyethyl, hydroxyethyl, 4-methoxy-benzyloxymethyl, methoxymethyl, cyclopropyl and —CF₃; and pharmaceutically acceptable derivatives thereof.

20. Compound of claim 14 wherein R² is selected from H, halo, (C₁-C₃)alkyl, —NR⁵₂, —OR⁶, —(C₁-C₃)alkyl-OR⁵, —(C₁-C₃)alkyl-NR⁵₂, —C(O)N(R⁵) 2, —CO₂R⁵, —(CH₂)₁₋₃-(5-6 membered saturated or partially unsaturated)heterocyclyl, 5-6 membered saturated or partially unsaturated heterocyclyl, —NHC(O)R⁵, and —C(O)R⁵; wherein R⁵ is independently selected from H, C₁-C₅-alkyl, optionally substituted phenyl, optionally substituted benzyl, optionally substituted pyridyl-(C₁-C₃)-alkyl, optionally substituted thienyl-(C₁-C₃)-alkyl, optionally substituted piperazinyl-(C₁-C₃)-alkyl, 4-morpholinyl-(C₁-C₃)-alkyl, optionally substituted pyrrolidinyl-(C₁-C₃)-alkyl, optionally substituted piperidinyl-(C₁-C₃)-alkyl, optionally substituted C₃-C₆ cycloalkyl-(C₁-C₃)-alkyl, amino-(C₁-C₄)-alkyl-, benzylamino-(C₁-C₃)-alkyl-, [N—(C₁-C₃)-alkyl-N-benzylamino]-(C₁-C₃)-alkyl-, —(C₁-C₃)-alkyl-N-((C₁-C₃)-alkyl)₂, —(C₁-C₃)-alkyl-NH—(C₁-C₃)-alkyl and optionally substituted heterocyclyl selected from piperazinyl, morpholinyl, pyrrolidinyl and piperidinyl; and pharmaceutically acceptable derivatives thereof.

21. Compound of claim 20 wherein R² is selected from H, bromo, methyl, hydroxymethyl, 1,2,5,6-tetrahydro-1-pyridylmethyl, 1-piperidinylmethyl, 1-methyl-4-piperazinylmethyl, (N-diethylaminoethyl-N-methyl)aminomethyl, (N-dimethylaminoethyl-N-ethyl)aminomethyl, 4,5-dihydro-oxazol-2-yl, 5-methyl-4,5-dihydro-oxazol-2-yl, 2-furyl, amino, isobutylamino, 3-methylbutylamino, ethylcarbonyl, aminocarbonyl, 4-methoxybenzylaminocarbonyl, 2-pyridylmethylaminocarbonyl, 4-pyridylmethylaminocarbonyl, dimethylaminocarbonyl, ethylaminoethylaminocarbonyl, isopropylaminoethylaminocarbonyl, cyclopropylmethylaminocarbonyl, isobutylaminocarbonyl, ethoxycarbonyl, propoxycarbonyl, 1-methylpropoxycarbonyl, butoxycarbonyl, iso-butoxycarbonyl, tert-butoxycarbonyl, 2-thienylethoxycarbonyl, 4-morpholinylethoxycarbonyl, (4-piperidinyl)methoxycarbonyl, (1-piperazinyl)ethoxycarbonyl, (1-methyl-piperidin-3-yl)oxycarbonyl, (1-methyl-piperidin-4-yl)oxycarbonyl, (1-ethyl-piperidin-3-yl)oxycarbonyl, (1-methyl-pyrrolidin-3-yl)oxycarbonyl, 1-pyrrolidinylethoxycarbonyl, 2-oxo-pyrrolidin-1-ylethoxycarbonyl, 2-oxo-pyrrolidin-1-ylpropoxycarbonyl, 1-methyl-2-pyrrolidinylethoxycarbonyl, 1-piperidinylethoxycarbonyl, diethylaminoethoxycarbonyl, di-isopropylaminoethoxycarbonyl, (N-ethyl-N-benzylamino)ethoxycarbonyl, diethylaminopropoxycarbonyl, dimethylaminoethoxycarbonyl, 2-(dimethylamino)-1-(methyl)ethoxycarbonyl, 2-(diethylamino)-1-(methyl)ethoxycarbonyl, carboxyl, methylcarbonylamino, isobutylcarbonylamino, methylaminomethylcarbonylamino, dimethylaminomethylcarbonylamino, tert-butylaminomethylcarbonylamino, (1-amino-2-methylpropyl)carbonylamino, 1-piperidinylmethylcarbonylamino, 1-piperidinylethylcarbonylamino, 1-piperidinylpropylcarbonylamino, aminomethylcarbonylamino and 1-methyl-4-piperazinylcarbonyl; and pharmaceutically acceptable derivatives thereof.

22. Compound of claim 14 wherein R¹ and R² may be joined together with the pyridone ring to form optionally substituted 2-oxo-1,5,7,8-tetrahydro-2H-[1,6]naphthyridine, optionally substituted 5,6,7,8-tetrahydro-1H-[1,6]naphthyridin-2-one, optionally substituted 5,6,7,8-tetrahydro-1H-[1,7]naphthyridin-2-one, optionally substituted 5,6,7,8-tetrahydro-1H-quinolin-2-one, optionally substituted 7,8-dihydro-1H-quinolin-2-one, 7,8-dihydro-(1H,6H)-quinoline-2,5-dione or 1,5,7,8-tetrahydro-pyrano[4,3-b]pyridin-2-one; and pharmaceutically acceptable derivatives thereof.

23. Compound of claim 22, wherein R¹ and R² are joined together with the pyridone ring to form 6-benzyloxycarbonyl-2-oxo-1,5,7,8-tetrahydro-2H-[1,6]naphthyridine, 5,6,7,8-tetrahydro-1H-[1,6]naphthyridin-2-one, 7-Boc-5,6,7,8-tetrahydro-1H-[1,7]naphthyridin-2-one, 7-ethyl-5,6,7,8-tetrahydro-1H-[1,7]naphthyridin-2-one, 5-methyl-7,8-dihydro-1H-quinolin-2-one, 5-propylamino-5,6,7,8-tetrahydro-1H-quinolin-2-one, 5-propylimino-5,6,7,8-tetrahydro-1H-quinolin-2-one, 7,8-dihydro-(1H,6H)-quinoline-2,5-dione or 1,5,7,8-tetrahydro-pyrano[4,3-b]pyridin-2-one; and pharmaceutically acceptable derivatives thereof.

24. Compound of claim 14 wherein R³ is H; and pharmaceutically acceptable derivatives thereof.

25. Compound of claim 14 wherein A is O; and pharmaceutically acceptable derivatives thereof.

26. Compound of claim 14, and pharmaceutically acceptable derivatives thereof, wherein A is O; wherein Q is selected from N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl, phenyl substituted
with one or more substituents selected from chloro, fluoro, and —O—CH₂—O—, unsubstituted pyridyl, and
4-pyridyl substituted with one or more substituents selected from chloro, fluoro, —NH₂, methoxy, ethoxy, methyl, ethyl, phenoxyethylamino, methylamino, dimethylamino, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl;
wherein R¹ is selected from methyl, ethyl, propyl, isopropyl, dimethylaminomethyl, hydroxyethyl, benzyloxymethyl, 4-methoxy-benzyloxymethyl, methoxymethyl, cyclopropyl, and —CF₃;
wherein R² is selected from H, bromo, methyl, amino, isobutylamino, hydroxymethyl, aminocarbonyl, 4-methoxybenzylaminocarbonyl, 2-pyridylmethylaminocarbonyl, ethylaminoethylaminocarbonyl, isopropylaminoethylaminocarbonyl, cyclopropylmethylaminocarbonyl, isobutylaminocarbonyl, ethoxycarbonyl, tert-butoxycarbonyl, 4-morpholinylethoxycarbonyl, 1-pyrrolidinylethoxycarbonyl, 1-piperidinylethoxycarbonyl, diethylaminopropoxycarbonyl, carboxyl, 1,2,5,6-tetrahydro-1-pyridylmethyl, 1-piperidinylmethyl, 1-methyl-4-piperazinylmethyl, methylcarbonylamino, isobutylcarbonylamino, and 1-methyl-4-piperazinylcarbonyl;
wherein R¹ and R² may be joined together with the pyridone ring to form 6-benzyloxycarbonyl-2-oxo-1,5,7,8-tetrahydro-2H-[1,6]naphthyridine, 5,6,7,8-tetrahydro-1H-[1,6]naphthyridin-2-one, 7-Boc-5,6,7,8-tetrahydro-1H-[1,7]naphthyridin-2-one, 7-ethyl-5,6,7,8-tetrahydro-1H-[1,7]naphthyridin-2-one, 5-methyl-7,8-dihydro-1H-quinolin-2-one, 5-propylamino-5,6,7,8-tetrahydro-1H-quinolin-2-one, 5-propylimino-5,6,7,8-tetrahydro-1H-quinolin-2-one, 7,8-dihydro-(1H,6H)-quinoline-2,5-dione or 1,5,7,8-tetrahydro-pyrano[4,3-b]pyridin-2-one; and
wherein R³ is H.

27. A compound of claim 14 having Formula II′ 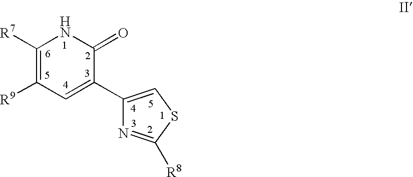
wherein R⁷ is selected from —(C₁-C₃)alkyl, —(C₁-C₃)alkyl-N(R¹⁰)₂, —(C₁-C₃)alkyl-OR¹⁰, —(C₃-C₅)cycloalkyl, and —CF₃;
wherein R⁸ is selected from R¹⁰SO₂—(C₁-C₆)alkyl-, R¹¹SO₂NH— 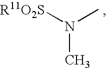
substituted phenyl, and substituted or unsubstituted 5-6 membered heteroaryl;
wherein R⁹ is selected from H, halo, (C₁-C₃)alkyl, —NR¹⁰₂, —(C₁-C₃)alkyl-OR¹⁰, —C(O)N(R¹⁰) 2, —CO₂R¹⁰, (CH₂)₁₋₃-(5-6 membered saturated or partially unsaturated heterocyclyl, —NHC(O)R¹⁰, and —C(O)R¹⁰;
wherein R¹⁰ is independently selected from H, (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl- and optionally substituted heterocyclyl selected from pyridyl and thienyl; and
wherein R¹¹ is independently selected from (C₁-C₄)alkyl, optionally substituted phenyl, optionally substituted phenyl-(C₁-C₂)alkyl, optionally substituted furyl-(C₁-C₂)-alkyl, optionally substituted C₃-C₆ cycloalkyl-(C₁-C₂)-alkyl, (C₁-C₃)alkylamino-(C₁-C₃)-alkyl-, phenyloxy-(C₁-C₃)alkyl-, (C₁-C₂)alkylcarbonyl-(C₁-C₂)alkyl, and optionally substituted heterocyclyl selected from pyridyl and thienyl;
and pharmaceutically acceptable derivatives thereof;
provided R⁷ is not CF₃ when R⁹ is ethoxycarbonyl and when R⁸ is 4-pyridyl or 2-chloro-4-pyridyl.

28. Compound of claim 27 wherein R⁷ is selected from methyl, ethyl, propyl, isopropyl, dimethylaminomethyl, 1-pyrrolidinyltheyl, benzyloxymethyl, benzyloxyethyl, hydroxyethyl, 4-methoxy-benzyloxymethyl, methoxymethyl, cyclopropyl and —CF₃; wherein R⁸ is selected from N-methyl-N-(phenylsulfonyl)amino, 2-pyridylsulfonylmethyl, 2-thienylsulfonylmethyl, phenylsulfonylmethyl, (1-methyl)-1-(phenylsulfonyl)ethyl, 4-chlorophenyl-sulfonylmethyl, 2-furylmethylsulfonylmethyl, methylsulfonylmethyl, tert-butyl-sulfonylmethyl, 4-fluorobenzylsulfonylmethyl, 2-thienyl, phenyl substituted with one or more substituents selected from
chloro, fluoro, and —O—CH₂—O—,
unsubstituted pyridyl, and
4-pyridyl substituted with one or more substituents selected from chloro, fluoro, —NH₂, methoxy, ethoxy, methyl, ethyl, phenoxyethylamino, methylamino, butylamino, isobutylamino, dimethylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl; and
wherein R⁹ is selected from H, bromo, methyl, hydroxymethyl, 1,2,5,6-tetrahydro-1-pyridylmethyl, 1-piperidinylmethyl, 1-methyl-4-piperazinylmethyl, (N-diethylaminoethyl-N-methyl)aminomethyl, (N-dimethylaminoethyl-N-ethyl)aminomethyl, 4,5-dihydro-oxazol-2-yl, 5-methyl-4,5-dihydro-oxazol-2-yl, 2-furyl, amino, isobutylamino, 3-methylbutylamino, ethylcarbonyl, aminocarbonyl, 4-methoxybenzylaminocarbonyl, 2-pyridylmethylaminocarbonyl, 4-pyridylmethylaminocarbonyl, dimethylaminocarbonyl, ethylaminoethylaminocarbonyl, isopropylaminoethylaminocarbonyl, cyclopropylmethylaminocarbonyl, isobutylaminocarbonyl, ethoxycarbonyl, propoxycarbonyl, 1-methylpropoxycarbonyl, butoxycarbonyl, iso-butoxycarbonyl, tert-butoxycarbonyl, 2-thienylethoxycarbonyl, 4-morpholinylethoxycarbonyl, (4-piperidinyl)methoxycarbonyl, (1-piperidinyl)ethoxycarbonyl, (1-piperazinyl)ethoxycarbonyl, (1-methyl-piperidin-3-yl)oxycarbonyl, (1-methyl-piperidin-4-yl)oxycarbonyl, (1-ethyl-piperidin-3-yl)oxycarbonyl, (1-methyl-pyrrolidin-3-yl)oxycarbonyl, 1-pyrrolidinylethoxycarbonyl, 2-oxo-pyrrolidin-1-ylethoxycarbonyl, 2-oxo-pyrrolidin-1-ylpropoxycarbonyl, 1-methyl-2-pyrrolidinylethoxycarbonyl, 1-piperidinylethoxycarbonyl, diethylaminoethoxycarbonyl, di-isopropylaminoethoxycarbonyl, (N-ethyl-N-benzylamino)ethoxycarbonyl, diethylaminopropoxycarbonyl, dimethylaminoethoxycarbonyl, 2-(dimethylamino)-1-(methyl)ethoxycarbonyl, 2-(diethylamino)-1-(methyl)ethoxycarbonyl, carboxyl, methylcarbonylamino, isobutylcarbonylamino, methylaminomethylcarbonylamino, dimethylaminomethylcarbonylamino, tert-butylaminomethylcarbonylamino, (1-amino-2-methylpropyl)carbonylamino, 1-piperidinylmethylcarbonylamino, 1-piperidinylethylcarbonylamino, 1-piperidinylpropylcarbonylamino, aminomethylcarbonylamino and 1-methyl-4-piperazinylcarbonyl; and pharmaceutically acceptable derivatives thereof.

29. Compound of claim 27 wherein R⁷ is selected from methyl, ethyl, propyl, and isopropyl.

30. Compound of claim 27 wherein R⁸ is selected from phenylsulfonylmethyl and 4-pyridyl substituted with one or more substituents selected from chloro, fluoro, —NH₂, methoxy, ethoxy, phenoxyethylamino, methylamino, dimethylamino, methyl, ethyl, butylamino, isobutylamino, benzylamino, 4-fluorobenzylamino, 2-thienylethylamino, 3-pyridylmethylamino, 2-pyridylmethylamino, 2-furylmethylamino, 4-methoxybenzylamino, diethylamino, cyclopropylmethylamino, cyclopentylmethylamino, ethylaminoethylamino, diethylaminoethylamino, isopropylaminoethylamino, methylcarbonylaminoethylamino, methylcarbonylmethylamino, pyrrolidinyl, piperazinyl, piperidinyl, morpholinyl and azetidinyl.

31. Compound of claim 27 wherein R⁹ is selected from methyl, hydroxymethyl, 1,2,5,6-tetrahydro-1-pyridylmethyl, 1-piperidinylmethyl, 1-methyl-4-piperazinylmethyl, (N-diethylaminoethyl-N-methyl)aminomethyl, (N-dimethylaminoethyl-N-ethyl)aminomethyl, 4,5-dihydro-oxazol-2-yl, 5-methyl-4,5-dihydro-oxazol-2-yl, 2-furyl, amino, isobutylamino, 3-methylbutylamino, ethylcarbonyl, aminocarbonyl, 4-methoxybenzylaminocarbonyl, 2-pyridylmethylaminocarbonyl, 4-pyridylmethylaminocarbonyl, dimethylaminocarbonyl, ethylaminoethylaminocarbonyl, isopropylaminoethylaminocarbonyl, cyclopropylmethylaminocarbonyl, isobutylaminocarbonyl, ethoxycarbonyl, propoxycarbonyl, 1-methylpropoxycarbonyl, butoxycarbonyl, iso-butoxycarbonyl, tert-butoxycarbonyl, 2-thienylethoxycarbonyl, 4-morpholinylethoxycarbonyl, (4-piperidinyl)methoxycarbonyl, (1-piperidinyl)ethoxycarbonyl, (1-piperazinyl)ethoxycarbonyl, (1-methyl-piperidin-3-yl)oxycarbonyl, (1-methyl-piperidin-4-yl)oxycarbonyl, (1-ethyl-piperidin-3-yl)oxycarbonyl, (1-methyl-pyrrolidin-3-yl)oxycarbonyl, 1-pyrrolidinylethoxycarbonyl, 2-oxo-pyrrolidin-1-ylethoxycarbonyl, 2-oxo-pyrrolidin-1-ylpropoxycarbonyl, 1-methyl-2-pyrrolidinylethoxycarbonyl, 1-piperidinylethoxycarbonyl, diethylaminoethoxycarbonyl, di-isopropylaminoethoxycarbonyl, (N-ethyl-N-benzylamino)ethoxycarbonyl, diethylaminopropoxycarbonyl, dimethylaminoethoxycarbonyl, 2-(dimethylamino)-1-(methyl)ethoxycarbonyl, 2-(diethylamino)-1-(methyl)ethoxycarbonyl, carboxyl, methylcarbonylamino, isobutylcarbonylamino, methylaminomethylcarbonylamino, dimethylaminomethylcarbonylamino, tert-butylaminomethylcarbonylamino, (1-amino-2-methylpropyl)carbonylamino, 1-piperidinylmethylcarbonylamino, 1-piperidinylethylcarbonylamino, 1-piperidinylpropylcarbonylamino, aminomethylcarbonylamino and 1-methyl-4-piperazinylcarbonyl; and pharmaceutically acceptable derivatives thereof.

32. Compound of claim 27 and pharmaceutically acceptable derivatives thereof selected from:
6-Isopropyl-5-methyl-3-(2-pyrindin-4-yl-thiazol-4-yl)-1H-pyridin-2-one;
6-Ethyl-5-isopropionyl-3-(2-pyridin-4-yl-thiazol-4-yl)-1H-pyridin-2-one;
2-Isopropyl-6-oxo-5-(2-pyridin-4-yl-thiazol-4-yl)-1,6-dihydro-pyridine-3-carboxylic acid 2-(2-oxo-pyrrolidin-1-yl)-ethyl ester;
2-Isopropyl-6-oxo-5-(2-pyridin-4-yl-thiazol-4-yl)-1,6-dihydro-pyridine-3-carboxylic acid 2-diethylamino-ethyl ester;
2-Isopropyl-6-oxo-5-(2-pyridin-4-yl-thiazol-4-yl)-1,6-dihydro-pyridine-3-carboxylic acid 2-pyrrolidin-1-yl-ethyl ester;
2-Isopropyl-6-oxo-5-(2-pyridin-4-yl-thiazol-4-yl)-1,6-dihydro-pyridine-3-carboxylic acid 2-diethylamino-1-methyl-ethyl ester;
2-Isopropyl-6-oxo-5-(2-pyridin-4-yl-thiazol-4-yl)-1,6-dihydro-pyridine-3-carboxylic acid 1-ethyl-piperidin-3-yl ester;
2-Isopropyl-6-oxo-5-(2-pyridin-4-yl-thiazol-4-yl)-1,6-dihydro-pyridine-3-carboxylic acid 2-dimethylamino-ethyl ester;
2-Isopropyl-6-oxo-5-(2-pyridin-4-yl-thiazol-4-yl)-1,6-dihydro-pyridine-3-carboxylic acid 2-dimethylamino-1-methyl-ethyl ester;
2-Isopropyl-6-oxo-5-(2-pyridin-4-yl-thiazol-4-yl)-1,6-dihydro-pyridine-3-carboxylic acid 1-methyl-piperidin-3-yl ester;
2-Isopropyl-6-oxo-5-(2-pyridin-4-yl-thiazol-4-yl)-1,6-dihydro-pyridine-3-carboxylic acid 1-ethyl-pyrrolidin-3-yl ester;
5-(2-Benzenesulfonylmethyl-thiazol-4-yl)-2-isopropyl-6-oxo-1,6-pyridine-3-carboxylic acid 2-diethylamino-ethyl ester;
2-Isopropyl-6-oxo-5-(2-pyridin-4-yl-thiazol-4-yl)-1,6-dihydro-pyridine-3-carboxylic acid piperidin-4-ylmethyl ester;
5-(2-Benzenesulfonylmethyl-thiazol-4-yl)-2-isopropyl-6-oxo-1,6-pyridine-3-carboxylic acid 2-diethylamino-1-methyl-ethyl ester;
2-Isopropyl-6-oxo-5-(2-pyridin-4-yl-thiazol-4-yl)-1,6-dihydro-pyridine-3-carboxylic acid 2-(benzyl-methyl-amino)-ethyl ester;
5-(2-Benzenesulfonylmethyl-thiazol-4-yl)-2-isopropyl-6-oxo-1,6-pyridine-3-carboxylic acid 2-diethylamino-propyl ester;
5-(2-Benzenesulfonylmethyl-thiazol-4-yl)-2-isopropyl-6-oxo-1,6-pyridine-3-carboxylic acid 2-(1-methyl-pyrrolidin-2-yl)-ethyl ester;
5-[2-(2-Dimethylamino-pyridin-4-yl)-thiazol-4-yl]-2-isopropyl-6-oxo-1,6-dihydro-pyridine-3-carboxylic acid ethyl ester;
2-Isopropyl-6-oxo-5-(2-pyridin-4-yl-thiazol-4-yl)-1,6-dihydro-pyridine-3-carboxylic acid 2-piperazin-1-yl-ethyl ester;
2-Isopropyl-6-oxo-5-(2-pyridin-4-yl-thiazol-4-yl)-1,6-dihydro-pyridine-3-carboxylic acid 2-(2-oxo-pyrrolidin-1-yl)-propyl ester;
2-Isopropyl-6-oxo-5-(2-pyridin-4-yl-thiazol-4-yl)-1,6-dihydro-pyridine-3-carboxylic acid 1-methyl-pyrrolidin-3-yl ester;
3-(2-Benzenesulfonylmethyl-thiazol-4-yl)-6-isopropyl-5-methyl-1H-pyridin-2-one;
3-(2-Benzenesulfonylmethyl-thiazol-4yl)-6-ethyl-5-propionyl-1H-pyridin-2-one;
2-Isopropyl-6-oxo-5-(2-pyridin-4-yl-thiazol-4-yl)-1,6-dihydro-pyridine-3-carboxylic acid 2-morpholin-4-yl-ethyl ester;
2-Isopropyl-6-oxo-5-(2-pyridin-4-yl-thiazol-4-yl)-1,6-dihydro-pyridine-3-carboxylic acid phenethyl ester;
2-Isopropyl-6-oxo-5-(2-pyridin-4-yl-thiazol-4-yl)-1,6-dihydro-pyridine-3-carboxylic acid piperidin-4-ylmethyl ester;
2-Isopropyl-6-oxo-5-(2-pyridin-4-yl-thiazol-4-yl)-1,6-dihydro-pyridine-3-carboxylic acid 2-thiophen-2-yl-ethyl ester;
5-(4,5-Dihydro-oxazol-2-yl)-6-isopropyl-3-(2-pyridin-4-yl-thiazol-4-yl)-1H-pyridin-2-one;
5-{[(2-Dimethylamino-ethyl)-ethyl-amino]-methyl}-6-ethyl-3-(2-pyridin-4-yl-thiazol-4-yl)-1H-pyridin-2-one;
2-Isopropyl-6-oxo-5-(2-pyridin-4-yl-thiazol-4-yl)-1,6-dihydro-pyridine-3-carboxylic acid 2-piperidin-1-yl-ethyl ester;
5-{[(2-Diethylamino-ethyl)-methyl-amino]-methyl}-6-ethyl-3-(2-pyridin-4-yl-thiazol-4-yl)-1H-pyridin-2-one;
2-(2-Hydroxy-ethyl)-6-oxo-5-(2-pyridin-4-yl-thiazol-4-yl)-1,6-dihydro-pyridine-3-carboxylic acid ethyl ester;
2-Amino-N-[2-ethyl-6-oxo-5-(2-pyridin-4-yl-thiazol-4-yl)-1,6-dihydro-pyridin-3-yl]-acetamide;
2-tert-Butylamino-N-[2-ethyl-6-oxo-5-(2-pyridin-4-yl-thiazol-4-yl)-1,6-dihydro-pyridin-3-yl]-acetamide;
6-Ethyl-5-(3-methyl-butylamino)-3-(2-pyridin-4-yl-thiazol-4-yl)-1H-pyridin-2-one;
Ethyl 2-ethyl-6-oxo-5-(2-(4-pyridinyl)-1,3-thiazol-4-yl)-1,6-dihydro-pyridine-3-carboxylate;
Ethyl-2-ethyl-6-oxo-5-{2-[(thienylsulfonyl)methyl](1,3-thiazol-4-yl)}-1,6-dihydro-pyridine-3-carboxylate;
Ethyl-2-ethyl-6-oxo-5-{2-[(phenylsulfonyl)methyl](1,3-thiazol-4-yl)}-1,6-dihydro-pyridine-3-carboxylate;
Ethyl-6-oxo-5-{2-[(phenylsulfonyl)methyl](1,3-thiazol-4-yl)}-2-(trifluoromethyl)-1,6-dihydro-pyridine-3-carboxylate;
Ethyl-6-oxo-5-{2-[(2-pyridylsulfonyl)methyl](1,3-thiazol-4-yl)}-2-(trifluoromethyl)-1,6-dihydro-pyridine-3-carboxylate;
Ethyl-6-oxo-5-{2-[(2-thienylsulfonyl)methyl](1,3-thiazol-4-yl)}-2-(trifluoromethyl)-1,6-dihydro-pyridine-3-carboxylate;
Ethyl 2-isopropyl-6-oxo-5-(2-(4-pyridyl)(1,3-thiazol-4-yl)-1,6-dihydro-pyridine-3-carboxylate;
Ethyl 2-isopropyl-6-oxo-5-{2-[(thienylsulfonyl)methyl](1,3-thiazol-4-yl)}-1,6-dihydro-pyridine-3-carboxylate;
Ethyl 2-isopropyl-6-oxo-5-{2-[(phenylsulfonyl)methyl](1,3-thiazol-4-yl)}-1,6-dihydro-pyridine-3-carboxylate;
Ethyl 2-propyl-6-oxo-5-(2-(4-pyridyl)(1,3-thiazol-4-yl)-1,6-dihydro-pyridine-3-carboxylate;
Ethyl 2-propyl-6-oxo-5-{2-[(phenylsulfonyl)methyl](1,3-thiazol-4-yl)}-1,6-dihydro-pyridine-3-carboxylate;
Ethyl 2-propyl-6-oxo-5-{2-[(thienylsulfonyl)methyl](1,3-thiazol-4-yl)}-1,6-dihydropyridine-3-carboxylate;
Ethyl 6-oxo-2-[(phenylmethoxy)methyl]-5-(2-(4-pyridyl)(1,3-thiazol-4-yl))-1,6-dihydropyridine-3-carboxylate;
Ethyl 6-oxo-2-[(phenylmethoxy)methyl]-5-{2-[(phenylsulfonyl)methyl](1,3-thiazol-4-yl)}-1,6-dihydropyridine-3-carboxylate;
Ethyl 2-methyl-6-oxo-5-{2-[(2-thienylsulfonyl)methyl](1,3-thiazol-4-yl)}-1,6-dihydropyridine-3-carboxylate;
Ethyl 5-[2-({[(4-fluorophenyl)methyl]sulfonyl}methyl)(1,3-thiazol-4-yl)]-2-methyl-6-oxo-1,6-dihydropyridine-3-carboxylate;
Ethyl 5-[2-({[(4-fluorophenyl)methyl]sulfonyl}methyl)(1,3-thiazol-4-yl)]-2-methyl-6-oxo-1,6-dihydropyridine-3-carboxylate;
(Ethyl 2-methyl-6-oxo-5-{2-[(2-thienylsulfonyl)methyl]methyl](1,3-thiazol-4-yl)}-1,6-dihydropyridine-3-carboxylate;
Ethyl 2-methyl-6-oxo-5-{2-(phenylthiomethyl)(1,3-thiazol-4-yl)}-1,6-dihydropyridine-3-carboxylate;
Ethyl 5-[2-(2-chloro(4-pyridyl))(1,3-thiazol-4-yl)-2-methyl-6-oxo-1,6-dihydropyridine-3-carboxylate;
Ethyl 5-(2-{[(2-furylmethyl)sulfonyl]methyl}(1,3-thiazol-4-yl))-2-methyl-6-oxo-1,6-dihydropyridine-3-carboxylate;
Ethyl 5-(2-{[(2-furylmethyl)sulfonyl]methyl}(1,3-thiazol-4-yl))-2-methyl-6-oxo-1,6-dihydropyridine-3-carboxylate;
Ethyl 5-[2-(2-ethyl(4-pyridyl))(1,3-thiazol-4-yl)-2-methyl-6-oxo-1,6-dihydropyridine-3-carboxylate;
Ethyl 2-methyl-5-(2-(2-((2-methylpropyl)amino)-4-pyridinyl)-1,3-thiazol-4-yl)-6-oxo-1,6-dihydropyridine-3-carboxylate;
Ethyl 2-methyl-6-oxo-5-(2-(2-((3-pyridinylmethyl)amino)-4-pyridinyl)-1,3-thiazol-4-yl)-1,6-dihydropyridine-3-carboxylate;
Ethyl 2-methyl-6-oxo-5-(2-(2-((phenylmethyl)amino)-4-pyridinyl)-1,3-thiazol-4-yl)-1,6-dihydropyridine-3-carboxylate;
Ethyl 2-methyl-5-(2-(2-((2-((1-methylethyl)amino)ethyl)amino)-4-pyridinyl)-1,3-thiazol-4-yl)-6-oxo-1,6-dihydropyridine-3-carboxylate;
Ethyl 5-(2-(2-((2-(diethylamino)ethyl)amino)-4-pyridinyl)-1,3-thiazol-4-yl)-2-methyl-6-oxo-1,6-dihydropyridine-3-carboxylate;
Ethyl 5-(2-{2-[(fur-2-ylmethyl)-amino]-pyridin-4-yl}-thiazol-4-yl)-2-methyl-6-oxo-1,6-dihydropyridine-3-carboxylate;
Ethyl 5-{2-[2-(2-thien-2-yl-ethylamino)-pyridin-4-yl]-thiazol-4-yl}-2-methyl-6-oxo-1,6-dihydropyridine-3-carboxylate;
Ethyl 5-[2-(2-butylamino-pyridin-4-yl)-thiazol-4-yl]-2-methyl-6-oxo-1,6-dihydropyridine-3-carboxylate;
Ethyl 5-{2-[2-(carbamoylmethyl-amino)-pyridin-4-yl]-thiazol-4-yl}-2-methyl-6-oxo-1,6-dihydropyridine-3-carboxylate;
Ethyl 5-{2-[2-acetylamino-ethylamino)-pyridin-4-yl]-thiazol-4-yl}-2-methyl-6-oxo-1,6-dihydropyridine-3-carboxylate;
5-{2-[2-(Cyclopropylmethylamino)-pyridin-4-yl]-thiazol-4-yl}-2-methyl-6-oxo-1,6-dihydropyridine-3-carboxylic acid cyclopropyl-methyl amide;
Ethyl 5-(2-[2-(cyclopropylmethyl-amino)-pyridin-4-yl]-thiazol-4-yl)-2-methyl-6-oxo-1,6-dihydropyridine-3-carboxylate;
5-{2-[2-(Cyclopentyl)methylamino-pyridin-4-yl]-thiazol-4-yl}-2-methyl-6-oxo-1,6-dihydropyridine-3-carboxylate;
5-{2-[2-(4-Methoxybenzylamino)-pyridin-4-yl]-thiazol-4-yl}-2-methyl-6-oxo-1,6-dihydropyridine-3-carboxylic acid 4-methoxy-benzylamide;
Ethyl 2-methyl-6-oxo-5-(2-(2-amino-4-pyridinyl)-1,3-thiazol-4-yl)-1,6-dihydropyridine-3-carboxylate;
Ethyl 2-methyl-5-[2-(methylamino)(1,3-thiazol-4-yl)]-6-oxo-1,6-dihydropyridine-3-carboxylate;
6-Methyl-3-(2-(4-pyridyl)(1,3-thiazol-4-yl))hydropyridin-2-one;
Ethyl 2-methyl-5-(2-(2-(methyloxy)-4-pyridinyl)-1,3-thiazol-4-yl)-6-oxo-1,6-dihydropyridine-3-carboxylate;
Ethyl 2-methyl-6-oxo-5-{2-[(phenylsulfonyl)methyl](1,3-thiazol-4-yl)}-1,6-dihydropyridine-3-carboxylate;
Ethyl 2-methyl-6-oxo-5-{2-(4-pyridyl)(1,3-thiazol-4-yl)}-1,6-dihydropyridine-3-carboxylate;
Ethyl 2-methyl-6-oxo-5-{2-[(2-pyridylsulfonyl)methyl](1,3-thiazol-4-yl)}-1,6-dihydropyridine-3-carboxylate;
Ethyl 2-methyl-5-(2-(1-methyl-1-(phenylsulfonyl)ethyl)-1,3-thiazol-4-yl)-6-oxo-1,6-dihydropyridine-3-carboxylate;
Ethyl 2-cyclopropyl-6-oxo-5-(2-(4-pyridinyl)-1,3-thiazol-4-yl)-1,6-dihydropyridine-3-carboxylate;
Ethyl 2-cyclopropyl-6-oxo-5-(2-((phenylsulfonyl)methyl)-1,3-thiazol-4-yl)-1,6-dihydropyridine-3-carboxylate;
5-Bromo-6-methyl-3-(2-(4-pyridinyl)-1,3-thiazol-4-yl)-2(1H)-pyridinone;
Ethyl 2-methyl-5-(2-(2-(methylamino)-4-pyridinyl)-1,3-thiazol-4-yl)-6-oxo-1,6-dihydropyridine-3-carboxylate;
5-Amino-6-ethyl-3-(2-(4-pyridinyl)-1,3-thiazol-4-yl)-2(1H)-pyridinone;
6-Methyl-3-(2-(2-((2-pyridinylmethyl)amino)-4-pyridinyl)-1,3-thiazol-4-yl)-2(1H)-pyridinone;
Ethyl 2-methyl-6-oxo-5-(2-(2-((2-pyridinylmethyl)amino)-4-pyridinyl)-1,3-thiazol-4-yl)-1,6-dihydropyridine-3-carboxylate;
Ethyl 5-[2-(methylamino-pyridin-4-yl)-thiazol-4-yl]-2-isopropyl-6-oxo-1,6-dihydropyridine-3-carboxylate;
1,1-Dimethylethyl 2-methyl-6-oxo-5-(2-(4-pyridinyl)-1,3-thiazol-4-yl)-1,6-dihydropyridine-3-carboxylate;
2-(1-Pyrrolidinyl)ethyl 2-ethyl-6-oxo-5-(2-(4-pyridinyl)-1,3-thiazol-4-yl)-1,6-dihydropyridine-3-carboxylate;
6-Ethyl-3-(2-pyridin-4-yl-thiazol-4-yl)-1H-pyridin-2-one;
6-Isopropyl-3-(2-pyridin-4-yl-thiazol-4-yl)-1H-pyridin-2-one;
3-(Diethylamino)propyl 2-ethyl-6-oxo-5-(2-(4-pyridinyl)-1,3-thiazol-4-yl)-1,6-dihydropyridine-3-carboxylate;
3-(Diethylamino)propyl 2-(1-methylethyl)-6-oxo-5-(2-(4-pyridinyl)-1,3-thiazol-4-yl)-1,6-dihydropyridine-3-carboxylate; and
5-Hydroxymethyl-6-methyl-3-(2-pyridin-4-yl-thiazol-4-yl)-1H-pyridin-2-one.

33. Compound of claim 27 and pharmaceutically acceptable derivatives thereof selected from:
6-Isopropyl-5-methyl-3-(2-pyrindin-4-yl-thiazol-4-yl)-1H-pyridin-2-one;
3-(2-Benzenesulfonylmethyl-thiazol-4-yl)-6-isopropyl-5-methyl-1H-pyridin-2-one;
6-Ethyl-5-isopropionyl-3-(2-pyridin-4-yl-thiazol-4-yl)-1H-pyridin-2-one;
3-(2-Benzenesulfonylmethyl-thiazol-4yl)-6-ethyl-5-propionyl-1H-pyridin-2-one;
2-Isopropyl-6-oxo-5-(2-pyridin-4-yl-thiazol-4-yl)-1,6-dihydro-pyridine-3-carboxylic acid 2-pyrrolidin-1-yl-ethyl ester;
2-Isopropyl-6-oxo-5-(2-pyridin-4-yl-thiazol-4-yl)-1,6-dihydro-pyridine-3-carboxylic acid 2-(2-oxo-pyrrolidin-1-yl)-ethyl ester;
2-Isopropyl-6-oxo-5-(2-pyridin-4-yl-thiazol-4-yl)-1,6-dihydro-pyridine-3-carboxylic acid 2-diethylamino-ethyl ester;
2-Isopropyl-6-oxo-5-(2-pyridin-4-yl-thiazol-4-yl)-1,6-dihydro-pyridine-3-carboxylic acid 1-ethyl-piperidin-3-yl ester;
2-Isopropyl-6-oxo-5-(2-pyridin-4-yl-thiazol-4-yl)-1,6-dihydro-pyridine-3-carboxylic acid 1-methyl-piperidin-3-yl ester;
2-Isopropyl-6-oxo-5-(2-pyridin-4-yl-thiazol-4-yl)-1,6-dihydro-pyridine-3-carboxylic acid 2-dimethylamino-1-methyl-ethyl ester;
2-Isopropyl-6-oxo-5-(2-pyridin-4-yl-thiazol-4-yl)-1,6-dihydro-pyridine-3-carboxylic acid 2-diethylamino-1-methyl-ethyl ester;
2-Isopropyl-6-oxo-5-(2-pyridin-4-yl-thiazol-4-yl)-1,6-dihydro-pyridine-3-carboxylic acid 2-(benzyl-methyl-amino)-ethyl ester;
2-Isopropyl-6-oxo-5-(2-pyridin-4-yl-thiazol-4-yl)-1,6-dihydro-pyridine-3-carboxylic acid 1-methyl-piperidin-4-yl ester;
2-Isopropyl-6-oxo-5-(2-pyridin-4-yl-thiazol-4-yl)-1,6-dihydro-pyridine-3-carboxylic acid 2-(2-oxo-pyrrolidin-1-yl)-propyl ester;
2-Isopropyl-6-oxo-5-(2-pyridin-4-yl-thiazol-4-yl)-1,6-dihydro-pyridine-3-carboxylic acid phenethyl ester;
2-Isopropyl-6-oxo-5-(2-pyridin-4-yl-thiazol-4-yl)-1,6-dihydro-pyridine-3-carboxylic acid 2-thiophen-2-yl-ethyl ester;
5-(2-Benzenesulfonylmethyl-thiazol-4-yl)-2-isopropyl-6-oxo-1,6-pyridine-3-carboxylic acid 2-diethylamino-ethyl ester;
5-(2-Benzenesulfonylmethyl-thiazol-4-yl)-2-isopropyl-6-oxo-1,6-pyridine-3-carboxylic acid 2-diethylamino-1-methyl-ethyl ester;
5-(2-Benzenesulfonylmethyl-thiazol-4-yl)-2-isopropyl-6-oxo-1,6-pyridine-3-carboxylic acid 2-diethylamino-propyl ester;
5-(2-Benzenesulfonylmethyl-thiazol-4-yl)-2-isopropyl-6-oxo-1,6-pyridine-3-carboxylic acid 2-(1-methyl-pyrrolidin-2-yl)-ethyl ester;
2-Isopropyl-6-oxo-5-(2-pyridin-4-yl-thiazol-4-yl)-1,6-dihydro-pyridine-3-carboxylic acid methyl ester;
2-Isopropyl-6-oxo-5-(2-pyridin-4-yl-thiazol-4-yl)-1,6-dihydro-pyridine-3-carboxylic acid propyl ester;
2-Isopropyl-6-oxo-5-(2-pyridin-4-yl-thiazol-4-yl)-1,6-dihydro-pyridine-3-carboxylic acid butyl ester;
2-Isopropyl-6-oxo-5-(2-pyridin-4-yl-thiazol-4-yl)-1,6-dihydro-pyridine-3-carboxylic acid isobutyl ester;
2-Isopropyl-6-oxo-5-(2-pyridin-4-yl-thiazol-4-yl)-1,6-dihydro-pyridine-3-carboxylic acid sec-butyl ester;
5-([(2-Diethylamino-ethyl)-methyl-amino]-methyl)-6-ethyl-3-(2-pyridin-4-yl-thiazol-4-yl)-1H-pyridin-2-one;
5-[2-(2-Dimethylamino-pyridin-4-yl)-thiazol-4-yl]-2-isopropyl-6-oxo-1,6-dihydro-pyridine-3-carboxylic acid ethyl ester;
Ethyl 2-ethyl-6-oxo-5-(2-(4-pyridinyl)-1,3-thiazol-4-yl)-1,6-dihydropyridine-3-carboxylate;
Ethyl 2-ethyl-6-oxo-5-{2-[(thienylsulfonyl)methyl](1,3-thiazol-4-yl)}-1,6-dihydropyridine-3-carboxylate;
Ethyl 2-ethyl-6-oxo-5-{2-[(phenylsulfonyl)methyl](1,3-thiazol-4-yl)}-1,6-dihydropyridine-3-carboxylate;
Ethyl 2-isopropyl-6-oxo-5-(2-(4-pyridyl)(1,3-thiazol-4-yl)-1,6-dihydropyridine-3-carboxylate;
Ethyl 2-isopropyl-6-oxo-5-{2-[(thienylsulfonyl)methyl](1,3-thiazol-4-yl)}-1,6-dihydropyridine-3-carboxylate;
Ethyl 2-isopropyl-6-oxo-5-{2-[(phenylsulfonyl)methyl](1,3-thiazol-4-yl)}-1,6-dihydropyridine-3-carboxylate;
Ethyl 2-propyl-6-oxo-5-(2-(4-pyridyl)(1,3-thiazol-4-yl)-1,6-dihydropyridine-3-carboxylate;
Ethyl 2-propyl-6-oxo-5-{2-[(phenylsulfonyl)methyl](1,3-thiazol-4-yl)}-1,6-dihydropyridine-3-carboxylate;
Ethyl 2-propyl-6-oxo-5-{2-[(thienylsulfonyl)methyl](1,3-thiazol-4-yl)}-1,6-dihydropyridine-3-carboxylate;
Ethyl 6-oxo-2-[(phenylmethoxy)methyl]-5-(2-(4-pyridyl)(1,3-thiazol-4-yl))-1,6-dihydropyridine-3-carboxylate;
Ethyl 6-oxo-2-[(phenylmethoxy)methyl]-5-{2-[(phenylsulfonyl)methyl](1,3-thiazol-4-yl)}-1,6-dihydropyridine-3-carboxylate;
Ethyl 2-methyl-6-oxo-5-{2-[(2-thienylsulfonyl)methyl](1,3-thiazol-4-yl)}-1,6-dihydropyridine-3-carboxylate;
Ethyl 5-[2-({[(4-fluorophenyl)methyl]sulfonyl}methyl)(1,3-thiazol-4-yl)]-2-methyl-6-oxo-1,6-dihydropyridine-3-carboxylate;
Ethyl 5-[2-({[(4-fluorophenyl)methyl]sulfonyl}methyl)(1,3-thiazol-4-yl)]-2-methyl-6-oxo-1,6-dihydropyridine-3-carboxylate;
Ethyl 2-methyl-6-oxo-5-{2-(phenylthiomethyl)(1,3-thiazol-4-yl)}-1,6-dihydropyridine-3-carboxylate;
Ethyl 5-[2-(2-ethyl(4-pyridyl))(1,3-thiazol-4-yl)-2-methyl-6-oxo-1,6-dihydropyridine-3-carboxylate;
Ethyl 5-[2-(2-chloro(4-pyridyl))(1,3-thiazol-4-yl)-2-methyl-6-oxo-1,6-dihydropyridine-3-carboxylate;
Ethyl 5-[2-(3,5-Dichloro-pyridin-4-yl)-thiazol-4-yl]-2-methyl-6-oxo-1,6-dihydropyridine-3-carboxylate;
Ethyl 2-methyl-5-(2-(2-((2-methylpropyl)amino)-4-pyridinyl)-1,3-thiazol-4-yl)-6-oxo-1,6-dihydropyridine-3-carboxylate;
Ethyl 2-methyl-6-oxo-5-(2-(2-((3-pyridinylmethyl)amino)-4-pyridinyl)-1,3-thiazol-4-yl)-1,6-dihydropyridine-3-carboxylate;
Ethyl 2-methyl-6-oxo-5-(2-(2-((phenylmethyl)amino)-4-pyridinyl)-1,3-thiazol-4-yl)-1,6-dihydropyridine-3-carboxylate;
Ethyl 2-methyl-5-(2-(2-((2-((1-methylethyl)amino)ethyl)amino)-4-pyridinyl)-1,3-thiazol-4-yl)-6-oxo-1,6-dihydropyridine-3-carboxylate;
Ethyl 5-(2-(2-((2-(diethylamino)ethyl)amino)-4-pyridinyl)-1,3-thiazol-4-yl)-2-methyl-6-oxo-1,6-dihydropyridine-3-carboxylate;
Ethyl 5-(2-{2-[(fur-2-ylmethyl)-amino]-pyridin-4-yl}-thiazol-4-yl)-2-methyl-6-oxo-1,6-dihydropyridine-3-carboxylate;
Ethyl 5-{2-[2-(2-thien-2-yl-ethylamino)-pyridin-4-yl]-thiazol-4-yl}-2-methyl-6-oxo-1,6-dihydropyridine-3-carboxylate;
Ethyl 5-[2-(2-butylamino-pyridin-4-yl)-thiazol-4-yl]-2-methyl-6-oxo-1,6-dihydropyridine-3-carboxylate;
Ethyl 5-{2-[2-(carbamoylmethyl-amino)-pyridin-4-yl]-thiazol-4-yl}-2-methyl-6-oxo-1,6-dihydropyridine-3-carboxylate;
Ethyl 5-{2-[2-acetylamino-ethylamino)-pyridin-4-yl]-thiazol-4-yl}-2-methyl-6-oxo-1,6-dihydropyridine-3-carboxylate;
5-{2-[2-(Cyclopropylmethylamino)-pyridin-4-yl]-thiazol-4-yl}-2-methyl-6-oxohydro-pyridine-3-carboxylic acid cyclopropyl-methyl amide;
Ethyl 5-{2-[2-(cyclopropylmethyl-amino)-pyridin-4-yl]-thiazol-4-yl}-2-methyl-6-oxo-1,6-dihydropyridine-3-carboxylate;
Ethyl 5-{2-[2-(cyclopentyl)methylamino-pyridin-4-yl]-thiazol-4-yl}-2-methyl-6-oxo-1,6-dihydropyridine-3-carboxylate;
Ethyl 2-methyl-6-oxo-5-(2-(2-(amino)-4-pyridinyl)-1,3-thiazol-4-yl)-1,6-dihydropyridine-3-carboxylate;
Ethyl 2-methyl-5-[2-(methylamino)(1,3-thiazol-4-yl)]-6-oxo-1,6-dihydropyridine-3-carboxylate;
Ethyl 2-methyl-6-oxo-5-{2-[(phenylsulfonyl)methyl](1,3-thiazol-4-yl)}-1,6-dihydropyridine-3-carboxylate;
Ethyl 2-methyl-6-oxo-5-(2-(4-pyridyl)(1,3-thiazol-4-yl))-1,6-dihydropyridine-3-carboxylate;
Ethyl 2-methyl-6-oxo-5-{2-[(2-pyridylsulfonyl)methyl](1,3-thiazol-4-yl)}-1,6-dihydropyridine-3-carboxylate;
Ethyl 2-methyl-5-(2-(1-methyl-1-(phenylsulfonyl)ethyl)-1,3-thiazol-4-yl)-6-oxo-1,6-dihydropyridine-3-carboxylate;
Ethyl 2-cyclopropyl-6-oxo-5-(2-((phenylsulfonyl)methyl)-1,3-thiazol-4-yl)-1,6-dihydropyridine-3-carboxylate;
5-Bromo-6-methyl-3-(2-(4-pyridinyl)-1,3-thiazol-4-yl)-2(1H)-pyridinone;
Ethyl 2-methyl-5-(2-(2-(methylamino)-4-pyridinyl)-1,3-thiazol-4-yl)-6-oxo-1,6-dihydropyridine-3-carboxylate;
2-Methyl-6-oxo-N-(2-pyridinylmethyl)-5-(2-(2-((2-pyridinylmethyl)amino)-4-pyridinyl)-1,3-thiazol-4-yl)-1,6-dihydropyridine-3-carboxamide;
Ethyl 2-methyl-6-oxo-5-(2-(2-((2-pyridinylmethyl)amino)-4-pyridinyl)-1,3-thiazol-4-yl)-1,6-dihydropyridine-3-carboxylate;
Ethyl 5-[2-(methylamino-pyridin-4-yl)-thiazol-4-yl]-2-isopropyl-6-oxo-1,6-dihydropyridine-3-carboxylate;
1,1-Dimethylethyl 2-methyl-6-oxo-5-(2-(4-pyridinyl)-1,3-thiazol-4-yl)-1,6-dihydropyridine-3-carboxylate;
2-(1-Pyrrolidinyl)ethyl 2-ethyl-6-oxo-5-(2-(4-pyridinyl)-1,3-thiazol-4-yl)-1,6-dihydropyridine-3-carboxylate;
6-Ethyl-3-(2-pyridin-4-yl-thiazol-4-yl)-1H-pyridin-2-one;
6-Isopropyl-3-(2-pyridin-4-yl-thiazol-4-yl)-1H-pyridin-2-one;
3-(Diethylamino)propyl 2-ethyl-6-oxo-5-(2-(4-pyridinyl)-1,3-thiazol-4-yl)-1,6-dihydropyridine-3-carboxylate; and
3-(Diethylamino)propyl 2-(1-methylethyl)-6-oxo-5-(2-(4-pyridinyl)-1,3-thiazol-4-yl)-1,6-dihydropyridine-3-carboxylate.

34. A pharmaceutical composition comprising a pharmaceutically acceptable carrier and a compound of claim 1.

35. A method of inhibiting cell proliferation which comprises administering an effective amount of a compound of claim 1 and ethyl 2-trifluoromethyl-6-oxo-5-(2-(4-pyridyl)(1,3-thiazol-4-yl)-1,6-dihydro-3-pyridinecarboxylate.

36. A method of treating cancer which comprises administering an effective amount of a compound of claim 1 and ethyl 2-trifluoromethyl-6-oxo-5-(2-(4-pyridyl)(1,3-thiazol-4-yl)-1,6-dihydro-3-pyridinecarboxylate.

37. A method of inhibiting a serine/threonine kinase which comprises administering an effective amount a compound of claim 1 and ethyl 2-trifluoromethyl-6-oxo-5-(2-(4-pyridyl)(1,3-thiazol-4-yl)-1,6-dihydro-3-pyridinecarboxylate.

38. A method of treating a neurological disorder which comprises administering an effective amount a compound of claim 1 and ethyl 2-trifluoromethyl-6-oxo-5-(2-(4-pyridyl)(1,3-thiazol-4-yl)-1,6-dihydro-3-pyridinecarboxylate.

39. A method of treating apoptosis comprising administering an effective amount a compound of claim 1 and ethyl 2-trifluoromethyl-6-oxo-5-(2-(4-pyridyl)(1,3-thiazol-4-yl)-1,6-dihydro-3-pyridinecarboxylate.
